(19) United States
(12) Patent Application Publication
(10) Pub. No.: US 2002/0169101 A1
(43) Pub. Date: Nov. 14, 2002

(54) Treatment of sexual dysfunction

(76) Inventors: Maria Isabel Gonzalez, Cambridge (GB); Michael Higginbottom, Cambridge (GB); Herman Thijs Stock, Wijchen (NL); Martyn Clive Pritchard, Huntingdon (GB); Robert Denham Pinnock, Cambridgshire (GB); Pieter Hadewijn Van Der Graaf, Kent (GB); Alisdair Mark Naylor, Kent (GB); Christopher Peter Wayman, Kent (GB)

Correspondence Address: WARNER-LAMBERT COMPANY, 2800 PLYMOUTH ROAD, ANN ARBOR, MI, 48107, US

(21) Appl. No.: 09/999,284

(22) Filed: Nov. 15, 2001

Related U.S. Application Data
(63) Continuation-in-part of application No. 09/759,777, filed on Jan. 12, 2001.
Application No. 09/759,777 is a continuation-in-part of application No. 09/700,165, filed on Nov. 9, 2000.
Application No. 09/700,165 is a 371 of international application No. PCT/GB00/01787, filed on May 10, 2000.
(60) Provisional application No. 60/133,355, filed on May 10, 1999.

(30) Foreign Application Priority Data
Apr. 23, 2001 (GB) 01 09910.0
May 4, 2001 (GB) 01 11037.8

(51) Int. Cl. A61K 31/00

(52) U.S. Cl. 514/1

(57) ABSTRACT

Bombesin receptor antagonists have been found to be useful in the treatment of sexual dysfunction in both males and females. They may be selective BB1 antagonists or mixed BB1/BB2 antagonists. Combinations are disclosed of bombesin receptor antagonists with a range of other active compounds, for example PDE5 inhibitors, NEP inhibitors and lasofoxifene.

### FIELD OF THE INVENTION

The present invention relates to methods for the treatment of sexual dysfunction and to the preparation of medicaments for the treatment of sexual dysfunction.

### BACKGROUND TO THE INVENTION

Both males and females can suffer from sexual dysfunction. Sexual dysfunctions are relatively common in the general population (see O'Donohue, 1997). The disorder may relate to seeking sexual behaviour (proceptivity) and/or to acceptance of sexual behaviour, accompanied by sexual arousal (receptivity). The prevalence of sexual problems is higher in populations receiving medicaments, in particular antidepressants and antihypertensives. A need for pharmacotherapy for sexual dysfunction is increasing, but there has been very little research effort directed at finding drugs to treat sexual dysfunction.

Sexual dysfunctions include erectile dysfunctions of organic and psychogenic origin (Benet, 1995) as well as hypoactive sexual desire disorders, sexual arousal disorders, anorgasmy and sexual pain disorders (Berman, 1999, Urology).

In males, impotence can be defined as an inability to achieve penile erection or ejaculation. Its prevalence is claimed to be between 2% and 7% of the human male population, increasing with age up to 50 years and between 18% and 80% between 55 and 80 years of age. In the USA alone, for example, it has been estimated that there are up to 10 million impotent males, with the majority suffering from problems of organic rather than of psychogenic origin. Although many different drugs have been shown to induce penile erection, they were only effective after direct injection into the penis e.g. intraurethrally or intracavernosally (i.c.) and were not approved for erectile dysfunction. U.S. Pat. No. 5,576,290 discloses peptides which are stated to induce erection, but they have to be given subcutaneously e.g. by injection, and if an excessive dose is given they produce an exaggerated erectile response and stomach discomfort. Impotence treatment was revolutionized by the unexpected discovery that cGMP PDE inhibitors, e.g. pyrazolo[4,3-d]pyrimidin-7-ones were useful in the treatment of erectile dysfunction and could be administered orally, therefore obviating the disadvantages associated with i.c. administration. One such compound that is currently being manufactured is sildenafil (Viagra).

Thirty to 50% of American women complain of sexual dysfunction. Ageing, menopause, and decline in circulating oestrogen levels significantly increase the incidence of sexual complaints. In a recent publication, Berman J. R. et al. (1999, Int. J. Impot. Res.) describe methodology for evaluating physiologic and subjective components of the female sexual response in the clinical setting and determine the effects of age and oestrogen status on them. Low or absent sexual drive/desire constitutes the commonest problem in the female population (Laumann et al., 1999), but no therapy is available other than psychotherapy or empirical approaches. In a further publication (Bonney R. C et al., 2000) the causes and management of female sexual dysfunction are discussed, including the use of tibolone (Livial; Organon) which is a synthetic steroid that mimics the effects of oestrogen and has been reported to have mild androgenic properties, and the use of testosterone.

So far in the UK and the USA no drug has been licensed by the Department of Health specifically for the treatment of female sexual dysfunction, hence there is an unmet medical need in the treatment of female sexual dysfunction, especially sexual drive problems.

### SUMMARY OF THE INVENTION

This invention is based on the realisation that substances that act as bombesin receptor antagonists have utility in the treatment of sexual dysfunction, including the behavioural component thereof, in both male and female subjects. In other words, they can provide a treatment, in males, for erectile dysfunctions of organic and psychogenic origin as well as hypoactive sexual desire disorders, sexual arousal disorders, anorgasmy and sexual pain disorders in females.

The invention therefore provides a method of treating sexual dysfunction which comprises administering to a subject suffering therefrom and in need of treatment an effective amount of a bombesin (BB) receptor antagonist.

The invention further provides the use of a bombesin receptor antagonist in the manufacture of a medicament for preventing or treating male sexual dysfunction or female sexual dysfunction.

Furthermore, many of the compounds that can be used in this invention have both the property of binding to bombesin receptors and the property that an effective dose can be administered orally.

The bombesin antagonists preferably have a Ki against the bombesin receptor of less than 1000 nM, preferably less than 500 nM, more preferably less than 100 nM, preferably less than 50 nM and most preferably less than 10 nM. Preferably the bombesin antagonists are selective for BB₁ over the other bombesin receptor subtypes (preferably a selectivity of greater than 10, and more preferably a selectivity greater than 30 and most preferably greater than 100 measurable in vitro by the ratio of their IC50 or Ki values against the BB1 and BB2 receptors respectively) and has a Ki against the BB₁ receptor of less than 1000 nM, preferably less than 500 nM, more preferably less than 100 nM, preferably less than 50 nM and most preferably less than 10 nM. Compounds having the potencies set out above can be identified by the in vitro screen described below.

Thus the invention provides a method of treating drug induced sexual dysfunction (particularly but not exclusively dysfunction induced by antidepressants) in a male comprising administering to a male suffering therefrom an effective amount of a bombesin BB1 antagonist or a mixed BB1/BB2 antagonist.

The invention further provides a method of treating drug induced sexual dysfunction (particularly but not exclusively disfunction induced by antidepressants) in a female comprising administering to a female suffering therefrom an effective amount of a bombesin BB1 antagonist or a mixed BB1/BB2 antagonist.

The invention also provides a method for treating erectile dysfunction in a male comprising administering to a male suffering therefrom an effective amount of a bombesin BB1 antagonist or a mixed BB1/BB2 antagonist.

The invention also provides a method for treating a female patient suffering from hypoactive sexual desire disorder comprising administering to the female patient an effective amount of a bombesin BB1 antagonist or a mixed BB1/BB2 antagonist.

The invention also provides a method for treating a female patient suffering from sexual arousal disorder and/or orgasmic disorder comprising administering to the female patient an effective amount of a bombesin BB1 antagonist or a mixed BB1/BB2 antagonist.

The present invention additionally comprises the combination of a bombesin receptor antagonist (which may have one of the preferred range of potencies indicated above) for the treatment of male sexual dysfunction as outlined herein (more particularly male erectile dysfunction) and female sexual dysfunction (as outlined herein, more particularly female sexual arousal disorder or female sexual desire disorder) with one or more of the following auxiliary active agents. The combination provides a treatment for erectile dysfunctions of organic, neurogenic and/or psychogenic origin as well as hypoactive sexual desire disorders, sexual arousal disorders, anorgasmic and sexual pain disorders.

Thus a further aspect of the invention provides a pharmaceutical combination (for simultaneous, separate or sequential administration) of a bombesin receptor antagonist according to the invention and one or more materials selected from (1) to (34) below:

(1) naturally occurring or synthetic prostaglandins or esters thereof;

(2) α-adrenergic receptor antagonist compounds also known as α-adrenoceptor antaginists or α-receptor antagonists or α-blockers;

(3) NO-donor (NO-agonist) compounds;

(4) potassium channel openers or modulators;

(5) dopaminergic agents;

(6) vasodilator agents;

(7) thromboxane A2 agonists;

(8) CNS active agents;

(9) ergot alkaloids;

(10) compounds which modulate the action of natriuretic factors;

(11) angiotensin receptor antagonists such as losartan;

(12) substrates for NO-synthase;

(13) calcium channel blockers;

(14) cholesterol lowering agents;

(15) antiplatelet and antithrombotic agents;

(16) insulin sensitising agents and hypoglycaemic agents;

(17) L-DOPA or carbidopa;

(18) acetylcholinesterase inhibitors;

(19) steroidal or non-steroidal anti-inflammatory agents;

(20) estrogen receptor modulators and/or estrogen agonists and/or estrogen antagonists, and pharmaceutically acceptable salts thereof,

(21) PDE inhibitors;

(22) NPY (neuropeptide Y) inhibitors;

(23) NEP inhibitors;

(24) vasoactive intestinal proteins (VIP), VIP mimetics, VIP analogues, VIP receptor agonists or VIP analogues or VIP fragments, or cc-adrenoceptor antagonists with VIP combinations;

(25) melanocortin receptor agonists or modulators or melanocortin ehancers;

(26) serotonin receptor agonists, antagonists or modulators;

(27) testosterone replacement agents, testostemone, dihydrotestosterone or a testosterone implant;

(28) estrogen, estrogen and medroxyprogesterone or medroxyprogesterone acetate (MPA) (i.e. as a combination), or estrogen and methyl testosterone hormone replacement therapy agents;

(29) modulators of transporters for noradrenaline, dopamine and/or serotonin;

(30) purinergic receptor agonists and/or modulators;

(31) neurokinin (NK) receptor antagonists;

(32) opioid receptor agonists, antagonista or modulators;

(33) agonists or modulators for oxytocin/vasopressin receptors; and

(34) modulators of cannabinoid receptors.

In particular the invention includes a pharmaceutical composition or a unit dosage form comprising an effective amount of a bombesin receptor antagonist and an effective amount of any of the materials selected from (1) to (34) above.

In the above methods, and in the above combination, composition or dosage form, said antagonists preferably have a Ki against BB1, of of less than 1000 nM, preferably less than 500 nM, more preferably less than 100 nM, preferably less than 50 nM and most preferably less than 10 nM and/or a selectivity for BB₁ over the other bombesin receptor subtypes greater than 10, and more preferably greater than 30 and most preferably greater than 100 measurable in vitro by the ratio of their IC50 or Ki values against the BB1 and BB2 receptors respectively.

### BRIEF DESCRIPTION OF FIGURES

FIG. 1: Effect of (S) 3-(1H-Indol-3-yl)-N-[1-(5-methoxy-pyridin-2-yl)-cyclohexylmethyl]-2-methyl-2-[3-(4-nitro-phenyl)-ureido]-propionamide (Compound (1)) on female rat sexual proceptivity.

FIG. 2: Effect of Compound (1) on female rat sexual receptivity.

FIG. 3: Effect of repeated administration of Compound (1) on female rat proceptivity.

FIG. 4: Effect of intracerebroventricular administration of Compound (1) on female rat sexual proceptivity.

FIG. 5: Inhibitory effect of NMB on female rat sexual proceptivity and antagonism of this effect by Compound (1).

FIG. 6: Results of an investigation to show whether the effect of Compound (1) on female sexual behaviour is mediated through progesterone.

FIG. 7: Results of an investigation to show whether the effect of Compound (1) on female sexual behaviour is mediated through oestradiol.

FIG. 8: Results of an investigation to show whether the effect of Compound (1) on female sexual behaviour is mediated through prolactin.

FIG. 9: Results of an investigation to show whether the effect of Compound on female sexual behaviour is mediated through LH.

FIG. 10: Results of an investigation to show whether the effect of Compound (1) on female sexual behaviour is mediated through FSH.

FIG. 11: Effect of Compound (1) on the sexual behaviour of normal male rats (Mount Latency).

FIG. 12: Effect of Compound (1) on the sexual behaviour of normal male rats (Intromission Latency).

FIG. 13: Effect of Compound (1) on the sexual behaviour of normal male rats (Number of Mounts+Intromission).

FIG. 14: Effect of Compound (1) on the sexual behaviour of normal male rats (Ejaculation Latency).

FIG. 15: Effect of Compound (1) on the sexual behaviour of normal male rats (Refractory Period).

FIG. 16: Effect of Compound (1) on the sexual behaviour of sexually dysfunctional male rats (Mount Latency).

FIG. 17: Effect of Compound (1) on the sexual behaviour of sexually dysfunctional male rats (Ejaculation Latency).

FIG. 18: Effect of Compound (1) on the sexual behaviour of sexually dysfunctional male rats (% animals ejaculating).

FIG. 19: Effect of (S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide (Compound (2)) in PEG 200 on female rat sexual proceptivity.

FIG. 20: Effect of Compound (2) in methylcellulose on female rat sexual proceptivity.

FIG. 21: Effect of Compound (2) in PEG 200 on female rat sexual receptivity.

FIG. 22: Effect of compound 1 on basal and pelvic nerve-stimulated increases in female genital blood flow in the anaesthetised rabbit model of female sexual arousal.

FIG. 23: Effect of (2S)-N-{[1-(4-aminophenyl)cyclohexyl]methyl}-3-(1H-indol-3-yl)-2-methyl-2-{[(4-nitroanilino)carbonyl]amino}propanamide (Compound 3) on basal and pelvic nerve-stimulated increases female genital blood flow in the anaesthetised rabbit model of female sexual arousal.

FIG. 24: Effect of compound 1 on penile intracavemosal pressure in the conscious male rat.

FIG. 25: Effect of compound 3 on penile intracavernosal pressure in the conscious male rat model of penile erection.

FIG. 26: Effect of compound 3 alone and in combination with a phosphodiesterase type five inhibitor on basal and pelvic nerve-stimulated increases penile intracavernosal pressure in the anaesthetised rabbit model of penile erection.

### DESCRIPTION OF PREFERRED EMBODIMENTS

Suitable Subjects

As previously explained the invention provides combinations, compositions and methods for the treatment of male sexual dysfunction or female sexual dysfunction. The inventors believe that there are common mechanisms underlying the pathologies of male and female psychogenic sexual dysfunctions.

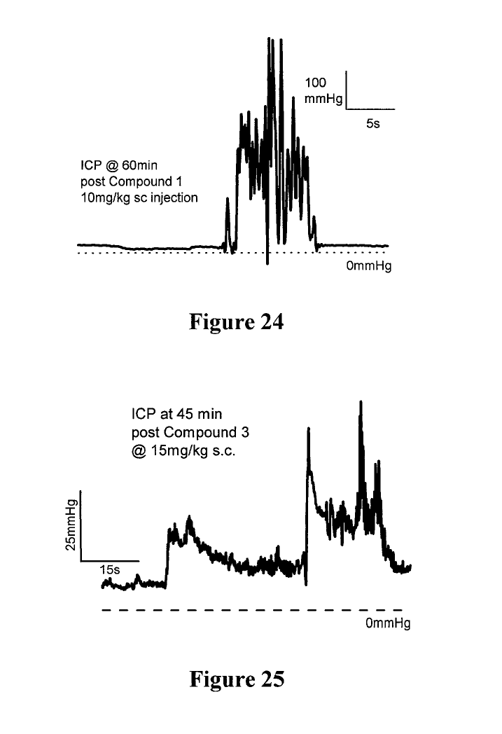

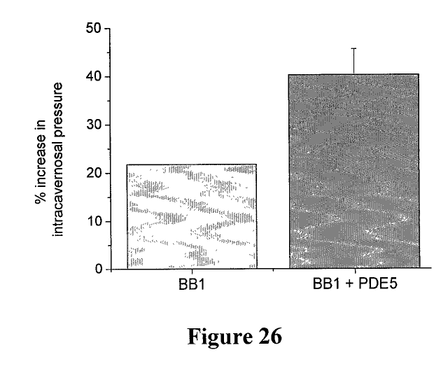

Male sexual dysfunction includes male erectile dysfunction (MED). Patients with mild to moderate MED should benefit from treatment with a bombesin antagonist and patients with severe MED should also respond.the ability of bombesin antagonists to return intracavernosal pressure to normal levels in a conscious rat model of penile erection (Example 170, FIGS. 24 and 25) and in a pelvic nerve stimulation model (Example 171, FIG. 26) has been demonstrated using telemetry. However, early investigations suggest that the response rate of patients with mild, moderate and severe MED will be greater with a bombesin antagonist/PDE5 inhibitor combination (see Example 171 and FIG. 26). Mild, moderate and severe MED will be terms known to the man skilled in the art, but guidance can be found in The Journal of Urology, vol 151, 54-61 (January 1994).

Our investigations suggest the below mentioned male sexual dysfunction/MED patient groups should benefit from treatment with a bombesin antagonist and/or a bombesin antagonist with a phosphoesterase type 5 inhibitor (PDE5i) or other combination set out herein. These patient groups which are described in more detail in Clinical Andrology vol 23,no.4, p773-782, and chapter 3 of the book by I. Eardley and K. Sethia “Erectile Dysfunction-Current Investigation and Management”, published by Mosby-Wolfe are as follows: psychogenic, endocrinologic, neurogenic, arteriogenic, drug-induced sexual dysfunction and sexual dysfunction related to cavemosal factors, particularly venogenic causes. The invention finds application in the following sub-populations of patients with sexual dysfunction/MED: the young, the elderly including ageing-related decline in sexual arousability. More particularly, the invention finds application in patients with male sexual dyfunction such as MED arising from:

(i) Arteriogenic/vasculogenic etiologies eg cardiovascular or atherosclerotic diseases, hypercholesterolemia, cigarette smoking, diabetes, hypertension, radiation and perineal trauma, traumatic injury to the iliohypogastric pudendal vacular system.

(ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery.

(iii) Hormonal/endocrine etiologies such as dysfunction of the hypothalamic/pituitary/gonadal axis, dysfunction of the pancreas, surgical or medical castration, androgen deficiency, high circulating levels of prolactin eg hyperprolactinemia, hyper and hypothyroidism.

(iv) Psychogenic etiologies such as depression, obsessive-compulsive disorder, anxiety disorder, emotional and relational issues, performance anxiety, marital discord, dysfunctional attitudes, sexual phobias, religious inhibition or a traumatic past experiences.

(v) Drug-induced sexual dysfunction resulting from therapy with selective serotonin reuptake inhibitors (SSRis) and other antidepressant therapies (tricyclics and major tranquillizers), anti-hypertensive therapies, and sympatholytic drugs.

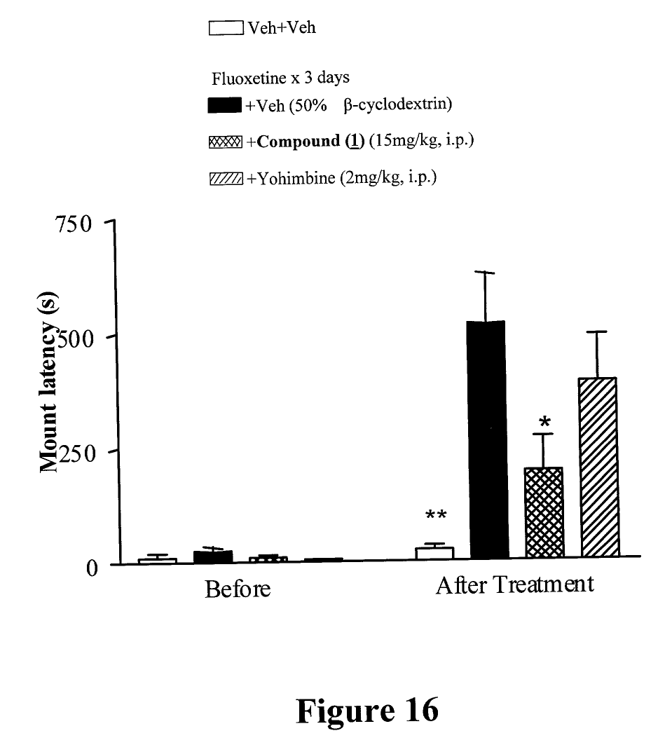

Drug-induced sexual disfunction in males includes patients whose drug treatment/therapy leads to delayed ejaculation/orgasm, reduced libido and/or erectile dysfunction. The bombesin antagonists of the invention (more particularly BB1 antagonists) restore ejaculatory/orgasmic, libido and erectile function to normal physiological “levels”. This is supported by the experiments described in Example 8 and FIGS. 16-18 below.

When the erectile dysfunction is other than drug induced, the bombesin antagonists (more particularly BB1 antagonists) can also be used to treat the erectile dysfunction by potentiating the normal endogenous erectiogenic mechanisms of the male (during normal sexual stimulation) and restoring the erectile function to normal levels during sexual stimulation. Thus Examples 170 and 171 and FIGS. 24, 25 and 26 hereinafter illustrate that bombesin antagonists (more particularly BB1 antagonists) or a bombesin antagonist with a phosphodiesterase type 5 inhibitor potentiate erectiogenic mechanisms in animal models by enhancing intercavemosal pressure and potentiating the effect of the pelvic nerve stimulation-induced increases in intracavemosal pressure.

Early investigations also show that that the invention will help restore the libido/desire in males to normal levels where the desire dysfunction is not drug induced (e.g. psychogenic).

The psychogenic component of male sexual dysfunction has been classified by the nomenclature committee of the International Society for Impotence Research (and is illustrated by Sachs B. D., 2000) as generalised type, characterised by a general unresponsiveness or primary lack of sexual arousal, and ageing-related decline in sexual arousability, characterised by generalised inhibition or chronic disorders of sexual intimacy.

The compounds of this invention are useful in the treatment of male sexual dysfunction, especially drug-induced male sexual dysfunction and psychogenic male sexual dysfunction associated with generalised unresponsiveness and ageing-related decline in sexual arousability.

Female sexual dysfunction can be grouped into four classes (Scrip's Complete Guide to Women's Healthcare, p.194-205, 2000), which include:

Hypoactive sexual desire disorders, which can be characterised as persistent or recurrent lack of sexual thoughts/fantasies and lack of receptivity to sexual activity, causing personal distress.

Sexual arousal disorders, which can be can be characterised as persistent or recurrent inability to achieve or maintain adequate sexual excitement, causing personal distress. The normal sexual arousal response consists of a number of physiological responses that are observed during sexual excitement. These changes such as vaginal, labial and clitoral engorgement result from increases in genital blood flow. Engorgement leads to increased vaginal lubrication via plasma transudation, increased vaginal compliance (relaxation of vaginal smooth muscle) and increases in vaginal and clitoral sensitivity. Female sexual arousal disorder (FSAD) is a highly prevalent sexual disorder affecting up to 40% of pre-, peri- and postmenopausal (±HRT) women. The primary consequence of FSAD is reduced genital engorgement or swelling which manifests itself as a lack of vaginal lubrication and a lack of pleasurable genital sensation. Secondary consequences include reduced sexual desire, pain during intercourse and difficulty in achieving orgasm. The most common cause of FSAD is decreased genital blood flow resulting in reduced vaginal, labial and clitoral engorgement. (Park, 1997; Goldstein, 1998; Berman, 1999, Werbin, 1999).

Orgasmic disorders can be characterised as persistent or recurrent difficulty or delay in attaining orgasm after adequate sexual stimulation and arousal, causing personal distress.

Sexual pain disorders can be characterised by dyspareunia, (characterised by recurrent or persistent genital pain associated with sexual intercourse), vaginismus (characterised by recurrent or persistent involuntary spasm of the muscles of the outer third of the vagina which interferes with vaginal penetration, causing personal distress) and other pain disorders (characterised by recurrent or persistent genital pain induced by non coital sexual stimulation).

The compounds of this invention are useful in the treatment of female sexual dysfunction (FSD), and this includes pre-, peri- and post-menopausal female sexual dysfunction associated with hypoactive sexual desire disorders, sexual arousal disorders, orgasmic disorders or anorgasmy, or sexual pain disorders.

Early investigations suggest the below mentioned female sexual dysfunction (FSD) patient groups should benefit from treatement with a bombesin antagonist or a bombesin antagonist and a PDE5i (or other combination set out hereinafter). These patient groups are described in more detail in Berman et al (Urology, 1999). The invention finds application in the following sub-populations of patients with FSD: the young, the elderly (ageing-related sexual dysfunction), pre-menopausal, peri-menopausal, post-menopausal women with or without hormone replacement therapy. More particularly the invention finds application in patients with FSD arising from:

(i) Arteriogenic/vasculogenic etiologies eg cardiovascular or atherosclerotic diseases, hypercholesterolemia, cigarette smoking, diabetes, hypertension, radiation and perineal trauma, traumatic injury to the iliohypogastric pudendal vacular system.

(ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery.

(iii) Hormonal/endocrine etiologies such as dysfunction of the hypothalamic/pituitary/gonadal axis, or dysfunction of the ovaries, dysfunction of the pancreas, surgical or medical castration, androgen deficiency, high circulating levels of prolactin eg hyperprolactinemia, natural menopause, premature ovarian failure, hyper and hypothyroidism.

(iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or a traumatic past experiences.

(v) Drug-induced sexual dysfunction resulting from therapy with selective serotonin reuptake inhibitors (SSRis) and other antidepressant therapies (tricyclics and major tranquillizers), anti-hypertensive therapies, sympatholytic drugs, chronic oral contraceptive pill therapy.

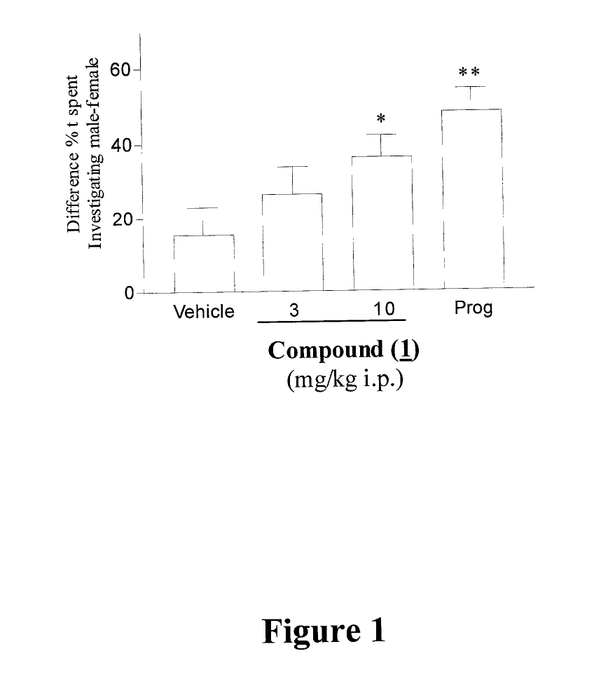

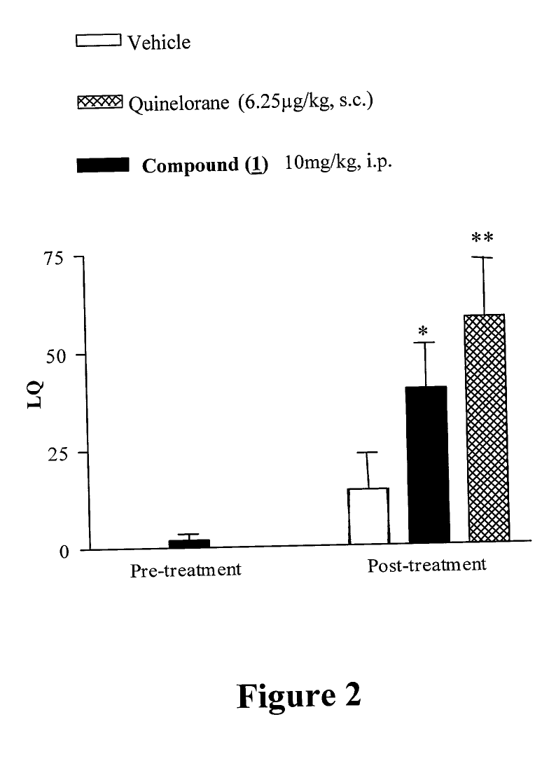

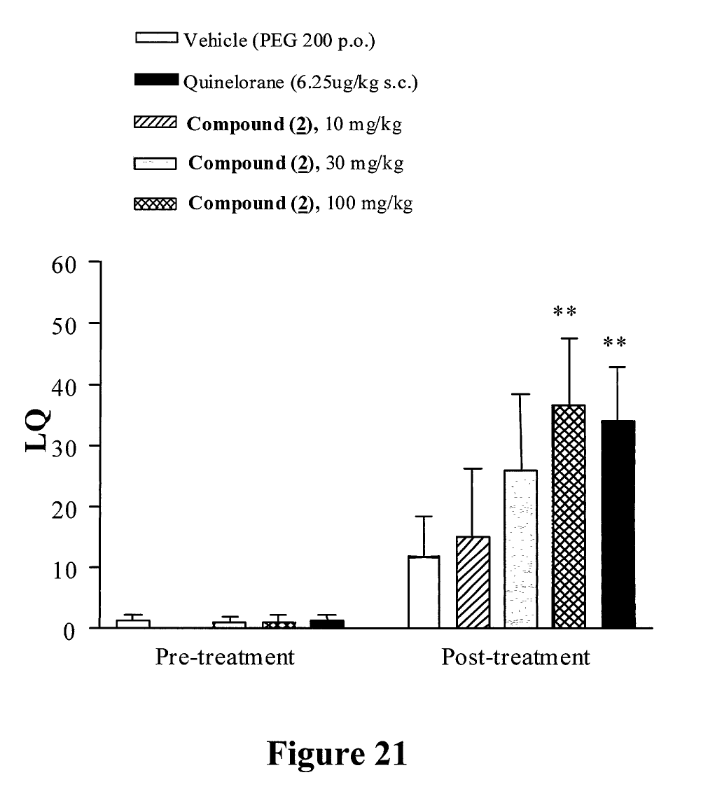

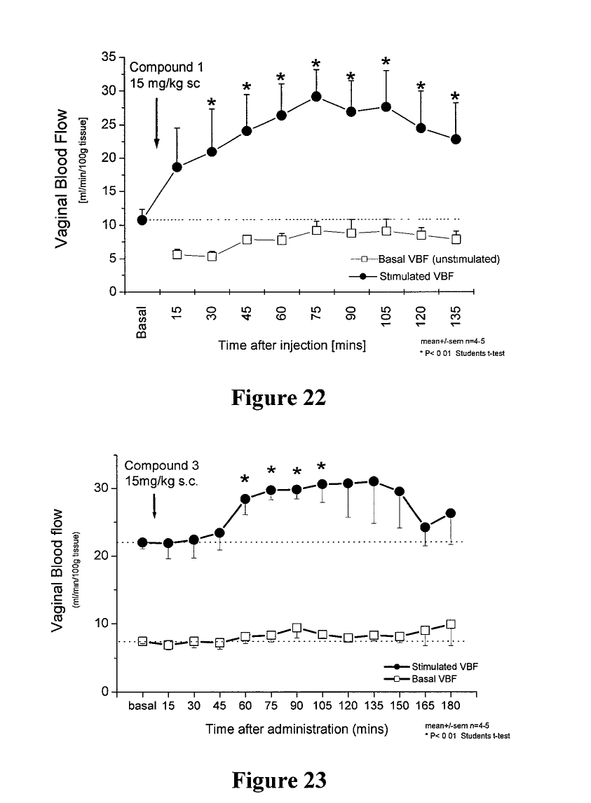

By drug-induced sexual dysfunction in females we mean to include cases where the drug treatment/therapy leads to delayed orgasm or inability to achieve an orgasm, reduced libido and FSD. The bombesin antagonists (more particularly BB1 antagonists) help restore orgasm, libido and female sexual function to normal physiological “levels”. Furthermore, since bombesin antagonists have been shown to have beneficial effects on sexual function in ovary intact and in ovarectomised animals, it is apparent that bombesin antagonists (more particularly BB1 antagonists) can also be used to treat female sexual arousal disorders (FSAD), hypoactive sexual desire disorders (HSDD) and anorgasmy (FOD) and also sexual pain disorders, especially where these are secondary to arousal disorders. In particular, Example 2 and FIGS. 2 and 21 and Example 169 and FIGS. 22 and 23 illustrate that the combinations and methods of treatment of the invention can enhance receptive behaviour and arousal via increased genital blood flow in women with FSAD and FOD respectively. Also, Examples 1, 4 and 5 and FIGS. 1, 3, 4 and 5 illustrate that the combinations and methods of treatment of the invention can increase proceptive behaviour and restore normal desire/libido in women with HSDD.

Bombesin Receptor Antagonists—General

Bombesin receptors are present in hypothalamic areas. We have found that they can exert a neuromodulatory effect on sexual behaviour.

We have tested compounds that are bombesin receptor antagonists using animal models that we have refined and believe are reliable and predictive, in particular with the capacity to make predictions for females. In rodents proceptive behaviour is under hormonal control, progesterone being essential for induction of proceptive behaviour in combination with oestrogen (Johnson M, 1988). The evidence for the hormonal control of proceptive behaviour in primates is conflicting, but on the whole oestrogens and/or androgens appear to enhance proceptive behaviour (Baum M. J, 1983). The behavioural manifestations of proceptive behaviour in the rat include “hopping and darting” movement, with rapid vibration of the ears. Tests to assess the eagerness to seek sexual contact (sexual motivation) have been reported as the most appropriate way to measure proceptivity (Meyerson, 1973). Receptivity, in the rat, is demonstrated when the female assumes a lordotic position. This occurs when, on mounting, the male exerts pressure with his forepaws on the flanks of the receptive female. The main sites of neuronal control for this behaviour are the ventromedial nucleus (VMN) and the midbrain central grey area (MCG) (for review, see Wilson C. A., 1993).

Bombesin is a 14-amino acid peptide originally isolated from the skin of the European frog Bombina bombina (Anastasi A., 1971). It belongs to a class of peptides which share structural homology in their C-terminal decapeptide region (Dutta A. S., Small Peptides; Chemistry, Biology, and Clinical Studies). At present, two mammalian bombesin-like peptides have been identified, the decapeptide neuromedin B (NMB) and a 23-residue amino acid, gastrin-releasing peptide (GRP).

Bombesin evokes a number of central effects through actions at a heterogeneous population of receptors. The BB₁ receptor binds neuromedin B (NMB) with higher affinity than gastrin-related peptide (GRP) and neuromedin C (NMC) and BB₂ receptors bind GRP and NMC with greater affinity than NMB. More recently evidence has emerged of two more receptor subtypes denoted BB₃ and BB₄ but due to limited pharmacology, little is known of their function at present. BB₁ and BB₂ receptors have a heterogeneous distribution within the central nervous system indicating that the endogenous ligands for these receptors may differentially modulate neurotransmission. Among other areas, BB₁ receptors are present in the ventromedial hypothalamus (Ladenheim E. E, 1990).

Bombesin-like immunoreactivity and mRNA have been detected in mammalian brain (Braun M., et al., 1978, Battey J., et al.1991). NMB and GRP are believed to mediate a variety of biological actions (for a review, see WO 98/07718).

The following patent applications disclose compounds capable of antagonising the effects of NMB and/or GRP at bombesin receptors: CA 2030212, EP 0309297, EP 0315367, EP 0339193, EP 0345990, EP 0402852, EP 0428700, EP 0438519, EP 0468497, EP 0559756, EP 0737691, EP 0835662, JP 07258081, UK 2231051, U.S. Pat. No. 4,943,561, U.S. Pat. No. 5,019,647, U.S. Pat. No. 5,028,692, U.S. Pat. No. 5,047,502, U.S. Pat. No. 5,068,222, U.S. Pat. No. 5,084,555, U.S. Pat. No. 5,162,497, U.S. Pat. No. 5,244,883, U.S. Pat. No. 5,439,884, U.S. Pat. No. 5,620,955, U.S. Pat. No. 5,620,959, U.S. Pat. No. 5,650,395, U.S. Pat. No. 5,723,578, U.S. Pat. No. 5,750,646, U.S. Pat. No. 5,767,236, U.S. Pat. No. 5,877,277, U.S. Pat. No. 5,985,834, WO 88/07551, WO 89/02897, WO 89/09232, WO 90/01037, WO 90/03980, WO 91/02746, WO 91/04040, WO 91/06563, WO 92/02545, WO 92/07830, WO 92/09626, WO 92/20363, WO 92/20707, WO 93/16105, WO 94/02018, WO 94/02163, WO 94/21674, WO 95/00542, WO 96/17617, WO 96/28214, WO 97/09347, WO 98/07718, WO 00/09115, WO 00/09116. We believe that compounds disclosed in these applications can be used in the prevention or treatment of male and/or female sexual dysfunction, which is an indication that is not disclosed or suggested by the aforesaid applications, or indeed in any previous scientific publication concerning bombesin receptors.

Bombesin receptor antagonists to which this invention is applicable include both non-peptide compounds and peptide compounds. Compounds that can be formulated into compositions for oral administration, especially human oral administration, without substantial loss of activity are preferred. Many non-peptide compounds having the desired properties fall into this category.

A) Non-Peptide Bombesin Receptor Antagonists

One preferred genus of compounds for use in the invention comprises bombesin receptor antagonists of the formula (I)

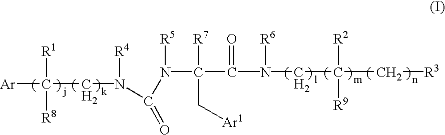

and pharmaceutically acceptable salts thereof, wherein:

j is 0or 1;

k is 0 or 1;

l is 0, 1, 2,or 3;

m is 0 or 1;

n is 0, 1 or 2;

Ar is phenyl, pyridyl or pyrimidyl, each unsubstituted or substituted by from 1 to 3 substituents selected from alkyl, halogen, alkoxy, acetyl, nitro, amino, —CH₂NR¹⁰R¹¹, cyano, —CF₃, —NHCONH₂, and —CO₂R¹²;

R¹ is hydrogen or straight, branched, or cyclic alkyl of from 1 to 7 carbon atoms;

R⁸ is hydrogen or forms a ring with R¹ of from 3 to 7 carbon atoms;

R² is hydrogen or straight, branched, or cyclic alkyl of from 1 to 8 carbon atoms which can also contain 1 to 2 oxygen or nitrogen atoms;

R⁹ is hydrogen or forms with R² a ring of from 3 to 7 carbon atoms which can contain an oxygen or nitrogen atom; or R² and R⁹ can together be a carbonyl;

Ar¹ can be independently selected from Ar and can also include pyridyl-N-oxide, indolyl, imidazolyl, and pyridyl;

R⁴, R⁵, R⁶, and R⁷ are each independently selected from hydrogen and lower alkyl; R⁴ can also form with R⁵ a covalent link of 2 to 3 atoms which may include an oxygen or a nitrogen atom;

R³ can be independently selected from Ar or is hydrogen, hydroxy, -NMe₂, N-methyl-pyrrolyl, imidazolyl, N-methyl-imidazolyl, tetrazolyl, N-methyl-tetrazolyl, thiazolyl, —CONR¹³R¹⁴, alkoxy,

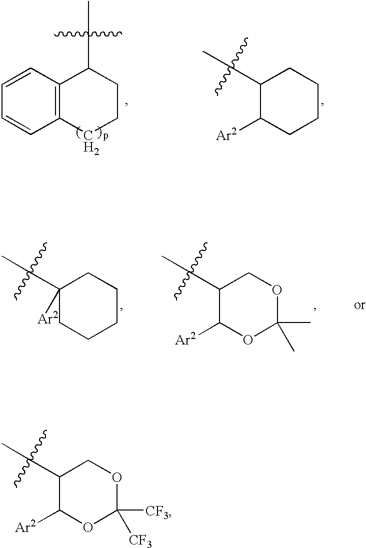

wherein p is 0, 1 or 2 and Ar² is phenyl or pyridyl;

R¹⁰, R¹¹, R¹², R¹³ and R¹⁴ are each independently selected from hydrogen or straight, branched, or cyclic alkyl of from 1 to 7 carbon atoms.

Preferred compounds are those of Formula (Ia)

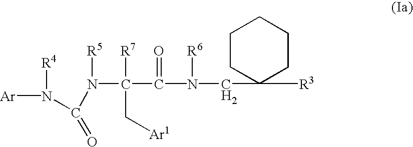

wherein

Ar is phenyl unsubstituted or substituted with 1 or 2 substituents selected from isopropyl, halo, nitro, and cyano;

R⁴, R⁵, and R⁶ are hydrogen;

R⁷ is methyl or hydrogen;

R³ is 2-pyridyl or hydroxy; and

Ar¹ is indolyl, pyridyl, pyridyl-N-oxide, or imidazolyl.

Other preferred compounds are those of Formula I wherein

Ar is unsubstituted phenyl;

R¹ is cyclopentyl or tert-butyl;

R⁴ and R⁵ are hydrogen;

R⁷ is methyl;

R⁶ is hydrogen;

R³ is phenyl with two isopropyl substituents, unsubstituted phenyl, or

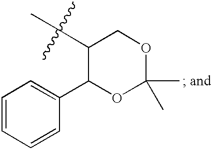

and

Ar¹ is indolyl.

Other preferred compounds are those of Formula I wherein

Ar is 2,6-diisopropyl-phenyl, 4-nitro-phenyl, and 4-cyano-phenyl;

R⁴, R⁵, and R⁶ are hydrogen;

R⁷ is methyl;

R² is hydrogen or cyclohexyl; and

R³ is hydroxyl, pyridyl,

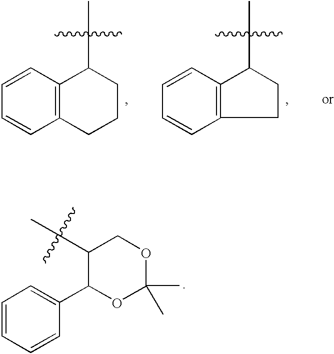

At present, most preferred of the compounds of formula (I) are (S) 3-(1H-Indol-3-yl)-N-[1-(5-methoxy-pyridin-2-yl)-cyclohexylmethyl]-2-methyl-2-[3-(4-nitro-phenyl)-ureido]-propionamide (also referred to as Compound 1) and its pharmacologically acceptable salts and (2S)-N-{[1-(4-aminophenyl)cyclohexyl]methyl}-3-(1H-indol-3-yl)-2-methyl-2-{[(4-nitroanilino)-carbonyl]amino}propanamide (also referred to as Compound 3) and its pharmacologically acceptable salts.

Other preferred compounds of Formula (I) are set out below and included also are their pharmaceutically acceptable salts:

(S) N-cyclohexylmethyl-2-[3-(2,6-diisopropyl-phenyl)-ureido]-3-(1H-indol-3-yl)-2-methyl-propionamide;

N-cyclohexylmethyl-2-[3-(2,6-diisopropyl-phenyl)-ureido]-3-(1H-indol-3-yl)-N-methyl-propionamide;

N-cyclohexylmethyl-2-[3-(2,6-diisopropyl-phenyl)-1-methyl-ureido]-3-(1H-indol-3-yl)-propionamide;

2-[3-(2,6-diisopropyl-phenyl)-ureido]-2-methyl-3-(1-oxy-pyridin-2-yl)-N-(1-pyridin-2-yl-cyclohexylmethyl)-propionamide;

2-[3-(2,6-diisopropyl-phenyl)-ureido]-2-methyl-3-pyridin-2-yl-N-(1-pyridin-2-yl-cyclohexylmethyl)-propionamide;

2-[3-(2-tert-butyl-phenyl)-ureido]-N-cyclohexylmethyl-3-(1H-indol-3-yl)-2-methyl-propionamide;

N-cyclohexylmethyl-2-[3-(2,6-dichloro-phenyl)ureido]-3-(1H-indol-3-yl)-2-methyl-propionamide;

N-cyclohexylmethyl-2-[3-(2,6-dimethoxy-phenyl)ureido]-3-(1H-indol-3-yl)-2-methyl-propionamide;

N-cyclohexylmethyl-2-[3-(2,6-dimethylamino-phenyl)-ureido]-3-(1Hindol-3-yl)-2-methyl-propionamide;

(S) N-cyclohexylmethyl-3-(1H-indol-3-yl)-2-methyl-2-[3-(4-nitro-phenyl)-ureido]-propionamide;

N-cyclohexylmethyl-2-[3-(2,2-dimethyl-1-phenyl)propyl)-ureido]-3-(1H-indol-3-yl)-2-methyl-propionamide;

[S-(R*, R*)] 3-(1H-indol-3-yl)-2-methyl-2-{3-[1-(4-nitro-phenyl)-ethyl]-ureido}-N-(1-pyridin-2-yl-cyclohexylmethyl)-propionamide;

N-(2,2-dimethyl-4-phenyl-[1,3]dioxan-5-yl)-3-(1H-indol-3-yl)-2-methyl-2-[3-(1-phenyl-cyclopentylmethyl)ureido]-propionamide;

(S)-N-(2,6-diisopropyl-phenyl)-2-[3-(2,2-dimethyl-1-phenyl-propyl)-ureido]-3-(1H-indol-3-yl)-propionamide;

(R)-N-(2,6-diisopropyl-phenyl)-2-[3-(2,2-dimethyl-1-phenyl-propyl)-ureido]-3-(1H-indol-3-yl)-propionamide;

2-[3-(2,6-diisopropyl-phenyl)-ureido]-N-(2,2-dimethyl-4-phenyl-[1,3]dioxan-5-yl)-3-(1H-indol-3-yl)-2-methyl-propionamide;

N-cyclohexyl-2-[3-(2,6-diisopropyl-phenyl)-ureido]-3-(1H-indol-3-yl)-2-methyl-propionamide;

N-(²-cyclohexyl-ethyl)-2-[3-(2,6-diisopropyl-phenyl)-ureido]-3-(1H-indol-3-yl)-2-methyl-propionamide;

2-[3-(2,6-diisopropyl-phenyl)-ureido]-3-(1H-indol-3-yl)-2-methyl-propionamide;

2-[3-(2,6-diisopropyl-phenyl)-ureido]-3-(1H-indol-3-yl)-2-methyl-N-(3-methyl-butyl)-propionamide;

2-[3-(2,6-diisopropyl-phenyl)-ureido]-3-(1H-indol-3-yl)-2-methyl-N-(3-phenyl-propyl)-propionamide;

2-[3-(2,6-diisopropyl-phenyl)-ureido]-3-(1H-indol-3-yl)-2-methyl-N-(1,2,3,4-tetrahydro-naphthalen-1-yl)-propionamide;

2-[3-(2,6-diisopropyl-phenyl)-ureido]-3-(1H-indol-3-yl)-2-methyl-N-(2-phenyl-cyclohexyl)-propionamide;

2-[3-(2,6-diisopropyl-phenyl)-ureido]-N-indan-1-yl-3-(1H-indol-3-yl)-2-methyl-propionamide;

2-[3-(2,6-diisopropyl-phenyl)-ureido]-N-(1-hydroxy-cyclohexylmethyl)-3-(1H-indol-3-yl)-2-methyl-propionamide;

2-[3-(2,6-diisopropyl-phenyl)-ureido]-3-(1H-indol-3-yl)-2-methyl-N-(1-pyridin-2-yl-cyclohexylmethyl)-propionamide;

2-[3-(2,6-diisopropyl-phenyl)-ureido]-3-(1H-indol-3-yl)-2-methyl-N-(6,7,8,9-tetrahydro-5H-benzocyclohepten-5-yl)-propionamide;

2-[3-(2,6-diisopropyl-phenyl)-ureido]-3-(1H-indol-3-yl)-2-methyl-N-phenyl-propionamide;

N-(1-hydroxy-cyclohexylmethyl)-3-(1H-indol-3-yl)-2-methyl-2-[3-(4-nitro-phenyl)-ureido]-propionamide;

2-[3-(4-cyano-phenyl)-ureido]-3-(1H-indol-3-yl)-2-methyl-N-(1-pyridin-2-yl-cyclohexylmethyl)-propionamide;

(S) 3-(1H-indol-3-yl)-2-methyl-2-[3-(4-nitro-phenyl)-ureido]-N-(1-pyridin-2-yl-cyclohexylmethyl)-propionamide;

(S) 3-(1H-indol-3-yl)-2-methyl-N-(1-pyridin-2-yl-cyclohexylmethyl)-2-[3-(4-trifluoromethyl-phenyl)-ureido]-propionamide;

(S) 4-(3-{2-(1H-indol-3-yl)-1-methyl-1-[(1-pyridin-2-yl-cyclohexylmethyl)-carbamoyl]-ethyl}-ureido)-benzoic acid ethyl ester;

2-[3-(2,6-diisopropyl-phenyl)-ureido]-3-(1H-imidazol-4-yl)-N-(1-pyridin-2-yl-cyclohexylmethyl)-propionamide;

2-[3-(2,6-diisopropyl-phenyl)-ureido]-2-methyl-N-(1-pyridin-2-yl-cyclohexylmethyl)-3-(2-trifluoromethyl-phenyl)-propionamide;

2-[3-(2,6-diisopropyl-phenyl)-ureido]-2-methyl-3-(2-nitro-phenyl)-N-(1-pyridin-2-yl-cyclohexylmethyl)-propionamide;

(S) 3-(1H-indol-3-yl)-N-[1-(5-methoxy-pyridin-2-yl)-cyclohexylmethyl]-2-methyl-2-[3-(4-nitro-phenyl)-ureido]-propionamide; and

N-cyclohexylmethyl-2-[3-(2,6-diisopropyl-phenyl)-ureido]-2-methyl-3-pyridin-2-yl-propionamide.

Another preferred genus of compounds which can be used for the present purpose is of formula (II) and includes pharmaceutically acceptable salts thereof:

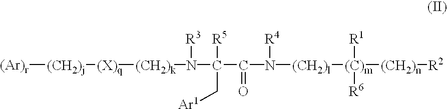

wherein:

j is 0, or 2;

k is 0 or 1;

l is 0, 1, 2, or 3;

m is 0 or 1;

n is 0, 1 or 2;

q is 0 or 1;

r is 0 or 1; when r is 0, Ar is replaced by hydrogen;

Ar is phenyl, pyridyl, pyrimidyl, thienyl, furyl, imidazolyl, pyrrolyl or thiazolyl each unsubstituted or substituted by from 1 to 3 substituents selected from acetyl, alkoxy, alkyl, amino, cyano, halo, hydroxy, nitro, sulfonamido, sulfonyl, —CF₃, —OCF₃, —CO₂H, —CH₂CN, —SO₂CF₃, —CH₂CO₂H and —(CH₂)ₛNR⁷R⁸ wherein s is 0, 1, 2 or 3 and R⁷ and R⁸ are each independently selected from H, straight or branched alkyl of up to 6 carbon atoms, or R⁷ and R⁸ together with the nitrogen atom to which they are linked can form a 5- to 7-membered aliphatic ring which may contain 1 or 2 oxygen atoms;

R is hydrogen, straight or branched alkyl of up to 6 carbon atoms or cycloalkyl of between 5 and 7 carbon atoms which may contain 1 or 2 nitrogen or oxygen atoms;

R⁶ is hydrogen, methyl, or forms with R¹ an aliphatic ring of from 3 to 7 atoms which can contain an oxygen or nitrogen atom, or together with R¹ is a carbonyl group;

Ar is independently selected from Ar or is indolyl or pyridyl-N-oxide;

R³, R⁴, and R⁵ are each independently selected from hydrogen and lower alkyl;

R² is independently selected from Ar or is hydrogen, hydroxy, alkoxy, —NMe₂, —CONR⁹R¹⁰ wherein R⁹ and R¹⁰ are each independently selected from hydrogen, straight or branched alkyl of up to 6 carbon atoms, or R⁹ and R¹⁰ together with the nitrogen atom to which they are linked can form a 5-to 7-membered aliphatic ring which may contain 1 or 2 oxygen or nitrogen atoms, or R² is

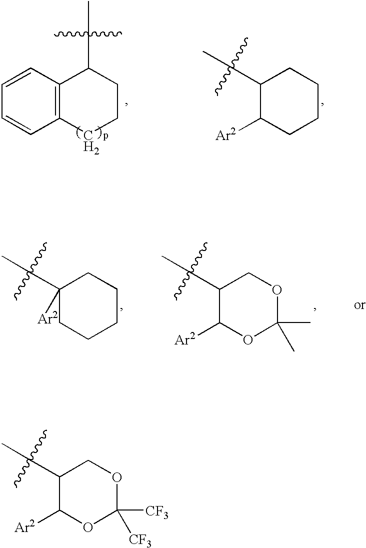

wherein p is 0, 1 or 2 and Ar² is phenyl or pyridyl;

X is a divalent radical derived from any of the following

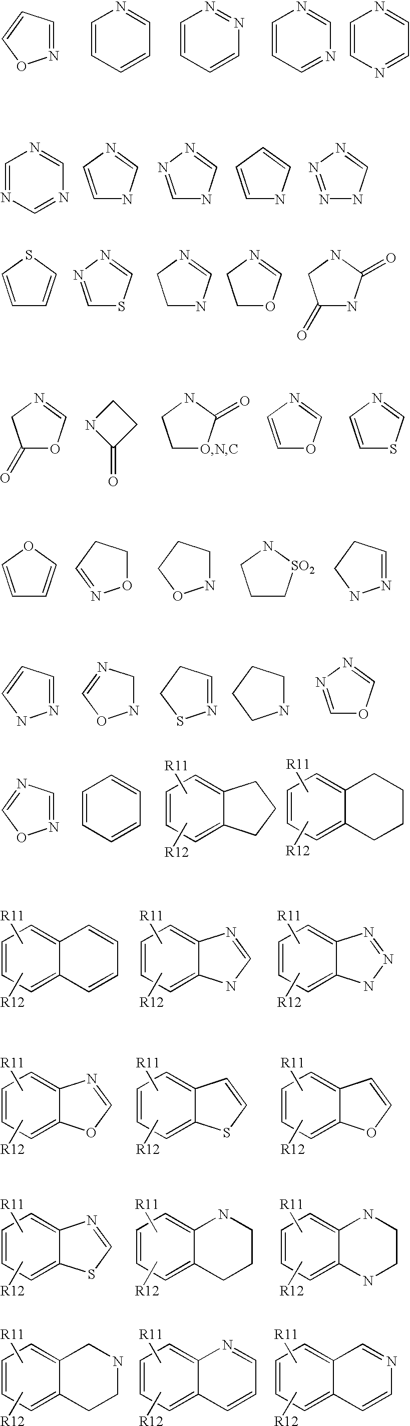

where the ring nitrogen atoms may have lower alkyl groups attached thereto, R¹¹ and R¹² are independently selected from H, halogen, hydroxy, alkoxy, acetyl, nitro, cyano, amino, CF₃ and —(CH₂)ₜNR¹³R¹⁴ where t can be 0 or 1, R¹³ and R¹⁴ are each independently selected from hydrogen, straight or branched alkyl of up to 6 carbon atoms or cycloalkyl of 5 to 7 carbon atoms, containing up to 2 oxygen or nitrogen atoms.

A preferred species of compounds within the genus defined by formula (II) is represented by the formula (IIa), and includes pharmaceutically acceptable salt thereof:

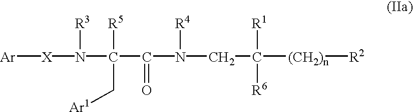

wherein:

n is 0 or 1;

Ar is phenyl or pyridyl which may be unsubstituted or substituted with from I to 3 substituents selected from halogen, alkoxy, nitro and cyano;

Ar¹ is independently selected from Ar or is pyridyl-N-oxide or indolyl;

R⁶ forms with R¹ an aliphatic ring of from 3 to 7 atoms which can contain an oxygen or nitrogen atom, or together with R¹ is a carbonyl group;

R² is independently selected from Ar or is hydrogen, hydroxy, alkoxy, dimethylamino, tetrazolyl or —CONR⁹R¹⁰ wherein R⁹ and R¹⁰ are each independently selected from hydrogen or methyl or R² is any of

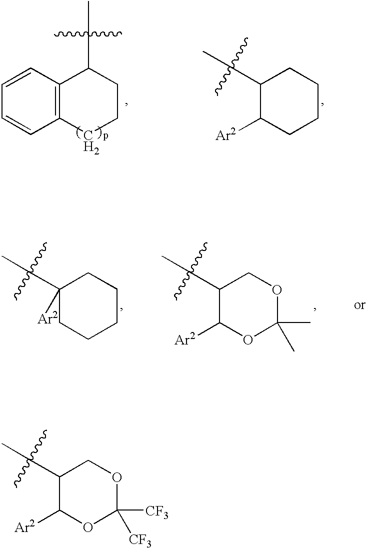

wherein p is 0, 1 or 2 and Ar² is phenyl or pyridyl;

R³, R⁴ and R⁵ are each independently selected from hydrogen and methyl; and

X is selected from:

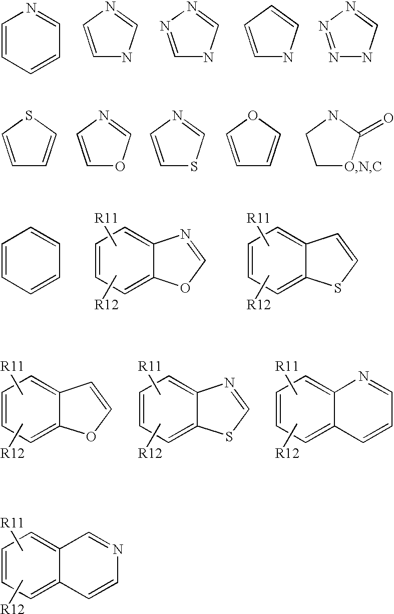

R¹¹ and R¹² being independently selected from H, halogen, hydroxy, alkoxy, acetyl, nitro, cyano, amino, CF₃ and (CH₂)ₜNR¹³R¹⁴ wherein t is 0 or 1 and R¹³ and R¹⁴ are independently selected from hydrogen and methyl.

A sub-species of preferred compounds within the general formula (II) has the formula (IIb) or (IIc):

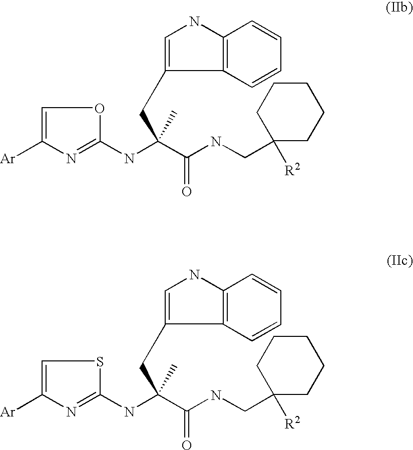

wherein Ar and R² independently represent phenyl or pyridyl which may be unsubstituted or substituted with from 1 to 3 substituents selected from halogen, alkoxy, nitro and cyano, and pharmaceutically acceptable salts thereof.

A particularly preferred compound falling within formula (II) is (S)-3-(1H-indol-3-yl)-N-[1-(5-methoxy-pyridin-2-yl)-cyclohexylmethyl]-2-methyl-2-[4-(4-nitro-phenyl)-oxazol-2-ylamino]-propionamide (also referred to as Compound 1) and its pharmaceutically acceptable salts.

Other preferred compounds falling within formula (II) are described below in Examples 10-27 and are included within the invention, as also are their pharmaceutically acceptable salts.

A third genus of bombesin receptor antagonists according to the invention has the formula (III) and include pharmaceutically acceptable salts thereof.

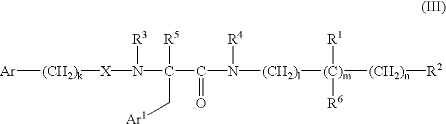

wherein:

k is 0, 1 or 2;

l is 1, 2 or 3;

m is 0 or 1;

n is 0, 1 or 2;

X is —CO—, —OCO, —SO— and —SO₂—;

Ar is benzimidazolyl, benzofuryl, benzothiadiazolyl, benzothiazolyl, benzothienyl, benzopyrazinyl, benzotriazolyl, benzoxadiazolyl, furyl, imidazolyl, indanyl, indolyl, isoquinolyl, isoxazolyl, naphthyl, oxazolyl, phenyl, pyrazinyl, pyrazolyl, pyridyl, pyridazinyl, pyrimidyl, pyrrolyl, quinolinyl, tetralinyl, tetrazolyl, thiazolyl, thienyl or triazolyl each unsubstituted or substituted with from 1 to 3 substituents selected from amino, acetyl, alkyl (straight chain or branched with from 1 to 6 carbon atoms), alkoxy, cyano, halogen, hydroxy, nitro, phenyl, pyridyl, pyrrolyl, isoxazolyl, phenoxy, tolyloxy, —CF₃, —OCF₃, —SO₂CF₃, —NHCONH₂, —CO₂H, —CH₂CO₂H, —CH₂CN, SO₂Me, SO₂NH₂, SO₂Ph, —(CH₂)_{q}NR⁷R⁸, —CONR⁹R¹⁰, and CO₂R¹¹, wherein q is 0, 1 or 2 and R⁷, R⁸, R⁹, R¹⁰, R¹¹ are each independently selected from hydrogen or straight or branched alkyl of up to 6 carbon atoms or cyclic alkyl of between 5 to 7 atoms which may contain 1 or 2 oxygen or nitrogen atoms or R⁷ and R⁸ or R⁹ and R¹⁰ together with the nitrogen atom to which they are linked can form a 5- to 7-membered aliphatic ring which may contain 1 or 2 oxygen or nitrogen atoms;

Ar¹ is independently selected from Ar and can also be pyridyl-N-oxide;

R¹ is hydrogen or straight or branched alkyl of up to 6 carbon atoms or cyclic alkyl of between 5 and 7 atoms which may contain 1 or 2 oxygen or nitrogen atoms;

R² is independently selected from Ar or is hydrogen, hydroxy, alkoxy, —NMe₂, —CONR¹²R¹³,

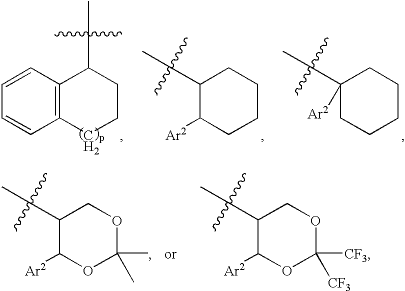

wherein p is 0, 1 or 2, Ar² is phenyl or pyridyl; and, R¹² and R¹³ are each independently selected from hydrogen, straight or branched alkyl of up to 6 carbon atoms or cyclic alkyl of between 5 and 7 carbon atoms;

R³, R⁴ and R⁵ are each independently selected from hydrogen and lower alkyl; and

R⁶ is hydrogen, methyl or forms with R¹ a ring of from 3 to 7 carbon atoms which can contain an oxygen or nitrogen atom, or R¹ and R⁶ can together be carbonyl.

In a preferred group of the compounds of formula (III):

k is 0 or 1;

l is 1;

m is 0 or 1;

n is 0 or 1;

X is —C(O)—, —OC(O)—, or —SO₂—;

Ar is benzofuryl, furyl, indolyl, isoquinolyl, naphthyl, phenyl, pyridyl, quinolyl or thienyl each unsubstituted or substituted with 1 or 2 substituents selected from alkoxy, cyano, halogen, nitro, phenyl, phenoxy, —CF₃, —(CH₂)_{q}NR⁷R⁸, wherein R⁷ and R⁸ can form a ring of between 5 to 7 atoms which may contain 1 or 2 oxygen or nitrogen atoms, or R⁷ and R⁸ can be independently selected from hydrogen, straight or branched alkyl of up to 4 carbon atoms or cyclic alkyl of 5 carbon atoms;

Ar¹ is independently selected from Ar, preferably indolyl, and can also be pyridyl-N-oxide;

R¹ and R⁶ can form a cyclic alkyl of from 5 to 7 carbon atoms or R¹ and R⁶ together are carbonyl;

R² is independently selected from unsubstituted or substituted pyridyl or is hydrogen, hydroxy, alkoxy, —NMe₂, —CONR¹²R¹³ wherein R¹² and R¹³ are each independently selected from H and CH₃;

R³, R⁴ and R⁵ are each independently selected from hydrogen and methyl.

In another preferred group of the compounds of Formula (III),

l is 1;

m is 1;

n is 0;

R² is 2-pyridyl;

R forms a cyclohexyl with R¹.

A particularly preferred group of compounds is of formula (IIa):

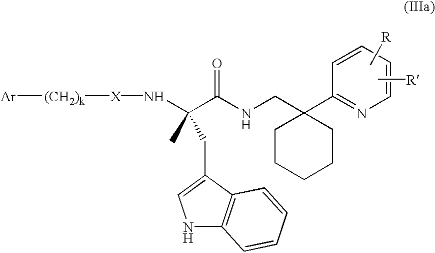

wherein Ar, k and X have the meanings given above in first, and the pyridine ring is optionally substituted by with 1 or 2 substituents, R and R′, independently selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷ and R⁸ can be independently selected from hydrogen or cyclic alkyl of between 5 to 7 carbon atoms, and their pharmaceutically acceptable salts thereof.

In a further set of compounds (IIIa), Ar is benzofuryl, furyl, indolyl, isoquinolyl, naphthyl, phenyl, pyridyl, quinolyl or thienyl each unsubstituted or substituted with 1 or 2 substituents selected from alkoxy, cyano, halogen, nitro, phenyl, phenoxy, —CF₃, —(CH₂)_{q}NR⁷R⁸, wherein R⁷and R⁸ can form a ring of between 5 to 7 atoms which may contain 1 or 2 oxygen or nitrogen atoms, or R⁷ or R⁸ can be a independently selected from hydrogen or cyclic alkyl of 5 carbon atoms, and X is —C(O)—, —OC(O)— or —SO₂.

Preferred N-Terminal Amide Derivatives of the Compounds of Formula (III)

Amongst N-terminal amide derivatives, i.e. compounds of formula (III) wherein X is —C(O)—, the following compounds are most preferred:

N-{(S)-2-(1H-indol-3-yl)-1-methyl-1-[(1-pyridin-2-yl-cyclohexylmethyl)-carbamoyl]-ethyl}-4-nitro-benzamide;

C-dimethylamino-N-{(S)-2-(1H-indol-3-yl)-1-methyl-1-[(1-pyridin-2-yl-cyclohexylmethyl)-carbamoyl]-ethyl}-benzamide;

1H-indole-2-carboxylic acid {(S)-2-(1H-indol-3-yl)-1-methyl-1-[(1-pyridin-2-yl-cyclohexylmethyl)-carbamoyl]-ethyl}-amide;

benzo[b]thiophene-2-carboxylic acid {(S)-2-(1H-indol-3-yl)-1-methyl-1-[(1-pyridin-2-yl-cyclohexylmethyl)-carbamoyl]-ethyl}-amide;

N-{(S)-2-(1H-Indol-3-yl)-1-methyl-1-[(1-pyridin-2-yl-cyclohexylmethyl)-carbamoyl]-ethyl}-2-pyrrol-1-yl-benzamide

1H-indole-5-carboxylic acid {(S)-2-(1H-indol-3-yl)-1-methyl-1-[(1-pyridin-2-yl-cyclohexylmethyl)-carbamoyl]-ethyl}-amide; and

1H-indole-2-carboxylic acid ((S)-2-(1H-indol-3-yl)-1-{[-(5-methoxy-pyridin-2-yl)-cyclohexylmethyl]-carbamoyl}-1-methyl-ethyl)-amide.

Other preferred N-terminal amide derivatives of formula (III) include the compounds of Examples 32-35, 37-47, 49-60, 62-80, 82-85 and N-{(S)-2-(1H-indol-3-yl)-1-methyl-1-[(1-pyridin-2-yl-cyclohexylmethyl)-carbamoyl]-ethyl}-2-pyrrol-1-yl-benzamide.

Preferred N-Terminal Urethane Derivatives of the Compounds of Formula (III)

Amongst N-terminal urethane derivatives, i.e. compounds of formula III wherein X is —OC(═O)—, the following compounds and theor pharmaceutically acceptable salts are particularly preferred:

{(S)-2-(1H-Indol-3-yl)-1-methyl-1-[(1-pyridin-2-yl-cyclohexylmethyl)-carbamoyl]-ethyl}-carbamic acid naphthalen-1-ylmethyl ester;

{(S)-2-(1H-Indol-3-yl)-1-methyl-1-[(1-pyridin-2-yl-cyclohexylmethyl)-carbamoyl]-ethyl}-carbamic acid 3,4-dichloro-benzyl ester;

{(S)-2-(1H-Indol-3-yl)-1-methyl-1-[(1-pyridin-2-yl-cyclohexylmethyl)-carbamoyl]-ethyl}-carbamic acid 4-nitro-benzyl ester;

{(S)-2-(1H-Indol-3-yl)-1-methyl-1-[(1-pyridin-2-yl-cyclohexylmethyl)-carbamoyl]-ethyl}-carbamic acid 3-nitro-benzyl ester;

{(S)-2-(1H-Indol-3-yl)-1-methyl-1-[(1-pyridin-2-yl-cyclohexylmethyl)-carbamoyl]-ethyl}-carbamic acid 3-cyano-benzyl ester;

{(S)-2-(1H-Indol-3-yl)-1-methyl-1-[(1-pyridin-2-yl-cyclohexylmethyl)-carbamoyl]-ethyl}-carbamic acid 3-trifluoromethyl-benzyl ester;

{(S)-2-(1H-Indol-3-yl)-1-methyl-1-[(1-pyridin-2-yl-cyclohexylmethyl)-carbamoyl]-ethyl}-carbamic acid 2,3-dichloro-benzyl ester; and

{(S)-2-(1H-Indol-3-yl)-1-methyl-1-[(1-pyridin-2-yl-cyclohexylmethyl)-carbamoyl]-ethyl}-carbamic acid quinolin-6-ylmethyl ester.

Other preferred N-terminal urethane derivatives of formula (III) include the compounds of Examples 88-90, 92-95, 97-98, 100-102, 104-106 and 108.

Preferred N-Terminal Sulfonamide Derivatives of the Compounds of Formula (III)

Amongst N-terminal sulfonamide derivatives of formula (III) (compounds of formula (III) wherein X is —SO₂—) the following compounds are particularly preferred:

(S)-3-(1H-indol-3-yl)-2-methyl-2-phenylmethanesulfonylamino-N-(1-pyridin-2-yl-cyclohexylmethyl)-propionamide;

(S)-2-(2-chloro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(naphthalene-1-sulfonylamino)-N-(1-pyridin-2-yl-cyclohexylmethyl)-propionamide;

(S)-3-(1H-indol-3-yl)-2-methyl-N-(1-pyridin-2-yl-cyclohexylmethyl)-2-(quinoline-8-sulfonylamino)-propionamide;

(S)-3-(1H-indol-3-yl)-2-methyl-N-(1-pyridin-2-yl-cyclohexylmethyl)-2-(2-trifluoromethyl-benzenesulfonylamino)-propionamide;

(S)-2-(biphenyl-2-sulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(5-methyl-2-phenoxy-benzenesulfonylamino)-N-(1-pyridin-2-yl-cyclohexylmethyl)-propionamide; and

(S)-3-(1H-indol-3-yl)-2-methyl-N-(1-pyridin-2-yl-cyclohexylmethyl)-2-(2-p-tolyloxy-benzenesulfonylamino)-propionamide.

Further preferred N-terminal sulfonamide derivatives of formula (III) include the compounds of Examples 112, 114, 116-119, 121-128, 130-151, 155-168 and the following:

(S)-3-(1H-indol-3-yl)-2-methanesulfonylamino-2-methyl-N-(1-pyridin-2-yl-cyclohexylmethyl)-propionamide; and

(S)-3-(1H-indol-3-yl)-2-methyl-N-(1-pyridin-2-yl-cyclohexylmethyl)-2-(2,2,2-trifluoro-ethanesulfonylamino)-propionamide.

The compounds of the general formulae above are optically active. The scope of the invention therefore also includes:

All stereoisomers of the compounds of the above general formulae.

The solvates, hydrates and polymorphs (different crystalline lattice descriptors) of the above compounds.

Pharmaceutical compositions of the above compounds.

Prodrugs of the above compounds such as would occur to a person skilled in the art; see Bundgaard et al (1987).

The alkyl groups contemplated by the invention include straight, branched, or cyclic carbon chains of from 1 to 8 carbon atoms except where specifically stated otherwise. Representative groups are methyl ethyl, propyl, isopropyl, n-propyl, n-butyl, iso-butyl, sec-butyl, tert-butyl, 2-methylhexyl, n-pentyl, 1-methylbutyl, 2,2-dimethylbutyl, 2-methylpentyl, 2,2-dimethylpropyl, n-hexyl, and the like.

The lower alkyl groups include carbon chains of up to 6 carbon atoms. The cycloalkyl groups contemplated by the invention comprise those having 3 to 7 carbon atoms including cyclopentyl and cyclohexyl. They may be substituted with from 1 to 3 groups selected from halogens, nitro, alkyl, and alkoxy.

The alkoxy groups contemplated by the invention comprise both straight and branched carbon chains of from 1 to 6 carbon atoms unless otherwise stated. Representative groups are methoxy, ethoxy, propoxy, i-propoxy, t-butoxy, and hexoxy.

The term “halogen” is intended to include fluorine, chlorine, bromine, and iodine.

The term “amine” is intended to include free amino, alkylated amines, and acylated amines.

The term “subject” includes animals, particularly mammals and more particularly humans.

Optical Isomers and Salts

The compounds of the above general formulae all have at least one chiral centre and some have multiple chiral centres depending on their structure. In particular, the compounds of the present invention may exist as diastereoisomers, mixtures of diastereoisomers, or as the mixed or the individual optical enantiomers. The present invention contemplates all such forms of the compounds. The mixtures of diastereoisomers are typically obtained as a result of the reactions described more fully below. Individual diastereoisomers may be separated from mixtures of the diastereoisomers by conventional techniques such as column chromatography or repetitive recrystallization. Individual enantiomers may be separated by conventional methods well known in the art such as conversion to a salt with an optically active compound, followed by separation by chromatography or recrystallization and reconversion to the non-salt form.

Where it is appropriate to form a salt, the pharmaceutically acceptable salts include acetate, benzenesulfonate, benzoate, bicarbonate, bitartrate, bromide, calcium acetate, camsylate, carbonate, chloride, citrate, dihydrochloride, edetate, edisylate, estolate, esylate, fumarate, gluceptate, gluconate, glutamate, glycoloylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, mucate, napsylate, nitrate, pamoate (embonate), pantothenate, phosphate/diphosphate, polygalacturonate, salicylate, stearate, subacetate, succinate, sulfate, tannate, tartrate, theoclate, triethiodide, benzathine, chloroprocaine, choline, diethanolamine, ethylenediamine, meglumine, procaine, aluminum, calcium, lithium, magnesium, potassium, sodium, and zinc.

Preferred salts are made from strong acids. Such salts include hydrochloride, mesylate, and sulfate.

Other Non-Peptide Bombesin Antagonists

Other non-peptide bombesin antagonists which are believed to be suitable for use in the present invention are described and claimed in the following documents, the contents of which are incorporated herein by reference: WO 00/09115, WO 00/09116, WO 92/07830, JP 07258081 and WO 98/07718.

Preparative Methods for the Compounds of Formula (I)

Preparation of the compounds of formula (I) is described in WO 98/07718, the disclosure of which is incorporated herein by reference.

Preparation of the closest reported analogues of Compound 3-(2S)-N-{[1-(4-aminophenyl)cyclohexyl]methyl}-3-(1H-indol-3-yl)-2-methyl-2-{[(4-nitroanilino)-carbonyl]amino}propanamide are described in Ashwood, V. Brownhill, M. Higginbottom, D. C. Horwell, J. Hughes, R. A. Lewthwaite, A. T. McKnight, R. D. Pinnock, M. C. Pritchard, N. Suman-Chauhan, C. Webb and S. C. Williams. Bioorg. Med. Chem. Lett., 1998, 8, 2589-2594.; J. E. Eden, M. D. Hall, D. C. Horwell, W. Howson, J. Hughes, R. E. Jordan, R. A. Lewthwaite, K. Martin, A. T. McKnight, J. O'Toole, R. D. Pinnock, M. C. Pritchard, N. Suman-Chauhan and S. C. Williams. Bioorg. Med. Chem. Lett., 1996, 6, 2617-2622. and in WO98/07718. Compound 3 can be synthesized using methods disclosed in the above publications.

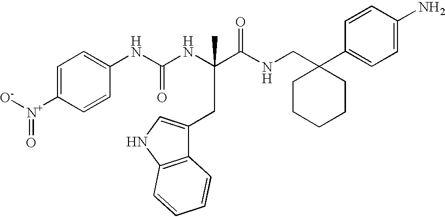

Preparative Methods for Compounds of Formula (II)

Throughout this application the following abbreviations have the meanings listed below:

| | |
|---|---|
| NEt₃ | triethylamine |
| THF | tetrahydrofuran |
| HBTU | O-benzotriazol-1-yl-N,N,N′,N′-tetramethyluronium |
| | hexafluorophosphate |
| DIPEA | N,N-diisopropylethylamine |
| DMF | N,N-dimethylformamide |
| TEBA | benzyltriethylammonium chloride |
| BOC2O | di-tert-butyl dicarbonate |
| TFA | trifluoroacetic acid |
| DMA | N,N-dimethylacetamide |
| EtOAc | ethyl acetate |
| MeOH | methanol |
| Trp | tryptophan |
| Ph | phenyl |
| HPLC | high pressure liquid chromatography |
| NP | normal phase |
| RP | reverse phase |
| DMAP | N,N-dimethyl-4-amino pyridine |
| OAc | acetate |
| OB | oestradiol benzoate |

The production of compounds of the formula (II) in which X is oxazolyl is shown in Scheme 1 which illustrates the synthesis of the compounds of Examples 9 to 12 in four steps via Intermediates 4a or 4b. The steps are:

Formation of the p-nitrophenylcarbamate of the methyl ester (Intermediate 1) and subsequent treatment with aqueous ammonia to give a primary urea (Intermediate 2).

Cyclisation of the primary urea with 2-bromo-1-(4-nitro-phenyl)-ethanone to form an oxazole ring (Intermediate 3).

Hydrolysis of the methyl-ester-protecting group gives Intermediates 4a or 4b.

Reaction of Intermediate 4a or 4b with the amine Z2, using HBTU to form an amide linkage, affords the desired compounds.

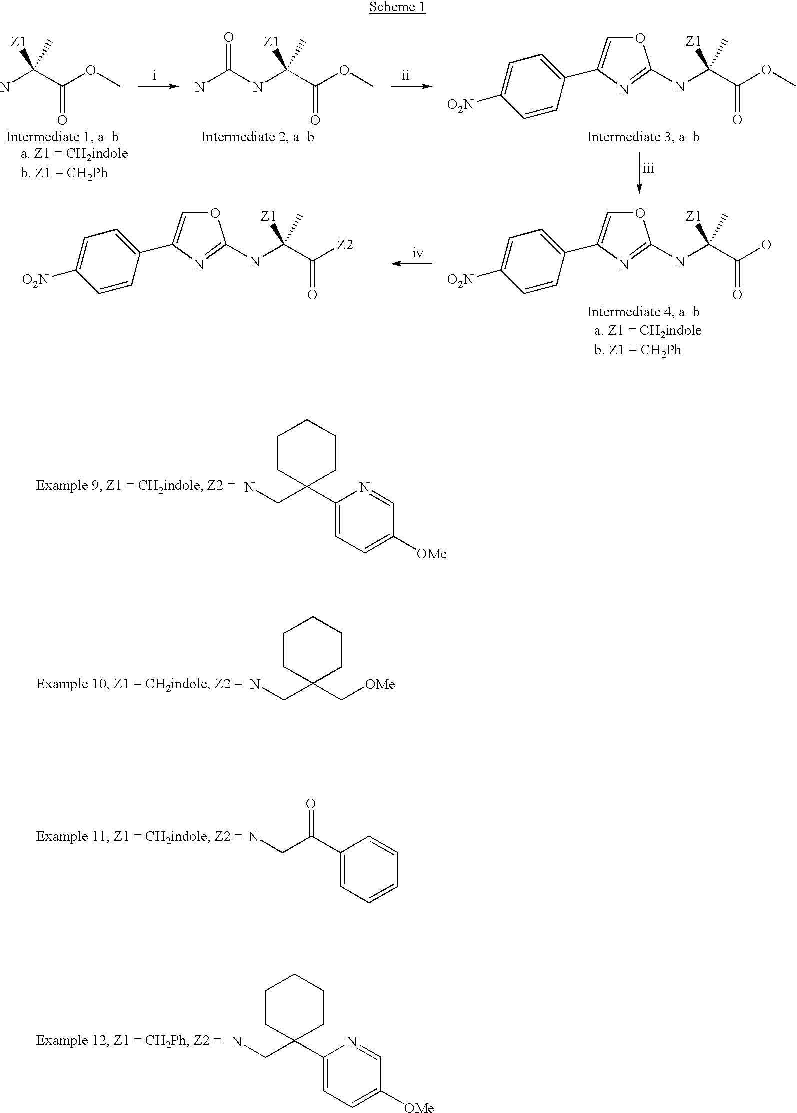

In the above scheme:

i) a) 4-Nitrophenylchloroformate, NEt₃, THF b) NH₃ aq.

ii) 2-bromo-1-(4-nitro-phenyl)-ethanone in either toluene/dioxan at reflux (3a) or 1,2-dichloroethane at reflux (3b)

iii) LiOH, dioxan, H₂O

iv) HBTU, DIPEA, DMF, Z2

Scheme 2 describes the synthesis of the compounds of Examples 13 to 15 from Intermediate 2a.

A primary urea 2a is cyclised with an appropriate bromomethyl ketone containing the group Z3 to form an oxazole ring (Intermediate 5).

Hydrolysis of the methyl ester protecting group of the resulting Intermediate Sa, 5b or 5c gives the Intermediates 6a-c.

Reaction of an Intermediate 6a, 6b or 6c with [1-(5-methoxy-2-pyridyl)cyclohexyl]methanamine in the presence of HBTU to form an amide bond affords the desired compounds.

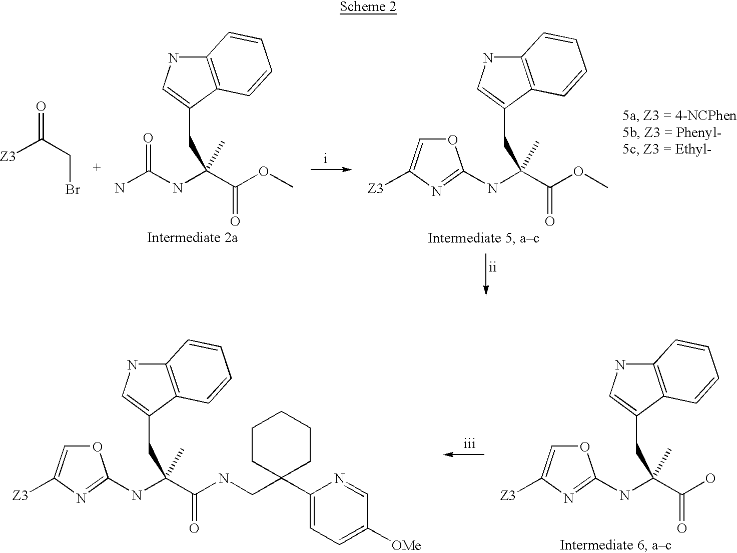

In the above scheme:

i) DMF at 30° C.

ii) LiOH, dioxan, H₂O

iii) HBTU, DIPEA, DMF, [1-(5-methoxy-2-pyridyl)cyclohexyl]methanamine (described in WO 98/07718)

Scheme 3 describes a two step synthesis for the compounds of Examples 16-23. The reactions are preferentially carried out as a “one-pot” process in which:

An aromatic ring of a compound Z5-Br or Z5-Cl is appended onto the N-terminal of the illustrated amino acid using a copper catalysed reaction.

Formation of an amide linkage between the resulting acid and [1-(5-methoxy-2-pyridyl)cyclohexyl]methanamine or [1-(2-pyridyl)cyclohexyl]methylamine in the presence of HBTU affords the desired compounds.

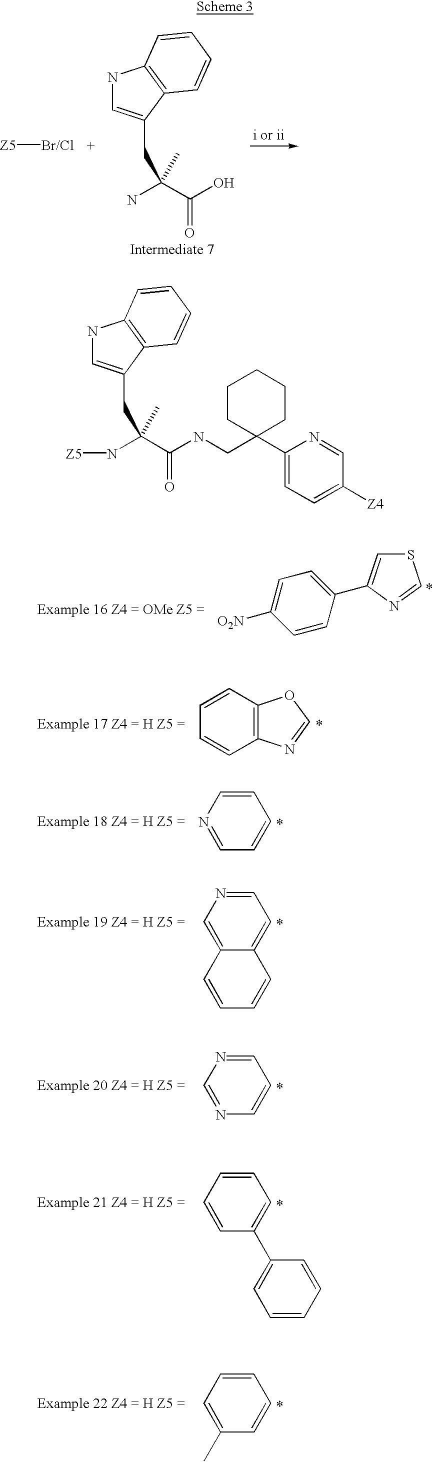

In the above scheme:

i) a) 10% Cul, K₂CO₃, DMF, 130° C.

b) HBTU, DIPEA, DMF, and [1-(5-methoxy-2-pyridyl)cyclohexyl]methanamine (described in WO 98/07718) or [1-(2-pyridyl)cyclohexyl]methylamine (described in WO 98/07718)

ii) a) 5-10% CuI, K₂CO₃, TEBA, Pd(P(o-tolyl)₃)C₁₂, DMF, 130° C.

b) HBTU, DIPEA, DMF, and [1-(5-methoxy-2-pyridyl)cyclohexyl]methanamine (described in WO 98/07718) or [1-(2-pyridyl)cyclohexyl]methylamine (described in WO 98/07718)

* represents the attachment point.

Scheme 4 describes the two step one-pot synthesis of the compound of Example 24:

The aromatic ring is appended onto the N-terminal of the amino acid (Intermediate 8) using a copper catalysed reaction and then an in situ HBTU amide bond formation reaction affords the desired compound.

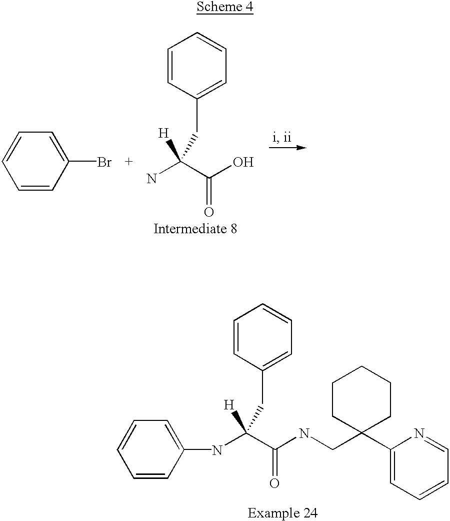

In the above scheme:

i) 10% Cul, K₂CO₃, DMA, 90° C.

ii) HBTU, NEt₃, DMA, [1-(2-pyridyl)cyclohexyl]methylamine (described in WO 98/07718)

Scheme 5 describes the synthesis of the compounds of Examples 25-27 via Intermediate 10 by the steps of:

N-BOC protection of the amino acid (Intermediate 7) which provides the groups R⁵ and Ar¹.

Reaction of the protected amino acid with an amine that provides the groups R¹, R², R⁴ and R⁶ using HBTU to form an amide linkage, and thereby give the Intermediate 9.

N-BOC deprotection of the Intermediate 9 to give Intermediate 10.

Reductive amination of Intermediate 10 with the appropriate aldehyde Z6CHO to give the desired compounds.

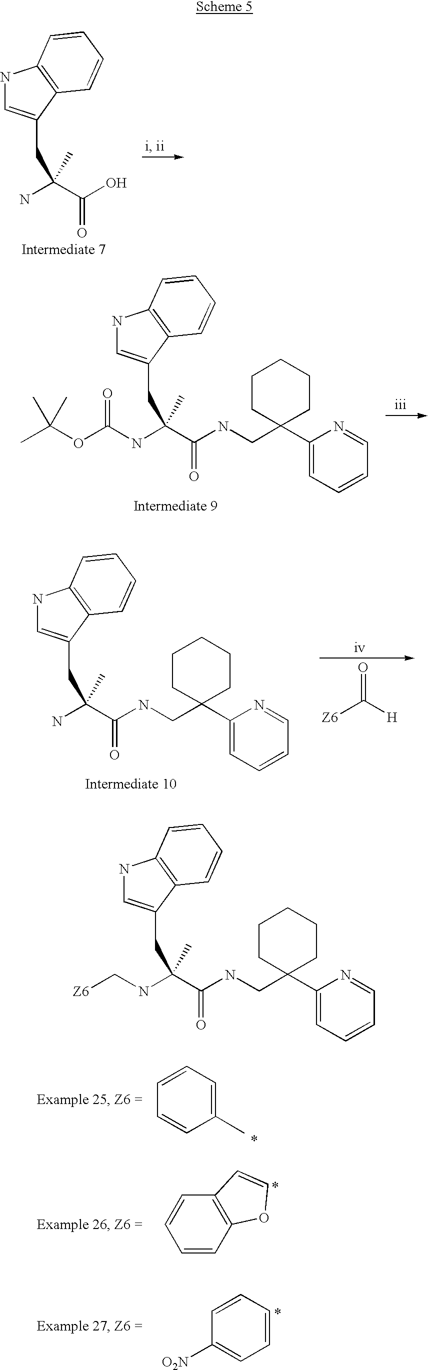

In the above scheme:

i) BOC₂O, K₂CO₃, dioxane, water

ii) HBTU, DIPEA, [1-(2-pyridyl)cyclohexyl]methylamine (described in WO 98/07718), DMF

iii) TFA, CH₂Cl₂

iv) NaBH(OAc)₃, 1,2-dichloroethane.

* represents the attachment point.

Scheme 6 describes the synthesis of Intermediate 13.

The alcohol 11 is methylated using sodium hydride.

The resulting nitrile is reduced using Raney nickel under an atmosphere of hydrogen.

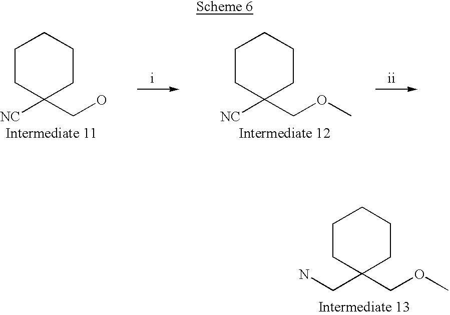

In the above scheme:

i) NaH, CH₃I, THF

ii) Raney nickel, ethanolic ammonia, H₂, 345 kPa

Intermediate 13

C-(1-methoxymethyl-cyclohexyl)-methylamine

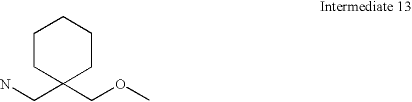

The above compound was prepared as shown in Scheme 6.

1. Sodium hydride (862 mg, 21.5 mmol, 60% in oil) was taken up in THF (50 ml) under argon at 0° C. To this was added a solution of methyl iodide (1.34 ml, 21.6 mmol) and 1-hydroxy-cyclohexanecarbonitrile (1.0 g, 7.18 mmol; see J. Fröhlich et al., 1994) in THF (30 ml) dropwise over 45 minutes. Once addition was complete the reaction mixture was stirred at room temperature overnight, and then quenched with i-propanol followed by water (100 ml). The mixture was then extracted with dichloromethane (2×150 ml). The combined organic phases were dried (MgSO₄) and solvent removed under reduced pressure. Residue was purified by chromatography using heptane/ethyl acetate (4:1). Removal of solvent under reduced pressure gave 1-methoxymethyl-cyclohexanecarbonitrile (1.1 g, 88%) as a pale yellow oil.

IR (film): 2934, 2861, 2832, 2235, 1476, 1452, 1385, 1211, 1187, 1185, 1126, 1102, 978, 932, 901, 849 cm⁻¹;

¹H NMR (CDCl₃): δ=1.13-1.33 (3H, m), 1.57-1.78 (5H, m), 1.94-2.02 (2H, m), 3.36 (1H, s), 3.42 (3H, s);

2. To the 1-methoxymethyl-cyclohexanecarbonitrile (1.1 g, 7.2 mmol) in ethanolic ammonia (60 ml) was added Raney nickel catalyst (0.55 g, pre-washed with water and ethanol). Reaction mixture was shaken for 16 hours under hydrogen (345 kPa) at 30° C. The catalyst was filtered off catalyst with extreme caution through a bed of Kieselguhr and washed with ethanol. Removal of the solvent under reduced pressure gave Intermediate 13 (1.12 g, 99%) as a yellow oil.

MS m/e (ES+): 158.2 (M⁺+H, 100%);

IR (film): 2926, 2857, 1572, 1452, 1378, 1316, 1190, 1140, 966 cm⁻¹;

¹H NMR (CDCl₃): δ=1.20-1.60 (12H, m), 2.62 (2H, s), 3.23 (2H, s), 3.32 (3H, s)

Preparative Methods for Compounds of Formula (III)

Compounds of the formula (III) in which X is —CO— can be prepared by condensing an acid of the formula (III-1)

Ar—(CH₂)ₖ—COOH (III-1)

or a derivative thereof with an amine of the formula (III-2)

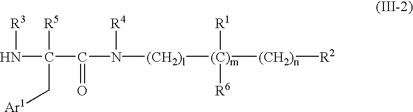

in an aprotic polar solvent in the presence of an appropriate catalyst, the values of the substituents Ar, Ar¹ and R¹ to R⁶ and the parameters k to n being as defined above with reference to formula (III), and optionally converting the resulting product to a pharmaceutically acceptable salt. For example, the condensation may be carried out in dimethylformamide using O-benzotriazol-1-yl-N,N,N′,N′-tetramethyluronium hexafluorophosphate (HBTU) and N,N-diisopropyl-ethylamine (DIPEA) as catalyst.

Compounds of the formula (III) in which X is —OC(═O)— can be prepared by forming a carbonate from an alcohol of the formula (III-3)

Ar—(CH₂)ₖ—OH (III-3)

and reacting the carbonate with an amine of the formula (III-2)

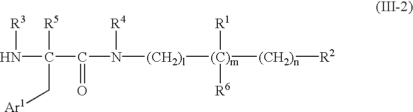

in an aprotic polar solvent in the presence of a base, the values of the substituents Ar, Ar¹ and R¹ to R⁶ and the parameters k to n being as defined above with reference to formula (III), and optionally converting the resulting product to a pharmaceutically acceptable salt. For example, the compound of formula (III-3) may be reacted with 4-nitrophenyl chloroformate in dichloromethane using pyridine as catalyst, and the resulting carbonate may be reacted with the amine of formula (III-2) in dimethyl formamide using N,N-dimethyl-4-amino pyridine as catalyst.

Compounds of the formula (III) in which X is —SO₂— can be prepared by condensing a sulfonyl chloride of the formula (III-4)

Ar—(CH₂)ₖ—SO₂Cl (III-4)

with an amine of the formula (III-2)

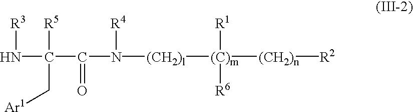

in an aprotic polar solvent in the presence of a base as catalyst, the values of the substituents Ar, Ar¹ and R¹ to R⁶ and the parameters k to n being as defined above with reference to formula (III), and optionally converting the resulting product to a pharmaceutically acceptable salt. For example, the condensation may be carried out in dimethylformamide in the presence of N,N-diisopropylethylamine and N,N-dimethyl-4-aminopyridine.

In the above methods, the amine of formula (III-2) is preferably a chiral amine of formula (III-5)

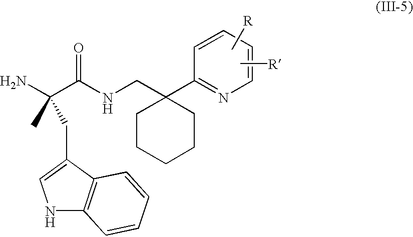

wherein the pyridine ring is optionally substituted by with 1 or 2 substituents R and R′ selected from alkoxy, cyano, halogen, nitro, phenyl, phenoxy, —CF₃, —(CH₂)_{q}NR⁷R⁸, wherein R⁷ and R⁸ can form a ring of between 5 to 7 atoms, which may contain 1 or 2 oxygen or nitrogen atoms, or R⁷and R⁸ can be independently selected from hydrogen or cyclic alkyl of from 1 to 5 carbon atoms, methoxy being a particularly preferred substituent, as in the chiral amine (III-6):

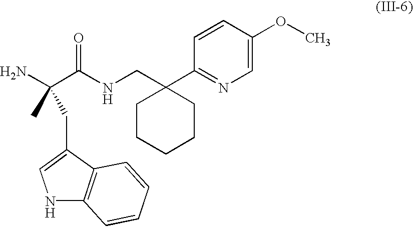

B) Peptide Bombesin Receptor Antagonists

Bombesin antagonists which are peptides and which are believed to be suitable for use in the present invention are described in the following documents, the contents of which are incorporated herein by reference:

| Publication | Publication |
|---|---|
| number | number |
| WO 97/09347 | EP 0835662 |
| US 5650395 | US 5439884 |
| WO 96/28214 | WO 95/00542 |
| EP 0737691 | US 5620955 |
| US 5767236 | WO 92/02545 |
| WO 91/04040 | EP 0468497 |
| EP 0309297 | CA 2030212 |
| EP 0438519 | WO 92/20707 |
| EP 0559756 | WO 93/16105 |
| WO 89/02897 | US 4943561 |
| WO 90/03980 | US 5019647 |
| WO 91/02746 | US 5028692 |
| WO 92/09626 | US 5047502 |
| WO 92/20363 | WO 94/02018 |
| WO 94/02163 | WO 88/07551 |
| WO 94/21674 | WO 89/09232 |
| WO 96/17617 | EP 0315367 |
| US 5084555 | EP 0345990 |
| US 5162497 | US 5068222 |
| US 5244883 | US 5620959 |
| US 5723578 | UK 2231051 |
| US 5750646 | EP 0339193 |
| US 5877277 | WO 90/01037 |
| US 5985834 | WO 91/06563 |
| EP 0428700 | EP 0402852 |

Pharmaceutical Compositions

For preparing pharmaceutical compositions from the compounds of this invention, inert, pharmaceutically acceptable carriers can be either solid or liquid. Solid form preparations include powders, tablets, dispersible granules, capsules, sachets, and suppositories.

A solid carrier can be one or more substances which may also act as diluents, flavouring agents, solubilizers, lubricants, suspending agents, binders, or tablet disintegrating agents; it can also be an encapsulating material. In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain 5% to about 70% of the active component. Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid form preparations include solutions, suspensions, and emulsions. Sterile water or water-propylene glycol solutions of the active compounds may be mentioned as an example of liquid preparations suitable for parenteral administration. Liquid preparations can also be formulated in solution in aqueous polyethylene glycol solution. Aqueous solutions for oral administration can be prepared by dissolving the active component in water and adding suitable colorants, flavouring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Preferably the pharmaceutical preparation is in unit dosage form. In such form, the preparation is divided into unit doses containing appropriate quantities of the active component. The unit dosage form can be a packaged preparation, the package containing discrete quantities of the preparation, for example, packeted tablets, capsules, and powders in vials or ampoules. The unit dosage form can also be a capsule, sachet, or tablet itself, or it can be the appropriate number of any of these packaged forms.

For preparing suppository preparations, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Compositions that are adapted for oral administration to humans are preferred, especially such compositions in unit dosage form.

Combination Therapy

Without wishing to be bound by any particular theory or teaching, the inventors believe that bombesin receptor antagonists could be used as part of a medicament in combination with one or more vasodilator, hormone therapy or neurotransmitter modulator. Such products are used or tested in the treatment of sexual dysfunction.

Vasodilators for the treatment of sexual dysfunctions of organic (rather than psychogenic) origin, act at the penis, clitoris or vagina level on local blood flow or lubricant secretions. Vasodilators useful for the treatment of sexual dysfunction include alprostadil or phentolamine, NO (nitric oxide) enhancers such as L-arginine, and PDE5 inhibitors such as sildenafil or a pharmaceutically acceptable salt thereof (Scrip's Complete Guide to Women's Healthcare, p.194-205, 2000)(Sachs B. D., 2000, Benet and Melman, 1995), VIP (Vaso Intestinal Peptide) enhancers (Scrip's Complete Guide to Women's Healthcare, p.194-205, 2000) or angiotensin-2 receptor antagonists such as losartan (American Heart Association meeting, New Orleans, 2000).

Hormone therapies useful in the treatment of sexual dysfunction of organic and psychogenic nature include modulators of steroid hormones, steroid hormones or hormone product (including synthetic hormones) including oestrogen (Scrip's Complete Guide to Women's Healthcare, p.194-205, 2000), or androgens such as testosterone (Scrip's Complete Guide to Women's Healthcare, p.194-205, 2000, Sachs B. D., 2000), which act in areas of the CNS associated with sexual desire and sexual arousal (Wilson C A., 1993).

Neurotransmitter modulators useful in the treatment of both psychogenic and organic sexual dysfunction include neurotransmitter agonists and antagonists such as catecholamine agonists such as the D₂ agonist quinelorane, 5HT₂ antagonists such as ritanserin, monoamine synthesis modifiers such as treatments that reduce endogenous 5HT activity, including inhibition of 5HT synthesis using para-chlorophenylalanine, monoamine metabolism or uptake modifiers that inhibit catecholamine metabolism or reuptake, such as tricyclic antidepressants, e.g. imipramine (Wilson C A., 1993).

The use of this combination therapy includes the preparation of therapies that would allow administration of both components of the medicament, i.e. bombesin receptor antagonists and a vasodilator, hormone therapy medicament or neurotransmitter modulator medicament in a single dose. A preferred formulation would allow oral administration. However, administration by suppository, cream, transdermal patch or injection is also part of this invention. Alternatively the inventors envisage formulations that allow administration of the bombesin receptor antagonist via a separate route to that of the vasodilator, hormone therapy medicament or neurotransmitter modulator medicament. Such routes could include for example oral administration of the bombesin receptor antagonist and transdermal patch application of the vasodilator. Thus there may be provided a kit in which unit doses of bombesin receptor antagonist occur in association with unit doses of the vasodilator, hormone therapy medicament or neurotransmitter modulator medicament. For example, in the case of a kit where bombesin receptor antagonist is formulated as a tablet capsule or other unit dosage form for oral administration and the vasodilator is provided as a transdermal patch, the two dosage forms could be provided in the form of a two-row tear-off strip in which compartments containing the tablets, etc. occur above compartments containing the transdermal patches. Other forms of packaging in which the two dosage forms are spatially associated so as to make it easy for patients to take them together and to be reminded when they have done so will readily occur to those skilled in the art. The kit will also contain instructions as to when and how the individual components of the kit should be administered.

More generally, the invention provides a pharmaceutical combination (for simultaneous, separate or sequential administration) of a bombesin receptor antagonist and one or more materials selected from (1) to (34) below:

(1) One or more naturally occurring or synthetic prostaglandins or esters thereof. Suitable prostaglandins for use herein include compounds such as alprostadil, prostaglandin E₁,prostaglandin E₀, 13, 14-dihydroprostaglandin E₁, prostaglandin E₂, eprostinol, natural synthetic and semi-synthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁α, 19-hydroxy PGA₁, 19-hydroxy-PGB₁, PGE₂, PGB₂, 19-hydroxy-PGA₂, 19-hydroxy-PGB₂, PGE₃α, carboprost, tromethamine, dinoprost, dinoprostone, iloprost, gemeprost, metenoprost, sulprostune, tiaprost and moxisylate.

(2) One or more α-adrenergic receptor antagonist compounds also known as α-adrenoceptor antagonists or α-receptor antagonists or α-blockers. Suitable compounds for use herein include: the α-adrenergic receptor blockers as described in PCT application WO99/30697 published on Jun. 14, 1998, the disclosure of which relating to α-adrenergic receptors incorporated herein by reference and include, selective α₁-adrenoceptor or α₂-adrenoceptor blockers and non-selective adrenoceptor blockers, Suitable α₁-adrenoceptor blockers include: phentolamine, phentolamine mesylate, trazodone, alfuzosin, indoramin, naftopidil, tamsulosin, dapiprazole, phenoxybenzamine, idazoxan, efarxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenarnine, tolazoline, trimazosin and dibenamine; α-adrenergic receptor antagonists as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cardiotonic agent such as pirxamine.

(3) One or more NO-donor (NO-agonist) compounds. Suitable NO-donor compounds for use herein include organic nitrates, such as mono- di or tri-nitrates or organic nitrate esters including glyceryl tririnitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine, molsidomine, S-nitroso-N-acetyl penicillamine (SNAP) S-nitroso-N-glutathione (SNO-GLU), N-hydroxy-L-arginine, amylnitrate, linsidomine, linsidomine hydrochloride, (SIN-1) S-nitroso-N-cysteine, diazenium diolates,(NONOates), 1,5-pentanedinitrate, L-arginine, ginseng, zizphi fructus, molsidomine, Re-2047, nitrosylated maxisylyte derivatives such as NMI-678-11 and NMI-937 as described in published PCT application WO 0012075 and/or

(4) One or more potassium channel openers or modulators. Suitable potassium channel openers/modulators for use herein include nicorandil, cromokalim, levcromakalim (lemakalim), pinacidil, diazoxide, minoxidil, charybdotoxin, glyburide, 4-aminopyridine, BaCl₂.

(5) One or more dopaminergic agents, preferably apomorphine or a selective D2, D3 or D2/D₃agonist such as, pramipexole and ropirinol (as claimed in WO-0023056),PNU95666 (as claimed in WO-0040226).

(6) One or more vasodilator agents. Suitable vasodilator agents for use herein include nimodepine, pinacidil, cyclandelate, isoxsuprine, chloropromazine, halo peridol, Rec 15/2739, trazodone.

(7) One or more thromboxane A2 agonists.

(8) One or more CNS active agents.

(9) One or more ergot alkaloids. Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride.

(10) One or more compounds which modulate the action of natriuretic factors in particular atrial natriuretic factor (also known as atrial natriuretic peptide), B type and C type natriuretic factors.

(11) One or more angiotensin receptor antagonists such as losartan.

(12) One or more substrates for NO-synthase, such as L-arginine.

(13) One or more calcium channel blockers such as amlodipine.

(14) One or more cholesterol lowering agents such as statins (e.g. atorvastatin/Lipitor-trade mark) and fibrates.

(15) One or more antiplatelet and antithrombotic agents, e.g. tPA, uPA, warfarin, hirudin and other thrombin inhibitors, heparin, thromboplastin activating factor inhibitors.

(16) One or more insulin sensitising agents such as triglitazone (rezulin) and hypoglycaemic agents such as glipizide.

(17) L-DOPA or carbidopa.

(18) One or more acetylcholinesterase inhibitors such as donepezil (Aricept).

(19) One or more steroidal or non-steroidal anti-inflammatory agents.

(20) One or more estrogen receptor modulators (SERM) and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656.

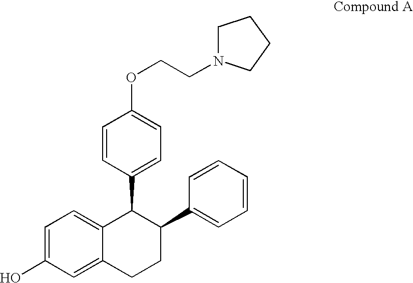

(21) One or more PDE inhibitors, more particularly a PDE 2, 5, 7 or 8 inhibitor (for oral or local administration), preferably PDE2 or PDE5 inhibitor and most preferably a PDE5 inhibitor (see hereinafter), said inhibitors preferably having an IC50 against the respective enzyme of less than 100 nM; and PDE 3, 4 inhibitor for local administation (e.g. intracavemosal injection).

(22) In the case where the combination is for the treatment or prophylaxis of female sexual dysfunction, one or more of an NPY (neuropeptide Y) inhibitor, more particularly NPY1 or NPY5 inhibitor, preferably NPY1 inhibitor. Preferably said NPY inhibitors (including NPYY1 and NPYY5) have an IC50 of less than 100 nM, more preferably less than 50 nM.

(23) One or more of a neutral endopeptidase (NEP) inhibitor preferably having an IC50 for NEP of less than 100 nM. Preferably the NEP inhibitor is selective for NEP and has a selectivity over the endothelin converitin enzyme (ECE) and angiotensin converting enzyme (ACE) of greater than 100. However, mixed/dual NEP/ECE and NEP/ACE inhibitors (such as ompatrilat) are still within the scope of the invention.

(24) One or more of vasoactive intestinal protein (VIP), VIP mimetic, VIP analogue, more particularly acting through one or more of the VIP receptor subtypes VPAC1,VPAC or PACAP (pituitory adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil).

(25) One or more of a melanocortin receptor agonist or modulator or melanocortin enhancer, such as melanotan II, PT-14, PT-141 or compounds claimed in WO-09964002, WO-00074679, WO-09955679, WO-00105401, WO-00058361, WO-00114879, WO-00113112, WO-09954358.

(26) One or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993.

(27) One or more of a testosterone replacement agent (including dehydroandrostendione), testosterone (Tostrelle), dihydrotestosterone or a testosterone implant.

(28) One or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone).

(29) One or more of a modulator of transporters for noradrenaline, dopamine and/or serotonin, such as bupropion, GW-320659.

(30) One or more of a purinergic receptor agonist and/or modulator.

(31) One or more of a neurokinin (NK) receptor antagonist, including those described in WO-09964008.

(32) One or more of an opioid receptor agonist, antagonist or modulator, preferably agonists for the ORL-1 receptor.

(33) One or more of an agonist or modulator for oxytocin/vasopressin receptors, preferably a selective oxytocin agonist or modulator.

(34) One or more modulators of cannabinoid receptors.

Auxiliary Agent PDE5 Inhibitor (I:PDE5):

PDE5 Inhibitors

Suitable PDE5i's for use in the pharmaceutical combinations according to the present invention are the cGMP PDE5i's hereinafter detailed. Particularly preferred for use herein are potent and selective cGMP PDE5i's. Suitable cGMP PDE5 inhibitors for the use according to the present invention include:

pyrazolo [4,3-d]pyrimidin-7-ones disclosed in EP-A-0463756;

pyrazolo [4,3-d]pyrimidin-7-ones disclosed in EP-A-0526004;

pyrazolo [4,3-d]pyrimidin-7-ones disclosed in published international patent application WO 93/06104;

isomeric pyrazolo [3,4-d]pyrimidin-4-ones disclosed in published international patent application WO 93/07149;

quinazolin-4-ones disclosed in published international patent application WO 93/12095;

pyrido [3,2-d]pyrimidin-4-ones disclosed in published international patent application WO 94/05661;

purin-6-ones disclosed in published international patent application WO 94/00453;

pyrazolo [4,3-d]pyrimidin-7-ones disclosed in published international patent application WO 98/49166;

pyrazolo [4,3-d]pyrimidin-7-ones disclosed in published international patent application WO 99/54333;

pyrazolo [4,3-d]pyrimidin-4-ones disclosed in EP-A-0995751;

pyrazolo [4,3-d]pyrimidin-7-ones disclosed in published international patent application WO 00/24745;

pyrazolo [4,3-d]pyrimidin-4-ones disclosed in EP-A-0995750;

the compounds disclosed in published international application WO95/19978;

the compounds disclosed in published international application WO 99/24433 and

the compounds disclosed in published international application WO 93/07124.

It is to be understood that the contents of the above published patent applications, and in particular the general formulae and exemplified compounds therein are incorporated herein in their entirety by reference thereto.

Preferred type V phosphodiesterase inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333 );

(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo [4,3-d] pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo [4,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

(6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo [5,1-fl-as-triazin-2-yl)-4-ethoxyphenyl]sulphonyl]-4-ethylpiperazine, i.e. the compound of examples 20, 19, 337 and 336 of published international application WO99/24433; and

the compound of example 11 of published international application WO93/07124 (EISAI); and

compounds 3 and 14 from Rotella D P, J. Med. Chem., 2000, 43, 1257.

Still other type cGMP PDE5 inhibitors useful in conjunction with the present invention include:

4-bromo-5-(pyridylmethylamino)-6-[3-(4-chlorophenyl)-propoxy]-3(2H)pyridazinone;

1-[4-[(1,3-benzodioxol-5-ylmethyl)amiono]-6-chloro-2-quinozolinyl]-4-piperidine-carboxylic acid, monosodium salt;

(+)-cis-5,6a,7,9,9,9a-hexahydro-2-[4-(trifluoromethyl)-phenylmethyl-5-methyl-cyclopent-4,5]imidazo [2,1-b]purin-4(3H)one;

furazlocillin;

cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate;

4-bromo-5-(3-pyridylmethylamino)-6-(3-(4-chlorophenyl) propoxy)-3-(2H)pyridazinone;

I-methyl-5(5-morpholinoacetyl-2-n-propoxyphenyl)-3-n-propyl-1,6-dihydro-7H-pyrazolo(4,3-d)pyrimidin-7-one;

1-[4-[(1,3-benzodioxol-5-ylmethyl)amino]-6-chloro-2-quinazolinyl]-4-piperidinecarboxylic acid, monosodium salt;

Pharmaprojects No. 4516 (Glaxo Wellcome);

Pharmaprojects No. 5051 (Bayer);

Pharmaprojects No. 5064 (Kyowa Hakko; see WO 96/26940);

Pharmaprojects No. 5069 (Schering Plough);

GF-196960 (Glaxo Wellcome);

E-8010 and E-4010 (Eisai); Bay-38-3045 & 38-9456 (Bayer); and

Sch-51866.

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the cGMP PDE5 inhibitors have an IC₅₀ at less than 100 nanomolar, more preferably, at less than 50 nanomolar, more preferably still at less than 10 nanomolar. IC₅₀ values for the cGMP PDE5 inhibitors may be determined using the PDE5 assay in the Test Methods Section hereinafter.

Preferably the cGMP PDE5 inhibitors used in the pharmaceutical combinations according to the present invention are selective for the PDE5 enzyme. Preferably they are selective over PDE3, more preferably over PDE3 and PDE4. Preferably, the cGMP PDE5 inhibitors of the invention have a selectivity ratio greater than 100 more preferably greater than 300, over PDE3 and more preferably over PDE3 and PDE4. Selectivity ratios may readily be determined by the skilled person. IC₅₀ values for the PDE3 and PDE4 enzyme may be determined using established literature methodology, see S A Ballard et al (1998) and as detailed hereinafter.

Auxiliary Agent: NEP Inhibitor (I:NEP)

NEP EC3.4.24.11 (FEBS Lett., 229(1), 206-210 (1988)), also known as enkephalinase or neprilysin, is a zinc-dependent neutral endopeptidase. This enzyme is involved in the breakdown of several bioactive oligopeptides, cleaving peptide bonds on the amino side of hydrophobic amino acid residues (Reviewed in Turner et al., 1997). The key neuronally released bioactive agents or neuropeptides metabolised by NEP include natriuretic peptides such as atrial natriuretic peptides (ANP) as well as brain natriuretic peptide and C-type natriuretic peptide, bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects. Background teachings on NEP have been presented by Victor A. McKusick et al on http://www3.ncbi.nlm.nih.gov/Omim/searchomim.htm. The following information concerning NEP has been extracted from that source.

“Common acute lymphocytic leukemia antigen is an important cell surface marker in the diagnosis of human acute lymphocytic leukemia (ALL). It is present on leukemic cells of pre-B phenotype, which represent 85% of cases of ALL. CALLA is not restricted to leukemic cells, however, and is found on a variety of normal tissues. CALLA is a glycoprotein that is particularly abundant in kidney, where it is present on the brush border of proximal tubules and on glomerular epithelium. Letarte et al. (1988) cloned a cDNA coding for CALLA and showed that the amino acid sequence deduced from the cDNA sequence is identical to that of human membrane-associated neutral endopeptidase (NEP; EC 3.4.24.11 ), also known as enkephalinase. NEP cleaves peptides at the amino side of hydrophobic residues and inactivates several peptide hormones including glucagon, enkephalins, substance P, neurotensin, oxytocin, and bradykinin. By cDNA transfection analysis, Shipp et al. (1989) confirmed that CALLA is a functional neutral endopeptidase of the type that has previously been called enkephalinase. Barker et al. (1989) demonstrated that the CALLA gene, which encodes a 100-kD type II transmembrane glycoprotein, exists in a single copy of greater than 45 kb which is not rearranged in malignancies expressing cell surface CALLA. The gene was located to human chromosome 3 by study of somatic cell hybrids and in situ hybridization regionalized the location to 3q21-q27. Tran-Paterson et al. (1989) also assigned the gene to chromosome 3 by Southern blot analysis of DNA from human-rodent somatic cell hybrids. D'Adamio et al. (1989) demonstrated that the CALLA gene spans more than 80 kb and is composed of 24 exons.”

Preferred for NEPi's for use as auxiliary agents in combination with bombesin receptor antagonists according to the present invention are:

(2R)-2-[(1-{[(5-ethyl-1,3,4-thiadiazol-2-yl)amino]carbonyl}cyclopentyl) methyl]pentanoic acid

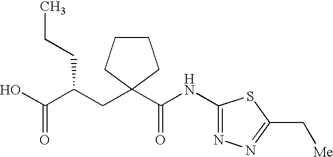

and

(2S)-2-[(1-{[(5-Ethyl-1,3,4-thiadiazol-2-yl)amino]carbonyl}cyclopentyl)-methyl]pentanoic acid

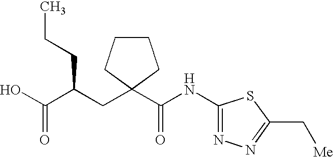

The title product from stage c) below (824 mg) was further purified by HPLC using an AD column and using hexane:iso-propanol:trifluoroacetic acid (85:15:0.2) as elutant to give (2R)-2-[(1-{[(5-ethyl-1,3,4-thiadiazol-2-yl)amino]carbonyl}-cyclopentyl) methyl]pentanoic acid as a white foam, 400 mg, 99.5% ee,

¹H NMR (CDCl₃, 400 MHz) δ: 0.90 (t, 3H), 1.36 (m, 6H), 1.50-1.80 (m, 9H), 2.19 (m, 1H), 2.30 (m, 1H), 2.44 (m, 1H), 2.60 (m, 1H), 2.98 (q, 2H), 12.10-12.30 (bs, 1H), LRMS: m/z 338 (MH⁻), [α]_{D}=−9.0° (c 0.1, methanol), and (2S)-2-[(1-{[(5-ethyl-1,3,4-thiadiazol-2-yl)amino]carbonyl}cyclopentyl)-methyl]pentanoic acid as a white foam, 386 mg, 99% ee, ¹H NMR (CDCl₃, 400 MHz) δ: 0.90 (t, 3H), 1.38 (m, 6H), 1.50-1.79 (m, 9H), 2.19 (m, 1H), 2.30 (m, 1H), 2.44 (m, 1H), 2.60 (m, 1H), 2.98 (q, 2H), 12.10-12.27 (bs, 1H); LRMS: m/z 338 (MH⁻); and [α]_{D}=+3.8° (c=0.1, methanol)

Preparation of Starting Materials

a) 1-[2-(tert-Butoxycarbonyl)-4-pentyl]-cyclopentane Carboxylic Acid

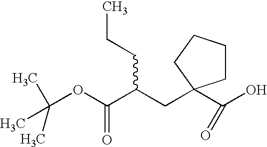

A mixture of 1-[2-(tert-butoxycarbonyl)-4-pentenyl]-cyclopentane carboxylic acid (EP 274234) (23 g, 81.5 mmol) and 10% palladium on charcoal (2 g) in dry ethanol (200 ml) was hydrogenated at 30 psi and room temperature for 18 hours. The reaction mixture was filtered through Arbocel®, and the filtrate evaporated under reduced pressure to give a yellow oil. The crude product was purified by column chromatography on silica gel, using ethyl acetate:pentane (40:60) as the eluant, to provide the desired product as a clear oil, 21 g, 91%; ¹H NMR (CDCl₃, 0.86 (t, 3H), 1.22-1.58 (m, 15H), 1.64 (m, 4H), 1.78 (dd, 1H), 2.00-2.18 (m, 3H), 2.24 (m, 1H); LRMS: m/z 283 (M−H)⁻

b) Tert-Butyl 2-[(1-{[(5-ethyl-1,3,4-thiadiazol-2-yl)amino]carbonyl}-cyclopentyl)methyl]pentanoate.

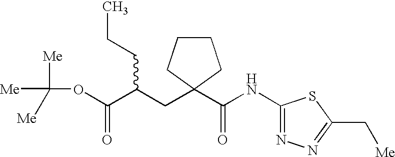

1-(3-Dimethylaminopropyl)-3-ethylcarbodiimide hydrochloride (0.21 mmol), 1-hydroxybenzotriazole hydrate (0.2 mmol), N-methylmorpholine (0.31 mmol) and 2-amino-5-ethyl-1,3,4-thiadiazole (0.22 mmol) were added to a solution of the product from stage a) above (150 mg, 0.53 mmol) in N,N-dimethylformamide (3 ml), and the reaction stirred at 90° C. for 18 hours. The cooled solution was diluted with ethyl acetate (90 ml), washed with water (3×25 ml), and brine (25 ml), then dried (MgSO₄) and evaporated under reduced pressure. The crude product was purified by chromatography on silica gel, using ethyl acetate:pentane (30:70) as the eluant to afford the title compound, 92%; ¹H NMR (CDCl₃, 300 MHz) δ: 0.82 (t, 3H), 1.20-1.80 (m, 22H), 1.84 (m, 1H), 2.20 (m, 4H), 3.04 (q, 2H), 9.10 (bs, 1H); LRMS : m/z 396.2 (MH⁺).

c) 2-1(1-1(5-ethyl-1,3,4-thiadiazol-2-yl)amino]carbonyl}cyclopentyl) methyl]pentanoic Acid.

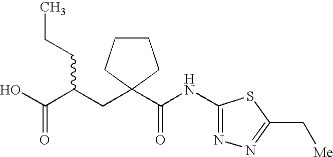

Trifluoroacetic acid (5 ml) was added to a solution of the title product from stage b) above (0.31 mmol) in dichloromethane (5 ml), and the solution stirred at room temperature for 4 hours. The reaction mixture was concentrated under reduced pressure and the residue azeotroped with toluene and dichloromethane to afford the title compound as a clear oil, 81%, ¹H NMR (CDCl₃, 400 MHz) δ: 0.92 (t, 3H), 1.35 (t, 3H), 1.25-1.80 (m, 11H), 2.20-2.50 (m, 4H), 2.95 (q, 2H), 12.10 (bs, 1H); LRMS m/z 339.8 (MH⁺); Anal. Found: C, 56.46; H, 7.46; N, 12.36. C₁₆H₂₅N₃O₃S requires C, 56.62; H, 7.44; N, 12.37%.

Details on a suitable assay system for identifying and/or studying an I:NEP are presented in the hereinafter in the section entitled NEP Assay. Further examples of NEP inhibitors are disclosed and discussed in the following review articles:

Pathol. Biol., 46(3), 1998, 191.

Current Pharm. Design, 2(5), 1996, 443.

Biochem. Soc. Trans., 21(3), 1993, 678.

Handbook Exp. Pharmacol., 104/1, 1993, 547.

TiPS, 11, 1990, 245.

Pharmacol. Rev., 45(1), 1993, 87.

Curr. Opin. Inves. Drugs, 2(11), 1993, 1175.

Antihypertens. Drugs, (1997), 113.

Chemtracts, (1997), 10(11), 804.

Zinc Metalloproteases Health Dis. (1996), 105.

Cardiovasc. Drug Rev., (1996), 14(2), 166.

Gen. Pharmacol., (1996), 27(4), 581.

Cardiovasc. Drug Rev., (1994), 12(4), 271.

Clin. Exp. Pharmacol. Physiol., (1995), 22(1), 63.

Cardiovasc. Drug Rev., (1991), 9(3), 285.

Exp. Opin. Ther. Patents (1996), 6(11), 1147.

Yet further examples of NEPi's are disclosed in the following documents:

EP-509442A

US-192435

US-4929641

EP-599444B

US-884664

EP-544620A

US-798684

J. Med. Chem. 1993, 3821.

Circulation 1993, 88(4), 1.

EP-136883

JP-85136554

US-4722810

Curr. Pharm. Design, 1996, 2, 443.

EP-640594

J. Med. Chem. 1993, 36(1), 87.

EP-738711-A

JP-270957

CAS # 115406-23-0

DE-19510566

DE-19638020

EP-830863

JP-98101565

EP-733642

WO9614293

JP-08245609

JP-96245609

WO9415908

JP05092948

WO-9309101

WO-9109840

EP-519738

EP-690070

J. Med. Chem. (1993), 36, 2420.

JP-95157459

Bioorg. Med. Chem. Letts., 1996, 6(1), 65.

Further I:NEPs are disclosed in the following documents:

EP-A-0274234

JP-88165353

Biochem.Biophys.Res. Comm.,1989, 164, 58

EP-629627-A

US-77978

Perspect. Med. Chem. (1993), 45.

EP-358398-B

Further examples of I:NEPs are selected from the following structures:

| | | Mode of Action |
|---|---|---|
| Compound | Structure | References |
| FXII | 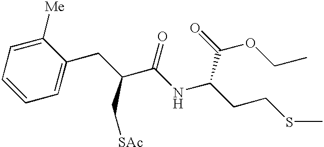 | I:NEP EP-509442A US-192435 US-4929641 |
| FXIII | 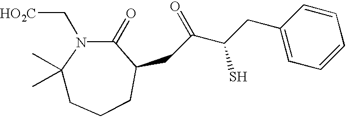 | I:NEP (also an ACE inhibitor) EP-599444B US-884664 |
| FXIV | 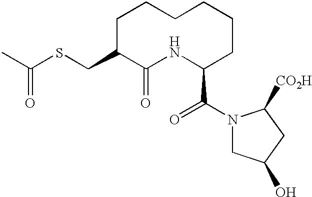 | I:NEP EP-544620A US-798684 J. Med. Chem. 1993, 3821. |
| FXV | 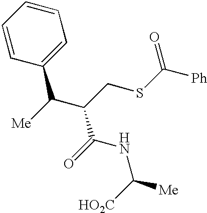 | I:NEP (also an ACE inhibitor) Mixanpril Circulation 1993, 88(4), 1. |
| FXVI | 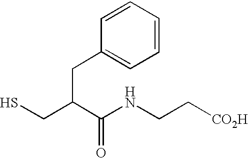 | I:NEP EP-136883 JP-85136554 US-4722810 |
| FXVII | 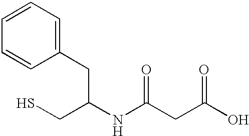 | I:NEP Retrothiorphan Curr. Pharm. Design, 1996, 2, 443. |
| FXVIII | 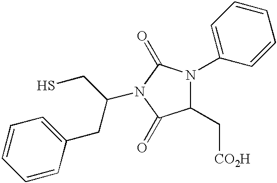 | I:NEP (also an ACE inhibitor) EP-640594 |
| FXIX | 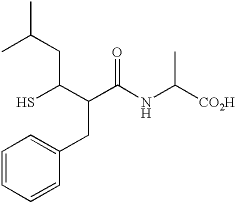 | I:NEP J. Med. Chem. 1993, 36(1), 87. |
| FXX | 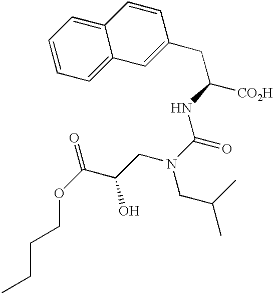 | I:NEP (also an ACE inhibitor) EP-738711-A JP-270957 |
| FXXI | 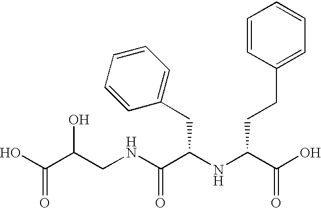 | I:NEP CAS #115406-23-0 |
| FXXII | 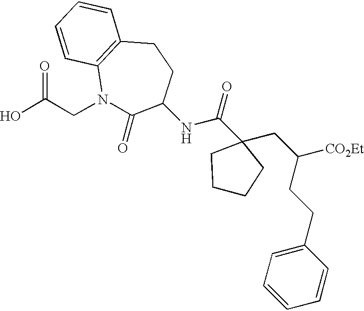 | I:NEP (also an ECE inhibitor) DE-19510566 DE-19638020 EP-830863 JP-98101565 |
| FXXIII | 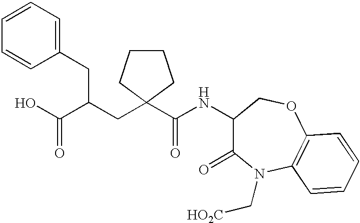 | I:NEP (also an ECE inhibitor) EP-733642 |
| FXXIV | 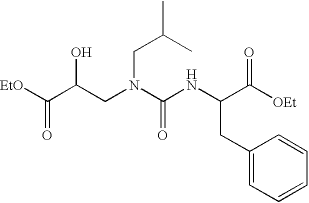 | I:NEP WO96/14293 |
| FXXV | 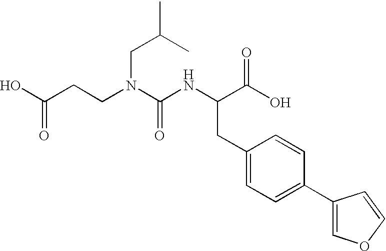 | I:NEP JP-08245609 JP-96245609 |
| FXXVI | 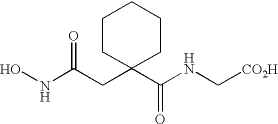 | I:NEP WO9415908 |
| FXXVII | 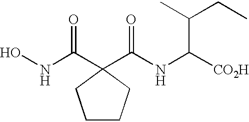 | I:NEP JP05092948 |
| FXXVIII | 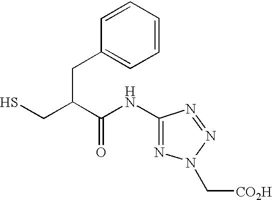 | I:NEP WO-9309101 |
| FXXIX | 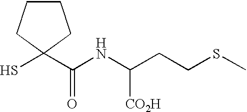 | I:NEP WO-9109840 |
| FXXXI | 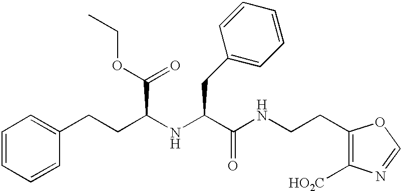 | I:NEP EP-519738 EP-690070 |
| FXXXII | 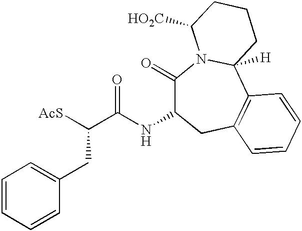 | I:NEP (also an ACE inhibitor) J. Med. Chem. (1993), 36, 2420. |
| FXXXIII | 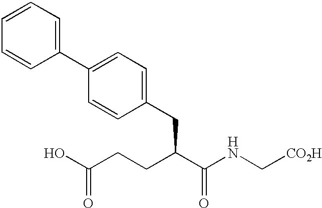 | I:NEP JP-95157459 Bioorg. Med. Chem. Letts., 1996, 6(1), 65. |

Preferred additional I:NEPs are selected from the following structures:

| | | Mode of Action |
|---|---|---|
| Compound | Structure | References |
| FV | 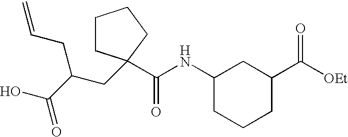 | I:NEP EP-A-0274234 (Example 300) |
| FVI | 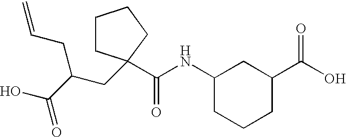 | I:NEP EP-A-0274234 (Example 379) |
| FVII | 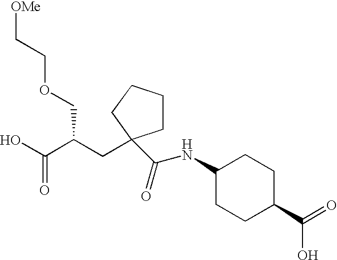 | I:NEP Candoxatrilat EP-274234 JP-88165353 Biochem.Biophys.Res. Comm., 1989, 164, 58 |
| FVIII | 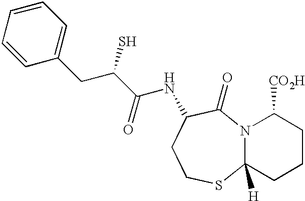 | I:NEP Omapatrilat (also an inhibitor of ACE) EP-0629627-A US-77978 |
| FIX | 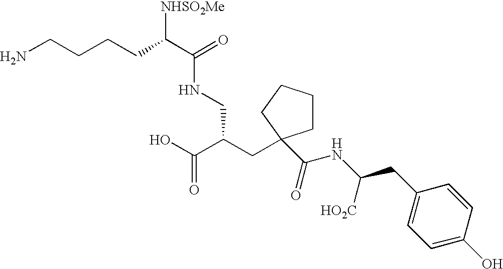 | I:NEP Sampatrilat (also an inhibitor of ACE) Perspect. Med. Chem. (1993), 45. EP-0358398-B |
| FX | 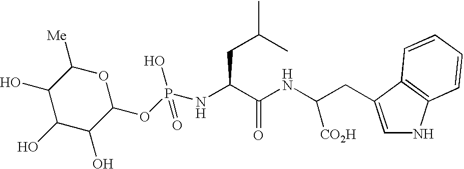 | I:NEP Phosphoramidon (which is commercially available) |
| FXI | 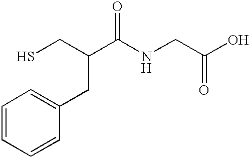 | I:NEP Thiorphan (which is commercially available) |

More preferred additional I:NEPs are selected from the following structures:

| COMPOUND | STRUCTURE |
|---|---|
| F57 | 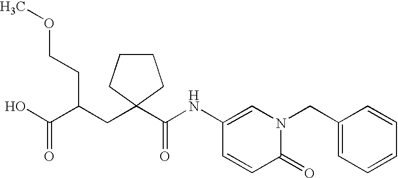 |
| F58 | 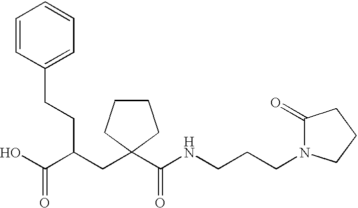 |
| F59 | 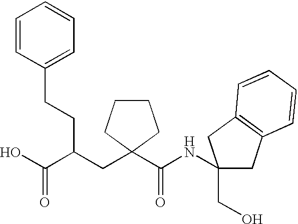 |
| F60 | 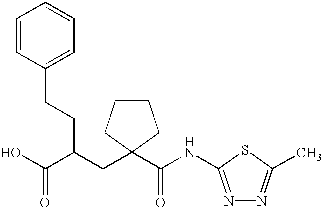 |
| F61 | 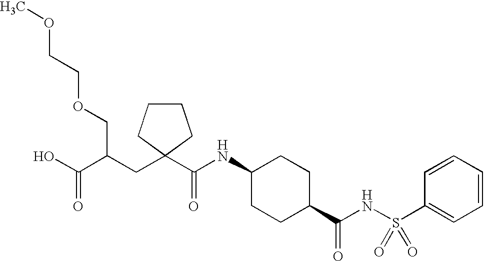 |
| F62 | 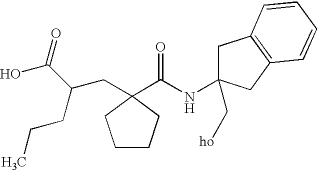 |
| F63 | 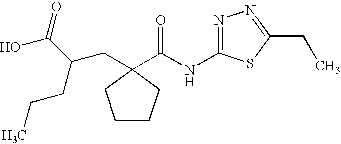 |
| F64 | 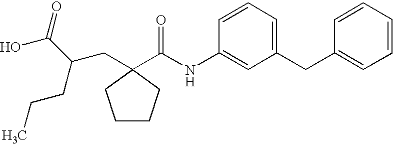 |
| F65 | 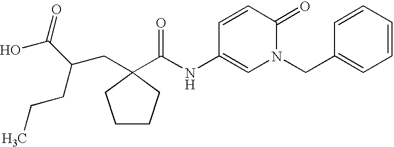 |
| F66 | 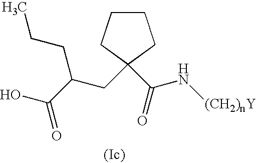 |

Bioavailability

Preferably, the compounds of the invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCo₂ add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600.

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stablity in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226.

By cross reference herein to compounds contained in patents which can be used in accordance with invention, we mean the therapeutically active compounds as defined in the claims (in particular of claim 1) and the specific examples (all of which is incorporated herein by reference).

How the invention may be put into effect will now be described, by way of example only, with reference to the following examples, some of which are preparative and others of which describe results of biological tests.

### EXAMPLE 1

Effect of (S) 3-(1H-Indol-3-yl)-N-[1-(5-methoxy-pyridin-2-yl)-cyclohexyl-methyl]-2-methyl-2-[3-(4-nitro-phenyl)-ureido]-propionamide (Compound (1)) on Female Rat Sexual Proceptivity

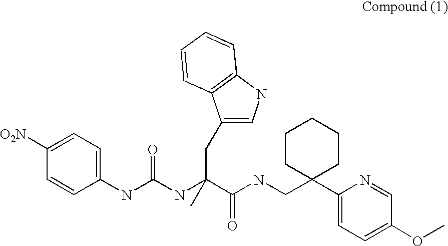

Ovariectomised adult female Sprague Dawley rats (180-200 g, from Charles River) were housed in groups of 6 in a reversed lighting system of 12 h light:dark (lights off 7.00-19.00 h). Two weeks after ovariectomy they were used for sexual activity tests. The experiments started at least 5 h into the dark period.

Tests were carried out in a circular arena of 90 cm diameter, surrounded by a 30 cm high wall. Two small cages with wire-mesh front (15×15 cm) are fixed into the wall such that the front of the cage is <<flush>> with the wall and the 2 cages are opposite each other. They contained two stimuli animals: an intact sexually experienced male and a receptive female (ovariectomised, primed with 5 μg oestradiol benzoate dissolved in corn oil and injected subcutaneously 48 hours before the test and with 0.5 mg of progesterone four hours before the test). Sexually naive test and control animals were used. Forty eight hours before the tests, both the test and control animals were primed with 5 μg oestradiol benzoate. For animals used as positive controls, progesterone (0.5 mg/0.1 ml) was dissolved in corn oil and administered subcutaneously (s.c.), 4 h before the test. Test and control animals were introduced one at a time for 10 minute periods into the arena. During the 10 min test, the time that the test or positive control animal spent investigating each stimulus animal was noted. The arena was thoroughly cleaned between animals. The position of the male/female stimuli boxes was randomised between animals, in order to avoid place preference. The difference in the percentage of time spent investigating the male minus the female stimuli was calculated, out of the total time spent investigating stimuli animals.

Compound (1) was dissolved in 100% β-cyclodextrin and then diluted with saline to a final solution of 50% 2-hydroxypropyl-β-cyclodextrin. It was administered intraperitoneally (i.p.) at doses of 3 and 10 mg/kg, in a dosing volume of 1 ml/kg, 1 h before tests. Progesterone (0.5 mg/0.1 ml) was dissolved in corn oil and administered subcutaneously (s.c.), 4 h before test, as a positive control.

Compound (1) dose-dependently (3 mg/kg-10 mg/kg) increased the percentage of time spent investigating the male stimulus, with a MED of 10 mg/kg (see FIG. 1). The effect of this dose was similar to the effect of progesterone (prog). (*P<0.05, **P<0.01 Kruskal-Wallis followed by Mann-Whitney test, vs vehicle).

### EXAMPLE 2

Effect of Compound (1) on Female Rat Sexual Receptivity

Ovariectomised adult female Sprague Dawley rats (180-200 g, from Charles River) were housed in groups of 6 in a reversed lighting system of 12 h light:dark (lights off 7.00-19.00 h). Two weeks after ovariectomy they were used for sexual activity tests. The experiments started at least 5 h into the dark period.

Compound (1) was dissolved in 100% β-cyclodextrin and then diluted with saline to a final solution of 50% 2-hydroxypropyl-β-cyclodextrin. It was administered intraperitoneally (i.p.) at a dose of 10 mg/kg, in a dosing volume of 1 ml/kg. Quinelorane (6.25 μg/kg) was dissolved in water and administered s.c. as positive control. Forty eight hours before testing, ovariectomised female rats (as described above), were primed with 5 μg oestradiol benzoate dissolved in corn oil and injected subcutaneously. This is a low dose of oestrogen that does not re-establish sexual behaviour in an ovariectomised female but provides a minimum hormonal background for pharmacological agents to stimulate sexual behaviour. The females were placed with a series of vigorous male rats and subjected to 10 mounts.

The lordotic response of the animal was recorded and expressed as a percentage of the mounts (i.e. lordosis quotient, LQ), as previously described. Animals showing LQ<20 were considered non-receptive and were included in the study. Each rat was tested prior to administration of the compound and then tested similarly post-injection. The pre-treatment times were 1 h for Compound (1) and vehicle (50% β-cyclodextrin, i.p.) or 90 min for quinelorane.

As shown in FIG. 2, a single administration of quinelorane (6.25 μg/kg, s.c.) significantly (P<0.01) increased the LQ, 90 min after administration, compared to the LQ shown before administration (paired t test). A single administration of Compound (1) (10 mg/kg, i.p.) also had a significant (P<0.05) stimulatory effect on the LQ, 1 h after administration, compared to the LQ shown before administration (paired t test).

### EXAMPLE 3

The Effect of Repeated Administration of Compound (1) on Female Rat Proceptivity

In the present study we have investigated whether the repeated administration of a higher dose of Compound (1) (15 mg/kg) still results in stimulation of proceptivity.

Ovariectomised adult female Sprague Dawley rats (180-200 g) were housed in groups of 5 in a reversed lighting system of 12 h light:dark (lights off 5.00-17.00 h). They were used for the experiments at least two weeks after ovariectomy. Forty eight hours before tests, the animals were primed with oestradiol benzoate (5 μg/0.0 ml in corn oil, s.c.). On day 1, progesterone (0.5 mg/0.1 ml, in corn oil, s.c.) was administered to one of the groups 4 h before tests, as a positive control. Compound (1) (15 mg/kg, i.p.) was administered in 50% 2-hydroxypropyl-β-cyclodextrin, 1 h before tests. The test lasted 10 minutes and was carried out as described before. The difference in the percentage of time spent investigating the male minus the female stimuli was calculated, out of the total time spent investigating stimuli animals. Animals were submitted to a test on day 1 and on day 15. From day 2 to 14 the Compound (1) group received a daily injection of the compound (15 mg/kg, i.p.), while both the vehicle and the progesterone groups received an injection of vehicle. On day 15 the test took place again, as described for day 1.

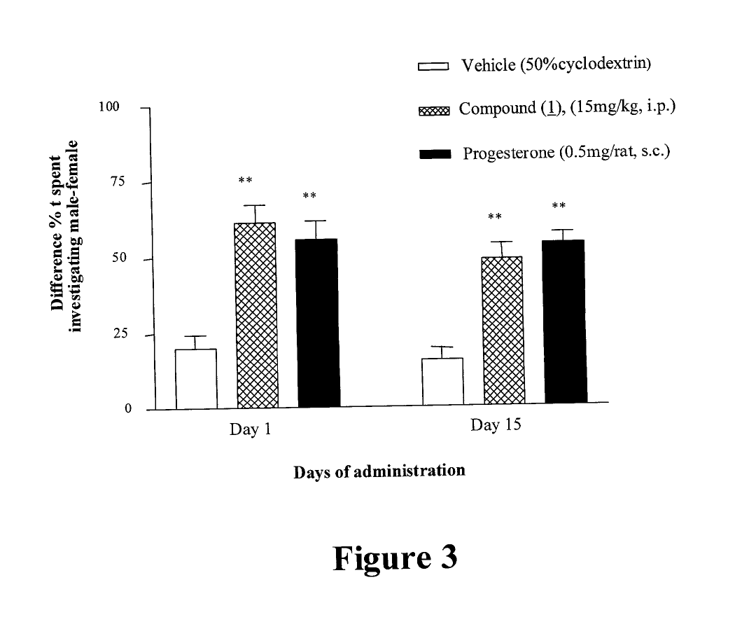

On day 1, both progesterone and Compound (1) had a stimulatory effect on proceptivity, compared to the vehicle group (**P<0.01, ANOVA followed by Dunnett's test). On day 15, a similar stimulatory effect was observed (**P<0.01, ANOVA followed by Dunnett's test) (see FIG. 3). No significant difference was observed between the effects on day 1 and day 15 for each treatment group (paired t test). The effects of progesterone and Compound (1) were statistically similar. There were no changes in body weight or general behaviour between groups along the experiment.

From this study we can conclude that Compound (1) (15 mg/kg, i.p.) has a stimulatory effect on proceptivity in the female rat, comparable to progesterone, and that such effect is unaffected by the repeated administration of the compound, which seems to be well tolerated.

### EXAMPLE 4

Effect of Intracerebroventricular Administration of Compound (1) on Female Rat Sexual Proceptivity

In order to elucidate the site of action for this effect we have administered the Compound (1) intracerebroventricularly (i.c.v.).

Ovariectomised female rats (Sprague Dawley, obtained from Charles River, UK) were stereotaxically implanted (coordinates 0.89 mm behind Bregma, 1.3 mm lateral and 2.5 mm vertical) with stainless steel cannulae (6 mm long, O.D. 0.75 mm), held in place with dental cement. Animals were housed in groups of three and returned to a reversed lighting system of 12 h light:dark (lights off 5.00-17.00 h). Correct placement of the cannulae was assessed post-mortem. Rats were used for tests two weeks after ovariectomy (one week after cannulation). The experiments started at least 5 h into the dark period. Forty eight hours before tests, the animals were primed with 5 μg oestradiol benzoate (s.c, in corn oil) and adapted to the apparatus (in the absence of stimuli animals) for 10 min on 2 consecutive days prior testing. The 10 min test was carried out as previously described. The difference in the percentage of time spent investigating the male minus the female stimuli was calculated, out of the total time spent investigating stimuli.

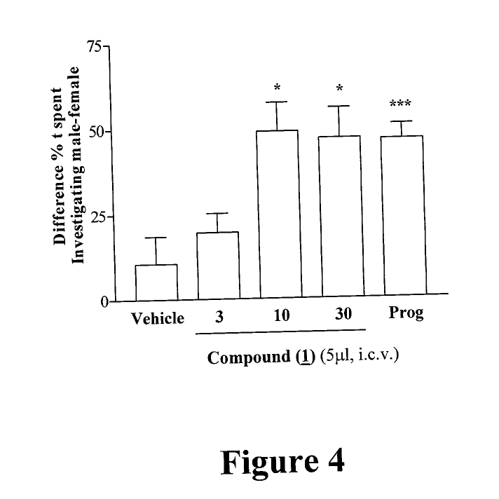

Compound (1) was dissolved in 50% 2-hydroxypropyl-β-cyclodextrin in saline. It was administered i.c.v. over a 30 sec period, with the aid of a pump set to deliver a flow of 10 μl/min. The dosing volume was 5 μl/rat. The compounds were administered 10 min before tests. Progesterone (0.5 mg/0.1 ml) was dissolved in corn oil and administered subcutaneously (s.c.), 4 h before test, as a positive control. As shown in FIG. 4, Compound (1) dose-dependently (3-30 μg/rat) increased the percentage of time spent investigating the male stimulus, with a MED of 10 μg. The effect of this dose was similar to the effect of progesterone.

From this study we can conclude that the effect of Compound (1) on female sexual proceptivity is centrally mediated.

In FIG. 4 bars represent percentage of time spent investigating male, minus the percentage of time spent investigating the female stimuli±SEM, (n=7-8 per group). *P<0.05, **P<0.01 vs vehicle (Kruskal-Wallis ANOVA test followed by Mann-Whitney's test).

### EXAMPLE 5

Inhibitory Effect of NMB on Female Rat Sexual Proceptivity and Antagonism of this Effect by Compound (1)

We have investigated the potentially inhibitory effect of the BB₁ agonist neuromedin B (NMB) on female rat sexual proceptivity.

Ovariectomised female rats (Sprague Dawley, obtained from Charles River, UK) were stereotaxically implanted (coordinates 0.89 mm behind Bregma, 1.3 mm lateral and 2.5 mm vertical) with stainless steel cannulae (6 mm long, O.D. 0.75 mm), held in place with dental cement. Animals were housed in groups of three and returned to a reversed lighting system of 12 h light:dark (lights off 5.00-17.00 h). Correct placement of the cannulae was assessed post-mortem. Rats were used for tests two weeks after ovariectomy (one week after cannulation). The experiments started at least 5 h into the dark period. Forty eight hours before tests, the animals were primed with 5 μg oestradiol benzoate (OB) (s.c, in corn oil) and adapted to the apparatus (in the absence of stimuli animals) for 10 min on 2 consecutive days prior testing. The 10 min test was carried out as previously described. The difference in the percentage of time spent investigating male minus female was calculated, out of the total time spent investigating stimuli.

Progesterone (Prog, 0.5 mg/0.1 ml) was dissolved in corn oil and administered subcutaneously (s.c.), 4 h before test, to induce proceptive behaviour. Compound (1) (15 mg/kg, i.p.) was dissolved in 50% 2-hydroxypropyl-β-cyclodextrin in saline and administered 1 h before the i.c.v. administration. Neuromedin B was obtained from Bachem, UK. It was dissolved in isotonic saline and administered i.c.v. over a 30 sec period, with the aid of a pump set to deliver a flow of 10 μl/min, 10 min before tests. The dosing volume was 5 μl/rat. Each rat received a total amount of 100 ng.

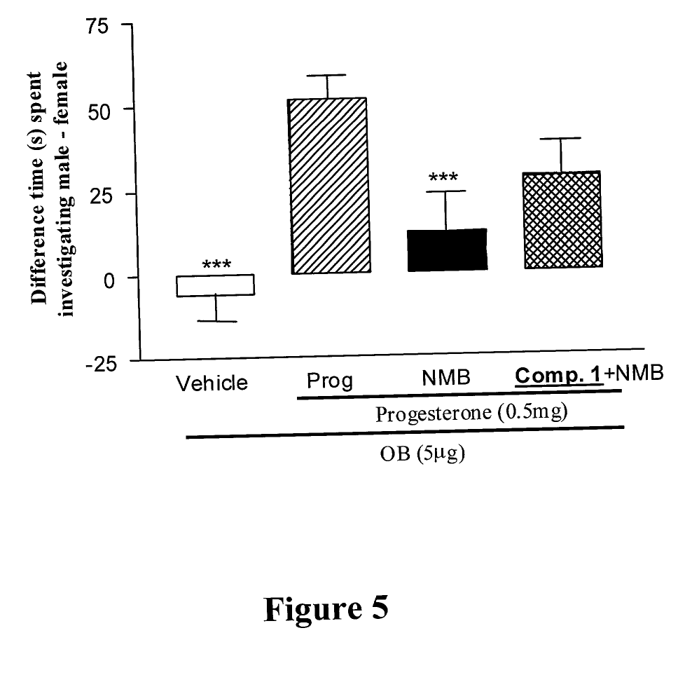

As shown in FIG. 5, progesterone (Prog) increased the percentage of time spent investigating the male stimulus, compared to the vehicle group, thus showing stimulation of proceptive behaviour. NMB (100 ng, i.c.v.) significantly reduced proceptivity in progesterone-treated rats. Moreover, pre-treatment with Compound (1) which acts as an antagonist (15 mg/kg, i.p.) prevented the inhibitory effect of NMB. However, the blockade obtained with the dose of Compound (1) used was not total.

From the present study we can conclude that stimulation of BB1 receptors with an agonist results in inhibition of proceptive behaviour. This inhibitory effect may be prevented by the presence of an antagonist. e.g. Compound (1) In FIG. 5 the bars represent percentage of time spent investigating male, minus the percentage of time spent investigating the female stimuli±SEM, (n=8-12 per group). ***P<0.001 vs progesterone (One-way ANOVA followed by Dunnett's test).

### EXAMPLE 6

Demonstration that the Effect of Compound (1) on Female Sexual Behaviour is Not Mediated Through Sexual Hormones

Previous examples have shown that Compound (1) (nanomolar affinity “mixed” BB₁/BB₂ receptor antagonist) has a dose-dependent stimulatory effect on sexual activity in the female rat, both on proceptivity and receptivity. Although the animals used in that study were ovariectomised, and therefore steroid hormones release can not be expected to occur in response to the compound, there is a possibility that the adrenal glands might secrete steroid hormones in response to Compound (1). If that was the case, the mediation of the stimulatory effects by progesterone would be relevant for rodents, but it would not be the case for primates. In the present study, we have investigated the potential effect of the bombesin receptor antagonist Compound (1) on secretion of progesterone. Oestradiol and pituitary hormones (Luteinising hormone (LH), follicle stimulating hormone (FSH) and prolactin) have also been analysed in the same animals.

Ovariectomised adult female Sprague Dawley rats (180-200 g) were housed in groups of 6 in a reversed lighting system of 12 h light:dark (lights off 7.00-19.00 h). They were used for the experiments at least two weeks after ovariectomy. Forty eight hours before tests, the animals were primed with oestradiol benzoate (5 μg/0.1 ml in corn oil, s.c.). Progesterone (0.5 mg/0.1 ml, in corn oil, s.c.) was administered 4 h before blood collection, as a positive control. Compound (1) (3-10 mg/kg, i.p.) was administered in 50% 2-hydroxypropyl-β-cyclodextrin, 1 h prior to blood collection. Blood was collected from the trunk, after decapitation. It was immediately centrifuged (3500 r.p.m., 4° C., 5 min) and the plasmas frozen until assayed for hormonal content, using commercially available radioimmunoassay kits (¹²⁵I-labelled hormones) for oestradiol, progesterone, LH, FSH and prolactin.

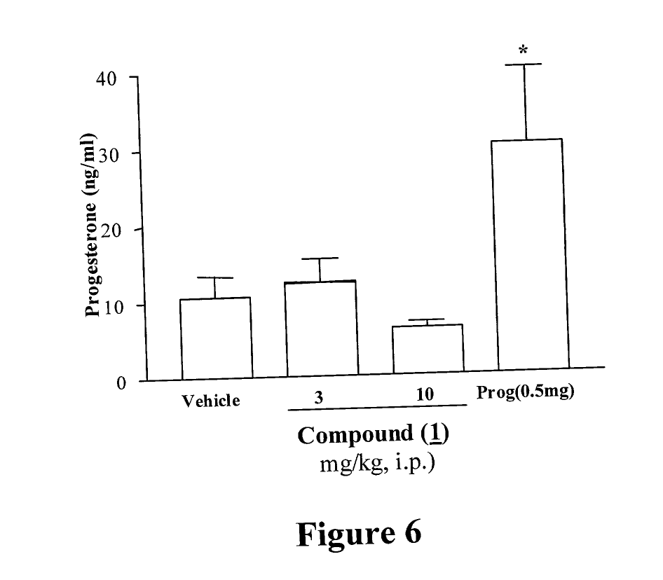

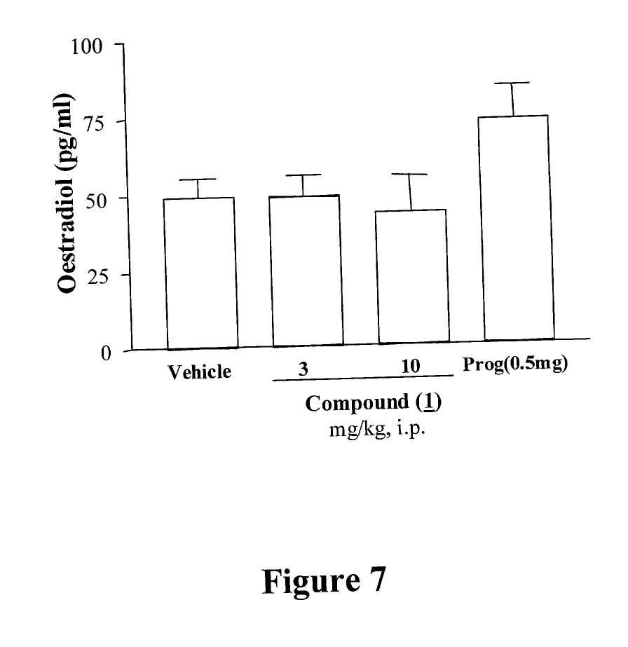

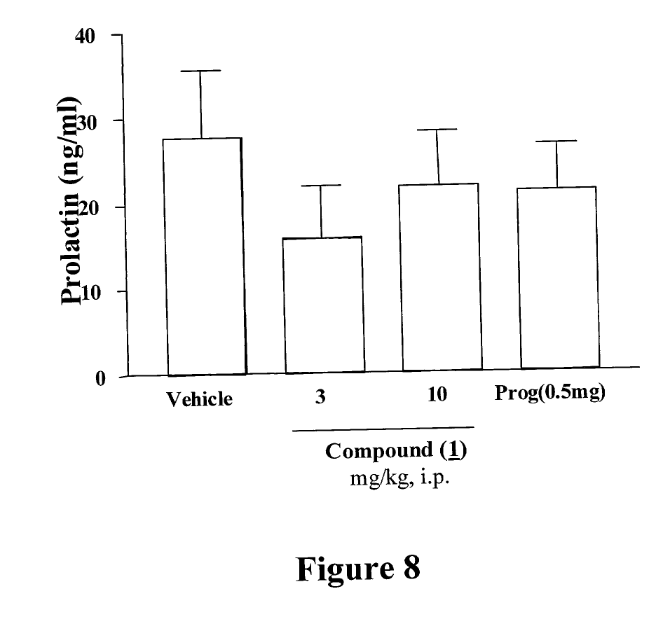

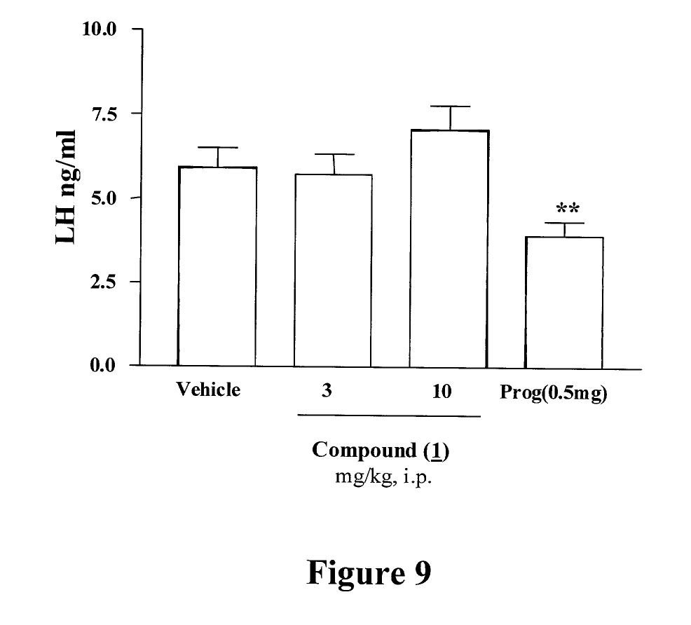

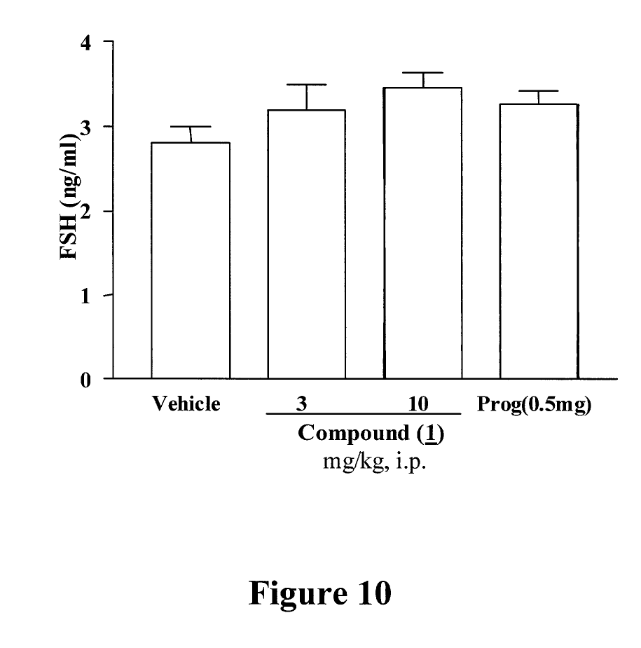

A single administration of progesterone resulted in a significant increase in the progesterone plasma levels (P<0.05), and a significant decrease in LH plasma levels (P<0.01), compared to animals injected with vehicle (Kruskal-Wallis followed by Mann-Whitney test). However, Compound (1) (3-10 mg/kg, i.p.) had no effect on the plasma levels of progesterone (FIG. 6, where animals were pre-treated with 5 g oestradiol benzoate, s.c., 48 h before the test. They were tested 1 h or 4 h post-injection of Compound (1) (3-10 mg/kg, p.o.) or progesterone (0.5 mg/0.1 ml, s.c.) respectively. Values represent mean±SEM, (n=9 per group). *P<0.05, vs vehicle (Kruskal-Wallis followed by Mann-Whitney test, vs vehicle)), oestradiol (FIG. 7, where animals were pre-treated with 5 μg oestradiol benzoate, s.c., 48 h before the test. They were tested 1 h or 4 h post-injection of Compound (1) (3-10 mg/kg, p.o.) or progesterone (0.5 mg/0.1 ml, s.c.) respectively. Values represent mean±SEM, (n=6-7 per group)), prolactin (FIG. 8, where animals were pre-treated with 5 μg oestradiol benzoate, s.c., 48 h before the test. They were tested 1 h or 4 h post-injection of Compound (1) (3-10 mg/kg, p.o.) or progesterone (0.5 mg/0.1 ml, s.c.) respectively. Values represent mean±SEM, (n=10 per group)) LH (FIG. 9, where animals were pre-treated with 5 μg oestradiol benzoate, s.c., 48 h before the test. They were tested 1 h or 4 h post-injection of Compound (1) (3-10 mg/kg, p.o.) or progesterone (0.5 mg/0.1 ml, s.c.) respectively. Values represent mean±SEM, (n=10 per group). **P<0.01, vs vehicle (Kruskal-Wallis followed by Mann-Whitney test, vs vehicle)) or FSH (FIG. 10, where animals were pre-treated with 5 μg oestradiol benzoate, s.c., 48 h before the test. They were tested 1 h or 4 h post-injection of Compound (1) (3-10 mg/kg, p.o.) or progesterone (0.5 mg/0.1 ml, s.c.) respectively. Values represent mean±SEM, (n=10 per group).

From this experiment we can conclude that Compound (1) did not have an effect on the secretion of sexual hormones, thus suggesting that the effects of the compound on female sexual activity must be mediated by different mechanisms, maybe involving neurotransmitters.

### EXAMPLE 7

Effect of Compound (1) on the Sexual Behaviour of Normal Male Rats

The potentially stimulatory effect of Compound (1) on male sexual behaviour has been tested on sexually vigorous rats. Sprague Dawley male rats (Charles River, UK) were kept, 4 rats per cage, in a reversed lighting regime (12:12 hours, lights off at 5.00 h), with free access to food and water. The rats were pre-selected by being presented with a receptive female at 4 days intervals, i.e. every third day (having 2 clear days between presentations) until completing 6-7 days of baseline determination. The animals showing consistently vigorous behaviour (ejaculatory latencies <300s) were chosen for further experiments (n—24). Animals were randomised into three groups. All animals received all three treatments following a latin-square design. Treatments were administered once a week, with a baseline test in between treatments (4 days intervals between baseline and test day). Treatments were Compound (1) (15 mg/kg, dissolved in 50% 2-hydroxypropyl-β-cyclodextrin in saline), vehicle, or Fluoxetine (20 mg/kg dissolved in 100% DMSO). All treatments were administered i.p. in a 1 ml/kg volume, 1 h before tests.

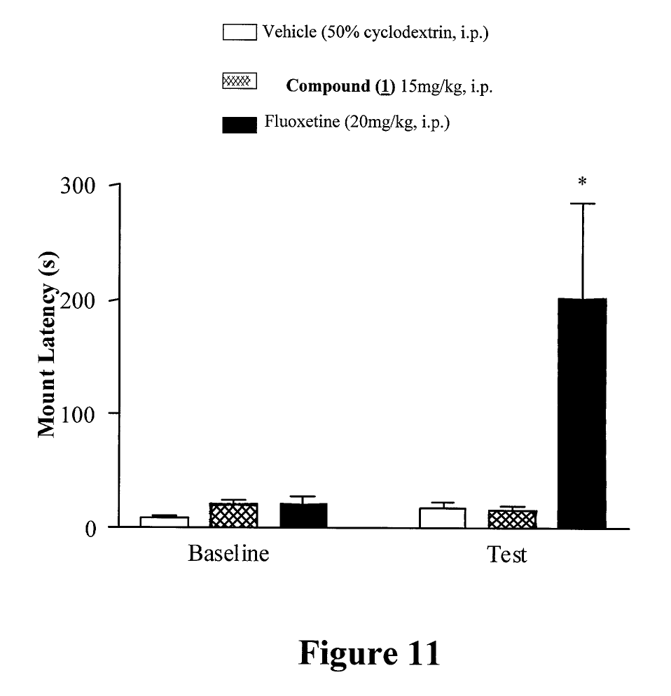

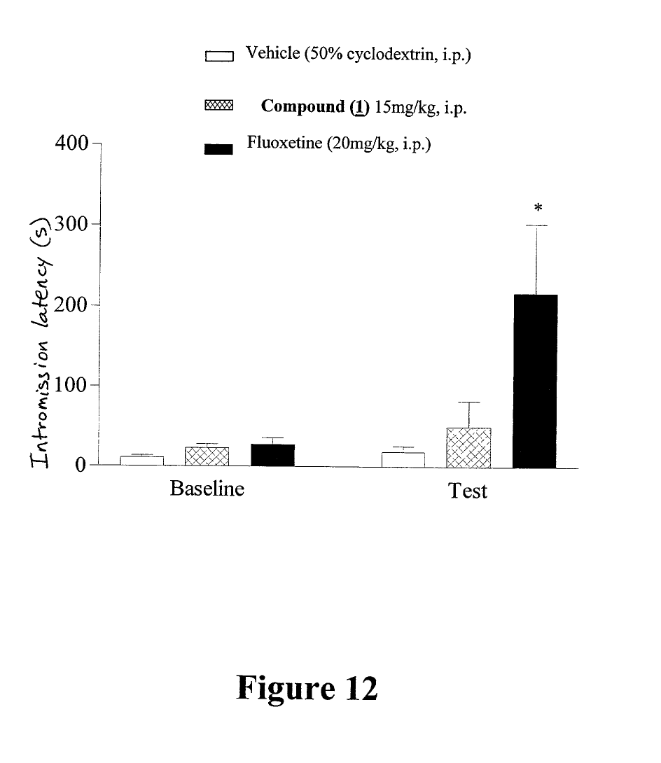

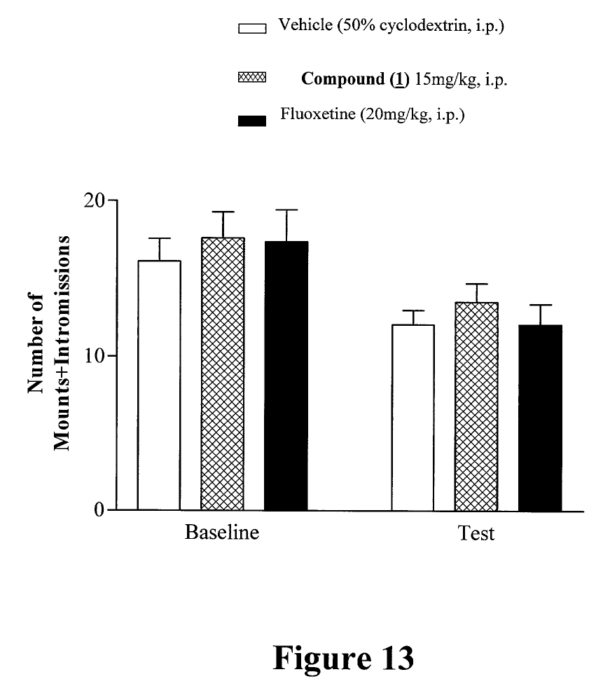

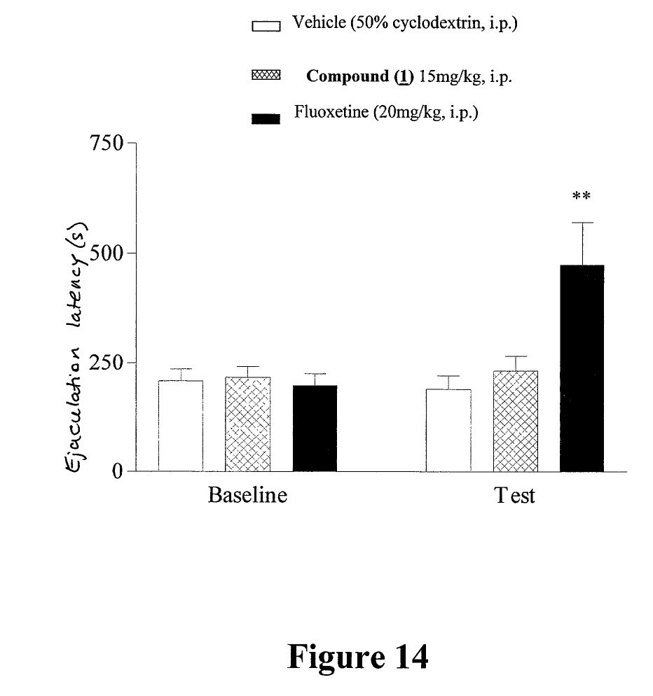

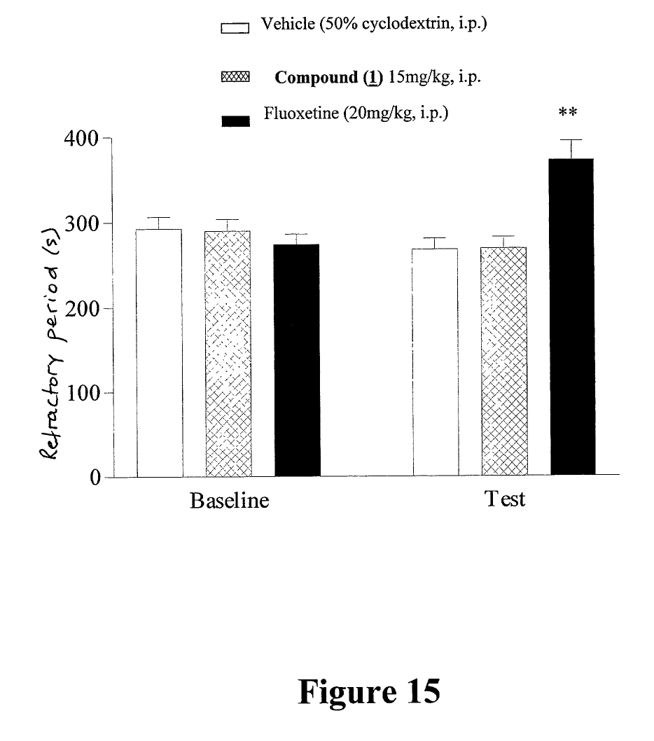

For all the sexual behaviour tests, the males were placed in an observation arena (50-60 cm diameter), starting 5 hours into the dark cycle and observed under red illumination. Three to 4 minutes after placing the male in the arena, a receptive female (ovariectomised, bearing a 7 mm Silastic implant of oestradiol benzoate) was introduced to the arena and the following parameters noted: Mount Latency: time (in seconds) taken between introduction of female and first mount. A maximum time of 15 minutes (900 seconds) was allowed, and the test terminated if no mounts were recorded within that time (FIG. 11), Intromission Latency: time (in seconds) taken between introduction of female and first intromission (FIG. 12), Number of Mounts: to reach ejaculation. When ejaculation was not reached, the number of mounts was not analyzed, Number of Intromissions: to reach ejaculation. When ejaculation was not reached, the number of intromissions was not analyzed (FIG. 13 is number of mounts+intromissions), Ejaculation Latency: time (in seconds) taken from first intromission to ejaculation. A maximum time of 30 minutes (1800 seconds) was given, and the test terminated if ejaculation was not achieved in that time (FIG. 14), and Refractory Period: time (in seconds) taken from ejaculation to the first mount of the next series of sexual activity. In those animals reaching ejaculation the test was terminated at the end of the refractory period, as indicated by the first mount of the next sexual cycle (FIG. 15).

A one-way ANOVA followed by Dunnett's t test was used to compare treated vs vehicle groups each day of testing, for all the sexual behaviour parameters. (*P<0.05, **P<0.01; n=15-16).

Mount latency and intromission latency were significantly increased in the fluoxetine-treated group compared to the vehicle group. Ejaculation latency and refractory period were also increased in this group, showing a decrease in sexual performance as well as the decreased arousal. No changes were seen in the number of mounts and intromissions required to achieve ejaculation. Unlike Fluoxetine, Compound (1) had no effect on any of the parameters studied, at a dose shown to be stimulatory in sexually dysfunctional males (see example 9). From the present study we can conclude that Compound (1) has no effect on sexual behaviour in sexually vigorous males.

### EXAMPLE 8

Effect of Compound (1) on the Sexual Behaviour of Sexually Dysfunctional Male Rats

Fluoxetine induces ejaculation delay, anorgasmy and loss of sexual desire in humans (Crenshaw and Goldberg, 1996). A model of male sexual dysfunction in the rat, induced by daily administration of fluoxetine until a significant detrimental effect on sexual behaviour (arousal and ejaculation) was established. The potentially stimulatory effect of Compound (1) on male sexual behaviour in these sexually dysfunctional male rats was examined. The effects of Compound (1) were compared to those of yohimbine. Preclinical and clinical studies suggest that yohimbine may be an effective treatment for sexual side-effects caused SSRI (Hollander, E., McCarley, A. (1993) J. Clin. Psychiatry 53:207-209. and Jacobsen).

Sprague Dawley male rats (Charles River, UK) were kept, 4 rats per cage, in a reversed lighting regime (12:12 hours, lights off at 5.00 h), with free access to food and water. The rats were pre-selected by being presented with a receptive female at 4 days intervals, i.e. every third day (having 2 clear days between presentations) until completing 6-7 trials of baseline determination. The animals showing consistently vigorous behaviour (ejaculatory latencies <300s) were chosen for further experiments. Animals were treated for 3 consecutive days with either vehicle (water) or fluoxetine (20 mg/kg, i.p., in a 2 ml/kg dosing volume). On the fourth day, the animals treated with water received vehicle (veh+veh) and the animals treated with fluoxetine received one of the three following treatments: Compound (1) (15 mg/kg, dissolved in 50% 2-hydroxypropyl-β-cyclodextrin in saline), vehicle (cyclodextrine), or yohimbine (2 mg/kg dissolved in water). All treatments were administered i.p. in a 1 ml/kg volume, 1 h before tests.

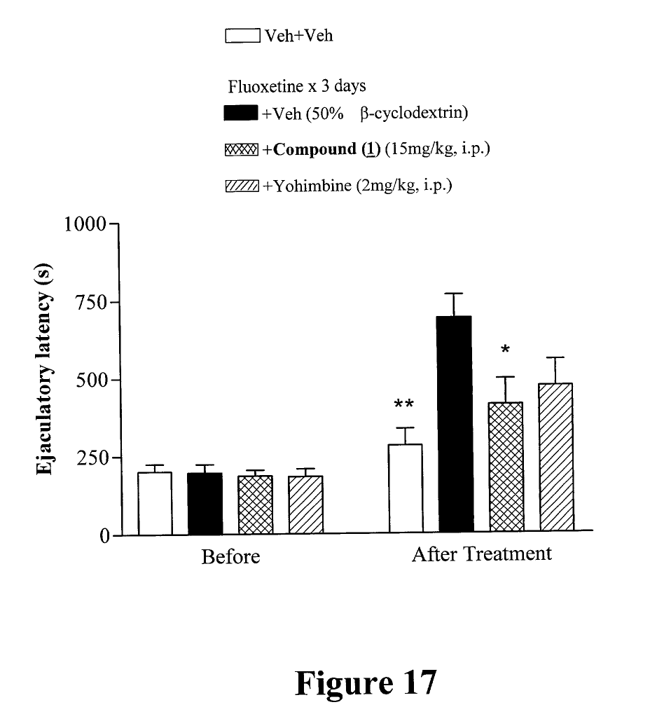

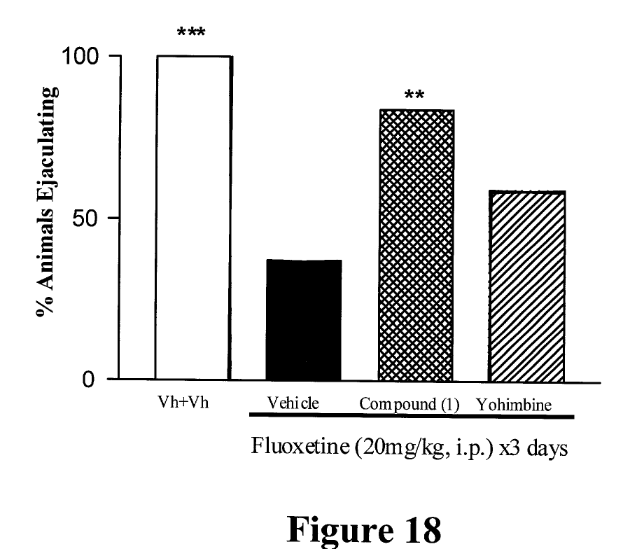

For all the sexual behaviour tests, the males were placed in an observation arena (50-60 cm diameter), starting 5 hours into the dark cycle and observed under red illumination. Three to 4 minutes after placing the male in the arena, a receptive female (ovariectomised, bearing a 7 mm Silastic implant of oestradiol benzoate) was introduced to the arena and the following parameters noted: Mount Latency: time (in seconds) taken between introduction of female and first mount. A maximum time of 15 minutes (900 seconds) was allowed, and the test terminated if no mounts were recorded within that time (FIG. 16), Ejaculation Latency: time (in seconds) taken from first intromission to ejaculation. A maximum time of 30 minutes (1800 seconds) was given, and the test terminated if ejaculation was not achieved in that time (FIG. 17), Percentage of males achieving ejaculation within 30 minutes was calculated (FIG. 18).

A one-way ANOVA followed by Dunnett's t test was used to compare the fluoxetine+vehicle group and other groups for mount and ejaculatory latencies. Percentage of animals ejaculating was analysed using a Chi-square test followed by Fisher's test. (*: P<0.05, **: P<0.01, ***: P<0.001; n=15-19).

Mount latency and ejaculation latency were significantly increased in the fluoxetine-treated groups compared to the vehicle+vehicle group, indicating a decrease in sexual desire as well as sexual performance in these groups. The number of animals ejaculating was significantly lower in the fluoxetine-treated groups, indicating anorgasmy. Compound (1) significantly decreased the mount and ejaculatory latencies at the same time as increasing the percentage of animals ejaculating in the animals rendered sexually dysfunctional by the fluoxetine treatment, to levels comparable to normal animals (veh+veh). Yohimbine followed a similar trend, although this did not reach significance.

From the present study we can conclude a stimulatory effect of Compound (1) on sexual behaviour in males suffering from sexual dysfunction, at the level of sexual desire, sexual performance and anorgasmy.

### EXAMPLE 9

(S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide

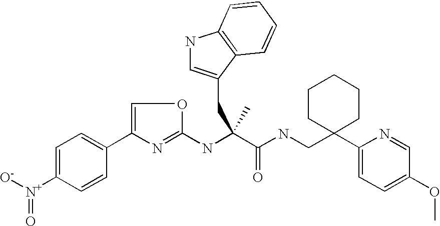

1. To a stirred solution of p-nitrophenylchloroformate (9.27 g, 46 mmol) in THF (200 ml) at 0° C. was added dropwise a solution of H-(S)-αMeTrp-OMe (1a) (10.7 g, 46 mmol) and triethylamine (6.4 ml, 46 mmol) in THF (100 ml) over 1 hour. Stirring was continued for a further 30 minutes at room temperature, after which aqueous ammonia (15 ml) was added. IR after 10 minutes indicated bands at 1732 and 1660 cm⁻¹. The THF was removed under reduced pressure, and the residue was taken UP in EtOAc and washed with 1N HCl (×2), Na₂CO₃ solution (until intense yellow colour subsided, ˜×8), brine, and dried (MgSO₄). The solvent was removed under reduced pressure to give 2a as a foam (110.3 g, 82%):MS m/e (AP+): 276.16 (M⁺+H, 100%);

MS m/e (AP−): 274.11 (M⁻−H, 100%);

IR (film): 3383, 1724, 1657, 1600, 1539, 1456, 1374, 1256, 1108, 743 cm⁻¹;

¹H NMR (CDCl₃): δ=1.70 (3H, s), 3.38 (1H, d, J=14.7 Hz), 3.59 (1H, d, J=14.7 Hz), 3.71 (3H, s), 4.22 (2H, s), 5.16 (1H, s), 6.99 (1H, d, J=2.2 Hz), 7.08-7.20 (2H, m), 7.34 (1H, d, J=8.1 Hz), 7.59 (1H, d, J=7.8 Hz), 8.09 (1H, s).

2. The urea (2a) (6.4 g, 23 mmol) and 2-bromo-1-(4-nitro-phenyl)-ethanone (6.0 g, 23 mmol) were stirred in toluene (500 ml)/dioxan (100 ml) and maintained under reflux for 30 hours, after which solvent was removed under reduced pressure and the residue was purified by chromatography using a 90 g Biotage cartridge. 10% EtOAc in heptane eluted the bromide starting material. 20% EtOAc eluted the desired product. Removal of solvent under reduced pressure gave 3a as a foam (840 mg, 9%):

MS m/e (ES+): 420.56 (M⁺, 100%);

IR (film): 3394, 1732, 1632, 1605, 1574, 1515, 1456, 1334, 1253, 1210, 1108, 1072, 940, 854, 734 cm⁻¹;

¹H NMR (CDCl₃): δ=1.91 (3H, s), 3.46 (1H, d, J=14.6 Hz), 3.69 (3H, s), 3.78 1H, d, J=14.6 Hz), 5.57 (1H, s), 6.89 (1H, d, J=2.2 Hz), 7.03-7.08 (1H, m), 7.14-7.18 (1H, m), 7.34 (1H, d, J=8.1 Hz), 7.41 (1H, d, J=8.1 Hz), 7.63 (1H, s), 7.85 (2H, d, J=9.0 Hz), 8.05 (1H, s), 8.24 (2H, d, J=8.6 Hz).

3. The ester (3a) (840 mg, 2 mmol) was dissolved in dioxan (50 ml) and LiOH.H₂O (336 mg, 8 mmol) in H₂O (25 ml) was added. The mixture was stirred vigorously overnight, and then neutralised with 1M HCl (8 ml, 8 mmol). The majority of the dioxan was removed under reduced pressure and the product was crystallised, filtered off, washed with water and dried under reduced pressure to give pure 4a (668 mg, 82%):

MS m/e (ES+): 407 (M⁺+H);

IR (film): 1633 cm⁻¹;

¹H NMR (DMSO-d₆) δ=1.49 (3H, s), 3.30-3.35 (1H, m, masked by H₂O), 3.59 (1H, d, J=14.7 Hz), 6.86-6.90 (1H, m), 6.99-7.03 (2H, m), 7.30-7.36 (2H, m), 7.48 (1H, s), 7.94 (2H, d, J=9.0 Hz), 8.27-8.30 (3H, m), 10.88 (1H, s), (CO₂H not seen). 4. The acid (4a) (1.148 g, 2.8 mmol), O-benzotriazol-1-yl-N,N,N′,N′-tetramethyluronium hexafluorophosphate (HBTU, 1.06 g, 2.8 mmol), and N,N-diisopropylethylamine (DIPEA, 490 μl, 2.8 mmol) were stirred in DMF (10 ml) for 5 minutes before adding DIPEA (490 μl, 2.8 mmol) and [1-(5-methoxy-2-pyridyl)cyclohexyl]-methanamine (see WO 98/07718, 678 mg, 3.1 mmol). HPLC indicated that reaction was complete within 1 hour. Solvent was removed under reduced pressure and the residue was taken up in EtOAc. The organic layer was washed with brine, saturated NaHCO₃ (×3), brine and dried (MgSO₄), after which solvent was removed under reduced pressure. The residue was purified by chromatography using RP silica with 65% MeOH in H₂O. Pure fractions were evaporated to give the desired product as an amorphous solid (1.12 g, 66%):

MPt: 100-105° C.;

MS m/e (ES+): 609.63 (M⁺+H, 100%);

IR (film): 3359, 3272, 3054, 2932, 2857, 1628, 1606, 1573, 1515, 1488, 1393, 1336, 1268, 1232, 1181, 1150, 1131, 1097, 1028, 1012, 962, 939, 900, 853, 831, 737 cm⁻¹;

¹H NMR (CDCl₃): δ=1.10-1.60 (8H, m), 1.72 (3H, s), 1.95-2.02 (2H, m), 3.31-3.42 (2H, m), 3.41 (1H, d, J=14.6 Hz), 3.50 (1H, d, J=14.6 Hz), 3.69 (3H, s), 5.34 (1H, s), 6.90-6.97 (2H, m), 7.04-7.09 (2H, m,) 7.14-7.19 (1H, m), 7.33 (1H, d, J=8.1 Hz), 7.46 (1H, d, J=7.8 Hz), 7.54 (1H, s), 7.77 (2H, d, J=8.8 Hz), 8.00 (1H, d, J=2.9 Hz), 8.04 (1H, s), 8.21 (2H, d, J=8.8 Hz); (amide masked by CHCl₃)

HPLC A: Rt. 11.86 min, 99.8/100% purity, 20-100% CH₃CN in H₂O (+0.1%TFA) over 15 min at 1 ml min⁻¹, Prodigy ODSIII 250×4.6 mm 5 μM, 215 and 254 nm;

HPLC B: Rt. 14.32 min, 100/100% purity, 80:20 methanol/Tris buffer at pH9, 1 mlmin⁻¹, Prodigy ODSIII 250×4.6 mm 5 μM, 215 and 254 nm.

### EXAMPLE 10

(S)-3-(1H-Indol-3-yl)-N-(1-methoxymethyl-cyclohexylmethyl)-2-methyl-2-[4-(4-nitro-phenyl)-oxazol-2-ylamino]-propionamide

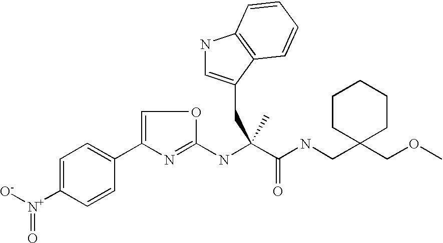

The above compound was synthesized from Intermediate 4a and Intermediate 13 using the same method as used for Example 9. The acid (4a) (203 mg, 0.5 mmol), HBTU (190 mg, 0.5 mmol), and DIPEA (87 μl, 0.5 mmol) were stirred in DMF (10 ml) for 5 minutes before adding DIPEA (87 μl×2, 1.0 mmol) and Intermediate 13 (94 mg, 0.5 mmol, Scheme 6). After 4 hours the solvent was removed under reduced pressure and residue taken up in EtOAc. The organic layer was washed with brine, saturated NaHCO₃ (×3), brine, dried (MgSO₄) and solvent removed under reduced pressure. The residue was heated to 60° C. in methanol and product filtered off. Drying under reduced pressure gave the desired product as a yellow crystalline solid (214 mg, 78%):

MPt: 189-192° C.;

MS m/e (ES+): 546.49 (M⁺+H, 100%);

IR (film): 3285, 2928, 2849, 1637, 1604, 1516, 1453, 1334, 1260, 1108, 1077, 860, 743, 729 cm⁻¹;

¹H NMR (DMSO-d₆): δ=1.10-1.35 (10H, m), 1.44 (3H, s), 2.91-3.01 (3H, m), 3.06-3.12 (1H, m), 3.07 (3H, s), 3.26-3.31 (1H, m), 3.64 (1H, d, J=14.4 Hz), 6.87-6.93 (2H, m), 7.01 (1H, t, J=7.4 Hz), 7.29-7.37 (3H, m), 7.44 (1H, s), 7.94 (2H, d, J=9.0 Hz), 8.26 (2H, d, J=8.8 Hz), 8.34 (1H, s), 10.84 (1H, s);

HPLC A: Rt. 17.07 min, 100/100% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 14.35 min, 100/100% purity, 80:20 methanol/Tris buffer at pH9, 1 mlmin⁻¹, Prodigy ODSIII 250×4.6 mm 5 μM, 215 and 254 nm.

### EXAMPLE 11

(S)-3-(1H-Indol-3-yl)-2-methyl-2-[4-(4-nitro-phenyl)-oxazol-2-ylamino]-N-(2-oxo-2-phenyl-ethyl)-propionamide.

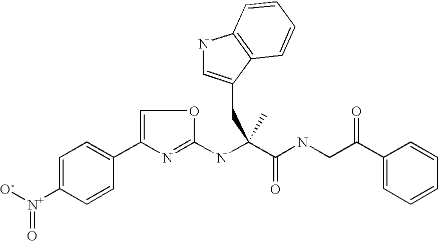

The above compound was synthesised from Intermediate 4a using the same method as used for Example 9. The acid (4a) (203 mg, 0.5 mmol), HBTU (190 mg, 0.5 mmol), and DIPEA (87 μl, 0.5 mmol) were stirred in DMF (10 ml) for 5 minutes before adding DIPEA (87 μl, 0.5 mmol) and 2-amino-1-phenyl-ethanone (103 mg, 0.6 mmol). After 4 hours the solvent was removed under reduced pressure and residue taken up in EtOAc, washed with brine, saturated NaHCO₃ (×3), brine, dried (MgSO₄) and solvent removed under reduced pressure. The residue was purified by chromatography using NP 20 g Mega Bond Elut cartridge and 40% ethyl acetate in heptane as eluent. Evaporation of pure fractions gave the desired product as a yellow amorphous solid (170 mg, 65%):

MPt: 80-90° C.;

MS m/e (AP+): 525.83 (16%), 524.44 (M⁺+H, 100%);

IR (film): 3396, 3059, 2983, 2932, 1694, 1628, 1605, 1575, 1514, 1449, 1336, 1284, 1264, 1225, 1181, 1154, 1096, 1072, 1010, 1001, 940, 853, 737 cm⁻¹;

¹H NMR (DMSO-d₆): δ=1.50 (3H, s), 3.39 (1H, d, J=14.7 Hz), 3.64 (1H, d, J=14.6 Hz), 4.53 (1H, d.d, J=18.1 and 5.4 Hz), 4.66 (1H, d.d, J=18.1 and 5.5 Hz), 6.87 (1H, t, J=7.4 Hz), 6.95 (1H, d, J=2.2 Hz), 7.00 (1H, t, J=7.4 Hz), 7.30 (1H, d, J=8.1 Hz), 7.34 (1H, d, J=8.1 Hz), 7.41 (1H, s), 7.50-7.55 (2H, m), 7.62-7.67 (1H,m), 7.94-7.99 (4H, m), 8.24 (1H, t, J=5.4 Hz), 8.27 (2H, d, J=9.0 Hz), 8.31 (1H, s), 10.86 (1H, s);

HPLC A: Rt. 20.83 min, 98.3/99.6% purity, 20-100% CH₃CN in H₂O (+0.1%TFA) over 25 min at 1 mlmin⁻¹, Prodigy ODSIII 250×4.6 mm 5 μM, 215 and 254 nm;

HPLC B: Rt. 6.82 min, 100/100% purity, 80:20 methanol/Tris buffer at pH9, 1 mlmin⁻¹, Prodigy ODSIII 250×4.6 mm 5 μM, 215 and 254 mn.

### EXAMPLE 12

(S)-N-[1-(5-Methoxy-pyridin-2-yl)-cyclohexylmethyl]-2-methyl-2-[4-(4-nitro-phenyl)-oxazol-2-ylamino]-3-phenyl-propionamide

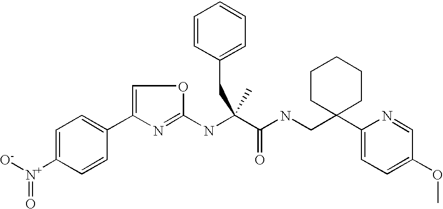

The above compound was synthesised from 1b and 4b using the same methods as used for Example 9. The acid (4b) (120 mg, 0.33 mmol), HBTU (124 mg, 0.33 mmol), and DIPEA (114 μl, 0.66 mmol), and [1-(5-methoxy-2-pyridyl)cyclohexyl]-methanaminel (86 mg, 0.4 mmol) were stirred in DMF (4 ml) for 18 hours. Solvent removed under reduced pressure and residue taken up in EtOAc. The organic layer was washed with brine, saturated NaHCO₃ (×3), brine, dried (MgSO₄) and solvent removed under reduced pressure. The residue was purified by chromatography using NP silica with 10-80% ethyl acetate in heptane. Pure fractions were evaporated to give the desired compound as a yellow amorphous solid (90 mg, 49%):

MS m/e (AP+): 570.23 (M⁺+H, 100%);

IR (film): 3363, 2930, 2856, 1658, 1651, 1628, 1574, 1515, 1488, 1334, 1268, 1232, 1073, 1030, 938, 852 cm⁻¹;

¹H NMR (DMSO-d₆): δ=0.94-1.46 (1H, m), 1.98-2.10 (2H, m), 3.04-3.14 (2H, m), 3.25-3.32 (1H, m), 3.57 (1H, d, J=13.6 Hz), 3.73 (3H, s), 6.95-7.00 (3H, m), 7.10-7.24 (5H, m), 7.44 (1H, s), 7.93 (2H, d, J=8.8 Hz), 8.14 (1H, d, J=2.8 Hz), 8.27 (2H, d, J=9.2 Hz), 8.36 (1H, s);

HPLC A: Rt. 5.49 min, 99.76% purity, 20-100% CH₃CN in H20 (+0.1%TFA) over 7 min at 1.5 mlmin⁻¹, Prodigy ODSIII 150×4.6 mm 3 μM at 40° C., 200-300 nm;

HPLC B: Rt. 5.72 min, 99.46% purity, 20-90% CH₃CN/Tris (1 mM) over 7 min at 2 mlmin⁻¹, Prodigy Phenyl-Ethyl, 100×4.6 mm 5 μM at 30° C., 200-300 nm.

### EXAMPLE 13

(S)-2-[4-(4-Cyano-phenyl)-oxazol-2-ylamino]-3-(1H-indol-3-yl)-N-[1-(5-methoxy-pyridin-2-yl)-cyclohexylmethyl]-2-methyl-propionamide

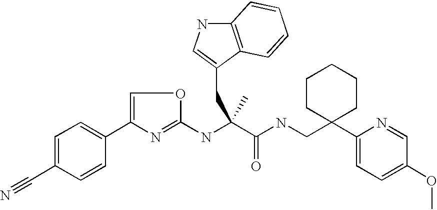

The above compound was synthesized from 2a via 6a as outlined in Scheme 2 using methods analogous to those used for Example 9. The acid (6a) (309 mg, 0.8 mmol), HBTU (303 mg, 0.8 mmol), DIPEA (140 μl, 0.8 mmol) were stirred in DMF (5 ml) for 5 minutes before adding DIPEA (140 μl, 0.8 mmol) and [1-(5-methoxy-2-pyridyl)cyclohexyl]-methanamine (WO 98/07718) (185 mg, 0.84 mmol). HPLC indicated reaction complete within 1 hour. Solvent removed under reduced pressure and residue taken up in EtOAc. Washed with brine, saturated NaHCO₃ (×3), brine, dried (MgSO₄) and solvent removed under reduced pressure. Residue purified by chromatography using RP silica with 65% MeOH in H₂O. Pure fractions were evaporated to give Example 13 as a white amorphous solid (320 mg, 68%):

MPt: 105-108° C.;

MS m/e (ES+): 589.32 (M⁺+H, 100%), 590.18 (62%);

IR (film): 3355, 2932, 2857, 2225, 1628, 1572, 1521, 1489, 1456, 1328, 1269, 1232, 1096, 1072, 1029, 938, 844, 741 cm⁻¹;

¹H NMR (CDCl₃): δ=1.20-1.60 (8H, m), 1.70 (3H, s), 1.93-2.03 (2H, m), 3.30-3.52 (4H, m), 3.68 (3H, s), 5.30 (1H, s), 6.89 (1H, d, J=2.4 Hz), 6.94 (1H, d.d, J=8.8 and 2.9 Hz), 7.03-7.09 (2H, m,) 7.14-7.19 (1H, m), 7.20-7.25 (1H, m), 7.33 (1H, d, J=8.1 Hz), 7.46 (1H, d, J=7.8 Hz), 7.50 (1H, s), 7.63 (2H, d, J=8.5 Hz), 7.72 (2H, d, J=8.3 Hz); 8.00 (1H, d, J=2.9 Hz), 8.05 (1H, s);

HPLC A: Rt. 11.63 min, 97.7/100% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 9.20 min, 100/100% purity, 80:20 methanol/Tris buffer at pH9, 1 mlmin⁻¹, Prodigy ODSIII 250×4.6 mm 5 μM, 215 and 254 nm.

### EXAMPLE 14

(S)-3-(1H-Indol-3-yl)-N-[1-(5-methoxy-pyridin-2-yl)-cyclohexylmethyl]-2-methyl-2-(4-phenyl-oxazol-2-ylamino)-propionamide

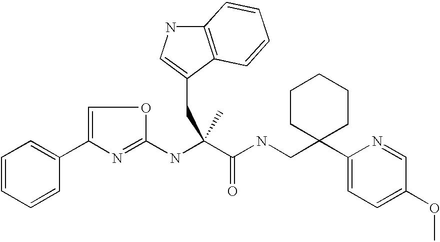

The above compound was synthesised from 2a via 6b as outlined in Scheme 2 using methods analogous to those used for Example 9. The acid (6b) (57 mg, 0.148 mmol), HBTU (56 mg, 0.48 mmol), DIPEA (26 μl, 0.148 mmol) were stirred in DMF (5 ml) for 5 minutes before adding DIPEA (261 μl, 0.148 mmol) and [1-(5-methoxy-2-pyridyl)cyclohexyl]-methanamine (see WO 98/07718, 34 mg, 0.148 mmol). HPLC indicated that the reaction was complete within 2 hours. Solvent was removed under reduced pressure and the residue was taken up in EtOAc, washed with brine, sat. NaHCO₃ (×3), brine, dried (MgSO₄) and solvent removed under reduced pressure. The residue was purified by chromatography using RP silica with 70% MeOHl in H₂O as eluent. Repurification using NP 8 g Biotage cartridge with 45% ethyl acetate in heptane as eluent gave the desired product as a glass (20 mg, 24%):

MPt: 85-90° C.;

MS m/e (ES+): 564.06 (M⁺, 87%), 564.96 (M⁺+H, 100%);

IR (film): 3289, 2931, 2857, 1627, 1569, 1520, 1488, 1456, 1337, 1267, 1233, 1072, 1072, 1030, 939, 739 cm⁻¹;

¹H NMR (DMSO-d₆): δ=0.95-1.45 (1H, m), 2.00-2.10 (2H, m), 3.10-3.25 (2H, m), 3.21 (1H, d, J=14.6 Hz), 3.59 (1H, d, J=14.6 Hz), 3.71 (3H, s), 6.84-7.14 (7H, m), 7.24-7.40 (5H, m,), 7.70 (2H, d, J=7.6 Hz), 8.05 (1H, s), 8.15 (1H, d, J=2.9 Hz), 10.82 (1H, s);

HPLC A: Rt. 12.01 min, 96.8/95.3% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 17.27 min, 100/100% purity, 80:20 methanol/Tris buffer at pH9, 1 mlmin⁻¹, Prodigy ODSIII 250×4.6 mm 5 μM, 215 and 254 rnm.

### EXAMPLE 15

(S)-2-(4-Ethyl-oxazol-2-ylamino)-3-(1H-indol-3-yl)-N-[1-(5-methoxy-pyridin-2-yl)-cyclohexylmethyl]-2-methyl-propionamide

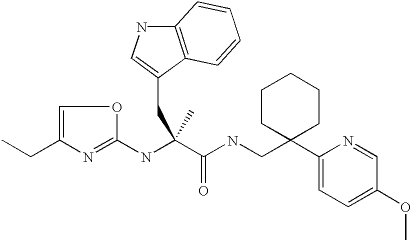

The above compound was synthesized from 2a via 6c as outlined in Scheme 2 using methods analogous to those used for Example 9. The acid (6c) (1 88 mg, 0.6 mmol), HBTU (228 mg, 0.6 mmol), and DIPEA (105 μl, 0.6 mmol) were stirred in DMF (10 ml) for 5 minutes before adding DIPEA (105 μl, 0.6 mmol) and [1-(5-methoxy-2-pyridyl)cyclohexyl]-methanamine (see WO 98/07718, 150 mg, 0.65 mmol). HPLC indicated that the reaction was complete within 4 hours. Solvent was removed under reduced pressure and residue was taken up in EtOAc, washed with brine, sat. NaHCO₃ (×3), brine, dried (MgSO₄) and solvent removed under reduced pressure. The residue was purified by chromatography using RP silica with 65% MeOH in H₂O. The product was repurified using 20 g Mega Bond Elut silica cartridge with 45% ethyl acetate in heptane as eluent. Pure fractions were evaporated to give the above compound as a glass (30 mg, 10%):

MPt: 60-65° C.;

MS m/e (ES+): 516.24 (M⁺+H, 47%), 517.01 (100%), 538.10 (M⁺+Na, 25%);

IR (film): 3272, 3054, 2931, 2856, 1651, 1622, 1596, 1573, 1520, 1489, 1457, 1358, 1268, 1232, 1206, 1131, 1083, 1028, 949, 830, 740 cm⁻¹;

¹H NMR (DMSO-d₆): δ=1.10-1.50 (8H, m), 1.11 (3H, t, J=7.4 Hz), 1.29 (3H, s), 2.05-2.15 (2H, m), 2.28-2.34 (2H, m), 3.08-3.18 (3H, m), 3.48 (1H, d, J=14.4 Hz), 3.79 (3H, s), 6.80-6.90 (3H, m), 6.97-7.04 (2H, m,), 7.10-7.20 (3H, m), 7.27-7.30 (2H, m), 8.17 (1H, d, J=2.9 Hz), 10.80 (1H, s);

LCMS: Rt. 1.36 min, 100% purity, 5-100% CH₃CN in H₂O (+0.1% Formic acid) over 2 min at 4 mlmin⁻¹, Prodigy ODSIII 50×4.6 mm 5 μM, 215 nm, MS m/e (ES+) 515.95 (100%);

HPLC B: Rt. 12.29 min, 100/100% purity, 80:20 methanol/Tris buffer at pH9, 1 mlmin⁻¹, Prodigy ODSIII 250×4.6 mm 5 μM, 215 and 254 nm;

### EXAMPLE 16

(S)-3-(1H-Indol-3-yl)-N-[1-(5-methoxy-pyridin-2-yl)-cyclohexylmethyl]-2-methyl-2-[4-(4-nitro-phenyl)-thiazol-2-ylaminol-propionamide

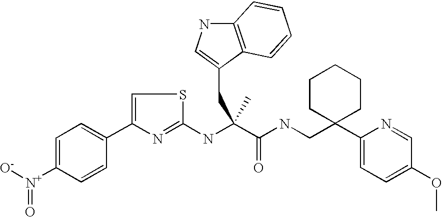

The above compound was synthesized using a one-pot procedure as outlined in Scheme 3. A suspension of H-S-αMeTrp-OH (Intermediate 7) (437 mg, 2 mmol), 2-chloro-4-(4-nitro-phenyl)-thiazole (see Peet, Norton P.; Sunder, Shyam. Reinvestigation of the reported preparation of 3-(4-nitrophenyl)thiazolo[2,3-c][1,2,4]triazepines, J. Heterocycl. Chem. (1986), 23(2), 593-5; 481 mg, 2 mmol), copper (I) iodide (38 mg, 0.2 mmol), and K₂CO₃ (415 mg, 3 mmol) in DMF (12 ml) under nitrogen was heated to 130° C. for 12 hours. The reaction mixture was cooled to ambient temperature before adding HBTU (759 mg, 2 mmol) and [1-(5-methoxy-2-pyridyl)cyclohexyl]-methanamine (see WO 98/07718; 441 mg, 2 mmol). The mixture was stirred overnight, then concentrated in vacuo, after which the residue was partitioned between water (20 ml) and dichloromethane (30 ml). The organic phase was separated and filtered through silica (3×12 cm) using 500 ml of dichloromethane and then 500 ml of dichloromethane-ether (1:1). Fractions containing product were concentrated under reduced pressure. The residue was absorbed onto 3.5 g silica and purified by chromatography (3×11 cm) using heptane-ethyl acetate (1:1.1). The product was repurified using RP chromatography (Biotage KP-C18-HS Flash 12M, 15 ml.min⁻¹, 60-100% methanol in water). Concentration under reduced pressure gave the desired compound as a pale yellow amorphous solid (27 mg, 2%):

MPt: 110-114° C.;

MS m/e (AP+): 624.88 (M⁺, 100%), 625.70 (M⁺+H, 52%);

IR (film): 3385, 3279, 2931, 2855, 1654, 1595, 1542, 1509, 1456, 1341, 1268, 1231, 1108, 1058, 908, 844, 731 cm⁻¹;

¹H NMR (CDCl₃): δ=1.15-1.55 (8H, m), 1.71 (3H, s), 1.90-2.00 (2H, m), 3.16-3.42 (2H, m), 3.46 (1H, d, J=14.9 Hz), 3.60 (1H, d, J=14.6 Hz), 3.70 (3H, s), 5.51 (1H, s), 6.89-6.93 (3H, m), 6.98 (1H, d, J=8.8 Hz), 7.05-7.10 (1H, m), 7.15-7.25 (2H, m), 7.34 (1H, d, J=8.3 Hz), 7.47 (1H, d, J=7.8 Hz), 7.90 (2H, d, J=9.0 Hz), 7.98 (iH, d, J=2.9 Hz), 9.05 (1H, s), 8.21 (2H, d, J=8.8 Hz);

HPLC A: Rt. 12.30 min, 99.4% purity, 20-100% CH₃CN in H₂O (+0.1%TFA) over 15 min at 1 mlmin⁻¹, Prodigy ODSIII 250×4.6 mm 5 μM, 200-300 nm; HPLC B: Rt. 15.38 min, 99.5% purity, 80:20 methanol/Tris buffer at pH9, 1 mlmin⁻¹, Prodigy ODSIII 250×4.6 mm 5 μM, 200-300 nm.

### EXAMPLE 17

(S)-2-(Benzooxazol-2-ylamino)-3-(1H-indol-3-yl)-2-methyl-N-(1-pyridin-2-yl-cyclohexylmethyl)-propionamide

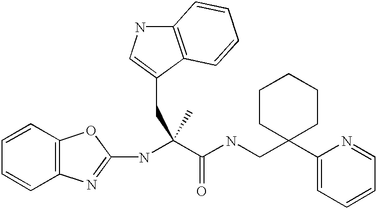

1. The following reagents were combined in the order that they are listed: Intermediate 7 (545 mg, 2.5 mmol), 2-chlorobenzoxazole (384 mg, 2.5 mmol), potassium carbonate (346 mg, 2.5 mmol), benzyltriethylammonium chloride (TEBA, 114 mg, 0.5 mmol), triethylamine (1.04 ml, 7.5 mmol), DMF (12.5 ml), deoxygenated water (1.25 ml), copper (I) iodide (24 mg, 0.125 mmol), trans-dichlorobis(tri-o-tolyl-phosphine)palladium(II) (99 mg, 0.125 mmol). After heating at 100° C. under nitrogen for 24 hours the DMF was removed under reduced pressure. The residue was taken up in ethyl acetate/water and the aqueous phase was acidified to pH 6-6.5 using citric acid. The aqueous phase was extracted with three further portions of ethyl acetate. The combined organic layers were dried (MgSO₄) and solvent was removed under reduced pressure. The residue was purified by chromatography using 10 g NP silica with 0-100% ethyl acetate in heptane. Crystallisation from dichloromethane gave (S)-2-(benzooxazol-2-ylamino)-3-(1H-indol-3-yl)-2-methyl-propionic acid (245 mg, 29%). MS m/e (ES+) 335.97 (M⁺+H, 100%), 336.69 (85%).

2. The propionic acid (234 mg, 0.7 mmol), HBTU (265 mg, 0.7 mmol), and DIPEA (122 μl, 0.7 mmol) were stirred in DMF (10 ml) for 5 minutes before adding DIPEA (122 μl, 0.7 mmol) and [1-(2-pyridyl)cyclohexyl]methylamine (WO 98/07718; 140 mg, 0.74 mmol). After 4 hours at ambient temperature the solvent was removed under reduced pressure. The residue was purified by chromatography using NP silica with 50% ethyl acetate in heptane as eluent. Pure fractions were evaporated to give the desired compound as fine needles (44 mg, 3%):

MPt: 198-200° C.;

MS m/e (ES+): 508.59 (100%, M⁺+H), 509.92 (10%);

IR (film): 3381, 3222, 3048, 2929, 2856, 1635, 1581, 1552, 1519, 1458, 1353, 1241, 1096, 742 cm⁻¹;

¹H NMR (CDCl₃): δ=1.20-1.60 (8H, m), 1.76 (3H, s), 1.95-2.05 (2H, m), 3.34 (1H, d.d, J=13.2 and 4.9 Hz), 3.45 (1H, d.d, J=13.2 and 5.6 Hz), 3.50 (2H, s), 5.67 (1H, s), 6.78-6.82 (1H, m), 6.89 (1H, d, J=2.2 Hz), 6.99-7.35 (1OH, m), 7.43 (1H, d, J=8.1 Hz), 8.01 (1H, s), 8.24 (1H, d, J=4.6 Hz);

HPLC A: Rt. 10.54 min, 100/100% purity, 20-100% CH₃CN in H20 (+0.1%TFA) over 15 min at 1 mlmin⁻¹, Prodigy ODSIII 250×4.6 mm 5 μM, 215 and 254 nm;

HPLC B: Rt. 10.67 min, 100/100% purity, 80:20 methanol/Tris buffer at pH9, 1 mlmin⁻¹, Prodigy ODSIII 250×4.6 mm 5 μM, 215 and 254 nm;

### EXAMPLE 18

(S)-3-(1H-Indol-3-yl)-2-methyl-2-(pyridin-4-ylamino)-N-(1-pyridin-2-yl-cyclohexylmethyl)-propionamide

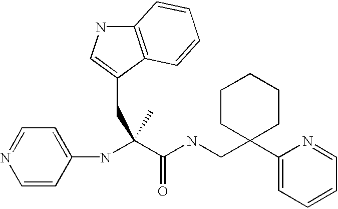

The above compound was prepared on the same scale and using an analogous method as used for Example 17.

1. The method of Example 17 was repeated except that 4-bromopyridine hydrochloride (486 mg, 2.5 mmol) was used.

2. The acid from step 1 (30 mg, 0.1 mmol), HBTU (38 mg, 0.1 mmol), and DIPEA (18 μl, 0.1 mmol) were stirred in DMF (10 ml) for 5 minutes before adding DIPEA (18 μl, 0.1 mmol) and [1-(2-pyridyl)cyclohexyl]methylamine (WO 98/07718; 19 mg, 0.1 mmol). After 2 hours at ambient temperature the solvent was removed under reduced pressure. The residue was taken up in ethyl acetate and washed with sodium bicarbonate solution (×2), brine, and dried (MgSO₄). The solvent was removed under reduced pressure. The crude product was purified by chromatography using 10 g ISCO Redisep cartridge with ethyl acetate as eluent. Repurification using 20 g RP-C 18 with 70% methanol in water and subsequent evaporation gave the desired product in crystalline form (6 mg, 13%):

MPt: 180-195° C.;

MS m/e (AP+): 468.12 (M⁺+H, 100%), 469.59 (M⁺+2H, 20%);

MS m/e (AP−): 467.56 (M⁻, 45%), 466.60 (M⁻−H, 100%), 465.64 (M⁻−2H, 88%);

IR (film): 3316, 2930,1651, 1602, 1515, 1430, 1106, 997, 816, 741 cm⁻¹;

NMR (CDCl₃): δ=1.25-1.70 (8H, m), 1.46 (3H, s), 2.00-2.10 (2H, m), 3.27 (1H, d, J=14.9 Hz), 3.30-3.48 (2H, m), 3.36 (1H, d, J=14.9 Hz), 4.43 (1H, s), 6.22 (2H, d, J=5.6 Hz), 6.85 (1H, d, J=2.0 Hz), 6.89-6.93 (1H, m), 7.11-7.37 (5H, m), 7.46-7.54 (2H, m), 8.08-8.13 (4H, m);

HPLC A: Rt. 7.21 min, 96.1/96.5% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 6.02 min, 99.1/100% purity, 80:20 methanol/Tris buffer at pH9, 1 mlmin⁻¹, Prodigy ODSIII 250×4.6 mm 5 μM, 215 and 254 nm.

### EXAMPLE 19

(S)-3-(1H-Indol-3-yl)-2-(isoquinolin-4-ylamino)-2-methyl-N-(1-pyridin-2-yl-cyclohexylmethyl)-propionamide

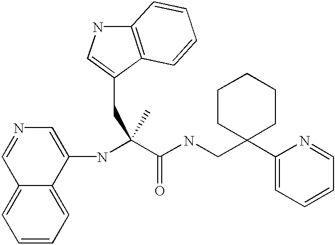

Example 19 was prepared on the same scale and using an analogous method as used for Example 17.

1. The method of Example 17 was followed except that 4-bromoisoquinoline (520 mg, 2.5 mmol) was used.

2. The acid from step 1 (40 mg, 0.12 mmol), HBTU (46 mg, 0.12 mmol), and DIPEA (21 μl, 0.12 mmol) were stirred in DMF (10 ml) for 5 minutes before adding DIPEA (21 μl, 0.12 mmol) and [1-(2-pyridyl)cyclohexyl]methylamine (WO 98/07718; 23 mg, 0.12 mmol). After 2 hours at ambient temperature the solvent was removed under reduced pressure. The residue was taken up in ethyl acetate and washed with sodium bicarbonate solution (×2) and brine and dried (MgSO₄). The solvent was removed under reduced pressure. The crude product was purified by chromatography using 10 g ISCO Redisep cartridge with 80% ethyl acetate in heptane as eluent. Repurification using 20 g RP-C 18 with 70% methanol in water and subsequent evaporation gave the desired product as a glass (9 mg, 14%):

MPt: 98-101° C.;

MS m/e (AP+): 518.28 (100%, M⁺+H), 517.40 (M⁺, 50%);

MS m/e (AP−): 516.53 (75%, M⁻), 515.63 (100%, M⁻−H);

IR (film): 3385, 3278, 3052, 2927, 2849, 1651, 1585, 1520, 1455, 1403, 1343, 781,740 cm⁻¹;

NMR (CDCl₃): δ=1.20-1.65 (1H, m), 1.93-2.10 (2H, m), 3.35 (1H, d, J=14.6 Hz), 3.39-3.52 (2H, m), 3.48 (1H, d, J=14.9 Hz), 4.62 (1H, s), 6.55-6.59 (1H, m), 6.90 (1H, d, J=2.0 Hz), 7.00 (1H, d, J=8.1 Hz), 7.17-7.28 (4H, m), 7.37-7.55 (4H, m), 7.62 (1H, s), 7.70 (1H, d, J=7.6 Hz), 7.74-7.76 (1H, m), 7.87 (1H, d, J=8.1 Hz), 8.15 (1H, s), 8.63 (1H, s) HPLC A: Rt. 7.52 min, 100/100% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 8.33 min, 99.7/100% purity, 80:20 methanol/Tris buffer at pH9, 1 mlmin⁻¹, Prodigy ODSIII 250×4.6 mm 5 μM, 215 and 254 nm;

### EXAMPLE 20

(S)-3-(1H-Indol-3-yl)-2-methyl-N-(1-pyridin-2-yl-cyclohexylmethyl)-2-(pyrimidin-5-ylamino)-propionamide

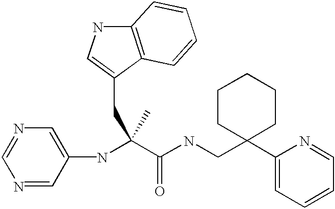

The above compound was prepared on the same scale and using an analogous method as used for Example 17.

1. The method of Example 17 was followed except that 5-bromopyrimidine (397 mg, 2.5 mmol) was used.

2. The acid from step 1 (150 mg, 0.5 mmol), HBTU (190 mg, 0.5 mmol), and DIPEA (87 μl, 0.5 mmol) were stirred in DMF (10 ml) for 5 minutes before adding DIPEA (87 μl, 0.5 mmol) and [1-(2-pyridyl)cyclohexyl]methylamine (WO 98/07718; 95 mg, 0.5 mmol). After 2 hours at ambient temperature the solvent was removed under reduced pressure. The residue was taken up in ethyl acetate and washed with sodium bicarbonate solution (×2) and brine and dried (MgSO₄). The solvent was removed under reduced pressure. The crude product was purified by chromatography using 10 g ISCO Redisep cartridge with 90% ethyl acetate in heptane as eluent. Removal of the solvent under reduced pressure gave the desired product as a foam (135 mg, 58%):

MPt: 95-98° C.;

MS m/e (AP+): 470.60 (25%), 469.58 (M⁺+H, 100%), 468.77 (M⁺, 92%);

MS m/e (AP−): 467.60 (M⁻−H, 70%), 466.85 (100%);

IR (film): 3291, 3052, 2931, 2857, 1651, 1575, 1519, 1470, 1455, 1427, 1357, 1306, 1265, 1237, 1194, 1156, 1106, 1010, 848, 788, 739 cm⁻¹;

NMR (CDCl₃): δ=1.20-1.65 (8H, m), 1.48 (3H, s), 2.00-2.10 (2H, m), 3.24-3.48 (4H, m), 4.14 (1H, s), 6.88-6.92 (2H, m), 7.13-7.24 (3H, m), 7.37 (1H, d, J=8.1 Hz), 7.48-7.55 (3H, m), 7.86 (2H, s), 8.08-8.10 (1H, m), 8.16 (1H, s), 8.57 (1H, s);

HPLC A: Rt. 8.94 min, 99.3/99.4% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 5.76 min, 95.1/98.7% purity, 80:20 methanol/Tris buffer at pH9, 1 mlmin⁻¹, Prodigy ODSIII 250×4.6 mm 5 μM, 215 and 254 nm.

### EXAMPLE 21

(S)-2-(Biphenyl-2-ylamino)-3-(1H-indol-3-yl)-2-methyl-N-(1-pyridin-2-yl-cyclohexylmethyl)-propionamide

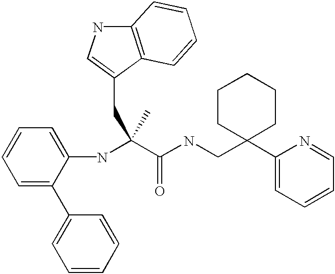

The above compound was prepared on the same scale and using an analogous method as used for Example 17.

1. The method of Example 18 except for the use of 2-bromo biphenyl (583 mg, 2.5 mmols).

2. The acid from step 1 (350 mg, 0.95 mmol), HBTU (400 mg, 1 mmol), NEt₃ (0.5 ml, 3.5 mmol), and 1-(2-pyridyl)cyclohexyl]methylamine (WO 98/07718; 200 mg, 1 mmol) were stirred in DMF (1 5 ml). After 1 hour at ambient temperature the reaction mixture was diluted with ethyl acetate (100 ml), washed with sodium bicarbonate solution (×2) and dried (MgSO₄). The solvent was removed under reduced pressure. The crude product was purified by chromatography using 0-50% ethyl acetate in heptane and then 0-30% dichloromethane in ether as eluent. Removal of the solvent under reduced pressure gave the desired product as a foam (98 mg, 19% for step 2):

MS m/e (AP+): 565 (M⁺+Na, 100%), 564 (80%), 542 (M⁺, 30%)

IR (KBr disc): 3404, 2928, 2855, 1650, 1584, 1508, 1489, 1458, 1432 cm⁻¹;

NMR (DMSO-d₆): δ=1.10-1.52 (8H, m), 1.27 (3H, s), 1.95-2.05 (2H, m), 2.95 (1H, d, J=14.4 Hz), 3.02-3.08 (1H, m), 3.08 (1H, d, J=14.6 Hz), 3.28-3.34 (1H, m), 4.36 (1H, s), 6.37 (1H, d, J=8 Hz), 6.49 (1H, d, J=2.2 Hz), 6.71-6.75 (1H, m), 6.82-6.86 (1H, m), 6.95-7.43 (13H, m), 7.52-7.57 (1H, m), 8.33 (1H, d, J=3.7 Hz), 10.81 (1H, s);

HPLC A: Rt. 12.65 min, 99.65% purity, 20-100% CH₃CN in H₂O (+0.1%TFA) 5 over 15 min at 1 mlmin⁻¹, Prodigy ODSIII 250×4.6 mm 5 μM, 200-300 nm;

HPLC B: Rt. 33.05 min, 99.89% purity, 80:20 methanol/Tris buffer at pH9, 1 mlmin⁻¹, Prodigy ODSIII 250×4.6 mm 5 μM, 200-300 nm.

### EXAMPLE 22

(S)-3-(1H-Indol-3-yl)-2-methyl-N-(1-pyridin-2-yl-cyclohexylmethyl)-2-m-tolylamino-propionamide

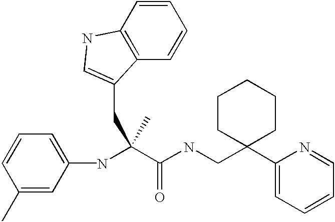

The above compound was prepared using a one-pot procedure analogous to the method used for Example 16. The synthesis was carried out on 1 mmol scale using 1-bromo-3-methyl-benzene (171 mg, 1 mmol). The crude product was purified by chromatography using 25 g NP silica with 25% ethyl acetate in heptane as eluent.

Removal of the solvent under reduced pressure gave the desired compound as a glass (260 mg, 54%):

MPt: 70-75° C.; 20 MS m/e (AP+): 481.33 (100%, M⁺+H), 482.37 (40%);

IR (film): 3385, 3291, 3049, 2929, 2857, 1652, 1607, 1590, 1513, 1456, 1431, 1341, 1302, 1264, 1237, 1177, 1155, 1104, 1010, 774, 741 cm⁻¹;

NMR (DMSO-d₆): δ=1.08-1.50 (8H, m), 1.19 (3H, s), 2.00-2.10 (2H, m), 2.16 (3H, s), 3.03 (1H, d.d, J=12.9 and 5.1 Hz), 3.10 (1H, d, J=14.7 Hz), 3.22 (1H, d, 25 J=14.6 Hz), 3.24-3.30 (1H, m), 5.43 (1H, s), 6.29 (1H, s), 6.30 and 6.44 (each 1H, each d, J=7.6 Hz), 6.87-7.07 (6H, m), 7.15-7.19 (1H, m), 7.29 (1H, d, J=8.0 Hz), 7.33 (1H, d, J=7.8 Hz), 7.48-7.54 (1H, m), 8.31-8.33 (1H, m), 10.81 (1H, s);

HPLC A: Rt. 11.04 min, 98.3% purity, 20-100% CH₃CN in H₂O (+0.1%TFA) over 15 min at 1 mlmin⁻¹, Prodigy ODSIII 250×4.6 mm 5 μM, 200-300 nm; HPLC B: Rt. 16.87 min, 99.5% purity, 80:20 methanol/Tris buffer at pH9, 1 mlmin⁻¹, Prodigy ODSIII 250×4.6 mm 5 μM, 200-300 nm.

### EXAMPLE 23

(S)-3-(1H-Indol-3-yl)-2-methyl-2-(6-phenyl-pyridin-2-ylamino)-N-(1-pyridin-2-yl-cyclohexylmethyl)-propionamide

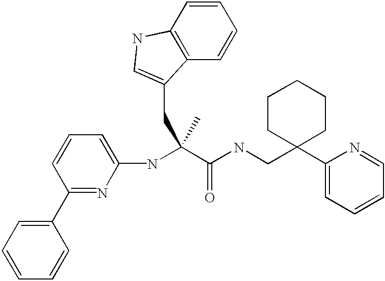

The above compound was prepared using a one-pot procedure analogous to the method used for Example 16. The synthesis was carried out on 0.4 mmol scale using 2-bromo-6-phenyl-pyridine (95 mg, 0.4 mmol). The crude product was purified by chromatography using 25 g NP silica with 55% ethyl acetate in heptane as eluent.

Removal of the solvent under reduced pressure gave the desired product as a foam (260 mg, 54%):

MS m/e (AP+) 544.31 (100%, M⁺+H), 545.35 (35%);

MS m/e (AP−) 542.29 (100%, M⁻−H), 543.31 (M⁻, 40%);

IR (film): 3407, 3276, 3056, 2930, 2857, 1651, 1595, 1576, 1519, 1486, 1467, 1455, 1439, 1339, 1264, 1180, 1157, 1105, 1028, 1009, 991, 804, 763, 739 cm⁻¹;

NMR (CDCl₃) δ=1.03-1.60 (8H, m), 1.53 (3H, s), 1.90-2.03 (2H, m), 3.32-3.45 (3H, m), 3.65 (1H, d, J=14.6 Hz), 4.67 (1H, s), 6.13 (1H, d, J=8.3 Hz), 6.77-7.50 (14H, m), 7.97 (2H, d, J=7.1 Hz), 8.02 (1H, s), 8.23-8.25 (1H, m);

HPLC A: Rt. 4.21 min, 96.8% purity, 20-100% CH₃CN in H₂O (+0.1%TFA) over 7 min at 1.5 mlmin⁻¹, Prodigy ODSIII 150×4.6 mm 5 μM, 200-300 nm.

### EXAMPLE 24

(R)-3-Phenyl-2-phenylamino-N-[1 1-pyridin-2-yl-cyclohexylmethyl)-propionamide

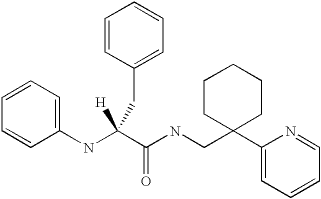

The above compound was synthesised as a two step process from Intermediate 8 as shown in Scheme 4.

1. To a solution of Intermediate 8 (0.5 g, 3 mmol) and bromobenzene (0.35 ml, 3.3 mmol) in DMA (5 ml) under nitrogen was added potassium carbonate (0.6 g, 4.3 mmol) and copper (I) iodide (50 mg, 0.26 mmol) after which the mixture was heated to 90° C. for 1.5 hours. Solvent was removed under reduced pressure and the residue was purified by flash chromatography eluting with 5% methanol in dichloromethane. Removal of solvent under reduced pressure gave (R)-3-phenyl-2-phenylamino-propionic acid as an oil (0.41 g, 56%):

MS m/e (AP+): 242 (M⁺+H, 100%).

2. The acid from step 1 (0.40 g, 1.66 mmol), HBTU (0.6 g, 1.8 mmol), and NEt₃ (0.5 ml, 3.5 mmol), and 1-(2-pyridyl)cyclohexyl]methylamine (WO 98/07718; 0.35 mg, 1.8 mmol) were stirred in DMF (15 ml). After 1 hour at ambient temperature the reaction mixture was diluted with ethyl acetate (100 ml), washed with sodium bicarbonate solution (×2) and dried (MgSO₄). The solvent was removed under reduced pressure. The crude product was purified by chromatography using 50% ethyl acetate in heptane and then RP C18 silica with 70% methanol in water as eluent. Removal of the solvent under reduced pressure gave the desired product as a white amorphous solid (0.15 g, 22%):

MPt: 113-115° C.;

MS m/e (AP+): 414.22 (M⁺+H, 100%);

IR (KBr disc): 3300, 2931, 2858, 1649, 1605, 1589, 1523, 1498, 1432, 1318, 748 cm⁻¹;

NMR (CDCl₃): δ=1.20-1.70 (8H, m), 1. 90-2.15 (2H, m), 2.91 (1H, d.d, J=14.2 and 8.8 Hz), 3.27 (1H, d.d, J=14.2 and 4.4 Hz), 3.38 (1H, d.d, J=13.2 and 5.5 Hz), 3.48 (1H, d.d, J=13.2 and 6.1 Hz), 3.80 (1H, d, J=3.4 Hz), 3.88-3.93 (1H, m), 6.44 (2H, d, J=7.8 Hz), 6.74 (1H, t, J11.3 Hz), 6.90-7.45 (11H,m), 8.28 (1H, d, J=3.6 Hz);

HPLC A: Rt. 4.51 min, 100% purity, 20-100% CH₃CN in H₂O (+0.1%TFA) over 10 min at 1.5 mlmin⁻¹, Prodigy ODSIII 250×4.6 mm 5 μM, 200-300 nm;

HPLC B: Rt. 13.15 min, 99.14% purity, 80:20 methanol/Tris buffer at pH9, 1 mlmin⁻¹, Prodigy ODSIII 250×4.6 mm 5 μM, 200-300 nm.

### EXAMPLE 25

(S)-3-(1H-Indol-3-yl)-2-methyl-2-phenylethylamino-N-(1-pyridin-2-yl-cyclohexylmethyl)-propionamide

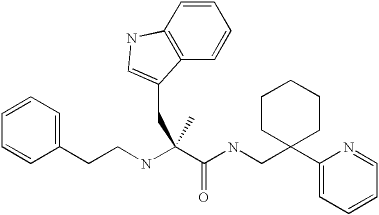

The above compound was prepared as shown in Scheme 5 via Intermediate 10.

1. To a stirred solution of H-(S)-αMeTrp-OH (7) (10 g, 46 mmol) and di-t-butyl-dicarbonate (10 g, 46 mmol) in dioxan (100 ml) was added water (20 ml) and potassium carbonate (10 g, 74 mmol). After 4 hours the reaction mixture was acidified with 2N hydrochloric acid (150 ml) and product was extracted with ethyl acetate (2×200 ml). The combined organic phases were dried (MgSO₄) and evaporated under reduced pressure. The residue was purified by flash chromatography using ethyl acetate as eluent. Removal of solvent under reduced pressure gave Boc-(S)-αMeTrp-OH as an orange oil (14.5 g, 99%). To a stirred solution of Boc-(S)-αMeTrp-OH (7 g, 22 mmol) in DMF (100 ml) was added HBTU (8.0 g, 22 mmol), triethylamine (5 ml, 35 mmol), and [1-(2-pyridyl)cyclohexyl]methylamine (WO 98/07718; 4.2 g, 22 mmol). After 1 hour the reaction mixture was diluted with ethyl acetate (300 ml), washed with 2N hydrochloric acid (2×200 ml), dried (MgSO₄) and evaporated under reduced pressure at 60° C. The residue was purified by flash chromatography. Elution with 5% methanol in dichloromethane and subsequent removal of solvent under reduced pressure gave intermediate 9 as yellow oil (8.3 g, 77%):

MS m/e (AP+): 491 (M⁺+H, 100%), 513 (M⁺+Na, 20%);

IR (film): 3339, 2929, 2858, 1704, 1659, 1651, 1589, 1519, 1487, 1366, 1249, 1164, 1070, 908, 737 cm⁻¹;

NMR (CDCl₃): δ=1.20-1.70 (20H, m), 2.00-2.12 (2H, m), 3.25-3.50 (4H, m), 5.05-5.20 (1H, br.s), 6.92 (1H, d, J=2.0 Hz), 7.02-7.32 (6H, m), 7.51 (1H, d, J=8.0 Hz), 7.59-7.64 (1H, m), 8.03 (1H, s), 8.48 (1H, d, J=4 Hz).

2. To a stirred solution of Intermediate 9 (8.2 g, 16.5 mmol) in dichloromethane (100 ml) was added trifluoroacetic acid (3.0 ml, 39 mmol). After 18 hours the solvent was removed under reduced pressure at 60° C. The residue was treated cautiously with saturated sodium carbonate solution (200 ml) before extracting with ethyl acetate (3×200 ml). The combined organic phases were dried (MgSO₄) and evaporated under reduced pressure at 60° C. The residue was purified by flash chromatography. Elution with 0-5% methanol in dichloromethane and subsequent removal of solvent under reduced pressure gave Intermediate 10 as white foam (4.85 g, 75%):

MPt: 65-68° C.;

MS m/e (AP+): 391 (M⁺+H, 100%);

IR (KBr disc): 3367, 2926, 2855, 1648, 1589, 1569, 1522, 1455, 1430, 1366, 1341, 1234, 842, 784, 742 cm⁻¹;

NMR (CDCl₃): δ=1.20-1.80 (13H, m), 1.98-2.20 (2H, m), 2.83 (1H, d, J=14.2 Hz), 3.33 (1H, d, J=14.2 Hz), 3.38 (2H, d, J=5.6 Hz), 6.98-7.20 (6H, m), 7.50-7.75 (3H, m), 8.05-8.15 (1H, s), 8.49-8.51 (1H, m);

3. To a stirred solution of Intermediate 10 (293 mg, 0.75 mmol) and phenacetaldehyde (90 mg, 0.75 mmol) in 1,2-dichloroethane (20 ml) was added solid sodium triacetoxyborohydride (316 mg, 1.5 mmol). After stirring overnight, saturated sodium bicarbonate solution was added—effervescence was observed. The aqueous phase was extracted with dichloromethane. The combined organic phases were dried (MgSO₄) and solvent was removed under reduced pressure. The residue was purified by chromatography using 20 g RP-C18 with 0-50% methanol in water followed by 20 g NP silica with 45% ethyl acetate in heptane. Removal of solvent under reduced pressure gave the desired compound as a glass (60 mg, 16%):

MS m/e (ES+): 496.56 (28%), 495.5 (52%, M⁺+H), 364.43 (22%), 269.34 (51%), 268.90 (88%), 248.37 (100%);

IR (film): 3274, 3058, 2928, 2856, 1651, 1588, 1568, 1519, 1469, 1454, 1431, 1355, 1263, 1236, 1155, 1117, 1053, 1030, 1009, 992, 930, 782, 742 cm⁻¹,

¹H NMR (CDCI₃): δ=1.20-1.65 (1H, m), 2.00-2.20 (2H, m), 2.40-2.75 (4H, m), 2.94 and 3.05 (each 1H, each d, J=14.4 Hz), 3.41 (2H, d, J=6.1 Hz), 6.74 (1H, d, J=2.2 Hz), 7.04-7.25 (9H, m), 7.32 (1H, d, J=7.8 Hz), 7.55-7.60 (3H, m), 7.90 (1H, s), 8.55-8.58 (1H, m);

HPLC A: Rt. 8.52 min, 99.0/98.6% purity, 20-100% CH₃CN in H₂O (+0.1%TFA) over 15 min at 1 mlmin⁻¹, Prodigy ODSIII 250×4.6 mm 5 μM, 215 and 254 nm;

HPLC B: Rt. 23.84 min, 99.6/100% purity, 80:20 methanol/Tris buffer at pH9, 1 mlmin⁻¹, Prodigy ODSIII 250×4.6 mm 5 μM, 215 and 254 nm.

### EXAMPLE 26

(S)-2-[(Benzofuran-2-ylmethyl)-aminol-3-(1H-indol-3-yl)-2-methyl-N-(1-pyridin-b 2-yl-cyclohexylmethyl)-propionamide

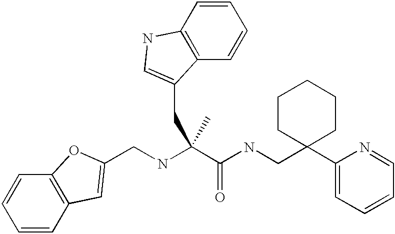

The above compound was prepared as shown in Scheme 5 via Intermediate 10.

To a stirred solution of Intermediate 10 (150 mg, 0.38 mmol) and benzofiran-2-carbaldehyde (56 mg, 0.38 mmol) in 1,2-dichloroethane (5 ml) was added solid sodium triacetoxyborohydride (162 mg, 0.77 mmol). After stirring at room temperature for 48 hours saturated sodium bicarbonate solution was added—effervescence was observed. The aqueous phase was extracted with ethyl acetate. The combined organic phases were dried (MgSO₄) and solvent removed under reduced pressure. The residue was purified by chromatography using 60% ethyl acetate in heptane. Removal of solvent under reduced pressure gave the desired product as an amorphous white solid (29 mg, 15%):

MS m/e (ES+): 521.08 (M⁺+H, 100%), 391.06 (50%);

IR (film): 3268, 3056, 2930, 2856, 1656, 1588, 1569, 1519, 1469, 1454, 1431, 1355, 1342, 1255, 1171, 1105, 1052, 1009, 909, 788, 740 cm⁻¹;

¹H NMR (CDCl₃): δ=1.20-2.20 (14H, m), 3.08 (1H, d, J=14.4 Hz), 3.14 (1H, d, J=14.8 Hz), 3.45-3.49 (2H, m), 3.66 (1H, d, J=14.4 Hz), 3.76 (1H, d, J=14.8 Hz), 6.33 (1H, s), 6.84-6.88 (1H, m), 7.00-7.65 (12H, m), 8.32 (1H, s), 8.39 (1H, d, J=4.0 Hz);

HPLC A: Rt. 8.86 min, 99.7/99.1% purity, 20-100% CH₃CN in H₂O (+0.1%TFA) over 15 min at 1 mlmin⁻¹, Prodigy ODSIII 250×4.6 mm 5 μM, 215 and 254 nm.

### EXAMPLE 27

(S)-3-(1H-Indol-3-yl)-2-methyl-2-(4-nitro-benzylamino)-N-(1-pyridin-2-yl-cyclohexylmethyl)-propionamide

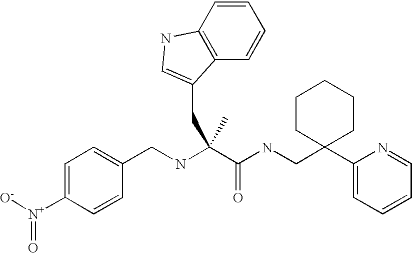

The above compound was prepared as shown in Scheme 5 via Intermediate 10. To a stirred solution of Intermediate 10 (150 mg, 0.38 mmol) and 4-nitrobenzaldehyde (58 mg, 0.38 mmol) in 1,2-dichloroethane (5 ml) was added solid sodium triacetoxyborohydride (114 mg, 0.54 mmol). After stirring at room temperature for 24 hours saturated sodium bicarbonate solution was added—effervescence was observed. The aqueous phase was extracted with ethyl acetate. The combined organic phases were dried (MgSO₄) and solvent removed under reduced pressure. The residue was purified by chromatography using 60% ethyl acetate in heptane. Repurifcation using RP silica with 45% methanol in water (+1% acetic acid) gave pure product. The pure fractions were combined, basified (sodium carbonate), and extracted with ethyl acetate. Removal of solvent under reduced pressure gave the desired compound as a glass (10.5 mg, 5%):

MPt: 58-60° C.;

MS m/e (ES+): 526.15 (M⁺+H, 100%), 527.14 (33%);

IR (film): 3365, 2924, 2856, 1652, 1513, 1429, 1346, 1257, 1048 cm^{—1};

¹H NMR (DMSO-d₆): δ=1.10-1.55 (8H, m), 1.19 (3H, s), 1.88-2.08 (2H, m), 2.25-2.30 (1H, m), 2.95-3.02 (2H, m), 3.10-3.20 (1H, m), 3.17-3.27 (1H, m), 3.50-3.80 (2H, m), 6.93-7.63 (1H, m), 8.12 (2H, d, J=8.8 Hz), 8.42 (1H, d, J=3.6 Hz), 10.86 (1H, s).

### EXAMPLE 28

BB₁ and BB₂ Binding Assays

In the following experiments, measurement of BB₁ and BB₂ binding was as follows. CHO-K1 cells stably expressing cloned human NMB (for (BB₁ assay) and GRP receptors (for BB₂ assay) were routinely grown in Ham's F12 culture medium supplemented with 10% foetal calf serum and 2 mM glutamine. For binding experiments, cells were harvested by trypsinization, and stored frozen at −70° C. in Ham's F12 culture medium containing 5% DMSO until required. On the day of use, cells were thawed rapidly, diluted with an excess of culture medium, and centrifuged for 5 minutes at 2000 g. Cells were resuspended in 50 mM Tris-HCl assay buffer (pH 7.4 at 21° C., containing 0.02% BSA, 40 μg/mL bacitracin, 2 μg/mL chymostatin, 4 μg/mL leupeptin, and 2 μM phosphoramidon), counted, and polytronned (setting 5, 10 sec) before centrifuging for 10 minutes at 28,000 g. The final pellet was resuspended in assay buffer to a final cell concentration of 1.5×10⁵/mL. For binding assays, 200L aliquots of membranes were incubated with [¹²⁵I][Tyr⁴]bombesin (<0.1 nM) in the presence and absence of test compounds (final assay volume 250 μL) for 60 minutes and 90 minutes for NMB and GRP receptors, respectively. Nonspecific binding was defined by 1 μM bombesin. Assays were terminated by rapid filtration under vacuum onto Whatman GF/C filters presoaked in 0.2% PEI for >2 hours, and washed 50 mM Tris-HCl (pH 6.9 at 21° C.; 6×1 mL). Radioactivity bound was determined using a gamma counter.

All competition data was analysed using nonlinear regression utilizing iterative curve-plotting procedures in Prism® (GraphPad Software Inc., San Diego, USA). IC₅₀ values were corrected to Kᵢ values using the Cheng-Prusoff equation (Cheng Y., Prusoff W. H., Biochem. Pharmacol. 22: 3099-3108, 1973). obtained are listed in Table 1.

TABLE 1
Human NMB and GRP receptor binding affinities

| Example No. | NMB Kᵢ(nM) | GRP Kⱼ(nM) |
|---|---|---|
| 9 | 4 | 24 |
| 10 | 469 | |
| 11 | 5580 | |
| 12 | 16 | 2820 |
| 13 | 19 | 1385 |
| 14 | 106 | 1190 |
| 15 | 213 | 1770 |
| 16 | 15 | |
| 17 | 2080 | |
| 18 | 303 | |
| 19 | 1249 | |
| 20 | 3163 | |
| 21 | 824 | |
| 22 | 653 | |
| 23 | 3371 | |
| 24 | 137 | |
| 25 | 616 | 2620 |
| 26 | 2400 | |
| 27 | 652 | |

### EXAMPLE 29

Effect of (S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide (Compound (2) in PEG200 on Female Rat Sexual Proceptivity

Ovariectomised adult female Sprague Dawley rats (1 80-200 g, from Charles River) were housed in groups of 6 in a reversed lighting system of 12 h light:dark (lights off 7.00-19.00 h). Two weeks after ovariectomy they were used for sexual activity tests. Animals were adapted to the apparatus (in the absence of stimuli animals) for 10 min on 2 consecutive days prior to testing. The experiments started at least 5 h into the dark period.

Tests were carried out in a circular arena of 90 cm diameter, surrounded by a 30 cm high wall. Two small cages with wire-mesh front (15×15 cm) are fixed into the wall such that the front of the cage is “flush” with the wall and the 2 cages are opposite each other. They contain two stimuli animals: an intact sexually experienced male and a receptive female (ovariectomised, primed with 5 μg oestradiol benzoate dissolved in corn oil and injected subcutaneously 48 hours before the test and with 0.5 mg of progesterone four hours before the test). Sexually naive test and control animals were used. Forty eight hours before the tests, both the test and control animals were primed with 5 μg oestradiol benzoate. Test animals were treated with the above compound (30-100 mg/kg) which was dissolved in PEG 200 vehicle and administered orally in a 1 ml/kg volume 1 h before each test. For animals used as positive controls, progesterone (0.5 mg/0.1 ml) was dissolved in corn oil and administered subcutaneously (s.c.), 4 h before the test. Test and control animals were introduced one at a time for 10 minute periods into the arena. During the 10 min test, the time that the test or positive control animal spent investigating each stimulus animal was noted. The arena was thoroughly cleaned between animals. The position of the male/female stimuli boxes was randomised between animals, in order to avoid place preference. The difference in the percentage of time spent investigating male minus female was calculated, out of the total time spent investigating stimuli animals.

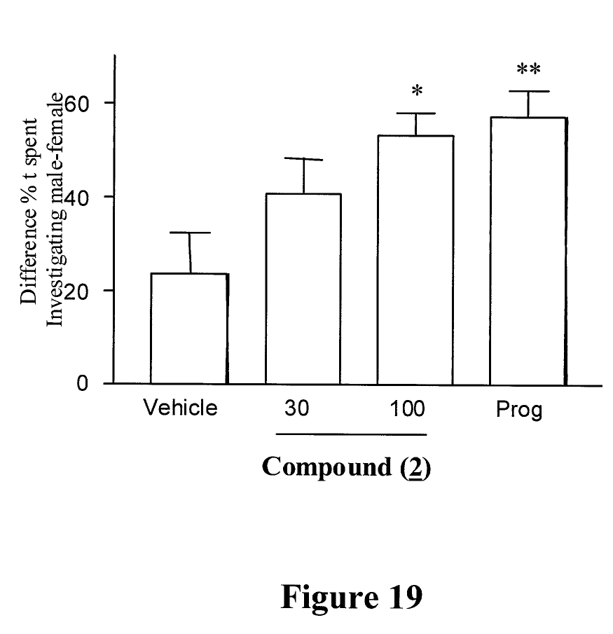

It was found (see FIG. 19) that the above compound dose-dependently (30-100) increased the percentage of time spent investigating the male stimulus, with a MED of 100 mg/kg (see below). The effect of this dose was similar to the effect of progesterone (maximal). (*P<0.05, **P<0.01 Kruskal-Wallis followed by Mann-Whitney test, vs vehicle).

### EXAMPLE 30

Effect of Compound (2) in Methyl Cellulose on Female Rat Sexual Proceptivity.

Example 29 was repeated except that the above compound (3-30 mg/kg) was dissolved in 0.5% methyl cellulose and was administered p.o. in a dosing volume of 3 ml/kg 1 h before tests. Progesterone, (0.5 mg/0.1 ml) was dissolved in corn oil and administered s.c., 4 h before test, as a positive control.

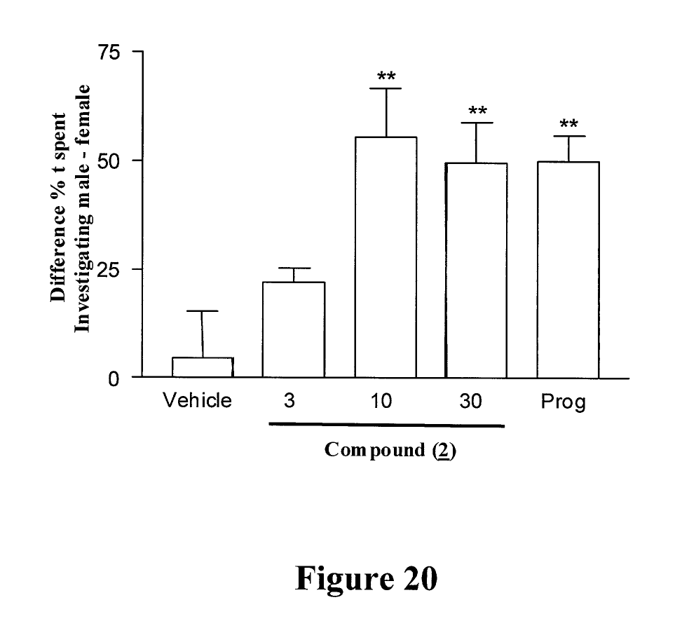

The above compound dose-dependently (3-30 mg/kg) increased the percentage of time spent investigating the male stimulus, with a MED of 10 mg/kg. This represents a 10-fold increase in potency compared to the oral results obtained in the PEG200 vehicle (MED=100 mg/kg). The results are shown in FIG. 20 in which bars represent percentage of time spent investigating male, minus the percentage of time spent investigating the female stimuli±SEM, (n=6-9 per group). *P<0.05, **P<0.01 vs vehicle (One-way ANOVA followed by Dunnett's test vs vehicle group).

### EXAMPLE 31

Effect of Compound (2) in PEG 200 on Female Rat Sexual Receptivity.

Ovariectomised adult female Sprague Dawley rats (180-200 g, from Charles River) were housed in groups of 6 in a reversed lighting system of 12 h light:dark (lights off 7.00-19.00 h). Two weeks after ovariectomy they were used for sexual activity tests. The experiments started at least 5 h into the dark period.

The above compound was dissolved in PEG200 vehicle and administered orally. Quinelorane dihydrochloride (LY 163,502, 6.25 μg/kg) was dissolved in water and administered subcutaneously (s.c.), as a positive control. Both compounds were administered in a 1 ml/kg volume.

Forty eight hours before tests, the animals were primed with 5 μg oestradiol benzoate (Sigma Chemical. Co. Ltd., UK) dissolved in corn oil and injected subcutaneously. The females were placed with a series of vigorous male rats and subjected to 10 mounts. The lordotic response of the animal was recorded and expressed as a percentage of the mounts (i.e. lordosis quotient, LQ). Treatment induced LQ=0-10% in most of the animals, which were considered non-receptive (NR). Animals showing higher LQ were not included in the study. Each rat was tested prior to administration of the compound and then tested similarly at 1 h and 90 min post-injection of the above compound or quinelorane respectively.

A single administration of quinelorane (6.25 μg/kg) significantly (P<0.01) increased the LQ, 90 min after administration, compared to the LQ shown before administration (paired t test). A single oral administration of the above compound dose-dependently (10-100 mg/kg) increased the LQ 1 h after administration, with a MED of 100 mg/kg (P<0.01) compared to the LQ shown before administration (paired t test). The effect of the above compound (100 mg/kg) was similar to the effect of quinelorane (6.25 μg/kg) as is shown in FIG. 21.

### Synthesis Example (Compounds of Formula (III))

(S)-2-Amino-3-(1H-indol-3-yl)-2-methyl-N-(1-pyridin-2-yl-cyclohexylmethyl)-propionamide (Intermediate III-7) and

(S)-2-Amino-3-(1H-indol-3-yI)-2-methyl-N-(1-(5-methoxy-pyridin-2-yl)-cyclohexylmethyl)-propionamide (Intermediate III-6)

In reaction scheme 7 below, Intermediates III-6 and III-7 are made by (i) protecting the amino group of the starting amino acid a with di-t-butyl carbonate and potassium carbonate in dioxane/water, (ii) forming an amide by reaction of the N-protected amino acid with an amine b1 or b2 in dimethylformamide in the presence of O-benzotriazol-1-yl-N,N,N′,N′-tetramethyluronium hexafluorophosphate (HBTU) and N,N-diisopropyl-ethylamine (DIPEA), and (iii) deprotecting the amino group of the product c1 or c2 by reaction with trifluoroacetic acid in dichloromethane.

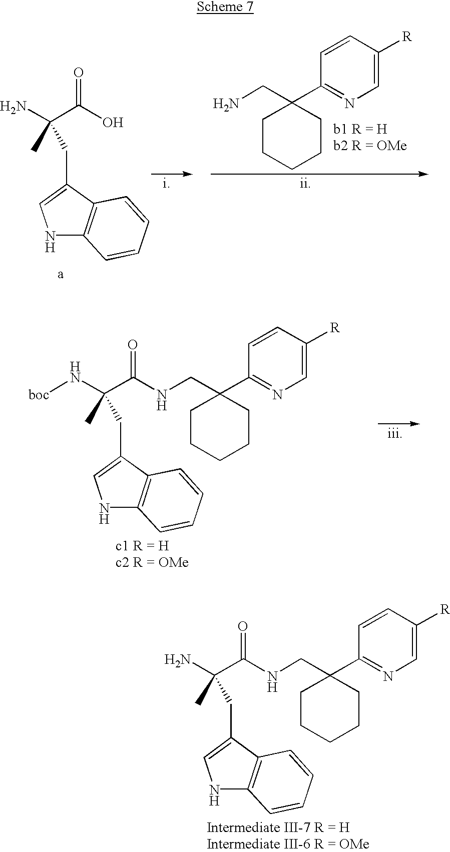

i. BOC₂O, K₂CO₃, dioxane, water

ii. HBTU, DIPEA, DMF

iii. TFA, CH₂Cl₂

{(S)-2-(1-H-Indol-3-yl)-1-methyl-1-[(1-pyridin-2-yl-cyclohexylmethyl)-carbamoyl]-ethyl}-carbamic Acid Tert-butyl Ester (c1)

(1) To a stirred solution of H-(S)-αMeTrp-OH (a) (10 g, 46 mmol) and di-t-butyl-dicarbonate (10 g, 46 mmol) in dioxane (100 ml) was added water (20 ml) and potassium carbonate (10 g, 74 mmol). After 4 hours the reaction mixture was acidified with 2N hydrochloric acid (150 ml) and product extracted with ethyl acetate (2×200 ml). The combined organic phases were dried (MgSO₄) and evaporated under reduced pressure. The residue was purified by flash chromatography, eluting with ethyl acetate. Removal of solvent under reduced pressure gave Boc-(S)-αMeTrp-OH as orange oil (14.5 g, 99%).

(2) To a stirred solution of Boc-(S)-αMeTrp-OH (7 g, 22 mmol) in DMF (100 ml) was added HBTU (8.0 g, 22 mmol), triethylamine (5 ml, 35 mmol), and [1-(2-pyridyl)cyclohexyl]methylamine (b1, 4.2 g, 22 mmol, described in WO 98/07718). After 1 hour the reaction mixture was diluted with ethyl acetate (300 ml) and washed with 2N hydrochloric acid (2×200 ml), dried (MgSO₄) and evaporated under reduced pressure at 60° C. The residue was purified by flash chromatography. Elution with 5% methanol in dichloromethane and subsequent removal of solvent under reduced pressure gave cl as yellow oil (8.3 g, 77%):

IR (film): 3339, 2929, 2858, 1704, 1659, 1651, 1589, 1519, 1487, 1366, 1249, 1164, 1070, 908, 737 cm⁻¹;

NMR (CDCl₃): δ=1.20-1.70 (20H, m), 2.00-2.12 (2H, m), 3.25-3.50 (4H, m), 5.05-5.20 (1H, br.s), 6.92 (1H, d, J=2.0 Hz), 7.02-7.32 (6H, m), 7.51 (1H, d, J=8.0 Hz), 7.59-7.64 (1H, m), 8.03 (1H, s), 8.48 (1H, d, J=4 Hz);

MS m/e (AP+): 491 (M⁺+H, 100%), 513 (M⁺+Na, 20%).

(3) (S)-2-Amino-3-(1H-indol-3-yl)-2-methyl-N-(1-pyridin-2-yl-cyclohexyl methyl)-propionamide (Intermediate III-7)

To a stirred solution of cl (8.2 g, 16.5 mmol) in dichloromethane (100 ml) was added trifluoroacetic acid (3.0 ml, 39 mmol). After 18 hours the solvent was removed under reduced pressure at 60° C. The residue was treated cautiously with saturated sodium carbonate solution (200 ml) before extracting with ethyl acetate (3×200 ml). The combined organic phases were dried (MgSO₄) and evaporated under reduced pressure at 60° C. The residue was purified by flash chromatography. Elution with 0-5% methanol in dichloromethane and subsequent removal of solvent under reduced pressure gave Intermediate III-7 as white foam (4.85 g, 75%).

MPt: 65-68° C.;

IR (KBr disc): 3367, 2926, 2855, 1648, 1589, 1569, 1522, 1455, 1430, 1366, 1341, 1234, 842, 784, 742 cm⁻¹;

NMR (CDCl₃): δ=1.20-1.80 (13H, m), 1.98-2.20 (2H, m), 2.83 (1H, d, J=14.2 Hz), 3.33 (1H, d, J=14.2 Hz), 3.38 (2H, d, J=5.6 Hz), 6.98-7.20 (6H, m), 7.50-7.75 (3H, m), 8.05-8.15 (1H, s), 8.49-8.51 (1H, m);

MS m/e (AP+): 391 (M⁺+H, 100%).

{(S)-2-(1-H-Indol-3-yl)-1-methyl-1-[(1-(5-methoxy-pyridin-2-yl)-cyclohexylmethyl)-carbamoyl]-ethyl}-carbamic Acid Tert-butyl Ester (c2)

To a stirred solution of Boc-(S)-αMeTrp-OH (1.44 g, 4.5 mmol) in DMF (50 ml) was added HBTU (1.72 g, 4.5 mmol), DIPEA (2.38 ml, 13.6 mmol), and [1-(5-methoxy-2-pyridyl)cyclohexyl]methanamine (1 g, 4.5 mmol). After over night the reaction mixture was diluted with ethyl acetate (300 ml) and water, dried (MgSO₄) and evaporated under reduced pressure. The residue was purified by flash chromatography. Elution with ethylacetate/heptane (1:1) and subsequent removal of solvent under reduced pressure gave c2 as an oil (2.207 g, 94%).

NMR (CDCl₃): δ=1.24-1.60 (8H, m), 1.39 (9H, s), 1.52 (3H, s), 2.00-2.18 (2H, m), 3.20-3.43 (4H, m), 3.82 (3H, s), 6.92 (1H, d, J=2.4 Hz), 7.02-7.20 (6H, m), 7.30 (1H, d, J=6.0 Hz), 7.51 (1H, d, J=8 Hz), 8.00 (1H, s), 8.17 (1H, d, J=2.8 Hz).

MS m/e (ES+): 521.36 (M⁺+H, 100%), 543.25 (M⁺+Na).

Intermediate III-6

To a stirred solution of c² (2.2 g, 4.2 mmol) in dichloromethane (10 ml) was added trifluoroacetic acid (5 ml, excess). After stirring over night the reaction mixture was taken up in 1N HCl and extracted with diethylether. Organic phase discarded. The aqueous phase was basified cautiously with saturated sodium carbonate solution before extracting with ethyl acetate (3×50 ml). The combined organic phases were dried (MgSO₄) and evaporated under reduced pressure at 60° C. to give Intermediate III-6 as a glass (1.253 g, 71%).

IR (film): 3272, 2930, 2857, 1651, 1595, 1573, 1520, 1489, 1478, 1455, 1393, 1358, 1291, 1268, 1232, 1181, 1150, 1131, 1030, 1012, 831, 741 cm⁻¹;

NMR (DMSO): δ=1.10-1.65 (13H, m), 1.80-1.90 (1H, m), 2.00-2.10 (1H, m), 2.70 (1H, d, J=13.9 Hz), 3.10 (1H, d, J=13.9 Hz), 3.10-3.22 (2H, m), 3.77 (3H,s), 6.93-7.07 (4H, m), 7.16-7.19 (1H, m), 7.32 (1H, d, J=8.1 Hz), 7.48-7.55 (2H, m), 8.21 (1H, d, J=3.2 Hz), 10.88 (1H, s);

MS m/e (ES+): 421.27 (M⁺+H, 100%), 443.26 (M⁺+Na).

### EXAMPLES 32-86

N-acyl derivatives of Intermediate III-6 and III-7

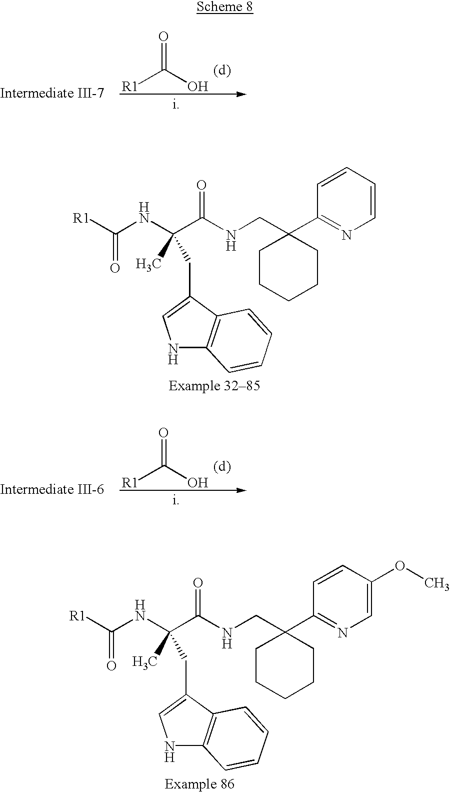

i. HBTU, DIPEA, DMF

In scheme 8, R1 represents the rest of the carboxylic acid d molecule. These intermediates d are listed in table 2.

N-acyl Derivatives of Intermediate III-7

To acid d (0.18 mmol) was added 0.50 M HBTU in DMF (300 μL, 0.15 mmol), 1.0 M diisopropylethylamine in DMF (300 μL, 0.30 mmol) and 0.40 M Intermediate III-7 in DMF (375 μL, 0.15 mmol). The solution was shaken on an orbital shaker at room temperature for 18 h. Water (1.0 mL) was added and the mixture was loaded onto a LC-18 SPE cartridge (0.5 g sorbent) and the cartridge was eluted with water (3 mL), 25% methanol/water (3 mL), 50% methanol/water (4 mL) and methanol (4.5 mL)). The methanol fraction was concentrated and analysed by LCMS. When the purity was <90% the product was further purified by prep. HPLC (column: Phenomenex primesphere 10μ C18-HC 110A, 100×21.20 mm; mobile phase: methanol/water 10 to 100% gradient). The products were characterised and analysed by LCMS (column: 50×4.6 mm Prodigy ODSIII (5μ) column; mobile phase: acetonitrile/water (0.1% formic acid) 5 to 100% gradient over 2 min, held at 100% acetonitrile for 1 min; flow rate 4 mL/min; UV detection at 215 nm; mass spec: 150-900 Da full scan APCI+ centroid data).

The following products were made by the above method, with the starting material listed in Table 2 and gave the test results indicated in Table 3:

TABLE 2

| Example | Intermediate d |
|---|---|
| 32 | Benzoic acid |
| 33 | 4-Methyl-benzoic acid |
| 34 | 4-Chloro-benzoic acid |
| 35 | 4-Methoxy-benzoic acid |
| 36 | 4-Nitro-benzoic acid |
| 37 | 4-Methanesulfonyl-benzoic acid |
| 38 | 3-Cyano-benzoic acid |
| 39 | 3-Chloro-benzoic acid |
| 40 | 3-Methoxy-benzoic acid |
| 41 | 3-Methanesulfonyl-benzoic acid |
| 42 | 3-Dimethylamino-benzoic acid |
| 43 | 3-Methyl-benzoic acid |
| 44 | 2-Chloro-benzoic acid |
| 45 | 2-Nitro-benzoic acid |
| 46 | 2-Methoxy-benzoic acid |
| 47 | 2-Methyl-benzoic acid |
| 48 | 2-Dimethylamino-benzoic acid |
| 49 | 2-Fluoro-benzoic acid |
| 50 | p-Tolyl-acetic acid |
| 51 | o-Tolyl-acetic acid |
| 52 | (4-Hydroxy-phenyl)-acetic acid |
| 53 | (3-Hydroxy-phenyl)-acetic acid |
| 54 | m-Tolyl-acetic acid |
| 55 | (2-Fluoro-phenyl)-acetic acid |
| 56 | Thiophen-3-yl-acetic acid |
| 57 | Pyridine-2-carboxylic acid |
| 58 | Isonicotinic acid |
| 59 | Furan-3-carboxylic acid |
| 60 | Furan-2-carboxylic acid |
| 61 | 1H-Indole-2-carboxylic acid |
| 62 | 5-Methyl-isoxazole-3-carboxylic acid |
| 63 | 1-Methyl-1H-pyrrole-2-carboxylic acid |
| 64 | Thiophene-2-carboxylic acid |
| 65 | Thiophene-3-carboxylic acid |
| 66 | 1H-Indole-6-carboxylic acid |
| 67 | 1H-Indole-5-carboxylic acid |
| 68 | 1H-Indole-4-carboxylic acid |
| 69 | 1H-Indole-7-carboxylic acid |
| 70 | 1-Methyl-1H-indole-2-carboxylic acid |
| 71 | Benzo[b]thiophene-2-carboxylic acid |
| 72 | Benzothiazole-6-carboxylic acid |
| 73 | 1H-Benzotriazole-5-carboxylic acid |
| 74 | 3-Methyl-thiophene-2-carboxylic acid |
| 75 | 5-Methyl-thiophene-2-carboxylic acid |
| 76 | 6-Methyl-pyridine-2-carboxylic acid |
| 77 | Isoquinoline-3-carboxylic acid |
| 78 | Quinoxaline-2-carboxylic acid |
| 79 | Quinoline-8-carboxylic acid |
| 80 | 5-Phenyl-oxazole-4-carboxylic acid |
| 81 | 2-Pyrrol-1-yl-benzoic acid |
| 82 | (4-Methoxy-phenyl)-acetic acid |
| 83 | (4-Dimethylamino-phenyl)-acetic acid |
| 84 | (2-Nitro-phenyl)-acetic acid |
| 85 | (2-Methoxy-phenyl)-acetic acid |
| 86 | 1H-Indole-2-carboxylic acid |

TABLE 3

| | | | | LCMS | BB1 | BB2 |
|---|---|---|---|---|---|---|
| Example | | | Purity | Ret | IC50 | IC50 |
| No | Product | MH⁺ | % | time (min) | (nM) | (nM) |
| 32 | N—{(S)-2-(1H-Indol-3-yl)-1-methyl- | 494,64 | 100 | 1.71 | 2499 | IA |
| | 1-[(1-pyridin-2-yl- | | | | | |
| | cyclohexylmethyl)-carbamoyl]- | | | | | |
| | ethyl}-benzamide | | | | | |
| 33 | N—{(S)-2-(1H-Indol-3-yl)-1-methyl- | 508,67 | 95 | 1.76 | 2499 | IA |
| | 1-[(1-pyridin-2-yl- | | | | | |
| | cyclohexylmethyl)-carbamoyl]- | | | | | |
| | ethyl}-4-methyl-benzamide | | | | | |
| 34 | 4-Chloro-N—{(S)-2-(1H-indol-3-yl)- | 529,09 | 94 | 1.84 | 1349 | IA |
| | 1-methyl-1-[(1-pyridin-2-yl- | | | | | |
| | cyclohexyl-methyl)-carbamoyl]- | | | | | |
| | ethyl}-benzamide | | | | | |
| 35 | N—{(S)-2-(1H-Indol-3-yl)-1-methyl- | 524,67 | 94 | 1.68 | 2879 | IA |
| | 1-[(1-pyridin-2-yl- | | | | | |
| | cyclohexylmethyl)-carbamoyl]- | | | | | |
| | ethyl}-4-methoxy-benzamide | | | | | |
| 36 | N—{(S)-2-(1H-Indol-3-yl)-1-methyl- | 539,64 | 80 | 1.79 | 343 | IA |
| | 1-[(1-pyridin-2-yl- | | | | | |
| | cyclohexylmethyl)-carbamoyl]- | | | | | |
| | ethyl}-4-nitro-benzamide | | | | | |
| 37 | N—{(S)-2-(1H-Indol-3-yl)-1-methyl- | 572,73 | 95 | 1.60 | 2272 | IA |
| | 1-[(1-pyridin-2-yl- | | | | | |
| | cyclohexylmethyl)-carbamoyl]- | | | | | |
| | ethyl}-4-methanesulfonyl-benzamide | | | | | |
| 38 | 3-Cyano-N—{(S)-2-(1H-indol-3-yl)-1- | 519,65 | 91 | 1.71 | 2042 | IA |
| | methyl-1-[(1-pyridin-2-yl- | | | | | |
| | cyclohexylmethyl)-carbamoyl]- | | | | | |
| | ethyl}-benzamide | | | | | |
| 39 | 3-Chloro-N—{(S)-2-(1H-indol-3-yl)- | 529,09 | 97 | 1.84 | 1269 | IA |
| | 1-methyl-1-[(1-pyridin-2-yl- | | | | | |
| | cyclohexyl-methyl)-carbamoyl]- | | | | | |
| | ethyl}-benzamide | | | | | |
| 40 | N—{(S)-2-(1H-Indol-3-yl)-1-methyl- | 524,67 | 98 | 1.73 | 2859 | IA |
| | 1-[(1-pyridin-2-yl- | | | | | |
| | cyclohexylmethyl)-carbamoyl]- | | | | | |
| | ethyl}-3-methoxy-benzamide | | | | | |
| 41 | N—{(S)-2-(1H-Indol-3-yl)-1-methyl- | 572,73 | 95 | 1.60 | 3051 | IA |
| | 1-[(1-pyridin-2-yl- | | | | | |
| | cyclohexylmethyl)-carbamoyl]- | | | | | |
| | ethyl}-3-methanesulfonyl-benzamide | | | | | |
| 42 | Dimethylamino-N—{(S)-2-(1H-indol- | 537,71 | 91 | 1.74 | 2518 | IA |
| | 3-yl)-1-methyl-1-[(1-pyridin-2-yl- | | | | | |
| | cyclohexyl-methyl)-carbamoyl]- | | | | | |
| | ethyl}-benzamide | | | | | |
| 43 | N—{(S)-2-(1H-Indol-3-yl)-1-methyl- | 508,67 | 100 | 1.79 | 2351 | IA |
| | 1-[(1-pyridin-2-yl- | | | | | |
| | cyclohexylmethyl)-carbamoyl]- | | | | | |
| | ethyl}-3-methyl-benzamide | | | | | |
| 44 | 2-Chloro-N—{(S)-2-(1H-indol-3-yl)- | 529,09 | 98 | 1.79 | 3229 | IA |
| | 1-methyl-1-[(1-pyridin-2-yl- | | | | | |
| | cyclohexyl-methyl)-carbamoyl]- | | | | | |
| | ethyl}-benzamide | | | | | |
| 45 | N—{(S)-2-(1H-Indol-3-yl)-1-methyl- | 539,64 | 91 | 1.71 | 4581 | IA |
| | 1-[(1-pyridin-2-yl- | | | | | |
| | cyclohexylmethyl)-carbamoyl]- | | | | | |
| | ethyl}-2-nitro-benzamide | | | | | |
| 46 | N—{(S)-2-(1H-Indol-3-yl)-1-methyl- | 524,67 | 100 | 1.73 | 2559 | IA |
| | 1-[(1-pyridin-2-yl- | | | | | |
| | cyclohexylmethyl)-carbamoyl]- | | | | | |
| | ethyl}-2-methoxy-benzamide | | | | | |
| 47 | N—{(S)-2-(1H-Indol-3-yl)-1-methyl- | 508,67 | 100 | 1.79 | 3283 | IA |
| | 1-[(1-pyridin-2-yl- | | | | | |
| | cyclohexylmethyl)-carbamoyl]- | | | | | |
| | ethyl}-2-methyl-benzamide | | | | | |
| 48 | C-Dimethylamino-N—{(S)-2-(1H- | 537,71 | 93 | 1.79 | 716 | IA |
| | indol-3-yl)-1-methyl-1-[(1-pyridin-2- | | | | | |
| | yl-cyclohexyl-methyl)-carbamoyl]- | | | | | |
| | ethyl}-benzamide | | | | | |
| 49 | 2-Fluoro-N—{(S)-2-(1H-indol-3-yl)- | 512,63 | 98 | 1.76 | 3949 | IA |
| | 1-methyl-1-[(1-pyridin-2-yl- | | | | | |
| | cyclohexylmethyl)-carbamoyl]- | | | | | |
| | ethyl}-benzamide | | | | | |
| 50 | (S)-3-(1H-Indol-3-yl)-2-methyl-N- | 522,70 | 94 | 1.76 | 944 | IA |
| | (1-pyridin-2-yl-cyclohexylmethyl)-2- | | | | | |
| | (2-p-tolyl-ethanoylamino)- | | | | | |
| | propionamide | | | | | |
| 51 | (S)-3-(1H-Indol-3-yl)-2-methyl-N- | 522,70 | 98 | 1.76 | 944 | IA |
| | (1-pyridin-2-yl-cyclohexylmethyl)-2- | | | | | |
| | (2-p-tolyl-ethanoylamino)- | | | | | |
| | propionamide | | | | | |
| 52 | (S)-2-[2-(4-Hydroxy-phenyl)- | 524,67 | 96 | 1.50 | 3135 | IA |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 53 | (S)-2-[2-(3-Hydroxy-phenyl)- | 524,67 | 90 | 1.52 | 1437 | IA |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 54 | (S)-3-(1H-Indol-3-yl)-2-methyl-N- | 522,70 | 95 | 1.76 | 817 | IA |
| | (1-pyridin-2-yl-cyclohexylmethyl)-2- | | | | | |
| | (2-m-tolyl-ethanoylamino)- | | | | | |
| | propionamide | | | | | |
| 55 | (S)-2-[2-(2-Fluoro-phenyl)- | 526,66 | 94 | 1.71 | 878 | 1546 |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 56 | (S)-3-(1H-Indol-3-yl)-2-methyl-N- | 514,70 | 93 | 1.65 | 1437 | IA |
| | (1-pyridin-2-yl-cyclohexylmethyl)-2- | | | | | |
| | (2-thiophen-3-yl-ethanoylamino)- | | | | | |
| | propionamide | | | | | |
| 57 | Pyridine-2-carboxylic acid {(S)-2- | 495,63 | 98 | 1.68 | 3709 | IA |
| | (1H-indol-3-yl)-1-methyl-1-[(1- | | | | | |
| | pyridin-2-yl-cyclohexylmethyl)- | | | | | |
| | carbamoyl]-ethyl)}-amide | | | | | |
| 58 | N—{(S)-2-(1H-Indol-3-yl)-1-methyl- | 495,63 | 98 | 1.47 | 1365 | IA |
| | 1-[(1-pyridin-2-yl- | | | | | |
| | cyclohexylmethyl)-carbamoyl]- | | | | | |
| | ethyl)}-isonicotinamide | | | | | |
| 59 | Furan-3-carboxylic acid {(S)-2-(1H- | 484,60 | 97 | 1.60 | 1204 | IA |
| | indol-3-yl)-1-methyl-1-[(1-pyridin-2- | | | | | |
| | yl-cyclohexylmethyl)-carbamoyl]- | | | | | |
| | ethyl)}-amide | | | | | |
| 60 | Furan-2-carboxylic acid {(S)-2-(1H- | 484,60 | 100 | 1.60 | 1204 | IA |
| | indol-3-yl)-1-methyl-1-[(1-pyridin-2- | | | | | |
| | yl-cyclohexylmethyl)-carbamoyl]- | | | | | |
| | ethyl)}-amide | | | | | |
| 61 | 1H-Indole-2-carboxylic acid {(S)-2- | 533,68 | 100 | 1.79 | 289 | 527 |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 62 | 5-Methyl-isoxazole-3-carboxylic | 499,62 | 94 | 1.46 | 4127 | IA |
| | acid {(S)-2-(1H-indol-3-yl)-1- | | | | | |
| | methyl-1-[(1-pyridin-2-yl- | | | | | |
| | cyclohexylmethyl)-carbamoyl]- | | | | | |
| | ethyl)}-amide | | | | | |
| 63 | 1-Methyl-1H-pyrrole-2-carboxylic | 497,65 | 96 | 1.46 | 4819 | — |
| | acid {(S)-2-(1H-indol-3-yl)-1- | | | | | |
| | methyl-1-[(1-pyridin-2-yl- | | | | | |
| | cyclohexylmethyl)-carbamoyl]- | | | | | |
| | ethyl}-amide | | | | | |
| 64 | Thiophene-2-carboxylic acid {(S)-2- | 500,67 | 100 | 1.42 | 1437 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 65 | Thiophene-3-carboxylic acid {(S)-2- | 500,67 | 100 | 1.39 | 2201 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 66 | 1H-Indole-6-carboxylic acid {(S)-2- | 533,68 | 100 | 1.42 | 1604 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 67 | 1H-Indole-5-carboxylic acid {(S)-2- | 533,68 | 100 | 1.35 | 1881 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 68 | 1H-Indole-4-carboxylic acid {(S)-2- | 533,68 | 99 | 1.35 | 4503 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 69 | 1H-Indole-7-carboxylic acid {(S)-2- | 533,68 | 100 | 1.60 | 1369 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 70 | 1-Methyl-1H-indole-2-carboxylic | 547,71 | 100 | 1.70 | 1233 | IA |
| | acid {(S)-2-(1H-indol-3-yl)-1- | | | | | |
| | methyl-1-[(1-pyridin-2-yl- | | | | | |
| | cyclohexylmethyl)-carbamoyl]- | | | | | |
| | ethyl}-amide | | | | | |
| 71 | Benzo[b]thiophene-2-carboxylic acid | 550,73 | 100 | 1.63 | 611 | IA |
| | {(S)-2-(1H-indol-3-yl)-1-methyl-1- | | | | | |
| | [(1-pyridin-2-yl-cyclohexylmethyl)- | | | | | |
| | carbamoyl]-ethyl}-amide | | | | | |
| 72 | Benzothiazole-6-carboxylic acid | 551,72 | 95 | 1.35 | 897 | 1495 |
| | {(S)-2-(1H-indol-3-yl)-1-methyl-1- | | | | | |
| | [(1-pyridin-2-yl-cyclohexylmethyl)- | | | | | |
| | carbamoyl]-ethyl}-amide | | | | | |
| 73 | 1H-Benzotriazole-5-carboxylic acid | 535,65 | 95 | 1.25 | 3167 | — |
| | {(S)-2-(1H-indol-3-yl)-1-methyl-1- | | | | | |
| | [(1-pyridin-2-yl-cyclohexylmethyl)- | | | | | |
| | carbamoyl]-ethyl}-amide | | | | | |
| 74 | 3-Methyl-thiophene-2-carboxylic | 514,70 | 100 | 1.53 | 744 | IA |
| | acid {(S)-2-(1H-indol-3-yl)-1- | | | | | |
| | methyl-1-[(1-pyridin-2-yl- | | | | | |
| | cyclohexylmethyl)-carbamoyl]- | | | | | |
| | ethyl}-amide | | | | | |
| 75 | 5-Methyl-thiophene-2-carboxylic | 514,70 | 100 | 1.60 | 1663 | IA |
| | acid {(S)-2-(1H-indol-3-yl)-1- | | | | | |
| | methyl-1-[(1-pyridin-2-yl- | | | | | |
| | cyclohexylmethyl)-carbamoyl]- | | | | | |
| | ethyl}-amide | | | | | |
| 76 | 6-Methyl-pyridine-2-carboxylic acid | 509,66 | 98 | 1.6 | 2816 | IA |
| | {(S)-2-(1H-indol-3-yl)-1-methyl-1- | | | | | |
| | [(1-pyridin-2-yl-cyclohexylmethyl)- | | | | | |
| | carbamoyl]-ethyl}-amide | | | | | |
| 77 | Isoquinoline-3-carboxylic acid {(S)- | 545,69 | 100 | 1.71 | 1363 | — |
| | 2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 78 | Quinoxaline-2-carboxyiic acid {(S)- | 546,68 | 94 | 1.67 | 1425 | IA |
| | 2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 79 | Quinoline-8-carboxylic acid {(S)-2- | 545,69 | 96 | 1.57 | 4479 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 80 | 5-Phenyl-oxazole-4-carboxylic acid | 561,69 | 95 | 1.81 | 2660 | IA |
| | {(S)-2-(1H-indol-3-yl)-1-methyl-1- | | | | | |
| | [(1-pyridin-2-yl-cyclohexylmethyl)- | | | | | |
| | carbamoyl]-ethyl}-amide | | | | | |
| 81 | N—{(S)-2-(1H-Indol-3-yl)-1-methyl- | 559,72 | 98 | 1.71 | 361 | IA |
| | 1-[(1-pyridin-2-yl- | | | | | |
| | cyclohexylmethyl)-carbamoyl]- | | | | | |
| | ethyl}-2-pyrrol-1-yl-benzamide | | | | | |
| 82 | (S)-3-(1H-Indol-3-yl)-2-[2-(4- | 538,70 | 98 | 1.71 | 1694 | IA |
| | methoxy-phenyl)-ethanoylamino]-2- | | | | | |
| | methyl-N-(1-pyridin-2-yl- | | | | | |
| | cyclohexylmethyl)-propionamide | | | | | |
| 83 | (S)-2-[2-(4-Dimethylamino-phenyl)- | 551,74 | 100 | 1.36 | 2708 | IA |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 84 | (S)-3-(1H-Indol-3-yl)-2-methyl-2-[2- | 553,67 | 95 | 1.5 | 1979 | IA |
| | (2-nitro-phenyl)-ethanoylamino]-N- | | | | | |
| | (1-pyridin-2-yl-cyclohexylmethyl)- | | | | | |
| | propionamide | | | | | |
| 85 | (S)-3-(1H-Indol-3-yl)-2-[2-(2- | 538,70 | 100 | 1.57 | 1326 | 2479 |
| | methoxy-phenyl)-ethanoylamino]-2- | | | | | |
| | methyl-N-(1-pyridin-2-yl- | | | | | |
| | cyclohexylmethyl)-propionamide | | | | | |

IA: IC50 > 10000 nM

### N-acyl Derivative of Intermediate III-6

### EXAMPLE 86

1H-Indole-2-carboxylic Acid ((S)-2-(1H-indol-3-yl)-1-{[1-(5-methoxy-pyridin-2-yl)-cyclohexylmethyl]-carbamoyl}-1-methyl-ethyl)-amide

To a solution of 1-H-Indole-2-carboxylic acid (38 mg, 0.24 mmol), Intermediate III-6 (100 mg, 0.19 mmol) and diisopropylethylamine (61 mg, 0.47 mmol) in DMF (5 mL) was added HBTU (90 mg, 0.24 mmol). The reaction mixture was stirred at room temperature for 16 h. The reaction mixture was concentrated under reduced pressure and the residue was diluted with ethyl acetate, washed with brine, dried (MgSO₄) and concentrated under reduced pressure. The residue was purified by column chromatography (60% ethyl acetate/heptane) to give Example 86 as an amorphous white solid (65 mg, 61%).

IR(film): 3285,2931,2855, 1651, 1537, 1489, 1456, 1420, 1342, 1310, 1267, 1028, 908, 744 cm⁻¹;

NMR (CDCl₃): δ=1.10-1.61 (1H, m), 1.95-2.04 (2H, m), 3.29-3.52 (4H, m), 3.43 (3H, s), 6.47 (1H,s), 6.86-6.90 (1H, m), 6.98-6.99 (2H, m), 7.09-7.42 (8H, m), 7.52-7.58 (2H, m), 7.73-7.74 (1H, m) 8.05 (1H, s), 9.11 (1H, s);

MS m/e (ES+): 564 (M⁺+H, 100%).

Binding studies of Example 86 to the bombesin receptors gave the following results (IC₅₀): BB1: 11 nM, BB2: 119 nM.

### EXAMPLES 87-110

### N-Terminal Urethane Derivatives of Intermediate III-7

Scheme 9 describes the synthesis of urethane derivatives of Intermediate III-7:

Conversion of alcohol into 4-nitrophenyl carbonates

N-terminal urethane formation

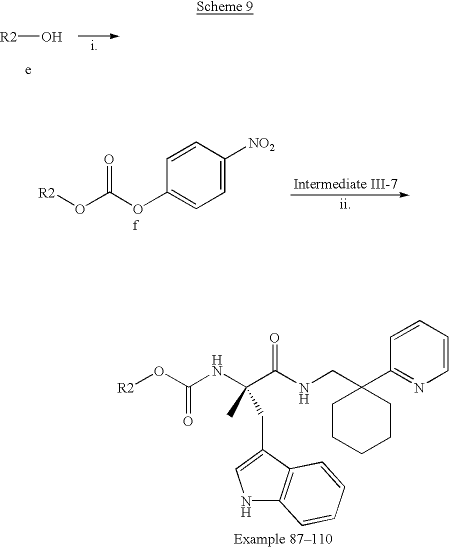

i. 4-nitrophenyl chloroformate, pyridine, THF

ii. DMAP, DMF

In scheme 9, R2 represents the rest of the intermediate e. These intermediates e are listed in table 4.

To a stirred solution of alcohol e (10 mmol) and 4-nitrophenyl chloroformate (2.01 g, 10 mmol) in dichloromethane (50 mL) at 0C was added dropwise a solution of pyridine (0.81 mL, 10 mmol) in dichloromethane (10 mL). The reaction mixture was allowed to slowly warm to room temperature and was stirred at room temperature for 16 h. The solvent was removed under reduced pressure and the residue was taken up in ethyl acetate (50 mL) and was washed successively with 10% citric acid (2×30 mL), water (30 mL), sat. NaHCO₃ solution (2×50 mL) and brine (50 mL). The organic phase was dried (MgSO₄) and was concentrated under reduced pressure. The crude product was recrystallised from typically ethyl acetate, diethyl ether or heptane to give pure carbonate f The product was characterised by IR (see Table 4 for carbonate signals).

To carbonate f (0.21 mmol) was added DMF (0.4 mL) followed by 0.50 M DMAP in DMF (400 μL, 0.20 mmol) and 0.50 M Intermediate 111-7 in DMF (200 μL, 0.10 mmol). The solution was shaken on an orbital shaker at room temperature for 42 h. Water (1.0 mL) was added and the mixture was loaded onto a LC-18 SPE cartridge (0.5 g sorbent) and the cartridge was eluted with 25% methanol/water (3.4 mL) and methanol (4 mL). The methanol fraction was concentrated and purified by prep. HPLC (column: Phenomenex primesphere 10μ C18-HC 110A, 100×21.20 mm; mobile phase: methanol/water 10 to 100% gradient). The products were characterised and analysed by LCMS (column: 50×4.6 mm Prodigy ODSIII (5μ) column; mobile phase: acetonitrile/water (0.1% formic acid) 5 to 100% gradient over 2 min, held at 100% acetonitrile for 1 min; flow rate 4 mL/min; UV detection at 215 nm; mass spec: 150-900 Da full scan APCI+ centroid data).

The following products were made by the above method, with the starting material listed in Table 4 and gave the test results indicated in Table 5:

TABLE 4

| | | intermediate f: |
|---|---|---|
| Example | intermediate e | IR (cm⁻¹) |
| 87 | Naphthalen-1-yl-methanol | 1754 |
| 88 | (3,4-Dimethoxy-phenyl)-methanol | 1754 |
| 89 | Naphthalen-2-yl-methanol | 1752 |
| 90 | Indan-2-ol | 1765 |
| 91 | (3,4-Dichloro-phenyl)-methanol | 1754 |
| 92 | (4-Methoxy-phenyl)-methanol | 1748 |
| 93 | (4-Chloro-phenyl)-methanol | 1761 |
| 94 | (2-Fluoro-phenyl)-methanol | 1752 |
| 95 | (2-Chloro-phenyl)-methanol | 1764 |
| 96 | (4-Nitro-phenyl)-methanol | 1761 |
| 97 | o-Tolyl-methanol | 1757 |
| 98 | (4-tert-Butyl-phenyl)-methanol | 1766 |
| 99 | (3-Nitro-phenyl)-methanol | 1769 |
| 100 | (2-Methoxy-phenyl)-methanol | 1766 |
| 101 | (4-Trifluoromethyl-phenyl)-methanol | 1763 |
| 102 | (3-Ethoxy-phenyl)-methanol | 1767 |
| 103 | 3-Hydroxymethyl-benzonitrile | 1769 |
| 104 | (2,4-Dichloro-phenyl)-methanol | 1768 |
| 105 | m-Tolyl-methanol | 1757 |
| 106 | (3-Phenoxy-phenyl)-methanol | 1766 |
| 107 | (3-Trifluoromethyl-phenyl)-methanol | 1770 |
| 108 | p-Tolyl-methanol | 1759 |
| 109 | (2,3-Dichloro-phenyl)-methanol | 1758 |
| 110 | Quinolin-6-yl-methanol | 1761 |

TABLE 4

| | | | | LCMS | BB1 | BB2 |
|---|---|---|---|---|---|---|
| Example | | | Purity | Ret | IC50 | IC50 |
| No | Product | MH⁺ | % | time (min) | (nM) | (nM) |
| 87 | {(S)-2-(1H-Indol-3-yl)-1-methyl-1- | 574,73 | 100 | 1.67 | 239 | IA |
| | [(1-pyridin-2-yl-cyclohexylmethyl)- | | | | | |
| | carbamoyl]-ethyl}-carbamic acid | | | | | |
| | naphthalen-1-ylmethyl ester | | | | | |
| 88 | {(S)-2-(1H-Indol-3-yl)-1-methyl-1- | 584,72 | 95 | 1.41 | 1758 | IA |
| | [(1-pyridin-2-yl-cyclohexylmethyl)- | | | | | |
| | carbamoyl]-ethyl}-carbamic acid 3,4- | | | | | |
| | dimethoxy-benzyl ester | | | | | |
| 89 | {(S)-2-(1H-Indol-3-yl)-1-methyl-1- | 574,73 | 100 | 1.67 | 1001 | IA |
| | [(1-pyridin-2-yl-cyclohexylmethyl)- | | | | | |
| | carbamoyl]-ethyl}-carbamic acid | | | | | |
| | naphthalen-2-ylmethyl ester | | | | | |
| 90 | {(S)-2-(1H-Indol-3-yl)-1-methyl-1- | 550,71 | 91 | 1.59 | 955 | IA |
| | [(1-pyridin-2-yl-cyclohexylmethyl)- | | | | | |
| | carbamoyl]-ethyl}-carbamic acid | | | | | |
| | indan-2-yl ester | | | | | |
| 91 | {(S)-2-(1H-Indol-3-yl)-1-methyl-1- | 593,56 | 93 | 1.73 | 202 | IA |
| | [(1-pyridin-2-yl-cyclohexylmethyl)- | | | | | |
| | carbamoyl]-ethyl}-carbamic acid 3,4- | | | | | |
| | dichloro-benzyl ester | | | | | |
| 92 | {(S)-2-(1H-Indol-3-yl)-1-methyl-1- | 554,70 | 93 | 1.49 | 1610 | IA |
| | [(1-pyridin-2-yl-cyclohexylmethyl)- | | | | | |
| | carbamoyl]-ethyl}-carbamic acid 4- | | | | | |
| | methoxy-benzyl ester | | | | | |
| 93 | {(S)-2-(1H-Indol-3-yl)-1-methyl-1- | 559,11 | 98 | 1.62 | 681 | IA |
| | [(1-pyridin-2-yl-cyclohexylmethyl)- | | | | | |
| | carbamoyl]-ethyl}-carbamic acid 4- | | | | | |
| | chloro-benzyl ester | | | | | |
| 94 | {(S)-2-(1H-Indol-3-yl)-1-methyl-1- | 542,66 | 91 | 1.52 | 923 | IA |
| | [(1-pyridin-2-yl-cyclohexylmethyl)- | | | | | |
| | carbamoyl]-ethyl}-carbamic acid 2- | | | | | |
| | fluoro-benzyl ester | | | | | |
| 95 | {(S)-2-(1H-Indol-3-yl)-1-methyl-1- | 559,11 | 89 | 1.62 | 624 | IA |
| | [(1-pyridin-2-yl-cyclohexylmethyl)- | | | | | |
| | carbamoyl]-ethyl}-carbamic acid 2- | | | | | |
| | chloro-benzyl ester | | | | | |
| 96 | {(S)-2-(1H-Indol-3-yl)-1-methyl-1- | 569,67 | 97 | 1.51 | 41 | 463 |
| | [(1-pyridin-2-yl-cyclohexylmethyl)- | | | | | |
| | carbamoyl]-ethyl}-carbamic acid 4- | | | | | |
| | nitro-benzyl ester | | | | | |
| 97 | {(S)-2-(1H-Indol-3-yl)-1-methyl-1- | 538,70 | 94 | 11.60 | 751 | IA |
| | [(1-pyridin-2-yl-cyclohexylmethyl)- | | | | | |
| | carbamoyl]-ethyl}-carbamic acid 2- | | | | | |
| | methyl-benzyl ester | | | | | |
| 98 | {(S)-2-(1H-Indol-3-yl)-1-methyl-1- | 580,78 | 100 | 1.86 | 1986 | IA |
| | [(1-pyridin-2-yl-cyclohexylmethyl)- | | | | | |
| | carbamoyl]-ethyl}-carbamic acid 4- | | | | | |
| | tert-butyl-benzyl ester | | | | | |
| 99 | {(S)-2-(1H-Indol-3-yl)-1-methyl-1- | 569,67 | 97 | 1.51 | 17 | 612 |
| | [(1-pyridin-2-yl-cyclohexylmethyl)- | | | | | |
| | carbamoyl]-ethyl}-carbamic acid 3- | | | | | |
| | nitro-benzyl ester | | | | | |
| 100 | {(S)-2-(1H-Indol-3-yl)-1-methyl-1- | 554,70 | 96 | 1.52 | 818 | IA |
| | [(1-pyridin-2-yl-cyclohexylmethyl)- | | | | | |
| | carbamoyl]-ethyl}-carbamic acid 2- | | | | | |
| | methoxy-benzyl ester | | | | | |
| 101 | {(S)-2-(1H-Indol-3-yl)-1-methyl-1- | 592,67 | 97 | 1.7 | 1102 | IA |
| | [(1-pyridin-2-yl-cyclohexylmethyl)- | | | | | |
| | carbamoyl]-ethyl}-carbamic acid 4- | | | | | |
| | trifluoromethyl-benzyl ester | | | | | |
| 102 | {(S)-2-(1H-Indol-3-yl)-1-methyl-1- | 568,72 | 89 | 1.60 | 1065 | IA |
| | [(1-pyridin-2-yl-cyclohexylmethyl)- | | | | | |
| | carbamoyl]-ethyl}-carbamic acid 3- | | | | | |
| | ethoxy-benzyl ester | | | | | |
| 103 | {(S)-2-(1H-Indol-3-yl)-1-methyl-1- | 549,68 | 99 | 1.43 | 85 | IA |
| | [(1-pyridin-2-yl-cyclohexylmethyl)- | | | | | |
| | carbamoyl]-ethyl}-carbamic acid 3- | | | | | |
| | cyano-benzyl ester | | | | | |
| 104 | {(S)-2-(1H-Indol-3-y1)-1-methyl-1- | 593,56 | 95 | 1.78 | 450 | IA |
| | [(1-pyridin-2-yl-cyclohexylmethyl)- | | | | | |
| | carbamoyl]-ethyl}-carbamic acid | | | | | |
| | 2,4-dichloro-benzyl ester | | | | | |
| 105 | {(S)-2-(1H-Indol-3-yl)-1-methyl-1- | 538,70 | 96 | 1.59 | 841 | IA |
| | [(1-pyridin-2-yl-cyclohexylmethyl)- | | | | | |
| | carbamoyl]-ethyl}-carbamic acid 3- | | | | | |
| | methyl-benzyl ester | | | | | |
| 106 | {(S)-2-(1H-Indol-3-yl)-1-methyl-1- | 616,77 | 96 | 1.78 | 1350 | IA |
| | [(1-pyridin-2-yl-cyclohexylmethyl)- | | | | | |
| | carbamoyl]-ethyl}-carbamic acid 3- | | | | | |
| | phenoxy-benzyl ester | | | | | |
| 107 | {(S)-2-(1H-Indol-3-yl)-1-methyl-1- | 592,67 | 96 | 1.67 | 182 | IA |
| | [(1-pyridin-2-yl-cyclohexylmethyl)- | | | | | |
| | carbamoyl]-ethyl}-carbamic acid 3- | | | | | |
| | trifluoromethyl-benzyl ester | | | | | |
| 108 | {(S)-2-(1H-Indol-3-yl)-1-methyl-1- | 538,70 | 97 | 1.60 | 1084 | IA |
| | [(1-pyridin-2-yl-cyclohexylmethyl)- | | | | | |
| | carbamoyl]-ethyl}-carbamic acid 4- | | | | | |
| | methyl-benzyl ester | | | | | |
| 109 | {(S)-2-(1H-Indol-3-yl)-1-methyl-1- | 593,56 | 94 | 1.73 | 152 | IA |
| | [(1-pyridin-2-yl-cyclohexylmethyl)- | | | | | |
| | carbamoyl]-ethyl}-carbamic acid | | | | | |
| | 2,3-dichloro-benzyl ester | | | | | |
| 110 | {(S)-2-(1H-Indol-3-y1)-1-methyl-1- | 575,72 | 97 | 1.22 | 171 | IA |
| | [(1-pyridin-2-yl-cyclohexylmethyl)- | | | | | |
| | carbamoyl]-ethyl}-carbamic acid | | | | | |
| | quinolin-6-ylmethyl ester | | | | | |

### EXAMPLES 111-168

### N-Terminal Sulfonamide Derivatives of Intermediate III-7

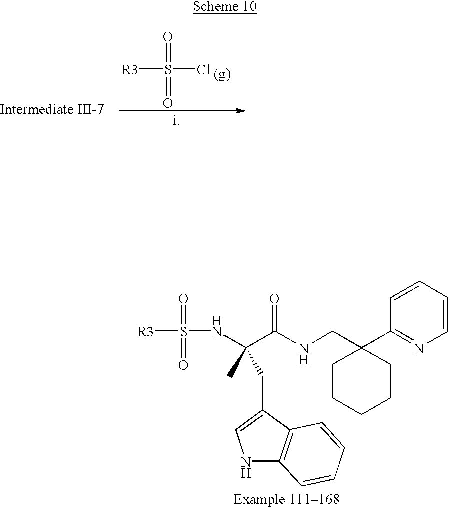

In scheme 10, R3 represents the rest of the intermediate g. These intermediates g are listed in table 6.

To sulfonyl chloride g (0.14 mmol) was added 0.143 M Intermediate III-7 in DMF (700 μL, 0.10 mmol) followed by 300 μL of a solution containing a mixture of diisopropylethylamine (0.667 M in DMF, 0.20 mmol) and 4-dimethylaminopyridine (0.033 M in DMF, 0.01 mmol). The reaction mixture was shaken in an orbital shaker at 70° C. for 16 h. The crude reaction mixture was loaded onto a 5 g silica cartridge and the cartridge was eluted with ethyl acetate in heptane (30 to 100% gradient). Removal of the solvent under reduced pressure gave the sulfonamides (Examples 111-168). The purity of the sulfonamide was checked by LCMS. Those samples that were less than 95% pure were further purified by prep HPLC (column: YMC-Pack ODS-AM, 5 μm, 150×20 mm; mobile phase: acetonitrile/water 40 to 100% gradient). The products were characterised and analysed by LCMS (column: 150×4.6 mm Prodigy ODS3 (3μ) column; mobile phase: acetonitrile (0.085% TFA)/water (0.1% TFA) 20 to 100% gradient over 7 min, held at 100% acetonitrile (0.085% TFA) for 1 min; flow rate 1.5 mL/min; detection: diode array 200-300 nm; mass spec: 150-900 Da full scan APCI+centroid data) (see Table 7).

The following examples were made by the above method, with the starting material listed in Table 6 and gave the test results indicated in Table 7:

TABLE 6

| Example | intermediate g |
|---|---|
| 111 | Phenyl-methanesulfonyl chloride |
| 112 | 4-Methyl-benzenesulfonyl chloride |
| 113 | 2-Chloro-benzenesulfonyl chloride |
| 114 | 2-Fluoro-benzenesulfonyl chloride |
| 115 | Naphthalene-1-sulfonyl chloride |
| 116 | 4-Chloro-benzenesulfonyl chloride |
| 117 | 5-Dimethylamino-naphthalene-1-sulfonyl chloride |
| 118 | Naphthalene-2-sulfonyl chloride |
| 119 | Thiophene-2-sulfonyl chloride |
| 120 | Quinoline-8-sulfonyl chloride |
| 121 | 3-Nitro-benzenesulfonyl chloride |
| 122 | 4-Fluoro-benzenesulfonyl chloride |
| 123 | 4-Nitro-benzenesulfonyl chloride |
| 124 | 3-Trifluoromethyl-benzenesulfonyl chloride |
| 125 | 3,4-Dichloro-benzenesulfonyl chloride |
| 126 | 3-Fluoro-benzenesulfonyl chloride |
| 127 | 4-Trifluoromethyl-benzenesulfonyl chloride |
| 128 | 5-Chloro-thiophene-2-sulfonyl chloride |
| 129 | 2-Trifluoromethyl-benzenesulfonyl chloride |
| 130 | 3-Chloro-benzenesulfonyl chloride |
| 131 | 3-Methyl-benzenesulfonyl chloride |
| 132 | 3,4-Dimethoxy-benzenesulfonyl chloride |
| 133 | 4-Cyano-benzenesulfonyl chloride |
| 134 | 2-Cyano-benzenesulfonyl chloride |
| 135 | 5-Chloro-1,3-dimethyl-1H-pyrazole-4-sulfonyl chloride |
| 136 | 3,5-Dimethyl-isoxazole-4-sulfonyl chloride |
| 137 | Benzo[1,2,5]thiadiazole-4-sulfonyl chloride |
| 138 | 1-Methyl-1H-imidazole-4-sulfonyl chloride |
| 139 | Benzo[1,2,5]oxadiazole-4-sulfonyl chloride |
| 140 | 3-Chlorosulfonyl-thiophene-2-carboxylic acid methyl |
| | ester |
| 141 | 5-Isoxazol-3-yl-thiophene-2-sulfonyl chloride |
| 142 | (2-Nitro-phenyl)-methanesulfonyl chloride |
| 143 | 3-Cyano-benzenesulfonyl chloride |
| 144 | 1,2-Dimethyl-1H-imidazole-4-sulfonyl chloride |
| 145 | 3-Methoxy-benzenesulfonyl chloride |
| 146 | 8-Nitro-naphthalene-1-sulfonyl chloride |
| 147 | 2-Chloro-5-nitro-benzenesulfonyl chloride |
| 148 | 2,4,6-Trichloro-benzenesulfonyl chloride |
| 149 | 4-Chloro-2-nitro-benzenesulfonyl chloride |
| 150 | 5-Benzenesulfonyl-thiophene-2-sulfonyl chloride |
| 151 | 4-Trifluoromethoxy-benzenesulfonyl chloride |
| 152 | 5-Methyl-2-phenoxy-benzenesulfonyl chloride |
| 153 | 2-p-Tolyloxy-benzenesulfonyl chloride |
| 154 | Biphenyl-2-sulfonyl chloride |
| 155 | 2-Chlorosulfonyl-benzoic acid methyl ester |
| 156 | 3-Chloro-4-fluoro-benzenesulfonyl chloride |
| 157 | 2,5-Dichloro-thiophene-3-sulfonyl chloride |
| 158 | 3-Chloro-4-methyl-benzenesulfonyl chloride |
| 159 | 2-Methoxy-4-methyl-benzenesulfonyl chloride |
| 160 | 5-Pyridin-2-yl-thiophene-2-sulfonyl chloride |
| 161 | 5-Bromo-6-chloro-pyridine-3-sulfonyl chloride |
| 162 | 2,4-Dinitro-benzenesulfonyl chloride |
| 163 | 4-Methanesulfonyl-benzenesulfonyl chloride |
| 164 | 4-tert-Butyl-benzenesulfonyl chloride |
| 165 | 2,4-Dichloro-5-methyl-benzenesulfonyl chloride |
| 166 | Chloro-trifluoromethyl-benzenesulfonyl chloride |
| 167 | Nitro-trifluoromethyl-benzenesulfonyl chloride |
| 168 | 4-Butyl-benzenesulfonyl chloride |

TABLE 7

| | | | | LCMS | BB1 | BB2 |
|---|---|---|---|---|---|---|
| Example | | | Purity | Ret | IC50 | IC50 |
| No | Product | MH⁺ | % | time (min) | (nm) | (nm) |
| 111 | (S)-3-(1H-Indol-3-yl)-2-methyl-2- | 544,72 | 100 | 4.64 | 186 | IA |
| | phenylmethanesulfonylamino-N-(1- | | | | | |
| | pyridin-2-yl-cyclohexylmethyl)- | | | | | |
| | propionamide | | | | | |
| 112 | (S)-3-(1H-Indol-3-yl)-2-methyl-N-(1- | 544,72 | 100 | 4.74 | 557 | IA |
| | pyridin-2-yl-cyclohexylmethyl)-2- | | | | | |
| | (toluene-4-sulfonylamino)- | | | | | |
| | propionamide | | | | | |
| 113 | (S)-2-(2-Chloro- | 565,14 | 100 | 4.71 | 257 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 114 | (S)-2-(2-Fluoro- | 548,68 | 100 | 4.54 | 267 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 115 | (S)-3-(1H-Indol-3-yl)-2-methyl-2- | 580,76 | 99 | 4.98 | 185 | 1576 |
| | (naphthalene-1-sulfonylamino)-N-(1- | | | | | |
| | pyridin-2-yl-cyclohexylmethyl)- | | | | | |
| | propionamide | | | | | |
| 116 | (S)-2-(4-Chloro- | 565,14 | 97 | 4.89 | 373 | 4386 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 117 | (S)-2-(5-Dimethylamino- | 623,82 | 100 | 4.39 | 1302 | IA |
| | napbthalene-1-sulfonylamino)-3-(1H- | | | | | |
| | indol-3-yl)-2-methyl-N-(1-pyridin-2- | | | | | |
| | yl-cyclohexylmethyl)-propionamide | | | | | |
| 118 | (S)-3-(1H-Indol-3-yl)-2-methyl-2- | 580,76 | 100 | 5.01 | 322 | IA |
| | (naphthalene-2-sulfonylamino)-N-(1- | | | | | |
| | pyridin-2-yl-cyclohexylmethyl)- | | | | | |
| | propionamide | | | | | |
| 119 | (S)-3-(1H-Indol-3-yl)-2-methyl-N-(1- | 536,72 | 99 | 4.39 | 232 | Ia |
| | pyridin-2-yl-cyclohexylmethyl)-2- | | | | | |
| | (thiophene-2-sulfonylamino)- | | | | | |
| | propionamide | | | | | |
| 120 | (S)-3-(1H-Indol-3-yl)-2-methyl-N-(1- | 581,74 | 99 | 4.53 | 108 | IA |
| | pyridin-2-yl-cyclohexylmethyl)-2- | | | | | |
| | (quino1ine-8-su1fonylamino)- | | | | | |
| | propionamide | | | | | |
| 121 | (S)-3-(1H-Indol-3-yl)-2-methyl-2-(3- | 575,69 | 99 | 4.58 | 208 | 1960 |
| | nitro-benzenesulfonylamino)-N-(1- | | | | | |
| | pyridin-2-yl-cyclohexylmethyl)- | | | | | |
| | propionamide | | | | | |
| 122 | (S)-2-(4-Fluoro- | 548,68 | 100 | 4.60 | 560 | 4165 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 123 | (S)-3-(1H-Indol-3-yl)-2-methyl-2-(4- | 575,69 | 98 | 4.65 | 515 | IA |
| | nitro-benzenesulfonylamino)-N-(1- | | | | | |
| | pyridin-2-yl-cyclohexylmethyl)- | | | | | |
| | propionamide | | | | | |
| 124 | (S)-3-(1H-Indol-3-yl)-2-methyl-N-(1- | 599,58 | 100 | 5.03 | 440 | 2246 |
| | pyridin-2-yl-cyclohexylmethyl)-2-(3- | | | | | |
| | trifluoromethyl- | | | | | |
| | benzenesulfonylamino)- | | | | | |
| | propionamide | | | | | |
| 125 | (S)-2-(3,4-Dichloro- | 599,58 | 99 | 5.47 | 216 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 126 | (S)-2-(3-Fluoro- | 548,68 | 100 | 4.65 | 407 | 2761 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 127 | (S)-3-(1H-Indol-3-yl)-2-methyl-N-(1- | 598,69 | 95 | 5.31 | 553 | IA |
| | pyridin-2-yl-cyclohexylmethyl)-2-(4- | | | | | |
| | trifluoromethyl- | | | | | |
| | benzenesulfonylamino)- | | | | | |
| | propionamide | | | | | |
| 128 | (S)-2-(5-Chloro-thiophene-2- | 571,17 | 99 | 4.94 | 404 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 129 | (S)-3-(1H-Indol-3-yl)-2-methyl-N-(1- | 598,69 | 99 | 5.11 | 134 | — |
| | pyridin-2-yl-cyclohexylmethyl)-2-(2- | | | | | |
| | trifluoromethyl- | | | | | |
| | benzenesulfonylamino)- | | | | | |
| | propionamide | | | | | |
| 130 | (S)-2-(3-Chloro- | 565,14 | 99 | 5.05 | 331 | 2687 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 131 | (S)-3-(1H-indol-3-yl)-2-methyl-N-(1- | 544,72 | 99 | 4.93 | 393 | 1019 |
| | pyridin-2-yl-cyclohexylmethyl)-2- | | | | | |
| | (toluene-3-sulfonylamino)- | | | | | |
| | propionamide | | | | | |
| 132 | (S)-2-(3,4-Dimethoxy- | 590,75 | 98 | 4.50 | 608 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 133 | (S)-2-(4-Cyano- | 555,70 | 99 | 4.61 | 766 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 134 | (S)-2-(2-Cyano- | 555,70 | 97 | 4.62 | 408 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 135 | (S)-2-(5-Chloro-1,3-dimethyl-1H- | 583,16 | 98 | 4.38 | 1252 | IA |
| | pyrazole-4-sulfonylamino)-3-(1H- | | | | | |
| | indol-3-yl)-2-methyl-N-(1-pyridin-2- | | | | | |
| | yl-cyclohexylmethyl)-propionamide | | | | | |
| 136 | (S)-2-(3,5-Dimethyl-isoxazole-4- | 549,70 | 96 | 4.54 | 515 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 137 | (S)-2-(Benzo[1,2,5]thiadiazole-4- | 588,76 | 97 | 4.67 | 256 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 138 | (S)-3-(1H-Indol-3-yl)-2-methyl-2-(1- | 534,69 | 100 | 3.60 | 3667 | IA |
| | methyl-1H-imidazole-4- | | | | | |
| | sulfonylamino)-N-(1-pyridin-2-yl- | | | | | |
| | cyclohexylmethyl)-propionamide | | | | | |
| 139 | (S)-2-(Benzo[1,2,5]oxadiazole-4- | 572,69 | 100 | 4.70 | 507 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 140 | 3-{(S)-2-(1H-Indol-3-yl)-1-methyl-1- | 594,76 | 100 | 4.79 | 167 | IA |
| | [(1-pyridin-2-yl-cyclohexylmethyl)- | | | | | |
| | carbamoyl]-ethylsulfamoyl}- | | | | | |
| | thiophene-2-carboxylic acid methyl | | | | | |
| | ester | | | | | |
| 141 | (S)-3-(1H-Indol-3-yl)-2-(5-isoxazol- | 603,77 | 98 | 4.60 | 534 | IA |
| | 3-yl-thiophene-2-sulfonylamino)-2- | | | | | |
| | methyl-N-(1-pyridin-2-yl- | | | | | |
| | cyclohexylmethyl)-propionamide | | | | | |
| 142 | (S)-3-(1H-Indol-3-yl)-2-methyl-2-(2- | 589,72 | 100 | 4.65 | 430 | IA |
| | nitro-phenylmethanesulfonylamino)- | | | | | |
| | N-(1-pyridin-2-yl- | | | | | |
| | cyclohexylmethyl)-propionamide | | | | | |
| 143 | (S)-2-(3-Cyano- | 555,70 | 99 | 4.55 | 460 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 144 | (S)-2-(1,2-Dimethyl-1H-imidazole-4- | 548,71 | 96 | 3.55 | 2482 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 145 | (S)-3-(1H-Indol-3-yl)-2-(3-methoxy- | 560,72 | 99 | 4.75 | 295 | 3686 |
| | benzenesulfonylamino)-2-methyl-N- | | | | | |
| | (1-pyridin-2-yl-cyclohexylmethyl)- | | | | | |
| | propionamide | | | | | |
| 146 | (S)-3-(1H-Indol-3-yl)-2-methyl-2-(8- | 625,75 | 99 | 4.89 | 177 | IA |
| | nitro-naphthalene-1-sulfonylamino)- | | | | | |
| | N-(1-pyridin-2-yl- | | | | | |
| | cyclohexylmethyl)-propionamide | | | | | |
| 147 | (S)-2-(2-Chloro-5-nitro- | 610,14 | 96 | 5.00 | 374 | Ia |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 148 | (S)-3-(1H-Indol-3-yl)-2-methyl-N-(1- | 634,03 | 100 | 5.45 | 215 | Ia |
| | pyridin-2-yl-cyclohexylmethyl)-2- | | | | | |
| | (2,4,6-trichloro- | | | | | |
| | benzenesulfonylamino) | | | | | |
| | propionamide | | | | | |
| 149 | (S)-2-(4-Chloro-2-nitro- | 610,14 | 100 | 5.13 | 513 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 150 | (S)-2-(5-Benzenesulfonyl-thiophene- | 676,88 | 100 | 5.03 | 297 | IA |
| | 2-sulfonylamino)-3-(1H-indol-3-yl)- | | | | | |
| | 2-methyl-N-(1-pyridin-2-yl- | | | | | |
| | cyclohexylmethyl)-propionamide | | | | | |
| 151 | (S)-3-(1H-Indol-3-yl)-2-methyl-N-(1- | 614,69 | 99 | 5.35 | 635 | IA |
| | pyridin-2-yl-cyclohexylmethyl)-2-(4- | | | | | |
| | trifluoromethoxy- | | | | | |
| | benzenesulfonylamino)-propion | | | | | |
| | amide | | | | | |
| 152 | (S)-3-(1H-Indol-3-yl)-2-methyl-2-(5- | 636,82 | 97 | 5.79 | 76 | IA |
| | methyl-2-phenoxy- | | | | | |
| | benzenesulfonylamino)-N-(1-pyridin- | | | | | |
| | 2-yl-cyclohexylmethyl)- | | | | | |
| | propionamide | | | | | |
| 153 | (S)-3-(1H-Indol-3-yl)-2-methyl-N-(1- | 636,82 | 97 | 5.79 | 90 | IA |
| | pyridin-2-yl-cyclohexylmethyl)-2-(2- | | | | | |
| | p-tolyloxy-benzenesulfonylamino)- | | | | | |
| | propionamide | | | | | |
| 154 | (S)-2-(Biphenyl-2-sulfonylamino)-3- | 606,79 | 97 | 5.52 | 166 | IA |
| | (1H-indol-3-yl)-2-methyl-N-(1- | | | | | |
| | pyridin-2-yl-cyclohexylmethyl)- | | | | | |
| | propionamide | | | | | |
| 155 | 2-{(S)-2-(1H-Indol-3-yl)-1-methyl-1- | 588,73 | 99 | 4.84 | 242 | IA |
| | [(1-pyridin-2-yl-cyclohexylmethyl)- | | | | | |
| | carbamoyl]-ethylsulfamoyl}-benzoic | | | | | |
| | acid methyl ester | | | | | |
| 156 | (S)-2-(3-Chloro-4-fluoro- | 583,13 | 95 | 5.12 | 284 | 1216 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 157 | (S)-2-(2,5-Dichloro-thiophene-3- | 605,61 | 99 | 5.23 | 214 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 158 | (S)-2-(3-Chloro-4-methyl- | 579,17 | 97 | 5.28 | 299 | 3939 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 159 | (S)-3-(1H-Indol-3-yl)-2-(2-methoxy- | 574,75 | 96 | 4.92 | 445 | IA |
| | 4-methyl-benzenesulfonylamino)-2- | | | | | |
| | methyl-N-(1-pyridin-2-yl- | | | | | |
| | cyclohexylmethyl)-propionamide | | | | | |
| 160 | (S)-3-(1H-Indol-3-yl)-2-methyl-N-(1- | 613,81 | 100 | 4.79 | 344 | IA |
| | pyridin-2-yl-cyclohexylmethyl)-2-(5- | | | | | |
| | pyridin-2-yl-thiophene-2- | | | | | |
| | sulfonylamino)-propionamide | | | | | |
| 161 | (S)-2-(5-Bromo-6-chloro-pyridine-3- | 645,02 | 95 | 5.09 | 187 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 162 | (S)-2-(2,4-Dinitro- | 620,69 | 100 | 4.97 | 475 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 163 | (S)-3-(1H-Indol-3-yl)-2-(4- | 608,78 | 98 | 4.20 | 1043 | IA |
| | methanesulfonyl- | | | | | |
| | benzenesulfonylamino)-2-methyl-N- | | | | | |
| | (1-pyridin-2-yl-cyclohexylmethyl)- | | | | | |
| | propionamide | | | | | |
| 164 | (S)-2-(4-tert-Butyl- | 586,80 | 96 | 5.65 | 406 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 165 | (S)-2-(2,4-Dichloro-5-methyl- | 613,61 | 97 | 5.64 | 172 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 166 | (S)-2-(Chloro-trifluoromethyl- | 633,14 | 100 | 5.33 | 627 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 167 | (S)-3-(1H-Indol-3-yl)-2-methyl-2- | 643,69 | 100 | 5.34 | 758 | IA |
| | (nitro-trifluoromethyl- | | | | | |
| | benzenesulfonylamino)-N-(1-pyridin- | | | | | |
| | 2-yl-cyclohexylmethyl)- | | | | | |
| | propionamide | | | | | |
| 168 | (S)-2-(4-Butyl- | 586,80 | 96 | 5.84 | 492 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |

### EXAMPLE 169

Bombesin Antagonists Potentiate Pelvic Nerve-Stimulated Increases in Female Genital Blood Flow in the Anaesthetised Rabbit Model of Sexual Arousal.

Bombesin Anatgonists Used=Compound 1 and (2S)-N-{[1-(4-aminophenyl) cyclohexyll methyl}-3-(1H-indol-3-yl)-2-methyl-2-{[(4-nitroanilino) carbonyl]amino}propanamide (Compound 3).

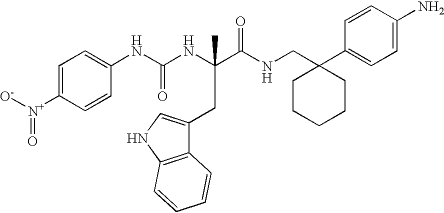

### hBB₁ Ki 0.25 nM

### hBB₂ Ki 46 nM

Female New Zealand rabbits (˜2.5kg) were pre-medicated with a combination of Medetomidine (Domitor®) 0.5 ml/kg i.m., and Ketamine (Vetalar®) 0.25 ml/kg im. whilst maintaining oxygen intake via a face mask. The rabbits were tracheotomised using a PortexTM uncuffed endotracheal tube 3 ID., connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane and ventilation continued with O₂ at 21/min. The right marginal ear vein was cannulated using a 23G or 24G catheter, and Lactated Ringer solution perfused at 0.5 ml/min. The rabbit was maintained at 3% Isoflurane during invasive surgery, dropping to 2% for maintenance anaesthesia.

The left groin area of the rabbit was shaved and a vertical incision was made approximately 5 cm in length along the thigh. The femoral vein and artery were exposed, isolated and then cannulated with a PVC catheter (17G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic x2 +systolic)÷3. Heart rate was measured via the pulse oxymeter and Po-ne-mah data acquisition software system (Ponemah Physiology Platform, Gould Instrument Systems Inc). A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in vaginal and clitoral blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:-5V, pulse width 0.5 ms, duration of stimulus 10 seconds and a frequency range of 2 to 16 Hz. Reproducible responses were obtained when the nerve was stimulated every 15-20 minutes. A frequency response curve was determined at the start of each experiment in order to determine the optimum frequency to use as a sub-maximal response, normally 4 Hz. A ventral midline incision was made, at the caudal end of the pubis, to expose the pubic area. Connective tissue was removed to expose the tunica of the clitoris, ensuring that the wall was free from small blood vessels. The external vaginal wall was also exposed by removing any connective tissue. One laser Doppler flow probe was inserted 3 cm into the vagina, so that half the probe shaft was still visible. A second probe was positioned so that it lay just above the external clitoral wall. The position of these probes was then adjusted until a signal was obtained. A second probe was placed just above the surface of a blood vessel on the external vaginal wall. Both probes were clamped in position.

Compound 1 and compound 3 were dissolved in 50% β-cyclodextrin in saline. They were administered at a dose of 15 mg/kg subcutaneously (sc). Vaginal and clitoral blood flow was recorded either as numbers directly from the Flowmeter using Po-ne-mah data acquisition software (Ponemah Physiology Platform, Gould Instrument Systems Inc), or indirectly from Gould chart recorder trace. Calibration was set at the beginning of the experiment (0-125 ml/min/100 g tissue). All data are reported as mean±s.e.m. Significant changes were identified using Student's t-tests.

The non-selective BB1/BB2 bombesin receptor antagonist (Compound 1; 15 mg/kg sc) acts as a potent enhancer of pelvic-nerve stimulated (PNS) increases in genital blood flow in the anaesthetised rabbit (FIG. 22). Compound 1 had no effect on basal genital blood flow in the absence of PNS (FIG. 22). This reinforces our view that antagonising/blocking BB1/BB2 receptors will enhance the arousal response by potentiating the central mechanism(s) that control sexual arousal/genital blood flow, and will not induce arousal in the absence of sexual stimulation.

The selective BB1 receptor antagonist (Compound 3; 15 mg/kg sc) acts as a potent enhancer of pelvic-nerve stimulated (PNS) increases in vaginal and clitoral blood flow in the anaesthetised rabbit (FIG. 23). The potentiation was significant 45 mins after sc dosing and remained elevated for circa 1 hr. Compound 3 had no effect on basal genital blood flow in the absence of PNS (FIG. 23). This reinforces our view that a selective BB1 receptor antagonist will enhance the arousal response by potentiating the central mechanism(s) that control sexual arousal/genital blood flow, thereby treating FSAD, and will not induce arousal in the absence of sexual stimulation. Since these agents also enhance clitoral blood flow it is likely that they will be effective in the treatment of orgasmic disorders.

At the level of the genitalia, the enhancement observed is similar to the beneficial effects observed with apomorphine, and consequently we believe that a centrally mediated potentiation of the descending neuronal pathways that control genital blood flow is responsible.

### EXAMPLE 170

Bombesin Antagonists (Compounds 1 and 3) Induce Increases in Penile Intracavernosal Pressure in the Conscious Male Rat.

Bombesin Anatgonists Used=Compound 1 and (2S)-N-{[1-(4-aminophenyl) cyclohexyl]methyl}-3-(1H-indol-3-yl)-2-methyl-2-{[(4-nitroanilino) carbonyl]amino}propanamide (Compound 3).

In addition to treating women with FSD, bombesin antagonists will be useful in treating male erectile dysfunction (MED. Both the non-selective BB1/2 antagonists (Compound 1; 10 mg/kg sc) and the selective BB1 receptor antagonist (Compound 3; 15 mg/kg sc) is pro-erectile in a conscious rat model of penile erection (FIGS. 24 and 25). Erectile responses were recorded by measuring intracavemosal pressure using surgically implanted telemetric device. The specific details the surgical procedures, data acquisition and analysis can be found in detail in Bernabe 1999.

Compound 1 and compound 3 were dissolved in 50% β-cyclodextrin in saline. They were administered at a dose of 15 mg/kg subcutaneously (sc). One hour after 10 mg/kg subcutaneous administration of Compound 1 and 45 minutes after a subcutaneous administration of 15 mg/kg compound 3 one observes significant increases in intracavernosal pressure. These increases equate to penile erection. Both Compounds 1 and 3 induced a number of erections in a similar manner to those observed with apomorphine or melanotan-II—both of which are clinically proven agents that are effective in the treatment of MED. Moreover the amplitude of the increases observed were similar to those observed with apomorphine or melanotan-II. The mechanism of action is thought to be similar to the effects on female genital flow ie CNS potentiation of the descending neuronal pathways that control penile erection.

### EXAMPLE 171

Concomitant Administration of a Bombesin Antagonists with a PDE5 Inhibitor Enhance Pelvic Nerve Stimulated Increases in Penile Intracavernosal Pressure in an Anaesthetised Rabbit Model of Erection

In addition to treating women with FSD, bombesin antagonists will be useful in treating male erectile dysfunction (MED) either alone or in combination with a selective PDE5 inhibitor.

Male New Zealand rabbits (˜2.5kg) were pre-medicated with a combination of Medetomidine (Domitor®) 0.5 ml/kg i.m., and Ketamine (Vetalar®)) 0.25 ml/kg i.m. whilst maintaining oxygen intake via a face mask. The rabbits were tracheotomised using a PorteXTM uncuffed endotracheal tube 3 ID., connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane and ventilation continued with O₂ at 21/min. The right marginal ear vein was cannulated using a 23G or 24G catheter, and Lactated Ringer solution perfused at 0.5 ml/min. The rabbit was maintained at 3% Isoflurane during invasive surgery, dropping to 2% for maintenance anaesthesia. The left jugular vein was exposed, isolated and then cannulated with a PVC catheter (17G) for the infusion of drugs and compounds.

The left groin area of the rabbit was shaved and a vertical incision was made approximately 5 cm in length along the thigh. The femoral vein and artery were exposed, isolated and then cannulated with a PVC catheter (17G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic x2+systolic)÷3. Heart rate was measured via the pulse oxymeter and Po-ne-mah data acquisition software system (Ponemah Physiology Platform, Gould Instrument Systems Inc).

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in intracavemosal pressure and cavemosal blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:-5V, pulse width 0.5 ms, duration of stimulus 20 seconds with a frequency of 16 Hz. Reproducible responses were obtained when the nerve was stimulated every 15-20 minutes. Several stimulations using the above parameters were performed to establish a mean control response. The compound(s) to be tested were infused, via the jugular vein, using a Harvard 22 infusion pump allowing a continuous 15 minute stimulation cycle. The skin and connective tissue around the penis was removed to expose the penis. A catheter set (Insyte-W, Becton-Dickinson 20 Gauge 1.1×48 mm) was inserted through the tunica albica into the left corpus cavernosal space and the needle removed, leaving a flexible catheter. This catheter was linked via a pressure transducer (Ohmeda 5299-04) to a Gould system to record intracavemosal pressure. Once an intracavemosal pressure was established, the catheter was sealed in place using Vetbond (tissue adhesive, 3M). Heart rate was measured via the pulse oxymeter and Po-ne-mah data acquisition software system (Ponemah Physiology Platform, Gould Instrument Systems Inc).

Intracavemosal blood flow was recorded either as numbers directly from the Flowmeter using Po-ne-mah data acquisition software (Ponemah Physiology Platform, Gould Instrument Systems Inc), or indirectly from Gould chart recorder trace. Calibration was set at the beginning of the experiment (0-125 ml/min/100 g tissue). All data are reported as mean±s.e.m. Significant changes were identified using Student's t-tests.

Compound 1 and compound 3 were dissolved in 50% β-cyclodextrin in saline. They were administered at a dose of 15 mg/kg subcutaneously (sc). Concomitant inhibition of BB1 receptors with compound 3 and PDE5 enzyme with a PDE5 inhibitor produced a marked enhancement of intracavernosal pressure, or the erectile process.

FIG. 26 demonstrate that concomitant inhibition of Compound 3 (10 mg/kg sc) and a selective inhibitor of PDE5 (3-ethyl-5-{5-[4-ethylpiperzino)sulphonyl-2-propoxyphenyl}-2-(2-pyridylmethyl)-6,7-dihydro-2H-pyrazolo[4,3-d]pyrimidin-7-one also known as 3-ethyl-5-{5-[4-ethylpiperzin-1-ylsulphonyl)-2-n-propoxyphenyl}-2-(2-pyridyl)methyl-2,6-dihydro-7H-pyrazolo[4,3-d]pyrimidin-7-one; See WO98/491066; 1 mg/kg iv) produced a marked enhancement of the ICP, or the erectile process, than was achievable with the same dose of the same Compound 3 inhibitor alone. BB1 antagonists and PDE5 inhibitors or combinations of the two, have no significant effect on un-stimulated intracavernosal pressure ie they do not induce an increase in the absence of sexual drive/arousal. This data illustrates that there are a number of clinical benefits of concomitant administration of a PDE5 inhibitor and a bombesin antagonist over PDE5 inhibitor therapy alone. These include increased efficacy and opportunities to treat MED subgroups that do not respond to PDE5 inhibitor therapy.

Test Assays: Auxiliary Compounds

NEP Enzyme Assay

The Preparation and Assay of Soluble (NEP) Neutral Endopeptidase from Canine, Rat, Rabbit and Human Kidney Cortex.

Soluble NEP is obtained from the kidney cortex and activity is assayed by measuring the rate of cleavage of the NEP substrate Abz-D-Arg-Arg-Leu-EDDnp to generate its fluorescent product, Abz-D-Arg-Arg.

Experimental Procedure:

1. Materials

All water is double de ionised.

1.1 Tissues

Human Kidney IIAM (Pennsylvania. U.S.A.)

Rat Kidney

Rabbit Kidney

Canine Kidney

1.2 Homogenisation Medium

100 mM Mannitol and 20 mM Tris@pH 7.1

2.42 g Tris (Fisher T/P630/60) is diluted in 1 litre of water and the pH adjusted to 7.1 using 6M HCl at room temperature. To this 18.22 g Mannitol (Sigma M-9546) is added.

1.3 Tris Buffer (NEP Buffer).

50 ml of 50 mM Tris pH 7.4 (Sigma T2663) is diluted in 950 ml of water.

1.4 Substrate (Abz-D-Arg-Arg-Leu-EDDnp)

Made to order from SNPE, and is stored as a powder at −20° C. A 2 mM stock is made by gently re-suspending the substrate in Tris buffer, this should not be vortexed or sonicated. 600 μl aliquots of the 2 mM stock are stored at −20 for up to one month. (Medeiros, M. A. S., Franca, M. S. F. et al., (1997), Brazilian Journal of Medical and Biological Research, 30, 1157-1162).

1.5 Total Product

Samples corresponding to 100% substrate to product conversion are included on the plate to enable the % substrate turnover to be determined. The total product is generated by incubating 1 ml of 2 mM substrate with 20 μl of enzyme stock for 24 hours at 37° C.

1.6 Stop Solution.

A 300 μM stock of Phosphoramidon (Sigma R7385) is made up in NEP buffer and stored in 50 μl aliquots at −20.

1.7 Dimethyl Sulphoxide (DMSO).

1.8 Magnesium Chloride-MgCl₂.6H₂O (Fisher M0600/53).

1.9 Black 96 Well Flat Bottom Assay Plates (Costar 3915).

1.10 Topseal A (Packard 6005185).

1.11 Centrifuge Tubes

2. Specific Equiptment

2.1 Sorvall RC-5B centrifuge (SS34 GSA rotor, pre-cooled to 4° C.).

2.2 Braun Miniprimer Mixer.

2.3 Beckman CS-6R Centrifuge.

2.4 Fluostar Galaxy.

2.5 Wesbart 1589 Shaking Incubator.

3. Methods

3.1 Tissue Preparation

3.2 Dog, rat, rabbit, and human NEP is obtained from the kidney cortex using a method adapted from Booth, A.G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer (1.2) using a Braun miniprimer (2.2).

3.5 Magnesium chloride (1.8) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer containing magnesium chloride (9 mg MgCl in Sml buffer per 1 g tissue).

3.9 The suspension is centrifuged at 2,200 g (4,630 rpm) for 12 minutes in a Beckman centrifuge (2.3) before discarding the pellet.

3.10 The supernatant is centrifuged at 15,000 g (12,100 rpm) for 12 minutes using the Sorvall centrifuge (2.1) and the supernatant is discarded.

3.11 The final pellet is resuspended in homogenisation buffer containing magnesium chloride (0.9 mg MgCl in 0.5 ml buffer per 1 g tissue). A homogenous suspension is obtained using a Braun miniprimer (2.2). This is then frozen down in 100 μl aliquots to be assayed for NEP activity.

4.0 Determination of NEP Activity

The activity of the previously aliquoted NEP is measured by its ability to cleave the NEP specific peptide substrate.

4.1 A 4% DMSO/NEP buffer solution is made (4 mls DMSO in 96 mls NEP buffer).

4.2 Substrate, total product, enzyme, and Phosphoramidon stocks are left on ice to thaw.

4.3 50 μl of 4% DMSO/NEP buffer solution is added to each well.

4.4 The 2 mM substrate stock is diluted 1:40 to make a 50 μM solution. 100 μl of 50 μM substrate is added to each well (final concentration 25 μM).

4.5 50 μl of a range of enzyme dilutions is added to initiate the reaction (usually 1:100, 1:200, 1:400, 1:800, 1:1600, and 1:3200 are used). 50 μl of NEP buffer is added to blank wells.

4.6 The 2 mM total product is diluted 1:80 to make a 25 μM solution. 200 μl of 25 μM product is added to the first four wells of a new plate.

4.7 Plates are incubated at 37° C. in a shaking incubator for 60 minutes. 4.8 The 300 μM Phosphoramidon stock is diluted 1:100 to 300 nM. The reaction is stopped by the addition of 100 μl 300 nM Phosphoramidon and incubated at 37° C. in a shaking incubator for 20 minutes before being read on the Fluostar (ex320/em420).

5. NEP Inhibition Assays

5.1 Substrate, total product, enzyme and Phoshoramidon stocks are left on ice to thaw.

5.2 Compound stocks are made up in 100% DMSO and diluted 1:25 in NEP buffer to give a 4% DMSO solution. All further dilutions are carried out in a 4% DMSO solution (4 mls DMSO in 96 mls NEP buffer).

5.3 50 μl of compound in duplicate is added to the 96 well plate and 50 μl of 4% DMSO/NEP buffer is added to control and blank wells.

5.4 The 2 mM substrate stock is diluted 1:40 in NEP buffer to make a 50 μM solution (275 μl 2 mM substrate to 10.73 ml buffer is enough for 1 plate).

5.5 The enzyme stock diluted in NEP buffer (determined from activity checks).

5.6 The 2 mM total product stock is diluted 1:80 in NEP buffer to make a 25 μM solution. 200 μl is added to the first four wells of a separate plate.

5.7 The 300 μM Phosphoramidon stock is diluted 1:1000 to make a 300 nM stock (11 μl Phosphoramidon to 10.99 ml NEP buffer.

5.8 To each well in the 96 well plate the following is added:

Table Reagents to be added to 96 well plate.

| | Compound/ | Tris | | NEP | Total |
|---|---|---|---|---|---|
| | DMSO | Buffer | Substrate | enzyme | product |
| Samples | 2 μl compound | 50 μl | 100 μl | 50 μl | None |
| Controls | 2 μl DMSO | 50 μl | 100 μl | 50 μl | None |
| Blanks | 2 μl DMSO | 100 μl | 100 μl | None | None |
| Totals | 2 μl DMSO | None | None | None | 20O μl |

5.9 The reaction is initiated by the addition of the NEP enzyme before incubating at 37° C. for 1 hour in a shaking incubator.

5.10 The reaction is stopped with 100 μl 300 nM Phosphoramidon and incubated at 37° C. for 20 minutes in a shaking incubator before being read on the Fluostar (ex320/em420).

6. Calculations

The activity of the NEP enzyme is determined in the presence and absence of compound and expressed as a percentage.

% Control Activity (Turnover of Enzyme):

$$\frac{Mean FU of controls - Mean FU of blanks}{Mean FU of totals - Mean FU of blanks} \times 100$$

% Activity With Inhibitor:

$$\frac{Mean FU of compound - Mean FU of blanks}{Mean FU of totals - Mean FU of blanks} \times 100$$

Activity expressed as % of control:

$$\frac{\% Activity with inhibitor}{\% Control activity} \times 100$$

A sigmoidal dose-response curve is fitted to the % activities (% of control) vs compound concentration and IC50 values calculated using LabStats fit-curve in Excel.

ACE Assay

The Preparation and Assay of Soluble Angiotensin Converting Enzyme (ACE), from Porcine and Human Kidney Cortex.

Soluble ACE activity is obtained from the kidney cortex and assayed by measuring the rate of cleavage of the ACE substrate Abz-Gly-p-nitro-Phe-Pro-OH to generate its fluorescent product, Abz-Gly.

1. Materials

All water is double de ionised.

1.1 Human Kidney IIAM (Pennsylvania. U.S.A.) or UK Human Tissue Bank (UK HTB)

1.2 Porcine kidney ACE Sigma (A2580)

1.3 Homogenisation buffer-1

100 MM Mannitol and 20 mM Tris@pH 7.1

2.42 g Tris (Fisher T/P630/60) is diluted in 1 litre of water and the pH adjusted to 7.1 using 6M HCl at room temperature. To this 18.22 g Mannitol (Sigma M-9546) is added.

1.4 Homogenisation Buffer-2

100 mM Mannitol, 20 mM Tris@pH7.1 and 10 mM MgCl₂.6H₂O (Fisher M0600/53) To 500 ml of the homogenisation buffer 1 (1.4) 1.017 g of MgCl₂ is added.

1.5 Tris Buffer (ACE Buffer).

50 mM Tris and 300 mM NaCl@pH 7.4 50 ml of 50 mM Tris pH 7.4 (Sigma T2663) and 17.52 g NaCl (Fisher S/3160/60) are made up to 1000 ml in water.

1.6 Substrate (Abz-D-Gly-p-nitro-Phe-Pro-OH) (Bachem M-1100)

ACE substrate is stored as a powder at −20° C. A 2 mM stock is made by gently re-suspending the substrate in ACE buffer, this must not be vortexed or sonicated. 400 μl aliquots of the 2 mM stock are stored at −20° C. for up to one month.

1.7 Total Product

Samples corresponding to 100% substrate to product conversion are included on the plate to enable the % substrate turnover to be determined (see calculations). The total product is generated by incubating 1 ml of 2 mM substrate with 20 μl of enzyme stock for 24 hours at 37° C.

1.8 Stop Solution.

0.5M EDTA (Promega CAS[6081/92/6]) is diluted 1:250 in ACE buffer to make a 2 mM solution.

1.9 Dimethyl Sulphoxide (DMSO).

1.10 Magnesium Chloride—MgCl₂.6H₂O (Fisher M0600/53).

1.11 Black 96 Well Flat Bottom Assay Plates (Costar 3915 or Packard).

1.12 Topseal A (Packard 6005185).

1.13 Centrifuge Tubes

2. Specific Equiptment

2.1 Sorvall RC-5B centrifuge (SS34 GSA rotor, pre-cooled to 4° C.).

2.2 Braun miniprimer mixer.

2.3 Beckman CS-6R centrifuge.

2.4 BMG Fluostar Galaxy.

2.5 Wesbart 1589 shaking incubator.

3. Methods

3.1 Tissue Preparation

3.3 Human ACE is obtained from the kidney cortex using a method adapted from Booth, A. G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer-1 (1.4) using a Braun miniprimer (2.2).

3.5 Magnesium chloride (1.11) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer-2 (1.5) (5 ml buffer per 1 g tissue).

3.9 The suspension is centrifuged at 2,200 g (4,630 rpm) for 12 minutes in a Beckman centrifuge before discarding the pellet.

3.10 The supernatant is centrifuged at 15,000 g (12,100 rpm) for 12 minutes using the Sorvall centrifuge and the supernatant is discarded.

3.11 The final pellet is resuspended in homogenisation buffer-2 (0.5 ml buffer per 1 g tissue). A homogenous suspension is obtained using a Braun miniprimer. This is then frozen down in 100 μl aliquots to be assayed for NEP activity.

4.0 Determination of ACE Activity

The activity of the previously aliquoted ACE is measured by its ability to cleave the ACE specific peptide substrate.

Porcine ACE (1.2) is defrosted and resuspended in ACE buffer (1.6) at 0.004U/μl, this is frozen down in 50 μl aliquots.

4.1 A 4% DMSO/ACE buffer solution is made (4 mls DMSO in 96 mls ACE buffer).

4.2 Substrate (1.7), total product (1.8) and enzyme (1.1, 1.2, 1.3), are left on ice to thaw.

4.3 50 μl of 4% DMSO/ACE buffer solution is added to each well.

4.4 The 2 mM substrate stock is diluted 1:100 to make a 20 μM solution. 100 μl of 20 μM substrate is added to each well (final concentration in the assay 10 μM).

4.5 50 μl of a range of enzyme dilutions is added to initiate the reaction (usually 1:100, 1:200, 1:400, 1:800, 1:1600, and 1:3200 are used). 50 μl of ACE buffer is added to blank wells.

4.6 The 2 mM total product is diluted 1:200 to make 10 μM solution. 200 μl 10 μM product is added to the first four wells of a new plate.

4.7 Plates are incubated at 37° C. in a shaking incubator for 60 minutes.

4.8 The enzyme reaction is stopped by the addition of 100 μl 2 mM EDTA in ACE buffer and incubated at 37° C. in a shaking incubator for 20 minutes before being read on the BMG Fluostar Galaxy (ex320/em420).

5. ACE Inhibition Assays

5.1 Substrate, total product, and enzyme stocks are left on ice to thaw.

5.2 Compound stocks are made up in 100% DMSO and diluted 1:25 in ACE buffer to give a 4% DMSO solution. All further dilutions are carried out in a 4% DMSO/ACE buffer solution (4 mls DMSO in 96 mls ACE buffer).

5.3 50 μl of compound, in duplicate, is added to the 96 well plate and 50 μl of 4% DMSO/ACE buffer is added to control and blank wells.

5.4 Steps 5.2 and 5.3 can be carried out either by hand or using the Packard multiprobe robots

5.5 The 2 mM substrate stock is diluted 1:100 in ACE buffer to make a 20 μM solution (10 μM final concentration in the assay) (110 μl of 2 mM substrate added to 10.89 ml buffer is enough for 1 plate).

5.6 The enzyme stock is diluted in ACE buffer, as determined from activity checks (4.0).

5.7 The 2 mM total product stock is diluted 1:200 in ACE buffer to make a 10 μM solution. 200 μl is added to the first four wells of a separate plate.

5.8 The 0.5 mM EDTA stock is diluted 1:250 to make a 2 mM stock (44 μl EDTA to 10.96 ml ACE buffer).

5.9 To each well of the 96 well plate the following reagents are added:

TABLE 1
Reagents added to 96 well plate

| | Compound/ | Tris | | ACE | Total |
|---|---|---|---|---|---|
| | DMSO | Buffer | Substrate | enzyme | product |
| Samples | 2 μl compound | 50 μl | 100 μl | 50 μl | None |
| Controls | 2 μl DMSO | 50 μl | 100 μl | 50 μl | None |
| Blanks | 2 μl DMSO | 100 μl | 100 μl | None | None |
| Totals | 2 μl DMSO | None | None | None | 20O μl |

5.10 50 μl of the highest concentration of each compound used in the assay is added in duplicate to the same 96 well plate as the totals (5.7). 150 μl of ACE buffer is added to determine any compound fluorescence.

5.11 The reaction is initiated by the addition of the ACE enzyme before incubating at 37° C. for 1 hour in a shaking incubator.

5.12 The reaction is stopped by the addition of 100 μl 2 mM EDTA and incubated at 37° C. for 20 minutes in a shaking incubator, before being read on the BMG Fluostar Galaxy (ex320/em420).

6. Calculations

The activity of the ACE enzyme is determined in the presence and absence of compound and expressed as a percentage.

FU=Fluorescence units

(i) % Control Activity (Turnover of Enzyme):

$$\frac{Mean FU of controls - Mean FU of blanks}{Mean FU of totals - Mean FU of blanks} \times 100$$

(ii) % Activity With Inhibitor:

$$\frac{Mean FU of compound - Mean FU of blanks}{Mean FU of totals - Mean FU of blanks} \times 100$$

(iii) Activity Expressed as % of Control:

$$\frac{\% Activity with inhibitor}{\% Control activity} \times 100$$

$$OR \frac{Mean FU of compound - Mean FU of blanks}{Mean FU of totals - Mean FU of blanks} \times 100$$

(iv) % Inhibition=100−% control

(v) For fluorescent compounds the mean FU of blanks containing compound (5.10) is deducted from the mean FU of compound values used to calculate the % Activity.

A sigmoidal dose-response curve is fitted to the % activities (% of control) vs compound concentration and IC₅₀ values calculated using LabStats fit-curve in Excel.

PDE5 Inhibitor—Test Methods

Phosphodiesterase (PDE) Inhibitory Activity

In vitro PDE inhibitory activities against cyclic guanosine 3′,5′-monophosphate (cGMP) and cyclic adenosine 3′,5′-monophosphate (cAMP) phosphodiesterases were determined by measurement of their IC₅₀ values (the concentration of compound required for 50% inhibition of enzyme activity).

The required PDE enzymes were isolated from a variety of sources, including human corpus cavemosum, human and rabbit platelets, human cardiac ventricle, human skeletal muscle and human and canine retina, essentially by the method of W. J. Thompson and M. M. Appleman (Biochem., 1971, 10, 311). In particular, the cGMP-specific PDE (PDE5) and the cGMP-inhibited cAMP PDE (PDE3) were obtained from human corpus cavemosum or human platelets; the cGMP-stimulated PDE (PDE2) was obtained from human corpus cavemosum and human platelets; the calcium/calmodulin (Ca/CAM)-dependent PDE (PDE1) from human cardiac ventricle; the cAMP-specific PDE (PDE4) from human skeletal muscle and human recombinant, expressed in SF9 cells; and the photoreceptor PDE (PDE6) from human or canine retina. Phosphodiesterases 7-11 were generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc ˜1/3 Km) such that IC₅₀≅Ki. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension (or in-house equivalent). Results from these tests show that the compounds of the present invention are inhibitors of cGMP-specific PDE5.

Functional Activity

This can be assessed in vitro by determining the capacity of a compound of the invention to enhance sodium nitroprusside or electrical field stimulation-induced relaxation of pre-contracted rabbit corpus cavernosum tissue strips, using methods based on that described by S. A. Ballard et al. (Brit. J. Pharmacol., 1996, 118 (suppl.), abstract 153P) or S. A. Ballard et al. (J. Urology, 1998, 159, 2164-2171).

In vivo Activity

Compounds can be screened in anaesthetised dogs to determine their capacity, after i.v. administration, to enhance the pressure rises in the corpora cavernosa of the penis induced by intracavemosal injection of sodium nitroprusside, using a method based on that described by Trigo-Rocha et al. (Neurourol. and Urodyn., 1994, 13, 71).

NPY Assay:

An assay for identifying NPY inhibitors is presented in WO-A-98/52890 (see page 96, lines 2 to 28).

### OTHER REFERENCES

Anastasi A. et al., Experientia, 1971; 27: 166

S A Ballard et al, Journal of Urology, 1998, vol. 159, pages 2164-2171

Barker, P. E.; Shipp, M. A.; D'Adamio, L.; Masteller, E. L.; Reinherz, E. L. The common acute lymphoblastic leukemia antigen gene maps to chromosomal region 3(q21-q27). J. Immun. 142: 283-287, 1989.

Battey J., et al., TINS, 1991;14:524

Baum M. J., J. Biosci., 1983; 33:578-582

Benet A. E and Melman A, 1995, Urol. Clin. N. Amer. 22: 699-709

Berman et al., 1999, hypoactive sexual desire disorders, sexual arousal disorders, anorgasmy and sexual pain disorders, Urology 54: 385-391.

Berman, J., Goldstein, I., Werbin, T. et al. (1999a). Double blind placebo controlled study with crossover to assess effect of sildenafil on physiological parameters of the female sexual response. J. Urol., 161, 805.

Berman J. R. et al. Int. J. Impot. Res., 1999, 11: S31-38

Bernabe J, Rampin O, Sachs B D, Giuliano F. Intracavernous pressure during erection in rats: an integrative approach based on telemetric recording. Am J Physiol. 1999 February;276(2 Pt 2):R441-9.

Bonney R. C et al., Scrip's Complete Guide to Women's Healthcare, PJB Publications Ltd, London, 2000 Braun M., et al., Life. Sci., 1978; 23: 2721

Bundgaard et al., Acta Pharm. Suec., 1987; 24: 233-246

D'Adamio, L.; Shipp, M. A.; Masteller, E. L.; Reinherz, E. L.: Organization of the gene encoding common acute lymphoblastic leukemia antigen (neutral endopeptidase 24.11): multiple miniexons and separate 5-prime untranslated regions. Proc. Nat. Acad. Sci. 86: 7103-7107, 1989.

Dutta A. S., Small Peptides; Chemistry, Biology, and Clinical Studies, Chapter 2, pp 66-82

J. Fröhlich et al., Heterocycles 1994, 37, 1879-91

Goldstein, I. & Berman, J. R. (1998). Vasculogenic female sexual dysfunction: vaginal engorgement and clitoral erectile insufficiency syndromes. Int. J. Impot. Res., 10, S84-S90.

Johnson M and Everitt B., Essential Reproduction (3^{rd} edn), Blackwell, Oxford, 1988.

Ladenheim E. E et al, Brain Res., 1990; 537: 233-240

Laumann et al., 1999 JAMA 281:537-544)

Letarte, M.; Vera, S.; Tran, R.; Addis, J. B. L.; Onizuka, R. J.; Quackenbush, E. J.; Jongeneel, C. V.; McInnes, R. R. : Common acute lymphocytic leukemia antigen is identical to neutral endopeptidase. J. Exp. Med. 168: 1247-1253, 1988.

Meyerson B.J, Lindstrom L. H., Acta Physiol. Scand., 1973; 389 (Suppl.): 1-80.

O'Donohue W, et al, Clin. Psychol. Rev. 1997; 17: 537-566.

Park, K., Goldstein, I., Andry, C., et al. (1997). Vasculogenic female sexual dysfunction: The hemodynamic basis for vaginal engorgement insufficiency and clitoral erectile insufficiency. Int. J. Impotence Res., 9, 27-37.

Sachs B. D., Neuroscience and Biobehavioral Review, 2000, 24 541-560.

Shipp, M. A.; Vijayaraghavan, J.; Schmidt, E. V.; Masteller, E. L.; D'Adamio, L.; Hersh, L. B.; Reinherz, E. L.: Common acute lymphoblastic leukemia antigen (CALLA) is active neutral endopeptidase 24.11 (‘enkephalinase’): direct evidence by cDNA transfection analysis. Proc. Nat. Acad. Sci. 86: 297-301, 1989.

Tran-Paterson, R.; Willard, H. F.; Letarte, M. : The common acute lymphoblastic leukemia antigen (neutral endopeptidase—3.4.24. 11) gene is located on human chromosome 3. Cancer Genet. Cytogenet. 42: 129-134, 1989.

Werbin, T., Salimpour, P., Berman, L., et al. (1999). Effect of sexual stimulation and age on genital blood flow in women with sexual stimulation. J. Urol., 161, 688.

Wilson C. A., Pharmacological targets for the control of male and female sexual behaviour, In: Sexual Pharmacology, Riley A. J. et al, (Eds), Clarendon Press, Oxford, 1993: 1-58

1. A method of treating sexual dysfunction which comprises administering to a subject suffering therefrom and in need of treatment an effective amount of a bombesin receptor antagonist.

2. The method of claim 1, wherein the subject is a human male.

3. The method of claim 2, wherein the subject is suffering from erectile dysfunction.

4. The method of claim 3, wherein the subject is suffering from erectile dysfunction that is psychogenic.

5. The method of claim 3, wherein the subject is suffering from erectile dysfunction that is hormonal or endocrinologic.

6. The method of claim 3, wherein the subject is suffering from erectile dysfunction that is neurogenic.

7. The method of claim 3, wherein the subject is suffering from erectile dysfunction that is drug-induced.

8. The method of claim 3, wherein the subject is suffering from erectile dysfunction that is arteriogenic and/or venogenic and/or related to cavernosal factors.

9. The method of claim 2, wherein the subject is suffering from hypoactive sexual desire.

10. The method of claim 2, wherein the subject is suffering from delayed orgasm disorder.

11. The method of claim 1, wherein the subject is a human female.

12. The method of claim 11, wherein the subject is suffering from sexual arousal disorder.

13. The method of claim 12, wherein the subject is suffering from sexual arousal disorder that is arteriogenic and/or vasculogenic.

14. The method of claim 12, wherein the subject is suffering from sexual arousal disorder that is neurogenic.

15. The method of claim 12, wherein the subject is suffering from sexual arousal disorder that is hormonal or endocrine.

16. The method of claim 12, wherein the subject is suffering from sexual arousal disorder that is psychogenic.

17. The method of claim 12, wherein the subject is suffering from sexual arousal disorder that is drug-induced.

18. The method of claim 11, wherein the subject is suffering from hypoactive sexual desire disorder.

19. The method of claim 11, wherein the subject is suffering from orgasmic disorder or anorgasmia.

20. The method of claim 11, wherein the subject is suffering from sexual pain disorder.

21. The method of claim 1, wherein the dysfunction is associated with generalised unresponsiveness and/or ageing-related decline in sexual arousability.

22. The method of claim 1, wherein the bombesin receptor antagonist is a selective bombesin BB1 antagonist.

23. The method of claim 22, wherein the bombesin BB1 antagonist has a selectivity for BB₁ over the other bombesin receptor subtypes greater than 10.

24. The method of claim 22, wherein the bombesin BB1 antagonist has a selectivity for BBI over the other bombesin receptor subtypes greater than 30.

25. The method of claim 22, wherein the bombesin BB1 antagonist has a selectivity for BB1 over the other bombesin receptor subtypes greater than 100.

26. The method of claim 1, wherein the bombesin receptor antagonist is a mixed BB1/BB2 antagonist.

27. The method of claim 1, wherein the bombesin receptor antagonist has a Ki against BB1 of less than 1000 nM.

28. The method of claim 1, wherein the bombesin receptor antagonist has a Ki against BB1 of less than 500 nM.

29. The method of claim 1, wherein the bombesin receptor antagonist has a Ki against BB1 of less than 100 nM.

30. The method of claim 1, wherein the bombesin receptor antagonist has a Ki against BB1 of less than 50 nM.

31. The method of claim 1, wherein the bombesin receptor antagonist has a Ki against BB1 of less than 10 nM.

32. The method of claim 1, wherein there is administered to the subject an effective amount of a non-peptide bombesin receptor antagonist.

33. The method of claim 32, wherein the non-peptide bombesin receptor antagonist is a compound that is absorbable when administered orally.

34. The method of claim 1, wherein there is administered to the subject an effective amount of a bombesin receptor antagonist which is a peptide.

35. A method of treating sexual dysfunction in a male or female subject suffering therefrom and in need of treatment which comprises administering to the subject an effective amount of a bombesin receptor antagonist and a PDE 5 inhibitor.

36. A method of treating sexual dysfunction in a male or female subject suffering therefrom and in need of treatment which comprises administering to the subject an effective amount of a bombesin receptor antagonist and a NEP inhibitor.

37. A method of treating sexual dysfunction in a female subject suffering therefrom and in need of treatment which comprises administering to the subject an effective amount of a bombesin receptor antagonist and one or more estrogen receptor modulators (SERM) and/or estrogen agonists and/or estrogen antagonists.

38. A method of treating sexual dysfunction in a male or female subject suffering therefrom and in need of treatment which comprises administering to the subject an effective amount of a bombesin receptor antagonist and lasofoxifene.

39. A pharmaceutical combination (for simultaneous, separate or sequential administration) of a bombesin receptor antagonist and one or more materials selected from (1) to (34) below:
(1) naturally occurring or synthetic prostaglandins or esters thereof;
(2) α-adrenergic receptor antagonist compounds also known as α-adrenoceptor antaginists or α-receptor antagonists or α-blockers;
(3) NO-donor (NO-agonist) compounds;
(4) potassium channel openers or modulators;
(5) dopaminergic agents;
(6) vasodilator agents;
(7) thromboxane A2 agonists;
(8) CNS active agents;
(9) ergot alkaloids;
(10) compounds which modulate the action of natriuretic factors;
(11) angiotensin receptor antagonists such as losartan.;
(12) substrates for NO-synthase;
(13) calcium channel blockers;
(14) cholesterol lowering agents;
(15) antiplatelet and antithrombotic agents;
(16) insulin sensitising agents and hypoglycaemic agents;.
(17) L-DOPA or carbidopa;
(18) acetylcholinesterase inhibitors;
(19) steroidal or non-steroidal anti-inflammatory agents;
(20) estrogen receptor modulators and/or estrogen agonists and/or estrogen antagonists, and pharmaceutically acceptable salts thereof;
(21) PDE inhibitors;
(22) NPY (neuropeptide Y) inhibitors;
(23) NEP inhibitors;
(24) vasoactive intestinal proteins (VIP), VIP mimetics, VIP analogues, VIP receptor agonists or VIP analogues or VIP fragments, or α-adrenoceptor antagonists with VIP combinations;
(25) melanocortin receptor agonists or modulators or melanocortin ehancers;
(26) serotonin receptor agonists, antagonists or modulators;
(27) testosterone replacement agents, testosternone, dihydrotestosterone or a testosterone implant;
(28) estrogen, estrogen and medroxyprogesterone or medroxyprogesterone acetate (MPA) (i.e. as a combination), or estrogen and methyl testosterone hormone replacement therapy agents;
(29) modulators of transporters for noradrenaline, dopamine and/or serotonin;
(30) purinergic receptor agonists and/or modulators;
(31) neurokinin (NK) receptor antagonists;
(32) opioid receptor agonists, antagonista or modulators;
(33) agonists or modulators for oxytocin/vasopressin receptors; and
(34) modulators of cannabinoid receptors.

40 The combination of claim 39, wherein the bombesin receptor antagonist is a compound of the formula (I) 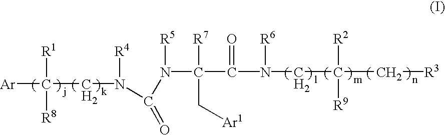
or a pharmaceutically acceptable salt thereof, wherein:
j is 0 or 1;
k is 0 or 1;
l is 0, 1, 2, or 3;
m is 0 or 1;
n is 0, 1 or 2;
Ar is phenyl, pyridyl or pyrimidyl, each unsubstituted or substituted by from 1 to 3 substituents selected from alkyl, halogen, alkoxy, acetyl, nitro, amino, —CH₂NR¹⁰R¹¹, cyano, —CF₃, —NHCONH₂, and —CO₂R¹²;
R¹is hydrogen or straight, branched, or cyclic alkyl of from 1 to 7 carbon atoms;
R⁸ is hydrogen or forms a ring with R¹ of from 3 to 7 carbon atoms;
R² is hydrogen or straight, branched, or cyclic alkyl of from 1 to 8 carbon atoms which can also contain 1 to 2 oxygen or nitrogen atoms;
R⁹ is hydrogen or forms with R² a ring of from 3 to 7 carbon atoms which can contain an oxygen or nitrogen atom; or R² and R⁹ can together be a carbonyl;
Arl can be independently selected from Ar and can also include pyridyl-N-oxide, indolyl, imidazolyl, and pyridyl;
R⁴, R⁵, R⁶, and R⁷ are each independently selected from hydrogen and lower alkyl; R⁴ can also form with R⁵ a covalent link of 2 to 3 atoms which may include an oxygen or a nitrogen atom;
R³ can be independently selected from Ar or is hydrogen, hydroxy, —NMe₂, N-methyl-pyrrolyl, imidazolyl, N-methyl-imidazolyl, tetrazolyl, N-methyl-tetrazolyl, thiazolyl, —CONR¹³R¹⁴, alkoxy, 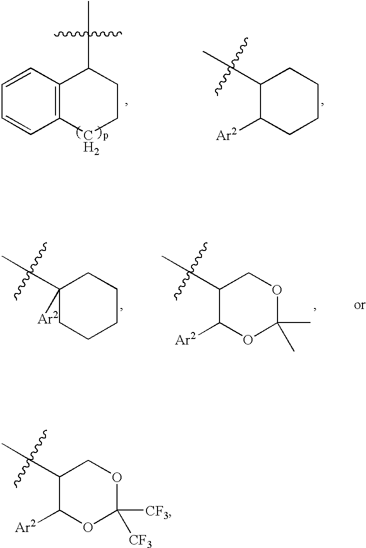
wherein p is 0, 1 or 2 and Ar² is phenyl or pyridyl;
R¹⁰, R¹¹, R¹², R¹³ and R¹⁴ are each independently selected from hydrogen or straight, branched, or cyclic alkyl of from 1 to 7 carbon atoms.

41. The combination of claim 39, wherein the bombesin receptor antagonist is a compoundof Formula (Ia) 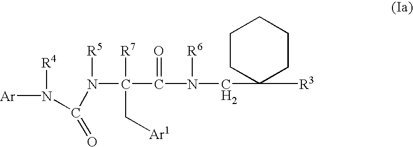
wherein
Ar is phenyl unsubstituted or substituted with 1 or 2 substituents selected from isopropyl, halo, nitro, and cyano;
R⁴, R⁵, and R⁶ are hydrogen;
R⁷ is methyl or hydrogen;
R³ is 2-pyridyl or hydroxy; and
Ar¹ is indolyl, pyridyl, pyridyl-N-oxide, or imidazolyl.

42. The combination of claim 40, wherein the bombesin receptor antagonist is a compound of Formula I wherein
Ar is unsubstituted phenyl;
R¹ is cyclopentyl or tert-butyl;
R⁴ and R⁵ are hydrogen;
R⁷ is methyl;
R⁶ is hydrogen;
R³ is phenyl with two isopropyl substituents, unsubstituted phenyl, or 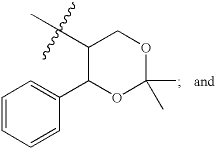
Ar¹ is indolyl.

43. The combination of claim 40, wherein the bombesin receptor antagonist is a compound of Formula I wherein
Ar is 2,6-diisopropyl-phenyl, 4-nitro-phenyl, and 4-cyano-phenyl;
R⁴, R⁵, and R⁶ are hydrogen;
R⁷ is methyl;
R² is hydrogen or cyclohexyl; and
R³ is hydroxyl, pyridyl,

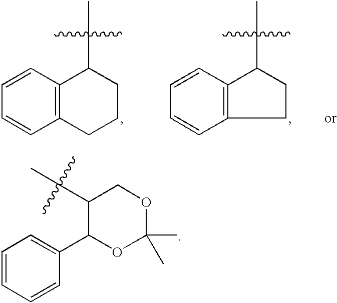

44. The combination of claim 39, wherein the bombesin receptor antagonist is
(S)3-(1H-Indol-3-yl)-N-[1-(5-methoxy-pyridin-2-yl)-cyclohexylmethyl]-2-methyl-2-[3-(4-nitro-phenyl)-ureido]-propionamide (also referred to as Compound 1) or one of its pharmaceutically acceptable salts or is
(2S)-N-{[1-(4-aminophenyl) cyclohexyl]methyl}-3-(1H-indol-3-yl)-2-methyl-2-{[(4-nitroanilino) carbonyl]amino} propanamide (also knowm as Compound 3) or one of its pharmaceutically acceptable salts.

45. The combination of claim 39, wherein the bombesin receptor antagonist is a compound set out below or a pharmaceutically acceptable salt thereof:
(S) N-cyclohexylmethyl-2-[3-(2,6-diisopropyl-phenyl)-ureido]-3-(1H-indol-3-yl)-2-methyl-propionamide;
N-cyclohexylmethyl-2-[3-(2,6-diisopropyl-phenyl)-ureido]-3-(1H-indol-3-yl)-N-methyl-propionamide;
N-cyclohexylmethyl-2-[3-(2,6-diisopropyl-phenyl)-1-methyl-ureido]-3-(1H-indol-3-yl)-propionamide;
2-[3-(2,6-diisopropyl-phenyl)-ureido]-2-methyl-3-(1-oxy-pyridin-2-yl)-N-(1-pyridin-2-yl-cyclohexylmethyl)-propionamide;
2-[3-(2,6-diisopropyl-phenyl)-ureido]-2-methyl-3-pyridin-2-yl-N-(1-pyridin-2-yl-cyclohexylmethyl)-propionamide;
2-[3-(2-tert-butyl-phenyl)-ureido]-N-cyclohexylmethyl-3-(1H-indol-3-yl)-2-methyl-propionamide;
N-cyclohexylmethyl-2-[3-(2,6-dichloro-phenyl)ureido]-3-(1H-indol-3-yl)-2-methyl-propionamide;
N-cyclohexylmethyl-2-[3-(2,6-dimethoxy-phenyl)ureido]-3-(1H-indol-3-yl)-2-methyl-propionamide;
N-cyclohexylmethyl-2-[3-(2,6-dimethylamino-phenyl)-ureido]-3-(1Hindol-3-yl)-2-methyl-propionamide;
(S) N-cyclohexylmethyl-3-(1H-indol-3-yl)-2-methyl-2-[3-(4-nitro-phenyl)-ureido]-propionamide;
N-cyclohexylmethyl-2-[3-(2,2-dimethyl-1-phenyl)propyl)-ureido]-3-(1H-indol-3-yl)-2-methyl-propionamide;
[S-(R*, R*)] 3-(1H-indol-3-yl)-2-methyl-2-{3-[1-(4-nitro-phenyl)-ethyl]-ureido}-N-(1-pyridin-2-yl-cyclohexylmethyl)-propionamide;
N-(2,2-dimethyl-4-phenyl-[1,3]dioxan-5-yl)-3-(1H-indol-3-yl)-2-methyl-2-[3-(1-phenyl-cyclopentylmethyl)ureido]-propionamide;
(S)-N-(2,6-diisopropyl-phenyl)-2-[3-(2,2-dimethyl-1-phenyl-propyl)-ureido]-3-(1H-indol-3-yl)-propionamide;
(R)-N-(2,6-diisopropyl-phenyl)-2-[3-(2,2-dimethyl-1-phenyl-propyl)-ureido]-3-(1H-indol-3-yl)-propionamide;
2-[3-(2,6-diisopropyl-phenyl)-ureido]-N-(2,2-dimethyl-4-phenyl-[1,3]dioxan-5-yl)-3-(1H-indol-3-yl)-2-methyl-propionamide;
N-cyclohexyl-2-[3-(2,6-diisopropyl-phenyl)-ureido]-3-(1H-indol-3-yl)-2-methyl-propionamide;
N-(2-cyclohexyl-ethyl)-2-[3-(2,6-diisopropyl-phenyl)-ureido]-3-(1H-indol-3-yl)-2-methyl-propionamide;
2-[3-(2,6-diisopropyl-phenyl)-ureido]-3-(1H-indol-3-yl)-2-methyl-propionamide;
2-[3-(2,6-diisopropyl-phenyl)-ureido]-3-(1H-indol-3-yl)-2-methyl-N-(3-methyl-butyl)-propionamide;
2-[3-(2,6-diisopropyl-phenyl)-ureido]-3-(1H-indol-3-yl)-2-methyl-N-(3-phenyl-propyl)-propionamide;
2-[3-(2,6-diisopropyl-phenyl)-ureido]-3-(1H-indol-3-yl)-2-methyl-N-(1,2,3,4-tetrahydro-naphthalen-1-yl)-propionamide;
2-[3-(2,6-diisopropyl-phenyl)-ureido]-3-(1H-indol-3-yl)-2-methyl-N-(2-phenyl-cyclohexyl)-propionamide;
2-[3-(2,6-diisopropyl-phenyl)-ureido]-N-indan-1-yl-3-(1H-indol-3-yl)-2-methyl-propionamide;
2-[3-(2,6-diisopropyl-phenyl)-ureido]-N-(1-hydroxy-cyclohexylmethyl)-3-(1H-indol-3-yl)-2-methyl-propionamide;
2-[3-(2,6-diisopropyl-phenyl)-ureido]-3-(1H-indol-3-yl)-2-methyl-N-(1-pyridin-2-yl-cyclohexylmethyl)-propionamide;
2-[3-(2,6-diisopropyl-phenyl)-ureido]-3-(1H-indol-3-yl)-2-methyl-N-(6,7,8,9-tetrahydro-5H-benzocyclohepten-5-yl)-propionamide; 2-[3-(2,6-diisopropyl-phenyl)-ureido]-3-(1H-indol-3-yl)-2-methyl-N-phenyl-propionamide;
N-(1-hydroxy-cyclohexylmethyl)-3-(1H-indol-3-yl)-2-methyl-2-[3-(4-nitro-phenyl)-ureido]-propionamide;
2-[3-(4-cyano-phenyl)-ureido]-3-(1H-indol-3-yl)-2-methyl-N-(1-pyridin-2-yl-cyclohexylmethyl)-propionamide;
(S) 3-(1H-indol-3-yl)-2-methyl-2-[3-(4-nitro-phenyl)-ureido]-N-(1-pyridin-2-yl-cyclohexylmethyl)-propionamide;
(S) 3-(1H-indol-3-yl)-2-methyl-N-(1-pyridin-2-yl-cyclohexylmethyl)-2-[3-(4-trifluoromethyl-phenyl)-ureido]-propionamide;
(S) 4-(3-{2-(1H-indol-3-yl)-1-methyl-1-[(1-pyridin-2-yl-cyclohexylmethyl)-carbamoyl]-ethyl}-ureido)-benzoic acid ethyl ester;
2-[3-(2,6-diisopropyl-phenyl)-ureido]-3-(1H-imidazol-4-yl)-N-(1-pyridin-2-yl-cyclohexylmethyl)-propionamide;
2-[3-(2,6-diisopropyl-phenyl)-ureido]-2-methyl-N-(1-pyridin-2-yl-cyclohexylmethyl)-3-(2-trifluoromethyl-phenyl)-propionamide;
2-[3-(2,6-diisopropyl-phenyl)-ureido]-2-methyl-3-(2-nitro-phenyl)-N-(1-pyridin-2-yl-cyclohexylmethyl)-propionamide;
(S) 3-(1H-indol-3-yl)-N-[1-(5-methoxy-pyridin-2-yl)-cyclohexylmethyl]-2-methyl-2-[3-(4-nitro-phenyl)-ureido]-propionamide; and
N-cyclohexylmethyl-2-[3-(2,6-diisopropyl-phenyl)-ureido]-2-methyl-3-pyridin-2-yl-propionamide.

46. The combination of claim 39, wherein the bombesin receptor antagonist is a compound of formula (II) or a pharmaceutically acceptable salt thereof: 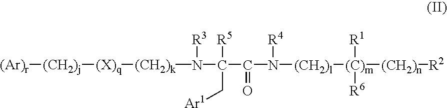
wherein:
j is 0, 1 or 2;
k is 0 or 1;
l is 0, 1, 2, or 3;
m is 0 or 1;
n is 0, 1 or 2;
q is 0 or 1;
r is 0 or 1; when r is 0, Ar is replaced by hydrogen;
Ar is phenyl, pyridyl, pyrimidyl, thienyl, furyl, imidazolyl, pyrrolyl or thiazolyl each unsubstituted or substituted by from 1 to 3 substituents selected from acetyl, alkoxy, alkyl, amino, cyano, halo, hydroxy, nitro, sulfonamido, sulfonyl, —CF₃, —OCF₃, —CO₂H, —CH₂CN, —SO₂CF₃, —CH₂CO₂H and —(CH₂)ₛNR⁷R⁸ wherein s is 0, 1, 2 or 3 and R⁷ and R⁸ are each independently selected from H, straight or branched alkyl of up to 6 carbon atoms, or R⁷ and R⁸ together with the nitrogen atom to which they are linked can form a 5- to 7-membered aliphatic ring which may contain 1 or 2 oxygen atoms;
R is hydrogen, straight or branched alkyl of up to 6 carbon atoms or cycloalkyl of between 5 and 7 carbon atoms which may contain 1 or 2 nitrogen or oxygen atoms;
R⁶ is hydrogen, methyl, or forms with R¹ an aliphatic ring of from 3 to 7 atoms which can contain an oxygen or nitrogen atom, or together with R¹ is a carbonyl group;
Ar is independently selected from Ar or is indolyl or pyridyl-N-oxide;
R³, R⁴, and R⁵ are each independently selected from hydrogen and lower alkyl;
R² is independently selected from Ar or is hydrogen, hydroxy, alkoxy, —NMe₂, —CONR⁹R¹⁰ wherein R⁹ and R¹⁰ are each independently selected from hydrogen, straight or branched alkyl of up to 6 carbon atoms, or R⁹ and R¹⁰ together with the nitrogen atom to which they are linked can form a 5-to 7-membered aliphatic ring which may contain 1 or 2 oxygen or nitrogen atoms, or R² is 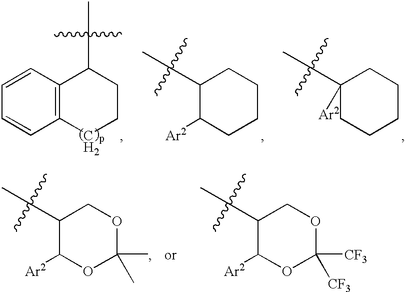
wherein p is 0, 1 or 2 and Ar² is phenyl or pyridyl;
X is a divalent radical derived from any of the following 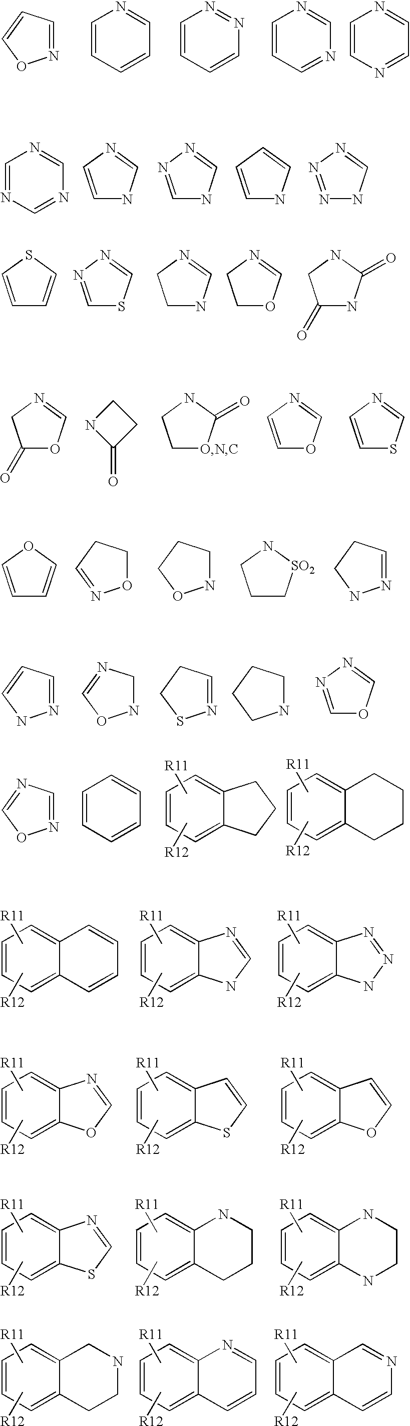
where the ring nitrogen atoms may have lower alkyl groups attached thereto, R¹¹ and R¹² are independently selected from H, halogen, hydroxy, alkoxy, acetyl, 5 nitro, cyano, amino, CF₃ and —(CH₂)ₜNR¹³R¹⁴ where t can be 0 or 1, R¹³ and R¹⁴ are each independently selected from hydrogen, straight or branched alkyl of up to 6 carbon atoms or cycloalkyl of 5 to 7 carbon atoms, containing up to 2 oxygen or nitrogen atoms.

47 The combination of claim 39, wherein the bombesin receptor antagonist is a compound of the formula (IIa), or a pharmaceutically acceptable salt thereof: 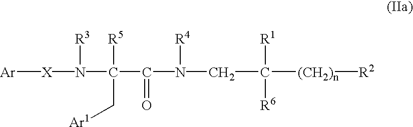
wherein:
n is 0 or 1;
Ar is phenyl or pyridyl which may be unsubstituted or substituted with from 1 to 3 substituents selected from halogen, alkoxy, nitro and cyano; Arl is independently selected from Ar or is pyridyl-N-oxide or indolyl;
R⁶ forms with R¹ an aliphatic ring of from 3 to 7 atoms which can contain an oxygen or nitrogen atom, or together with R¹ is a carbonyl group;
R² is independently selected from Ar or is hydrogen, hydroxy, alkoxy, dimethylamino, tetrazolyl or —CONR⁹R¹⁰ wherein R⁹ and R¹⁰ are each independently selected from hydrogen or methyl or R² is any of 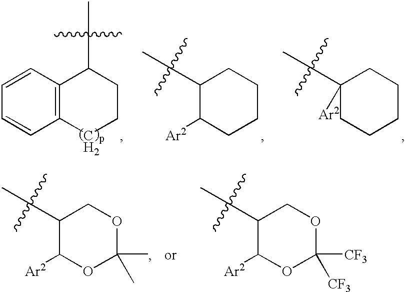
wherein p is 0, 1 or 2 and Ar2 is phenyl or pyridyl;
R³, R⁴ and R⁵ are each independently selected from hydrogen and methyl; and
X is selected from: 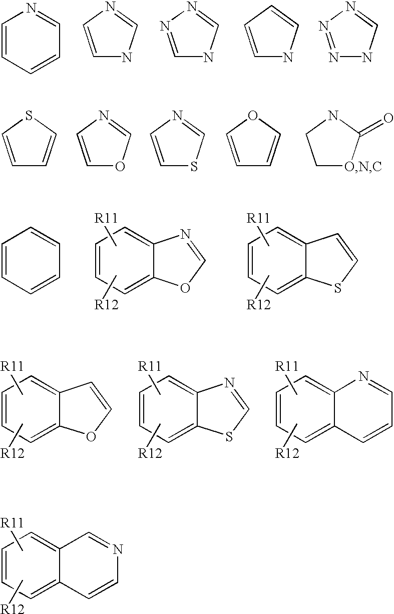
R¹¹ and R¹² being independently selected from H, halogen, hydroxy, alkoxy, acetyl, nitro, cyano, amino, CF₃ and (CH₂)ₜNR¹³R¹⁴ wherein t is 0 or 1 and R¹³ and R¹⁴ are independently selected from hydrogen and methyl.

48. The combination of claim 39, wherein the bombesin receptor antagonist is a compound has the formula (IIb) or (IIc) or is a pharmaceutically acceptable salt thereof: 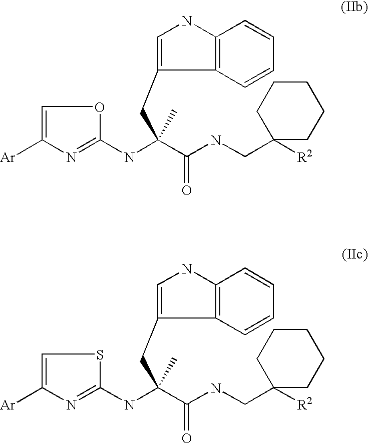
wherein Ar and R² independently represent phenyl or pyridyl which may be unsubstituted or substituted with from 1 to 3 substituents selected from halogen, alkoxy, nitro and cyano, and pharmaceutically acceptable salts thereof.

49. The combination of claim 39, wherein the bombesin receptor antagonist is
(S)-3-(1H-indol-3-yl)-N-[1-(5-methoxy-pyridin-2-yl)-cyclohexylmethyl]-2-methyl-2-[4-(4-nitro-phenyl)-oxazol-2-ylamino]-propionamide (also referred to as Compound 2) or a pharmaceutically acceptable salt.

50 The combination of claim 39, wherein the bombesin receptor antagonist is one of the following compounds or a pharmaceutically acceptable salt thereof:
(S)-3-(1H-indol-3-yl)-N-(1-methoxymethyl-cyclohexylmethyl)-2-methyl-2-[4-(4-nitro-phenyl)-oxazol-2-ylamino]-propionamide;
(S)-3-(1H-indol-3-yl)-2-methyl-2-[4-(4-nitro-phenyl)-oxazol-2-ylamino]-N-(2-oxo-2-phenyl-ethyl)-propionamide;
(S)-N-[1-(5-methoxy-pyridin-2-yl)-cyclohexylmethyl]-2-methyl-2-[4-(4-nitro-phenyl)-oxazol-2-ylamino]-3-phenyl-propionamide;
(S)-2-[4-(4-cyano-phenyl)-oxazol-2-ylamino]-3-(1H-indol-3-yl)-N-[1-(5-methoxy-pyridin-2-yl)-cyclohexylmethyl]-2-methyl-propionamide;
(S)-3-(1H-indol-3-yl)-N-[1-(5-methoxy-pyridin-2-yl)-cyclohexylmethyl]-2-methyl-2-(4-phenyl-oxazol-2-ylamino)-propionamide;
(S)-2-(4-ethyl-oxazol-2-ylamino)-3-(1H-indol-3-yl)-N-[1-(5-methoxy-pyridin-2-yl)-cyclohexylmethyl]-2-methyl-propionamide;
(S)-3-(1H-indol-3-yl)-N-[1-(5-methoxy-pyridin-2-yl)-cyclohexylmethyl]-2-methyl-2-[4-(4-nitro-phenyl)-thiazol-2-ylamino]-propionamide;
(S)-2-(benzooxazol-2-ylamino)-3-(1H-indol-3-yl)-2-methyl-N-(1-pyridin-2-yl-cyclohexylmethyl)-propionamide;
(S)-3-(1H-indol-3-yl)-2-methyl-2-(pyridin-4-ylamino)-N-(1-pyridin-2-yl-cyclohexylmethyl)-propionamide;
(S)-3-(1H-indol-3-yl)-2-(isoquinol-4-ylamino)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(pyrimidin-5-ylamino)-propionamide;
(S)-2-(biphenyl-2-ylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-m-tolylamino-propionamide;
(S)-3-(1H-indol-3-yl)-2-methyl-2-(6-phenyl-pyridin-2-ylamino)-N-(1-pyridin-2-yl-cyclohexylmethyl)-propionamide;
(R)-3-phenyl-2-phenylamino-N-(1-pyridin-2-yl-cyclohexylmethyl)-propionamide;
(S)-3-(1H-indol-3-yl)-2-methyl-2-phenylethylamino-N-(1-pyridin-2-yl-cyclohexylmethyl)-propionamide;
(S)-2-[(benzofuran-2-ylmethyl)-amino]-3-(1H-indol-3-yl)-2-methyl-N-(1-pyridin-2-yl-cyclohexylmethyl)-propionamide, and
(S)-3-(1H-indol-3-yl)-2-methyl-2-(4-nitro-benzylamino)-N-(1-pyridin-2-yl-cyclohexylmethyl)-propionamide.

51 The combination of claim 39, wherein the bombesin receptor antagonist is a compound of formula (III) or a pharmaceutically acceptable salt thereof: 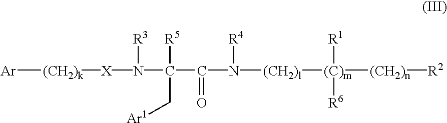
wherein:
k is 0, 1 or 2;
l is 0, 1, 2 or 3;
m is 0 or 1;
n is 0, 1 or 2;
X is —CO—, —OCO, —SO— and —SO₂—;
Ar is benzimidazolyl, benzofuryl, benzothiadiazolyl, benzothiazolyl, benzothienyl, benzopyrazinyl, benzotriazolyl, benzoxadiazolyl, furyl, imidazolyl, indanyl, indolyl, isoquinolyl, isoxazolyl, naphthyl, oxazolyl, phenyl, pyrazinyl, pyrazolyl, pyridyl, pyridazinyl, pyrimidyl, pyrrolyl, quinolinyl, tetralinyl, tetrazolyl, thiazolyl, thienyl or triazolyl each unsubstituted or substituted with from 1 to 3 substituents selected from amino, acetyl, alkyl (straight chain or branched with from 1 to 6 carbon atoms), alkoxy, cyano, halogen, hydroxy, nitro, phenyl, pyridyl, pyrrolyl, isoxazolyl, phenoxy, tolyloxy, —CF₃, —OCF₃, —SO₂CF₃, —NHCONH₂, —CO₂H, —CH₂CO₂H, —CH₂CN, SO₂Me, SO₂NH₂, SO₂Ph, —(CH₂)_{q}NR⁷R⁸, —CONR⁹R¹⁰, and CO₂R¹¹, wherein q is 0, 1 or 2 and R⁷, R⁸, R⁹, R¹⁰, R¹¹ are each independently selected from hydrogen or straight or branched alkyl of up to 6 carbon atoms or cyclic alkyl of between 5 to 7 atoms which may contain 1 or 2 oxygen or nitrogen atoms or R⁷ and R⁸ or R⁹ and R¹⁰ together with the nitrogen atom to which they are linked can form a 5- to 7-membered aliphatic ring which may contain 1 or 2 oxygen or nitrogen atoms;
Ar¹ is independently selected from Ar and can also be pyridyl-N-oxide;
R¹ is hydrogen or straight or branched alkyl of up to 6 carbon atoms or cyclic alkyl of between 5 and 7 atoms which may contain 1 or 2 oxygen or nitrogen atoms;
R² is independently selected from Ar or is hydrogen, hydroxy, alkoxy, —NMe₂, —CONR¹²¹³², 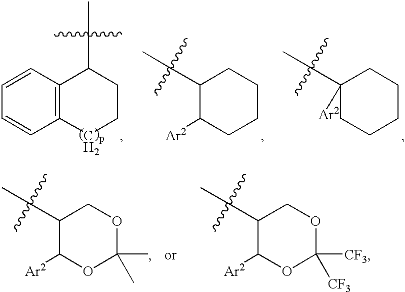
wherein p is 0, 1 or 2, Ar² is phenyl or pyridyl; and, R¹² and R¹³ are each independently selected from hydrogen, straight or branched alkyl of up to 6 carbon atoms or cyclic alkyl of between 5 and 7 carbon atoms;
R³, R⁴ and R⁵ are each independently selected from hydrogen and lower alkyl; and
R⁶ is hydrogen, methyl or forms with R¹ a ring of from 3 to 7 carbon atoms which can contain an oxygen or nitrogen atom, or R¹ and R6 can together be carbonyl.

52. The combination of claim 51, wherein the bombesin receptor antagonist is a compound formula (III) in which:
k is 0 or 1;
l is 1;
m is 0 or 1;
n is 0 or 1;
X is —C(O)—, —OC(O)—, or —SO₂—;
Ar is benzofuryl, furyl, indolyl, isoquinolyl, naphthyl, phenyl, pyridyl, quinolyl or thienyl each unsubstituted or substituted with 1 or 2 substituents selected from alkoxy, cyano, halogen, nitro, phenyl, phenoxy, —CF₃, —(CH₂)_{q}NR⁷R⁸, wherein R⁷ and R⁸ can form a ring of between 5 to 7 atoms which may contain 1 or 2 oxygen or nitrogen atoms, or R⁷ and R⁸ can be independently selected from hydrogen, straight or branched alkyl of up to 4 carbon atoms or cyclic alkyl of 5 carbon atoms;
Ar¹ is independently selected from Ar, preferably indolyl, and can also be pyridyl-N-oxide;
R¹ and R⁶ can form a cyclic alkyl of from 5 to 7 carbon atoms or R¹ and R⁶ together are carbonyl;
R² is independently selected from unsubstituted or substituted pyridyl or is hydrogen, hydroxy, alkoxy, —NMe₂, —CONR¹²R¹³ wherein R¹² and R¹³ are each independently selected from H and CH₃;
R³, R⁴ and R⁵ are each independently selected from hydrogen and methyl.

53. The combination of claim 51, wherein the bombesin receptor antagonist is a compound of Formula (III) in which,
l is 1;
m is 1;
n is 0;
R² is 2-pyridyl;
R⁶ forms a cyclohexyl with R¹.

54. The combination of claim 39, wherein the bombesin receptor antagonist is a compound of formula (IIIa) or a salt thereof: 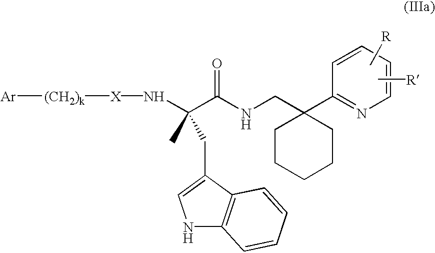
wherein Ar, k and X have the meanings given above in first, and the pyridine ring is optionally substituted by with 1 or 2 substituents, R and R′, independently selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷ and R⁸ can be independently selected from hydrogen or cyclic alkyl of between 5 to 7 carbon atoms, and their pharmaceutically acceptable salts thereof.

55. The combination of claim 39, wherein the bombesin receptor antagonist is one of the following compounds or a salt thereof:
N-{(S)-2-(1H-indol-3-yl)-1-methyl-I -[(1-pyridin-2-yl-cyclohexylmethyl)-carbamoyl]-ethyl}-4-nitro-benzamide;
C-dimethylamino-N-{(S)-2-(1H-indol-3-yl)-1-methyl-1-[(1-pyridin-2-yl-cyclohexylmethyl)-carbamoyl]-ethyl}-benzamide;
1H-indole-2-carboxylic acid {(S)-2-(1H-indol-3-yl)-1-methyl-1-[(1-pyridin-2-yl-cyclohexylmethyl)-carbamoyl]-ethyl}-amide;
benzo[b]thiophene-2-carboxylic acid {(S)-2-(1H-indol-3-yl)-1-methyl-1-[(1-pyridin-2-yl-cyclohexylmethyl)-carbamoyl]-ethyl}-amide;
N-{(S)-2-(1H-Indol-3-yl)-1-methyl-1-[(1-pyridin-2-yl-cyclohexylmethyl)-carbarnoyl]-ethyl}-2-pyrrol-1-yl-benzamide
1H-indole-5-carboxylic acid {(S)-2-(1H-indol-3-yl)-1-methyl-1-[(1-pyridin-2-yl-cyclohexylmethyl)-carbamoyl]-ethyl}-amide; and
1H-indole-2-carboxylic acid ((S)-2-(1H-indol-3-yl)-1-{[1-(5-methoxy-pyridin-2-yl)-cyclohexylmethyl]-carbamoyl}-1-methyl-ethyl)-amide.

56. The combination of claim 39, wherein the bombesin receptor antagonist is one of the following compounds or a salt thereof
N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-4-methyl-benzamide;
4-chloro-N-{(S)-2-(iH-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-4-methoxy-benzamide;
N-{(S)-2-(1H-indol-3-yl)-1-methyl-1-[(1-pyridin-2-yl-cyclohexylmethyl)-carbamoyl]-ethyl}-4-methanesulfonyl-benzamide;
3-cyano-N-{(S)-2-(1H-indol-3-yl)-1-methyl-1-[(1-pyridin-2-yl-cyclohexylmethyl)-carbamoyl]-ethyl}-benzamide;
3-chloro-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-3-methoxy-benzamide;
N-{(S)-2-(1H-indol-3-yl)-1-methyl-1-[(1-pyridin-2-yl-cyclohexylmethyl)-carbamoyl]-ethyl}-3-methanesulfonyl-benzamide;
dimethylamino-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-3-methyl-benzamide;
2-chloro-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-2-nitro-benzamide;
N-{(S)-2-(1H-indol-3-yl)-1-methyl-1-[(1-pyridin-2-yl-cyclohexylmethyl)-carbamoyl]-ethyl}-2-methoxy-benzamide;
N-{(S)-2-(1H-indol-3-yl)-1-methyl-1-[(1-pyridin-2-yl-cyclohexylmethyl)-carbamoyl]-ethyl}-2-methyl-benzamide;
2-fluoro-N-{(S)-2-(1H-indol-3-yl)-1-methyl-1-[(1-pyridin-2-yl-cyclohexylmethyl)-carbamoyl]-ethyl}-benzamide;
(S)-3-(1H-indol-3-yl)-2-methyl-N-(1-pyridin-2-yl-cyclohexylmethyl)-2-(2-p-tolyl-ethanoylamino)-propionamide;
(S)-3-(1H-indol-3-yl)-2-methyl-N-(1-pyridin-2-yl-cyclohexylmethyl)-2-(2-o-tolyl-ethanoylamino)-propionamide;
(S)-2-[2-(4-hydroxy-phenyl)-ethanoylamino]-3-(1H-indol-3-yl)-2-methyl-N-(1-pyridin-2-yl-cyclohexylmethyl)-propionamide;
(S)-2-[2-(3-hydroxy-phenyl)-ethanoylamino]-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2-m-tolyl-ethanoylamino)-propionamide;
(S)-2-[2-(2-fluoro-phenyl)-ethanoylamino]-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2-thiophen-3-yl-ethanoylamino)-propionamide;
pyridine-2-carboxylic acid {(S)-2-(1H-indol-3-yl)-1-methyl-1-[(1-pyridin-2-yl-cyclohexylmethyl)-carbamoyl]-ethyl}-amide
N-{(S)-2-(1H-indol-3-yl)-1-methyl-1-[(1-pyridin-2-yl-cyclohexylmethyl)-carbamoyl]-ethyl}-isonicotinamide;
furan-3-carboxylic acid {(S)-2-(1H-indol-3-yl)-1-methyl-1-[(1-pyridin-2-yl-cyclohexylmethyl)-carbamoyl]-ethyl}-amide;
furan-2-carboxylic acid {(S)-2-(1H-indol-3-yl)-1-methyl-1-[(1-pyridin-2-yl- cyclohexylmethyl)-carbamoyl]-ethyl}-amide;
5-methyl-isoxazole-3-carboxylic acid {(S)-2-(1H-indol-3-yl)-1-methyl-1-[(1-pyridin-2-yl-cyclohexylmethyl)-carbamoyl]-ethyl}-amide;
1-methyl-1H-pyrrole-2-carboxylic acid {(S)-2-(1H-indol-3-yl)-1-methyl-1-[(1-pyridin-2-yl-cyclohexylmethyl)-carbamoyl]-ethyl}-amide;
thiophene-2-carboxylic acid {(S)-2-(1H-indol-3-yl)-1-methyl-1-[(1-pyridin-2-yl-cyclohexylmethyl)-carbamoyl]-ethyl}-amide;
thiophene-3-carboxylic acid {(S)-2-(1H-indol-3-yl)-1-methyl-l -[(1-pyridin-2-yl-cyclohexylmethyl)-carbamoyl]-ethyl}-amide;
1H-indole-6-carboxylic acid {(S)-2-(1H-indol-3-yl)-1-methyl-1-[(1-pyridin-2-yl-cyclohexylmethyl)-carbamoyl]-ethyl}-amide;
1H-indole-5-carboxylic acid {(S)-2-(1H-indol-3-yl)-1-methyl-1-[(1-pyridin-2-yl-cyclohexylmethyl)-carbamoyl]-ethyl}-amide;
1H-indole-4-carboxylic acid {(S)-2-(1H-indol-3-yl)-1-methyl-1-[(1-pyridin-2-yl-cyclohexylmethyl)-carbamoyl]-ethyl}-amide;
1H-indole-7-carboxylic acid {(S)-2-(1H-indol-3-yl)-1-methyl-1-[(1-pyridin-2-yl-cyclohexylmethyl)-carbamoyl]-ethyl}-amide;
1-methyl-1H-indole-2-carboxylic acid {(S)-2-(1H-indol-3-yl)-1-methyl-1-[(1-pyridin-2-yl-cyclohexylmethyl)-carbamoyl]-ethyl}-amide;
benzothiazole-6-carboxylic acid {(S)-2-(1H-indol-3-yl)-1-methyl-1-[(1-pyridin-2-yl-cyclohexylmethyl)-carbamoyl]-ethyl}-amide;
1H-benzotriazole-5-carboxylic acid {(S)-2-(1H-indol-3-yl)-1-methyl-1-[(1-pyridin-2-yl-cyclohexylmethyl)-carbamoyl]-ethyl}-amide;
3-methyl-thiophene-2-carboxylic acid {(S)-2-(1H-indol-3-yl)-1-methyl-1-[(1-pyridin-2-yl-cyclohexylmethyl)-carbamoyl]-ethyl}-amide;
5-methyl-thiophene-2-carboxylic acid {(S)-2-(1H-indol-3-yl)-1-methyl-1-[(1-pyridin-2-yl-cyclohexylmethyl)-carbamoyl]-ethyl}-amide;
6-methyl-pyridine-2-carboxylic acid {(S)-2-(1H-indol-3-yl)-1-methyl-1-[(1-pyridin-2-yl-cyclohexylmethyl)-carbamoyl]-ethyl}-amide;
isoquinoline-3-carboxylic acid {(S)-2-(1H-indol-3-yl)-1-methyl-1-[(1-pyridin-2-yl-cyclohexylmethyl)-carbamoyl]-ethyl}-amide;
quinoxaline-2-carboxylic acid {(S)-2-(1H-indol-3-yl)-1-methyl-1-[(1-pyridin-2-yl-cyclohexylmethyl)-carbamoyl]-ethyl}-amide;
quinoline-8-carboxylic acid {(S)-2-(1H-indol-3-yl)-1-methyl-1-[(1-pyridin-2-yl-cyclohexylmethyl)-carbamoyl]-ethyl}-amide;
5-phenyl-oxazole-4-carboxylic acid {(S)-2-(1H-indol-3-yl)-1-methyl-1-[(1-pyridin-2-yl-cyclohexylmethyl)-carbamoyl]-ethyl}-amide;
(S)-3-(1H-indol-3-yl)-2-[2-(4-methoxy-phenyl)-ethanoylamino]-2-methyl-N-(1-pyridin-2-yl-cyclohexylmethyl)-propionamide;
(S)-2-[2-(4-dimethylamino-phenyl)-ethanoylamino]-3-(1H-indol-3-yl)-2-methyl-N-(1-pyridin-2-yl-cyclohexylmethyl)-propionamide;
(S)-3-(1H-indol-3-yl)-2-methyl-2-[2-(2-nitro-phenyl)-ethanoylamino]-N-(1-pyridin-2-yl-cyclohexylmethyl)-propionamide;
(S)-3-(1H-indol-3-yl)-2-[2-(2-methoxy-phenyl)-ethanoylamino]-2-methyl-N-(1-pyridin-2-yl-cyclohexylmethyl)-propionamide; and
N-{(S)-2-(1H-indol-3-yl)-1-methyl-1-[(1-pyridin-2-yl-cyclohexylmethyl)-carbamoyl]-ethyl}-2-pyrrol-1-yl-benzamide.

57. The combination of claim 39, wherein the bombesin receptor antagonist is one of the following compounds or a salt thereof
{(S)-2-(1H-indol-3-yl)-1-methyl-1-[(1-pyridin-2-yl-cyclohexylmethyl)-carbamoyl]-ethyl}-carbamic acid naphthalen-1-ylmethyl ester;
{(S)-2-(1H-indol-3-yl)-1-methyl-1-[(1-pyridin-2-yl-cyclohexylmethyl)-carbamoyl]-ethyl}-carbamic acid 3,4-dichloro-benzyl ester;
{(S)-2-(1H-indol-3-yl)-1-methyl-1-[(1-pyridin-2-yl-cyclohexylmethyl)-carbamoyl]-ethyl}-carbarnic acid 3-nitro-benzyl ester;
{(S)-2-(1H-indol-3-yl)-1-methyl-1-[(1-pyridin-2-yl-cyclohexylmethyl)-carbamoyl]-ethyl}-carbamic acid 3-trifluoromethyl-benzyl ester;
{(S)-2-(1H-indol-3-yl)-1-methyl-1-[(1-pyridin-2-yl-cyclohexylmethyl)-carbamoyl]-ethyl}-carbamic acid quinolin-6-ylmethyl ester;
{(S)-2-(1H-indol-3-yl)-1-methyl-1-[(1-pyridin-2-yl-cyclohexylmethyl)-carbamoyl]-ethyl}-carbamic acid 4-nitro-benzyl ester; and
{(S)-2-(1H-indol-3-yl)-1-methyl-1-[(1-pyridin-2-yl-cyclohexylmethyl)-carbamoyl]-ethyl}-carbamic acid 3-cyano-benzyl ester.

58. The combination of claim 39, wherein the bombesin receptor antagonist is one of the following compounds or a salt thereof:
{(S)-2-(1H-indol-3-yl)-1-methyl-I -[(1-pyridin-2-yl-cyclohexylmethyl)-carbamoyl]-ethyl}-carbamic acid 3,4-dimethoxy-benzyl ester;
{(S)-2-(1H-indol-3-yl)-1-methyl-1-[(1-pyridin-2-yl-cyclohexylmethyl)-carbamoyl]-ethyl}-carbamic acid naphthalen-2-ylmethyl ester;
{(S)-2-(1H-indol-3-yl)-1-methyl-1-[(1-pyridin-2-yl-cyclohexylmethyl)-carbamoyl]-ethyl}-carbamic acid indan-2-yl ester;
{(S)-2-(1H-indol-3-yl)-1-methyl-1-[(1-pyridin-2-yl-cyclohexylmethyl)-carbamoyl]-ethyl}-carbamic acid 4-methoxy-benzyl ester;
{(S)-2-(1H-indol-3-yl)-1-methyl-1-[(1-pyridin-2-yl-cyclohexylmethyl)-carbamoyl]-ethyl}-carbamic acid 4-chloro-benzyl ester;
{(S)-2-(1H-indol-3-yl)-1-methyl-1-[(1-pyridin-2-yl-cyclohexylmethyl)-carbamoyl]-ethyl}-carbamic acid 2-fluoro-benzyl ester;
{(S)-2-(1H-indol-3-yl)-1-methyl-1-[(1-pyridin-2-yl-cyclohexylmethyl)-carbamoyl]-ethyl}-carbamic acid 2-chloro-benzyl ester;
{(S)-2-(1H-indol-3-yl)-1-methyl-1-[(1-pyridin-2-yl-cyclohexylmethyl)-carbamoyl]-ethyl}-carbamic acid 2-methyl-benzyl ester;
{(S)-2-(1H-indol-3-yl)-1-methyl-I -[(1-pyridin-2-yl-cyclohexylmethyl)-carbamoyl]-ethyl}-carbamic acid 4-tert-butyl-benzyl ester;
{(S)-2-(1H-indol-3-yl)-1-methyl-1-[(1-pyridin-2-yl-cyclohexylmethyl)-carbamoyl]-ethyl}-carbamic acid 2-methoxy-benzyl ester;
{(S)-2-(1H-indol-3-yl)-1-methyl-1-[(1-pyridin-2-yl-cyclohexylmethyl)-carbamoyl]-ethyl}-carbamic acid 4-trifluoromethyl-benzyl ester;
{(S)-2-(1H-indol-3-yl)-1-methyl-1-[(1-pyridin-2-yl-cyclohexylmethyl)-carbamoyl]-ethyl}-carbamic acid 3-ethoxy-benzyl ester;
{(S)-2-(1H-indol-3-yl)-1-methyl-1-[(1-pyridin-2-yl-cyclohexylmethyl)-carbamoyl]-ethyl}-carbamic acid 2,4-dichloro-benzyl ester;
{(S)-2-(1H-indol-3-yl)-1-methyl-1-[(1-pyridin-2-yl-cyclohexylmethyl)-carbamoyl]-ethyl}-carbamic acid 3-methyl-benzyl ester;
{(S)-2-(1H-indol-3-yl)-1-methyl-1-[(1-pyridin-2-yl-cyclohexylmethyl)-carbamoyl]-ethyl}-carbamic acid 3-phenoxy-benzyl ester; and
{(S)-2-(1H-indol-3-yl)-1-methyl-1-[(1-pyridin-2-yl-cyclohexylmethyl)-carbamoyl]-ethyl}-carbamic acid 4-methyl-benzyl ester.

59 The combination of claim 39, wherein the bombesin receptor antagonist is one of the following compounds or a salt thereof:
(S)-3-(1H-indol-3-yl)-2-methyl-2-phenylmethanesulfonylamino-N-(1-pyridin-2-yl-cyclohexylmethyl)-propionamide;
(S)-2-(2-chloro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(naphthalene-1-sulfonylamino)-N-(1-pyridin-2-yl-cyclohexylmethyl)-propionamide;
(S)-3-(1H-indol-3-yl)-2-methyl-N-(1-pyridin-2-yl-cyclohexylmethyl)-2-(quinoline-8-sulfonylamino)-propionamide;
(S)-3-(1H-indol-3-yl)-2-methyl-N-(1-pyridin-2-yl-cyclohexylmethyl)-2-(2-trifluoromethyl-benzenesulfonylamino)-propionamide;
(S)-2-(biphenyl-2-sulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(5-methyl-2-phenoxy-benzenesulfonylamino)-N-(1-pyridin-2-yl-cyclohexylmethyl)-propionamide; and
(S)-3-(1H-indol-3-yl)-2-methyl-N-(1-pyridin-2-yl-cyclohexylmethyl)-2-(2-p-tolyloxy-benzenesulfonylamino)-propionamide.

60. The combination of claim 39, wherein the bombesin receptor antagonist is one of the following compounds or a salt thereof:
(S)-3-(1H-indol-3-yl)-2-methyl-N-(1-pyridin-2-yl-cyclohexylmethyl)-2-(toluene-4-sulfonylamino)-propionamide;
(S)-3-(1H-indol-3-yl)-2-methanesulfonylamino-2-methyl-N-(1-pyridin-2-yl-cyclohexylmethyl)-propionamide;
(S)-2-(2-fluoro-benzenesulfonylamino)-3-(1H-indol-3-yl)-2-methyl-N-(1-pyridin-2-yl-cyclohexylmethyl)-propionamide;
(S)-2-(4-chloro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2,2,2-trifluoro-ethanesulfonylamino)-propionamide;
(S)-2-(5-dimethylamino-naphthalene-1-sulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(naphthalene-2-sulfonylamino)-N-(1-pyridin-2-yl-cyclohexylmethyl)-propionamide;
(S)-3-(1H-indol-3-yl)-2-methyl-N-(l -pyridin-2-yl-cyclohexylmethyl)-2-(thiophene-2-sulfonylamino)-propionamide;
(S)-3-(1H-indol-3-yl)-2-methyl-2-(3-nitro-benzenesulfonylamino)-N-(1-pyridin-2-yl-cyclohexylmethyl)-propionamide;
(S)-2-(4-fluoro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(4-nitro-benzenesulfonylamino)-N-(1-pyridin-2-yl-cyclohexylmethyl)-propionamide;
(S)-3-(1H-indol-3-yl)-2-methyl-N-(1-pyridin-2-yl-cyclohexylmethyl)-2-(3-trifluoromethyl-benzenesulfonylamino)-propionamide;
(S)-2-(3,4-dichloro-benzenesulfonylamino)-3-(1H-indol-3-yl)-2-methyl-N-(1-pyridin-2-yl-cyclohexylmethyl)-propionamide;
(S)-2-(3-fluoro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(4-trifluoromethyl-benzenesulfonylamino)-propionamide;
(S)-2-(5-chloro-thiophene-2-sulfonylamino)-3-(1H-indol-3-yl)-2-methyl-N-(1-pyridin-2-yl-cyclohexylmethyl)-propionamide;
(S)-2-(3-chloro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(toluene-3-sulfonylamino)-propionamide;
(S)-2-(3 ,4-dimethoxy-benzenesulfonylamino)-3-(1H-indol-3-yl)-2-methyl-N-(1-pyridin-2-yl-cyclohexylmethyl)-propionamide;
(S)-2-(4-cyano-benzenesulfonylamino)-3-(1H-indol-3-yl)-2-methyl-N-(1-pyridin-2-yl-cyclohexylmethyl)-propionamide;
(S)-2-(2-cyano-benzenesulfonylamino)-3-(1H-indol-3-yl)-2-methyl-N-(1-pyridin-2-yl-cyclohexylmethyl)-propionamide;
(S)-2-(5-chloro-1,3-dimethyl-1H-pyrazole-4-sulfonylamino)-3-(1H-indol-3-yl)-2-methyl-N-(1-pyridin-2-yl-cyclohexylmethyl)-propionamide;
(S)-2-(3,5-dimethyl-isoxazole-4-sulfonylamino)-3-(1H-indol-3-yl)-2-methyl-N-(1-pyridin-2-yl-cyclohexylmethyl)-propionamide;
(S)-2-(benzo [1,2,5]thiadiazole-4-sulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(1-methyl-1H-imidazole-4-sulfonylamino)-N-(1-pyridin-2-yl-cyclohexylmethyl)-propionamide;
(S)-2-(benzo[1,2,5]oxadiazole-4-sulfonylamino)-3-(1H-indol-3-yl)-2-methyl-N-(1-pyridin-2-yl-cyclohexylmethyl)-propionamide;
3-{(S)-2-(1H-indol-3-yl)-1-methyl-1-[(1-pyridin-2-yl-cyclohexylmethyl)-carbamoyl]-ethylsulfamoyl}-thiophene-2-carboxylic acid methyl ester;
(S)-3-(1H-indol-3-yl)-2-(5-isoxazol-3-yl-thiophene-2-sulfonylamino)-2-methyl-N-(l -pyridin-2-yl-cyclohexylmethyl)-propionamide;
(S)-3-(1H-indol-3-yl)-2-methyl-2-(2-nitro-phenylmethanesulfonylamino)-N-(1-pyridin-2-yl-cyclohexylmethyl)-propionamide;
(S)-2-(3-cyano-benzenesulfonylamino)-3-(1H-indol-3-yl)-2-methyl-N-(1-pyridin-2-yl-cyclohexylmethyl)-propionamide;
(S)-2-(1,2-dimethyl-1H-imidazole-4-sulfonylamino)-3-(1H-indol-3-yl)-2-methyl-N-(1-pyridin-2-yl-cyclohexylmethyl)-propionamide;
(S)-3-(1H-indol-3-yl)-2-(3-methoxy-benzenesulfonylamino)-2-methyl-N-(1-pyridin-2-yl-cyclohexylmethyl)-propionamide;
(S)-3-(1H-indol-3-yl)-2-methyl-2-(8-nitro-naphthalene-1-sulfonylamino)-N-(1-pyridin-2-yl-cyclohexylmethyl)-propionamide;
(S)-2-(2-chloro-5-nitro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2,4,6-trichloro-benzenesulfonylamino)-propionamide;
(S)-2-(4-chloro-2-nitro-benzenesulfonylamino)-3-(1H-indol-3-yl)-2-methyl-N-(1-pyridin-2-yl-cyclohexylmethyl)-propionamide;
(S)-2-(5-benzenesulfonyl-thiophene-2-sulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(4-trifluoromethoxy-benzenesulfonylamino)-propionamide;
2-{(S)-2-(1H-indol-3-yl)-1-methyl-1-[(1-pyridin-2-yl-cyclohexylmethyl)-carbamoyl]-ethylsulfamoyl}-benzoic acid methyl ester;
(S)-2-(3-chloro-4-fluoro-benzenesulfonylamino)-3-(1H-indol-3-yl)-2-methyl-N-(1-pyridin-2-yl-cyclohexylmethyl)-propionamide;
(S)-2-(2,5-dichloro-thiophene-3-sulfonylamino)-3-(1H-indol-3-yl)-2-methyl-N-(1-pyridin-2-yl-cyclohexylmethyl)-propionamide;
(S)-2-(3-chloro-4-methyl-benzenesulfonylamino)-3-(1H-indol-3-yl)-2-methyl-N-(1-pyridin-2-yl-cyclohexylmethyl)-propionamide;
(S)-3-(1H-indol-3-yl)-2-(2-methoxy-4-methyl-benzenesulfonylamino)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(5-pyridin-2-yl-thiophene-2-sulfonylamino)-propionamide;
(S)-2-(5-bromo-6-chloro-pyridine-3-sulfonylamino)-3-(1H-indol-3-yl)-2-methyl-N-(1-pyridin-2-yl-cyclohexylmethyl)-propionamide;
(S)-2-(2,4-dinitro-benzenesulfonylamino)-3-(1H-indol-3-yl)-2-methyl-N-(1-pyridin-2-yl-cyclohexylmethyl)-propionamide;
(S)-3-(1H-indol-3-yl)-2-(4-methanesulfonyl-benzenesulfonylamino)-2-methyl-N-(1-pyridin-2-yl-cyclohexylmethyl)-propionamide;
(S)-2-(4-tert-butyl-benzenesulfonylamino)-3-(1H-indol-3-yl)-2-methyl-N-(1-pyridin-2-yl-cyclohexylmethyl)-propionamide;
(S)-2-(2,4-dichloro-5-methyl-benzenesulfonylamino)-3-(1H-indol-3-yl)-2-methyl-N-(1-pyridin-2-yl-cyclohexylmethyl)-propionamide;
(S)-2-(2-chloro-5-trifluoromethyl-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(2-nitro-4-trifluoromethyl-benzenesulfonylamino)-N-(1-pyridin-2-yl-cyclohexylmethyl)-propionamide; and
(S)-2-(4-butyl-benzenesulfonylamino)-3-(1H-indol-3-yl)-2-methyl-N-(1-pyridin-2-yl-cyclohexylmethyl)-propionamide.

61. The combination of claim 39, which is in the form of a pharmaceutical composition further comprising a pharmaceutically acceptable excipient.

62. The combination of claim 39, adapted for oral administration.

63. Use of a combination as claimed in claim 39 in the preparation of a medicament for the treatment or prophylaxis of male sexual dysfunction (more particularly male erectile dysfunction) and/or female sexual dysfunction (more particularly hypoactive sexual desire disorders, sexual arousal disorders, anorgasmic disorders or sexual pain disorders.

64. A pharmaceutical combination (for simultaneous, separate or sequential administration) of a bombesin receptor antagonist and a PDE 5 inhibitor.

65. A pharmaceutical combination (for simultaneous, separate or sequential administration) of a bombesin receptor antagonist and a NEP inhibitor.

66. A pharmaceutical combination (for simultaneous, separate or sequential administration) of a bombesin receptor antagonist and one or more estrogen receptor modulators (SERM) and/or estrogen agonists and/or estrogen antagonists.

67. A pharmaceutical combination (for simultaneous, separate or sequential administration) of a bombesin receptor antagonist and lasofoxifene.
